(19) United States
(12) Patent Application Publication
(10) Pub. No.: US 2025/0011777 A1
(43) Pub. Date: Jan. 9, 2025

(54) DELIVERY OF ANITSENSE OLIGOMERS BY MIRROR IMAGE PEPTIDES

(71) Applicants: SAREPTA THERAPEUTICS, INC., Cambridge, MA (US); MASSACHUSETTS INSTITUTE OF TECHNOLOGY, Cambridge, MA (US)

(72) Inventors: Carly K. SCHISSEL, Cambridge, MA (US); Charlotte E. FARQUHAR, Cambridge, MA (US); Annika B. MALMBERG, Cambridge, MA (US); Andrei LOAS, Cambridge, MA (US); Bradley L. PENTELUTE, Cambridge, MA (US)

(21) Appl. No.: 18/688,144

(22) PCT Filed: Sep. 1, 2022
(86) PCT No.: PCT/US2022/042365

§ 371 Date: Feb. 29, 2024

Related U.S. Application Data
(60) Provisional application No. 63/240,472, filed on Sep. 3, 2021.
(60) Provisional application No. 63/314,855, filed on Feb. 28, 2022.

(51) Int. Cl. C12N 15/113; A61K 47/64; C07K 14/00

(52) U.S. Cl. CPC C12N 15/113; A61K 47/645; C07K 14/001; C12N 2310/11; C12N 2310/314; C12N 2310/3233

(57) ABSTRACT

Provided herein are oligonucleotides, cell-penetrating peptides, and peptide-oligonucleotide-conjugates. Also provided herein are methods of treating a muscle disease, a viral infection, or a bacterial infection in a subject in need thereof, comprising administering to the subject oligonucleotides, peptides, and peptide-oligonucleotide-conjugates described herein.

### RELATED APPLICATIONS

This application claims priority to U.S. Provisional Application No. 63/240,472 filed on Sep. 3, 2021 and U.S. Provisional Application No. 63/314,855 filed on Feb. 28, 2022. The contents of each application are hereby incorporated by reference in their entireties.

### BACKGROUND

Antisense technology provides a means for modulating the expression of one or more specific gene products, including alternative splice products, and is uniquely useful in a number of therapeutic, diagnostic, and research applications. The principle behind antisense technology is that an antisense compound, e.g., an oligonucleotide, which hybridizes to a target nucleic acid, modulates gene expression activities such as transcription, splicing, or translation through any one of a number of antisense mechanisms. The sequence specificity of antisense compounds makes them attractive as tools for target validation and gene functionalization, as well as therapeutics, to selectively modulate the expression of genes involved in disease.

Although significant progress has been made in the field of antisense technology, there remains a need in the art for oligonucleotides and peptide-oligonucleotide-conjugates with improved antisense or antigene performance.

### SUMMARY OF THE INVENTION

Provided herein are oligonucleotide conjugates comprising an antisense oligomer and a covalently linked peptide. The antisense oligomer can be a phosphorodiamidate morpholino oligomer, and the peptide can be any of the peptides provided herein. The oligonucleotide conjugates are useful for the treatment for various diseases in a subject in need thereof, including, but not limited to, neuromuscular diseases such as Duchenne muscular dystrophy.

In an embodiment, the oligonucleotide conjugate is an oligonucleotide conjugate of Formula I:

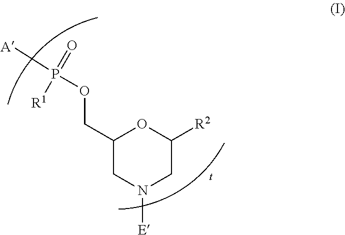

or a pharmaceutically acceptable salt thereof, wherein A′, E′, R¹, R², and t are as defined herein.

In an embodiment, the oligonucleotide conjugate is an oligonucleotide conjugate of Formula III, or a pharmaceutically acceptable salt thereof. In certain embodiments, the oligonucleotide conjugate of Formula I is an oligonucleotide conjugate selected from:

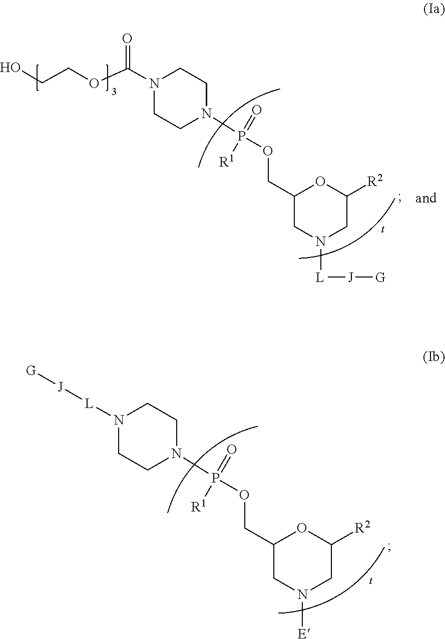

or pharmaceutically acceptable salts thereof, wherein E′, G, J, L, R¹, R², and t are as defined herein.

In certain embodiments, each R¹ is N(CH₃)₂. In certain embodiments, each R² is, independently at each occurrence, a nucleobase selected from adenine, guanine, cytosine, 5-methyl-cytosine, thymine, uracil, methylated guanine, and methylated adenine.

In an embodiment, J is selected from d-R₈, d-BPEP, d-DPV7, d-DPV6, d-penetratin, d-Bac7, d-MPG, d-Hel11-7, d-TAT, d-TATp, d-WR₈, d-WBPEP, d-WDPV7, d-WDPV6, d-WTAT, or d-WTATp. In a further embodiment, J is selected from d-DPV7, d-DPV6, d-penetratin, d-Bac7, d-MPG, d-Hel11-7, d-WR₈, d-WBPEP, d-WDPV7, d-WDPV6, d-WTAT, or d-WTATp. In still another embodiment, J is selected from WR₈, WTAT, WBPEP, WDPV7, WTATp, WDPV6, TATp, MPG, or Hel11-7, wherein all chiral amino acids of the peptide are in the L-configuration.

In an embodiment, J is a cell-penetrating peptide comprising 3 to 15 amino acids selected from unnatural amino acids or D-amino acids, further comprising a C-terminal sequence having a KWKK, KKWK, KWWKK, WWKK, WKK, KKKK, or KK motif. In another embodiment, J is a cell-penetrating peptide comprising 3 to 15 amino acids selected from unnatural amino acids or D-amino acids, further comprising a C-terminal sequence having a (d-K)(d-W)(d-K)(d-K), (d-K)(d-K)(d-W)(d-K), (d-K)(d-W)(d-W)(d-K)(d-K), (d-W)(d-W)(d-K)(d-K), (d-W)(d-K)(d-K), (d-K)(d-K)(d-K)(d-K), or (d-K)(d-K) motif.

In a further embodiment, the unnatural amino acids are selected from Abu (γ-aminobutyric acid), B (β-alanine), Hle (homoleucine), Nle (norleucine), Nap (naphthylalanine), Dpa (diphenylalanine), Dab (diaminobutyric acid), Pip (aminopiperidine-carboxylic acid), Amf (aminomethylphenylalanine), and Gba (2-amino-4-guanidinobutanoic acid).

In another embodiment, the cell penetrating peptide is selected from SEQ ID NOS: 33-669.

In an embodiment, L is —C(O)(CH₂)₁₋₈C(O)—C₇₋₁₈-heteroaromatic-((CH₂)₁₋₈C(O)—, —C(O)(CH₂)₁₋₈C(O)—C₇₋₁₈-heteroaromatic-(W)—; —C(O)(CH₂)₁₋₈C(O)—C₇₋₁₈-heteroaromatic-(W—W)—

In another aspect, provided herein is a pharmaceutical composition comprising an oligonucleotide conjugate provided herein and a pharmaceutically acceptable carrier.

Also provided herein is a method of treating a neuromuscular disease comprising administering to a subject in need thereof an oligonucleotide conjugate provided herein.

In some embodiments, the oligonucleotide conjugates as described herein can be used for treating muscular dystrophy in a patient suffering from Duchenne muscular dystrophy (DMD).

### BRIEF DESCRIPTION OF THE FIGURES

FIG. 1A) Shows the general construction of conjugates studied. Macromolecular cargo PMO IVS-654 is attached to the N-terminus of the peptides, along with a biotin handle for subsequent affinity capture. A trypsin-cleavable linker connects the cargo to the peptides. B) Shows activity data from the EGFP 654 assay conducted with D- and L-forms of several sequences at 5 μM. Each bar represents group mean±SD, N=3 distinct samples from a single biological replicate.

FIG. 2 Shows a schematic of how PMO-CPPs may enter the cell to perform exon-skipping activity.

FIG. 3A) Shows the construction and sequences of PMO-peptides used in subsequent experiments. B) Shows dose-response curves of D- and L-forms of several sequences at varying concentrations. Each point represents group mean±SD, N=3 distinct samples from a representative biological replicate. C) Mass spectra of PMO-D- and L-conjugates following incubation with human serum.

FIG. 4A) Shows the workflow of the uptake assay; cells are treated with PMO-D-CPPs, washed, and lysed to extract the whole cell lysate or the cytosol. B) Shows MALDI-TOF mass spectra displaying ions of intact biotinylated D-polyarginine peptides, isolated after internalization into HeLa cells. Spectra show ions corresponding to intact conjugates in the equimolar spike-in (black) and the whole cell lysate after treatment (red). C) Shows relative intensities in the equimolar standard, the response factor (F) was determined and used to calculate the fold change in concentration in the experimental samples, shown as bar graph, normalized to BKr₄. Also shown is the equation used to determine relative concentration: I (intensity), [X] (concentration), F (response factor), X (sample), S (standard).

FIG. 5A) Shows PMO activities from an EGFP 654 assay in which cells are treated in a pulse-chase format with chemical endocytosis inhibitors followed by PMO-conjugates at 5 μM. Chlorpromazine (CPZ) produces a dose-dependent inhibition of PMO activity. Bars represent group mean±SD, N=3 distinct samples from a single biological replicate. B) Shows a Western blot demonstrating extraction of whole cell lysate and cytosolic fraction with RIPA and digitonin buffer, respectively. Erk 1/2 is a cytosolic marker, whereas Rab5 is a late endosomal marker. Biotin-labeled conjugates are imaged using streptavidin-HRP, although the amounts of material are often too low to detect via Western blot. C) and D) Show example MALDI spectra following uptake analysis of lysates from B) containing PMO-D-BPEP and PMO-D-R8, respectively. Intact construct is detected in the whole cell (top) as well as cytosolic (bottom) fractions.

FIG. 6A) Shows concentrations of biotin-CPPs relative to BPEP in the whole cell and cytosolic extracts of C2C12 mouse myoblast cells FIG. 7A) Shows concentrations of PMO-biotin-CPPs relative to BPEP in the whole cell and cytosolic extracts of Hela cells. Relative concentration is normalized to BPEP. B) Shows relative efficiency (PMO activity/relative cytosolic concentration) of PMO-CPPs. Bars show group mean±SD, N=2 distinct samples from a single biological replicate, except for the whole cell condition of (A) in which N=2 distinct samples from two independent biological replicates

FIG. 8A) Shows the design of the library. B) Shows structures of the unnatural monomers used in the library. All natural-backbone monomers were in D-form. C) Shows a heat map of the quality control showing the relative abundance of the various amino acids of the sequence up to the isoseramox linker. Positions 7-10 show the KWKK motif, with positions 1-6 showing the varied composition of the variable region.

FIG. 9A) Shows HeLa 654 cells treated with 5 or 20 μM of PPMO-Library containing ˜1000, ˜2000, or ˜4000 members for 22 h prior to flow cytometry. Results are given as the mean EGFP fluorescence of cells treated with PMO-peptide relative to the fluorescence of cells alone. B) Shows HeLa 654 cells treated with 5 or 20 μM PPMO-Library for 22 h, then tested for LDH released into the cell media. Results are given as LDH release above vehicle relative to fully lysed cells.

FIG. 10A) Shows HeLa 654 cells treated with 20 μM PPMO-Library of varying member sizes or 20 μM PMO alone for 22 h prior to flow cytometry. Results are given relative to the fluorescence of PMO-treated cells. B) Shows HeLa 654 cells pre-incubated at 4° C. or 37° C. for 30 min prior to treatment with 20 μM PPMO-Library or 20 μM PMO alone for 2 h at the indicated temperature. Results are given relative to the fluorescence of PMO-treated cells.

FIG. 11 Shows the workflow of in-cell penetration selection-mass spectrometry.

FIG. 12 Shows the verification of the extraction of the cytosol via Western blot.

FIG. 13A) Shows HeLa 654 cells treated with 1, 2.5, 5, 10, 25, or 50 μM PMO-CPP for 22 h prior to flow cytometry. Results are given as the mean EGFP fluorescence of cells treated with PMO-peptide relative to the fluorescence of cells treated with vehicle only. B) Shows the results of cell supernatant from A) tested for LDH release. Results are given as percent LDH release above vehicle relative to fully lysed cells. C) Shows HeLa 654 cells treated with 0.5, 1, 2.5, 5, 10, 25, or 50 μM PMO-CPP for 22 h.

FIG. 14A) Shows HeLa 654 cells treated with 20 μM PPMO-Library or 19.9 μM PPMO-Library and 0.1 μM PMO-CPP for 22 h prior to flow cytometry. Results are given as the mean EGFP fluorescence of cells treated with PMO-peptide relative to the fluorescence of cells treated with vehicle only. B) Shows HeLa 654 cells treated with 1 or 5 μM PMO-CPP or a combined solution of 5 PMO-CPPs for 22 h prior to flow cytometry. Results are given as the mean EGFP fluorescence of cells treated with PMO-peptide relative to the fluorescence of cells treated with vehicle only. Indicated concentration represents the total PMO-CPP present in the sample.

FIG. 15A) Shows a plot of EGFP mean fluorescence intensity relative to PMO for cells treated with different endocytosis inhibitors. B) Shows the plot of EGFP mean fluorescence intensity relative to PMO for cells incubated with PMO-CPPs at 4° C. or 37° C.

FIG. 16 Shows A) Sequences of PMO-SulfoCy5-CPP constructs, with the N-terminal cargo fully drawn out (Z). Lowercase letters denote D-amino acids. B-D) Shows HeLa 654 cells treated with 1, 2.5, 5, 10, 25, or 50 μM PMO-CPP or PMO-SulfoCy5-CPP for 22 h prior to flow-cytometry. Results are given as the mean EGFP fluorescence of cells treated with PMO-peptide relative to the fluorescence of cells treated with vehicle only. B) Shows treatment with D-Bpep constructs. C) Shows treatment with Pep1a constructs. D) Treatment with Pep1c constructs.

FIG. 17A-C) Shows HeLa 654 cells treated with 1, 2.5, 5, 10, 25, or 50 μM PMO-SulfoCy5-CPP for 22 h prior to flow cytometry. Results are given as the mean fluorescence of cells treated with PMO-SulfoCy5-peptide relative to the fluorescence of cells treated with vehicle only for each channel. A) Shows treatment with D-Bpep constructs. B) Shows treatment with Pep1a constructs. C) Shows treatment with Pep1c constructs.

### DETAILED DESCRIPTION

Cell-penetrating peptides (CPPs) can help treat disease by enhancing the delivery of cell-impermeable cargo. CPPs are a class of peptides 5-30 amino acid residues in length that are capable of directly entering the cell cytosol (Wolfe Justin M.; Fadzen Colin M.; Holden Rebecca L.; Yao Monica; Hanson Gunnar J.; Pentelute Bradley L. Angew. Chem. Int. Ed. 2018, 57, 4756-4759.; Oehlke, J.; Scheller, A.; Wiesner, B.; Krause, E.; Beyermann, M.; Klauschenz, E.; Melzig, M.; Bienert, M. Biochim. Biophys. Acta BBA—Biomembr. 1998, 1414 (1), 127-139.; Margus, H.; Padari, K.; Pooga, M. Mol. Ther. J. Am. Soc. Gene Ther. 2012, 20 (3), 525-533). These sequences can deliver covalently bound cargo, offering therapeutic potential to macromolecules otherwise restricted to extracellular targets.

Although CPPs have been widely studied since their discovery, the field lacks robust methodology to quantify cell entry and penetration efficacy. This dearth of knowledge is due to the complicated mechanisms of CPP cell entry and the many variables that affect CPP efficacy in any given assay—such as peptide concentration, cell type, temperature, treatment time, and cargo (Reissmann Siegmund. J. Pept. Sci. 2014, 20 (10), 760-784) For the well-studied CPP penetratin (RQIKIWFQNRRMKWKK), the reported ratio between intracellular and extracellular concentration ranges from 0.6:1.0 to 95.0:1.0 (Fischer, R.; Waizenegger, T.; Köhler, K.; Brock, R. A Biochim. Biophys. Acta BBA—Biomembr. 2002, 1564 (2), 365-374; Lindgren, M. E.; Hällbrink, M. M.; Elmquist, A. M.; Langel, U. Biochem. J. 2004, 377 (Pt 1), 69-76.) In addition, it is challenging to determine subcellular localization once a peptide is internalized, despite advances in fluorescence, immunoblot, and mass spectrometry detection (Illien, F.; Rodriguez, N.; Amoura, M.; Joliot, A.; Pallerla, M.; Cribier, S.; Burlina, F.; Sagan, S. Sci. Rep. 2016, 6, 36938.). The choice of CPP cargo adds an additional confounding factor. The cell-penetrating ability of more than ten common CPPs differs when bound to a cyanine dye versus a macromolecular drug, with no discernable trend having been previously described (Wolfe, J. M.; Fadzen, C. M.; Choo, Z.-N.; Holden, R. L.; Yao, M.; Hanson, G. J.; Pentelute, B. L. ACS Cent. Sci. 2018, 4 (4), 512-520). As a result, effective development of CPPs require a new methodology for understanding CPP cell entry and subcellular localization that can be carried out on the CPP-cargo conjugate.

There are several limitations that have slowed the clinical advancement of CPPs. Historically, CPPs, also known as protein transduction domains (PTDs), were derived from transmembrane portions of viral and transcriptional proteins. For example, the polyarginine peptide TAT was derived from the HIV-transactivator of transcription protein and was found to penetrate the nucleus and target gene expression (Frankel, A. D.; Pabo, C. O. Cell 1988, 55 (6), 1189-1193; Green, M.; Loewenstein, P. M. Cell 1988, 55 (6), 1179-1188). From this and similar sequences, synthetic peptides could be designed, including some tailored for delivery of PMO cargo such as Bpep, which relies on arginine to trigger uptake and the unnatural residues β-alanine and 6-amino-hexanoic acid to trigger endosomal escape (Jearawiriyapaisarn, N. et al. Molecular Therapy 2008, 16 (9), 1624-1629). Beyond empirical design using derivatives of polyarginine sequences, the rational design of new sequences remains challenging. Methods involving some rational design include synthetic molecular evolution (Wimley, W. C. In Cell Penetrating Peptides: Methods and Protocols; Langel, Ü., Ed.; Methods in Molecular Biology; Springer US: New York, NY, 2022; pp 73-89; Kauffman, W. B. et al. Nature Communications 2018, 9 (1), 2568).

Other methods involving rational designs include in silico methods (Porosk, L. et al. Expert Opin Drug Discov 2021, 16 (5), 553-565; Lee, E. Y. et al. Bioorganic & medicinal chemistry 2018, 26 (10), 2708-2718.; Manavalan, B. et al. J. Proteome Res. 2018, 17 (8), 2715-2726); Pandey, P. et al. J. Proteome Res. 2018, 17 (9), 3214-3222).

Additional previously discovered methods leverage machine learning to design new sequences using a model trained with a combinatorial library tested for the desired activity: nuclear localization (Schissel, C. K. et al. Nat. Chem. 2021, 13 (10), 992-1000; López-Vidal, E. M. et al. JACS Au 2021; Wolfe, J. M. et al. ACS Cent Sci 2018, 4 (4), 512-520). Finally, another common strategy involves screening platforms employing libraries from phage or mRNA display. However, these methods have limited advancements for the discovery of peptides that deliver cargo to subcellular compartments.

An additional approach is a screening platform that identified several “phylomer” CPPs from bacterial and viral genomes that were then shown to deliver antisense cargo in vivo (Hoffmann, K. et al. Scientific Reports 2018, 8 (1)). Still, a persistent limitation with these approaches is the difficulty of incorporating D-chiral or unnatural amino acids, which would provide access to an augmented chemical space. Unnatural amino acids are more easily incorporated into synthetic one-bead one-compound (OBOC) libraries, although the discovery of CPPs by these methods often relies on synthetic vesicles (Carney, R. P. et al. ACS Comb. Sci. 2017, 19 (5), 299-307). Classic affinity selection involves screening peptide ligands from synthetic libraries (OBOC), phage or mRNA display against immobilized protein targets, and decoding hits (Quartararo, A. J. et al. Nat Commun 2020, 11 (1), 3183; Zuckermann, R. N. et al. Proc Natl Acad Sci USA 1992, 89 (10), 4505-4509).

These methods advanced to biologically relevant conditions in on-cell selection platforms for the discovery of new ligands with an affinity for the external surface of cells and tissues (Beck, S. et al. Biomaterials 2011, 32 (33), 8518-8528; Wu, C.-H. et al. Science Translational Medicine 2015, 7 (290); Wu, C.-H. et al. Journal of Biomedical Science 2016, 23 (1), 8). However, biological display techniques are restricted to the use of mostly natural amino acids, limiting the resulting library diversity and proteolytic stability (Ren, Y. et al. Mol Pharm 2018, 15 (2), 592-601; Wei, X. et al. Angew Chem Int Ed Engl 2015, 54 (10), 3023-3027), and even those mirror image techniques that allow D-peptide discovery still have difficulty incorporating non-canonical residues (Huang, L. et al. Mol Pharm 2017, 14 (5), 1742-1753; Eckert, D. M. et al. Cell 1999, 99 (1), 103-115).

Provided herein are methods for addressing the empirical design of novel, more efficient peptide sequences. By developing improved screening platforms using unnatural peptides for the discovery of enhanced CPPs, the method provided herein access greater chemical diversity and proteolytic stability, and by incorporating biologically relevant screening conditions into the protocol, such as in-cell selection and inclusion of the specific cargo to be delivered.

Also provided herein are methods of identifying a peptide capable of delivering a phosphorodiamidate morpholino oligomer (PMO) into a cell, the method to discover sequences that are present in the whole cell lysate, and the cytosol. This method allows for the discovery of peptides that are more active and more efficiently localized to the nucleus compared to peptides that are isolated from the whole cell extracts, which include endosomes. A therapeutic macromolecule that would benefit from enhanced delivery is phosphorodiamidate morpholino oligomer (PMO), which has recently reached the market as an antisense “exon skipping” therapy for Duchenne muscular dystrophy (DMD). The drug, Eteplirsen, is a 10 kDa synthetic antisense oligomer that must reach the nucleus and bind pre-mRNA for its therapeutic effect. However, because of Eteplirsen's poor cell permeability, high doses reaching 50 mg/kg are required, and the majority of the drug is cleared renally within 24 h of administration (Baker, D. E. Eteplirsen. Hosp. Pharm. 2017, 52 (4), 302-305.; Lim, K. R. Q.; Maruyama, R.; Yokota, T. Drug Des. Devel. Ther. 2017, 11, 533-545 (hereafter referred to as Lim et al.)). Several CPPs have been shown to increase PMO uptake, and recent clinical trial results have shown a 10-fold increase in exposure to PMO with attachment to CPP in DMD patients (Inc, S. T. Sarepta Therapeutics Announces Positive Clinical Results from MOMENTUM, a Phase 2 Clinical Trial of SRP-5051 in Patients with Duchenne Muscular Dystrophy Amenable to Skipping Exon 51. http://www.globenewswire.com/news-release/2020/12/07/2140613/0/en/Sarepta-Therapeutics-Announces-Positive-Clinical-Results-from-MOMENTUM-a-Phase-2-Clinical-Trial-of-SRP-5051-in-Patients-with-Duchenne-Muscular-Dystrophy-Amenable-to-Skipping-Exon-5.html (accessed 2020-12-07)). Although a wide variety of CPPs have been tested for PMO delivery, they have been limited to the native L-form and studied predominantly with an activity-based assay, forgoing quantitative information on the amount of material inside the cell (Kurrikoff, K.; Vunk, B.; Langel, Ü. Expert Opin. Biol. Ther. 2021, 21 (3), 361-370).

D-peptides have been explored as CPPs (Ma, Y.; Gong, C.; Ma, Y.; Fan, F.; Luo, M.; Yang, F.; Zhang, Y.-H. J. Controlled Release 2012, 162 (2), 286-294; Henriques, S. T.; Peacock, H.; Benfield, A. H.; Wang, C. K.; Craik, D. J. J. Am. Chem. Soc. 2019, 141 (51), 20460-20469). While some reports are contentious as to whether mutations to D amino acids are detrimental to CPP activity (Verdurmen, W. P. R.; Bovee-Geurts, P. H.; Wadhwani, P.; Ulrich, A. S.; Hallbrink, M.; van Kuppevelt, T. H.; Brock, R. Chem. Biol. 2011, 18 (8), 1000-1010 (hereafter referred to as “Verdurmen et al.”)), no study has been performed on delivery of PMO using mirror image CPPs. The proteolytic stability of mirror image peptides would allow for their characterization after uptake into cells, providing orthogonal information to activity-based assays. Currently, the main method used to characterize PMO-CPP internalization is an in vitro assay in which successful delivery of the active oligomer to the nucleus results in green fluorescence (Sazani, P.; Gemignani, F.; Kang, S.-H.; Maier, M. A.; Manoharan, M.; Persmark, M.; Bortner, D.; Kole, R. Nat. Biotechnol. 2002, 20 (12), 1228-1233). While this is an excellent assay to measure PMO-CPP activity, this assay does not give information on the quantity of material inside the cell. Especially for conjugates with a known endocytic mechanism, understanding endosomal escape is crucial. High concentrations of peptides trapped in the endosome would not be apparent by the activity assay alone, but this loss of active peptide could be of great therapeutic detriment (Erazo-Oliveras, A.; Muthukrishnan, N.; Baker, R.; Wang, T.-Y.; Pellois, J.-P. Pharmaceuticals 2012, 5 (11), 1177-1209). Therefore, an additional assay that reveals relative quantities of material in different parts of the cell would provide a valuable new metric of CPP delivery. Comparing activity and relative quantity would be a valuable metric for efficiency, where a high efficiency peptide is one with a high ratio of antisense activity to internal concentration.

Existing methods to quantify uptake into cells include fluorescence, immunoblot, and mass spectrometry, but it is still challenging to distinguish between endosomal and cytosolic localization. In addition, proteolytic instability of L-peptides complicates their characterization by mass spectrometry after internalization. Mass spectrometry is a direct quantification tool that would give information about the concentration of peptides recovered from biological mixtures. Past studies have illustrated how MALDI-TOF mass spectrometry is a practical tool for absolute and relative quantification of peptides and proteins. For example, using an internal standard of a similar molecular weight is sufficient for generation of a calibration curve (Duncan, M. W. Practical Quantitative Biomedical Applications of MALDI-TOF Mass Spectrometry). Quantitation of total uptake of L-CPPs was achieved using heavy atom-labeled internal standards (Burlina, F.; Sagan, S.; Bolbach, G.; Chassaing, G. Angew. Chem. Int. Ed. 2005, 44 (27), 4244-4247. (hereafter referred to as “Burlina et al. 2005”)).; Burlina, F.; Sagan, S.; Bolbach, G.; Chassaing, G. Nat. Protoc. 2006, 1 (1), 200. (hereafter referred to as “Burlina et al. 2006”)). While this assay provided information regarding whole cell uptake of CPPs and CPP-peptide conjugates, it is limited by the need for heavy-atom labeling and the rapid degradation of L-peptides (Aubry, S.; Aussedat, B.; Delaroche, D.; Jiao, C.-Y.; Bolbach, G.; Lavielle, S.; Chassaing, G.; Sagan, S.; Burlina, F. Biochim. Biophys. Acta BBA—Biomembr. 2010, 1798 (12), 2182-2189.). A method for circumnavigating the need for spike-in of heavy atom-labeled standards was developed for the relative quantification of phosphopeptides (Ho, H.-P.; Rathod, P.; Louis, M.; Tada, C. K.; Rahaman, S.; Mark, K. J.; Leng, J.; Dana, D.; Kumar, S.; Lichterfeld, M.; Chang, E. J. Rapid Commun. Mass Spectrom. RCM 2014, 28 (24), 2681-2689).

The proteolytic stability of the D-peptides provided herein permit their recovery and analysis from inside cells and animals, allowing for the use of a new metric of antisense delivery efficiency.

Provided herein are peptide-oligonucleotide-conjugates comprising an oligonucleotide covalently bound to L-peptides or D-peptides. Also provided herein are methods of treating a disease in a subject in need thereof, comprising administering to the subject a peptide-oligonucleotide-conjugate described herein. The D-peptides, and thereby the peptide-oligonucleotide-conjugates, described herein display increased proteolytic stability compared to L-peptides. This increased proteolytic stability allows for mass spectrometry-based characterization following cytosolic delivery. Cytosolic delivery can be quantified based on the recovery of intact constructs from inside the cell.

Additionally provided herein are peptide-oligonucleotide-conjugates comprising an oligonucleotide covalently bound to cell-penetrating peptides comprising unnatural amino acids or D-amino acids, and further comprising a C-terminal sequence having a KWKK, KKWK, KWWKK, WWKK, WKK, KKKK, or KK motif.

Also provided herein are peptide-oligonucleotide-conjugates comprising an oligonucleotide covalently bound to cell-penetrating peptides comprising unnatural amino acids or D-amino acids, and further comprising a C-terminal sequence having a (d-K)(d-W)(d-K)(d-K), (d-K)(d-K)(d-W)(d-K), (d-K)(d-W)(d-W)(d-K)(d-K), (d-W)(d-W)(d-K)(d-K), (d-W)(d-K)(d-K), (d-K)(d-K)(d-K)(d-K), or (d-K)(d-K) motif.

Peptides are a promising strategy to improve the delivery of PMO to the nucleus. Cell-penetrating peptides (CPPs) in particular are relatively short sequences of 5-40 amino acids that ideally access the cytosol and can promote the intracellular delivery of cargo. For example, when conjugated to PMO, oligoarginine peptides have been some of the most effective peptides in promoting PMO delivery.

Also provided herein are methods for determining the uptake of biotinylated CPPs and PMO-CPPs to determine their relative concentrations in the whole cell and cytosol using extraction and direct detection via MALDI-TOF. By comparing PMO delivery activity to relative internal concentration, the methods provided herein derive a new metric for cargo delivery efficiency useful for improved the delivery of PMO cargoes for DMD.

Also provided herein is a method for treating neuromuscular diseases using the oligonucleotide conjugates as described herein.

### I. Definitions

Unless defined otherwise, all technical and scientific terms used herein have the same meaning as commonly understood by those of ordinary skill in the art to which the disclosure belongs. Although any methods and materials similar or equivalent to those described herein can be used in the practice or testing of the subject matter of the present disclosure, preferred methods and materials are described. For the purposes of the present disclosure, the following terms are defined below.

The term “about” will be understood by persons of ordinary skill in the art and will vary to some extent on the context in which it is used. As used herein when referring to a measurable value such as an amount, a temporal duration, and the like, the term “about” is meant to encompass variations of ±10%, including ±5%, 1%, and ±0.1% from the specified value, as such variations are appropriate to perform the disclosed methods.

The term “alkyl” refers to saturated, straight- or branched-chain hydrocarbon moieties containing, in certain embodiments, between one and six, or one and eight carbon atoms, respectively. Examples of C₁₋₆-alkyl moieties include, but are not limited to, methyl, ethyl, propyl, isopropyl, n-butyl, tert-butyl, neopentyl, n-hexyl moieties; and examples of C₁₋₈-alkyl moieties include, but are not limited to, methyl, ethyl, propyl, isopropyl, n-butyl, tert-butyl, neopentyl, n-hexyl, heptyl, and octyl moieties.

The number of carbon atoms in an alkyl substituent can be indicated by the prefix “C_{x-y},” where x is the minimum and y is the maximum number of carbon atoms in the substituent. Likewise, a Cₓ chain means an alkyl chain containing x carbon atoms.

The term “heteroalkyl” by itself or in combination with another term means, unless otherwise stated, a stable straight or branched chain alkyl group consisting of the stated number of carbon atoms and one or two heteroatoms selected from the group consisting of O, N, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include: —O—CH₂—CH₂—CH₃, —CH₂—CH₂—CH₂—OH, —CH₂—CH₂—NH—CH₃, —CH₂—S—CH₂—CH₃, and —CH₂—CH₂—S(═O)—CH₃. Up to two heteroatoms may be consecutive, such as, for example, —CH₂—NH—OCH₃, or —CH₂—CH₂—S—S—CH₃.

The term “aryl,” employed alone or in combination with other terms, means, unless otherwise stated, a carbocyclic aromatic system containing one or more rings (typically one, two, or three rings), wherein such rings may be attached together in a pendent manner, such as a biphenyl, or may be fused, such as naphthalene. Examples of aryl groups include phenyl, anthracyl, and naphthyl. In various embodiments, examples of an aryl group may include phenyl (e.g., C₆-aryl) and biphenyl (e.g., C₁₂-aryl). In some embodiments, aryl groups have from six to sixteen carbon atoms. In some embodiments, aryl groups have from six to twelve carbon atoms (e.g., C₆₋₁₂-aryl). In some embodiments, aryl groups have six carbon atoms (e.g., C₆-aryl).

As used herein, the term “heteroaryl” or “heteroaromatic” refers to a heterocycle having aromatic character. Heteroaryl substituents may be defined by the number of carbon atoms, e.g., C₁₋₉-heteroaryl indicates the number of carbon atoms contained in the heteroaryl group without including the number of heteroatoms. For example, a C_{1_9}-heteroaryl will include an additional one to four heteroatoms. A polycyclic heteroaryl may include one or more rings that are partially saturated. Non-limiting examples of heteroaryls include pyridyl, pyrazinyl, pyrimidinyl (including, e.g., 2- and 4-pyrimidinyl), pyridazinyl, thienyl, furyl, pyrrolyl (including, e.g., 2-pyrrolyl), imidazolyl, thiazolyl, oxazolyl, pyrazolyl (including, e.g., 3- and 5-pyrazolyl), isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Non-limiting examples of polycyclic heterocycles and heteroaryls include indolyl (including, e.g., 3-, 4-, 5-, 6- and 7-indolyl), indolinyl, quinolyl, tetrahydroquinolyl, isoquinolyl (including, e.g., 1- and 5-isoquinolyl), 1,2,3,4-tetrahydroisoquinolyl, cinnolinyl, quinoxalinyl (including, e.g., 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (including, e.g., 3-, 4-, 5-, 6- and 7-benzofuryl), 2,3-dihydrobenzofuryl, 8,9-dihydro-3H-dibenzo[b,f][1,2,3]triazolo[4,5-d]azocine. 1,2-benzisoxazolyl, benzothienyl (including, e.g., 3—, 4-, 5-, 6-, and 7-benzothienyl), benzoxazolyl, benzothiazolyl (including, e.g., 2-benzothiazolyl and 5-benzothiazolyl), purinyl, benzimidazolyl (including, e.g., 2-benzimidazolyl), benzotriazolyl, thioxanthinyl, carbazolyl, carbolinyl, acridinyl, pyrrolizidinyl, and quinolizidinyl.

As used herein, the acronym DBCO refers to 8,9-dihydro-3H-dibenzo[b,f][1,2,3]triazolo[4,5-d]azocine.

The term “protecting group” or “chemical protecting group” refers to chemical moieties that block some or all reactive moieties of a compound and prevent such moieties from participating in chemical reactions until the protective group is removed, for example, those moieties listed and described in T.W. Greene, P.G.M. Wuts, Protective Groups in Organic Synthesis, 3rd ed. John Wiley & Sons (1999). It may be advantageous, where different protecting groups are employed, that each (different) protective group be removable by a different means. Protective groups that are cleaved under totally disparate reaction conditions allow differential removal of such protecting groups. For example, protective groups can be removed by acid, base, and hydrogenolysis. Groups such as trityl, monomethoxytrityl, dimethoxytrityl, acetal and tert-butyldimethylsilyl are acid labile and may be used to protect carboxy and hydroxy reactive moieties in the presence of amino groups protected with Cbz groups, which are removable by hydrogenolysis, and Fmoc groups, which are base labile. Carboxylic acid moieties may be blocked with base labile groups such as, without limitation, methyl, or ethyl, and hydroxy reactive moieties may be blocked with base labile groups such as acetyl in the presence of amines blocked with acid labile groups such as tert-butyl carbamate or with carbamates that are both acid and base stable but hydrolytically removable.

Carboxylic acid and hydroxyl reactive moieties may also be blocked with hydrolytically removable protective groups such as the benzyl group, while amine groups may be blocked with base labile groups such as Fmoc. A particularly useful amine protecting group for the synthesis of compounds of Formula (I) is the trifluoroacetamide. Carboxylic acid reactive moieties may be blocked with oxidatively-removable protective groups such as 2,4-dimethoxybenzyl, while coexisting amino groups may be blocked with fluoride labile silyl carbamates.

Allyl blocking groups are useful in the presence of acid- and base-protecting groups since the former are stable and can be subsequently removed by metal or pi-acid catalysts. For example, an allyl-blocked carboxylic acid can be deprotected with a palladium(0)-catalyzed reaction in the presence of acid labile t-butyl carbamate or base-labile acetate amine protecting groups. Yet another form of protecting group is a resin to which a compound or intermediate may be attached. As long as the residue is attached to the resin, that functional group is blocked and cannot react. Once released from the resin, the functional group is available to react.

The term “nucleobase,” “base pairing moiety,” “nucleobase-pairing moiety,” or “base” refers to the heterocyclic ring portion of a nucleoside, nucleotide, and/or morpholino subunit.

Nucleobases may be naturally occurring (e.g., uracil, thymine, adenine, cytosine, and guanine), or may be modified or analogs of these naturally occurring nucleobases, e.g., one or more nitrogen atoms of the nucleobase may be independently at each occurrence replaced by carbon. Exemplary analogs include hypoxanthine (the base component of the nucleoside inosine); 2, 6-diaminopurine; 5-methyl cytosine; C5-propynyl-modified pyrimidines; 10-(9-(aminoethoxy)phenoxazinyl) (G-clamp) and the like.

Further examples of base pairing moieties include, but are not limited to, uracil, thymine, adenine, cytosine, guanine and hypoxanthine having their respective amino groups protected by acyl protecting groups, 2-fluorouracil, 2-fluorocytosine, 5-bromouracil, 5-iodouracil, 2,6-diaminopurine, azacytosine, pyrimidine analogs such as pseudoisocytosine and pseudouracil and other modified nucleobases such as 8-substituted purines, xanthine, or hypoxanthine (the latter two being the natural degradation products). The modified nucleobases disclosed in Chiu and Rana (2003) RNA 9:1034-1048, Limbach et al. (1994) Nucleic Acids Res. 22:2183-2196 and Revankar and Rao, Comprehensive Natural Products Chemistry, vol. 7, 313, are also contemplated, the contents of which are incorporated herein by reference.

Further examples of base pairing moieties include, but are not limited to, expanded-size nucleobases in which one or more benzene rings has been added. Nucleic base replacements described in the Glen Research catalog (www.glenresearch.com); Krueger A T et al. (2007) Acc. Chem. Res. 40:141-150; Kool ET (2002) Acc. Chem. Res. 35:936-943; Benner S A et al. (2005) Nat. Rev. Genet. 6:553-543; Romesberg F E et al. (2003) Curr. Opin. Chem. Biol. 7:723-733; Hirao, I (2006) Curr. Opin. Chem. Biol. 10:622-627, the contents of which are incorporated herein by reference, are contemplated as useful for the synthesis of the oligomers described herein. Examples of expanded-size nucleobases are shown below:

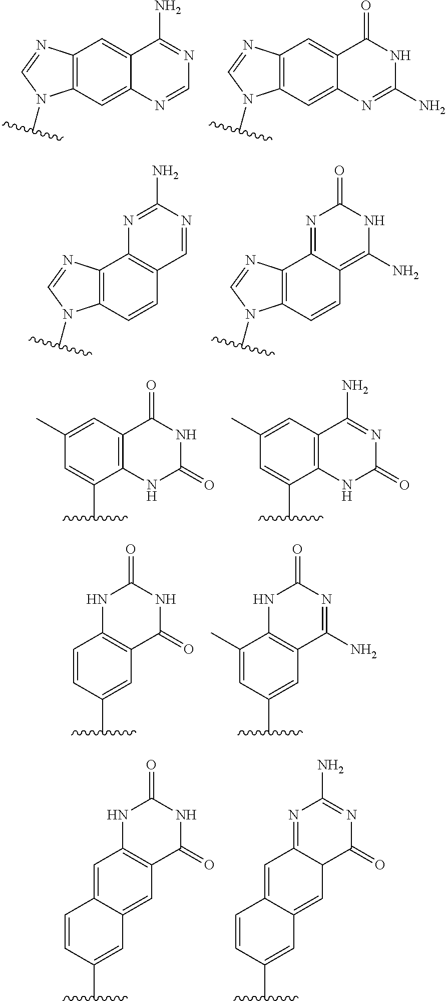

The terms “oligonucleotide” or “oligomer” refer to a compound comprising a plurality of linked nucleosides, nucleotides, or a combination of both nucleosides and nucleotides. In specific embodiments provided herein, an oligonucleotide is a morpholino oligonucleotide.

An antisense oligomer “specifically hybridizes” to a target polynucleotide if the oligomer hybridizes to the target under physiological conditions, with a Tm greater than 37° C., greater than 45° C., preferably at least 50° C., and typically 60° C.-80° C. or higher. The “Tm” of an oligomer is the temperature at which 50% hybridizes to a complementary polynucleotide. Tm is determined under standard conditions in physiological saline, as described, for example, in Miyada et al. (1987) Methods Enzymol. 154:94-107. Such hybridization may occur with “near” or “substantial” complementarity of the antisense oligomer to the target sequence, as well as with exact complementarity.

The terms “complementary” and “complementarity” refer to oligonucleotides (i.e., a sequence of nucleotides) related by base-pairing rules. For example, the sequence “T-G-A (5-3′)” is complementary to the sequence “T-C-A (5-3′).” Complementarity may be “partial,” in which only some of the nucleic acids' bases are matched according to base pairing rules. Or, there may be “complete,” “total,” or “perfect” (100%) complementarity between the nucleic acids. The degree of complementarity between nucleic acid strands has significant effects on the efficiency and strength of hybridization between nucleic acid strands. While perfect complementarity is often desired, some embodiments can include one or more but preferably 6, 5, 4, 3, 2, or 1 mismatches with respect to the target RNA. Such hybridization may occur with “near” or “substantial” complementarity of the antisense oligomer to the target sequence, as well as with exact complementarity. In some embodiments, an oligomer may hybridize to a target sequence at about 50%, 55%, 60%, 65%, 70%, 75%, 80%, 85%, 90%, 95%, 99% or 100% complementarity. Variations at any location within the oligomer are included. In certain embodiments, variations in sequence near the termini of an oligomer are generally preferable to variations in the interior, and if present are typically within about 6, 5, 4, 3, 2, or 1 nucleotides of the 5-terminus, 3′-terminus, or both termini.

Naturally occurring nucleotide bases include adenine, guanine, cytosine, thymine, and uracil, which have the symbols A, G, C, T, and U, respectively. Nucleotide bases can also encompass analogs of naturally occurring nucleotide bases. Base pairing typically occurs between purine A and pyrimidine T or U, and between purine G and pyrimidine C.

Oligonucleotides may also include nucleobase (often referred to in the art simply as “base”) modifications or substitutions. Oligonucleotides containing a modified or substituted base include oligonucleotides in which one or more purine or pyrimidine bases most commonly found in nucleic acids are replaced with less common or non-natural bases. In some embodiments, the nucleobase is covalently linked at the N9 atom of the purine base, or at the N1 atom of the pyrimidine base, to the morpholine ring of a nucleotide or nucleoside.

Purine bases comprise a pyrimidine ring fused to an imidazole ring, as described by the general formula:

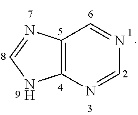

Adenine and guanine are the two purine nucleobases most commonly found in nucleic acids. These may be substituted with other naturally-occurring purines, including but not limited to N6-methyladenine, N2-methylguanine, hypoxanthine, and 7-methylguanine.

Pyrimidine bases comprise a six-membered pyrimidine ring as described by the general formula:

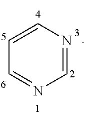

Cytosine, uracil, and thymine are the pyrimidine bases most commonly found in nucleic acids. These may be substituted with other naturally-occurring pyrimidines, including but not limited to 5-methylcytosine, 5-hydroxymethylcytosine, pseudouracil, and 4-thiouracil. In one embodiment, the oligonucleotides described herein contain thymine bases in place of uracil.

Other modified or substituted bases include, but are not limited to, 2,6-diaminopurine, orotic acid, agmatidine, lysidine, 2-thiopyrimidine (e.g. 2-thiouracil, 2-thiothymine), G-clamp and its derivatives, 5-substituted pyrimidine (e.g. 5-halouracil, 5-propynyluracil, 5-propynylcytosine, 5-aminomethyluracil, 5-hydroxymethyluracil, 5-aminomethylcytosine, 5-hydroxymethylcytosine, Super T), 7-deazaguanine, 7-deazaadenine, 7-aza-2,6-diaminopurine, 8-aza-7-deazaguanine, 8-aza-7-deazaadenine, 8-aza-7-deaza-2,6-diaminopurine, Super G, Super A, and N4-ethylcytosine, or derivatives thereof; N2-cyclopentylguanine (cPent-G), N2-cyclopentyl-2-aminopurine (cPent-AP), and N2-propyl-2-aminopurine (Pr-AP), pseudouracil or derivatives thereof; and degenerate or universal bases, like 2,6-difluorotoluene or absent bases like abasic sites (e.g. 1-deoxyribose, 1,2-dideoxyribose, 1-deoxy-2-O-methylribose; or pyrrolidine derivatives in which the ring oxygen has been replaced with nitrogen (azaribose)). Pseudouracil is a naturally occurring isomerized version of uracil, with a C-glycoside rather than the regular N-glycoside as in uridine.

Certain modified or substituted nucleobases are particularly useful for increasing the binding affinity of the antisense oligonucleotides of the disclosure. These include 5-substituted pyrimidines, 6-azapyrimidines and N-2, N-6 and 0-6 substituted purines, including 2-aminopropyladenine, 5-propynyluracil and 5-propynylcytosine. In various embodiments, nucleobases may include 5-methylcytosine substitutions, which have been shown to increase nucleic acid duplex stability by 0.6-1.2° C.

In some embodiments, modified or substituted nucleobases are useful for facilitating purification of antisense oligonucleotides. For example, in certain embodiments, antisense oligonucleotides may contain three or more (e.g., 3, 4, 5, 6 or more) consecutive guanine bases. In certain antisense oligonucleotides, a string of three or more consecutive guanine bases can result in aggregation of the oligonucleotides, complicating purification. In such antisense oligonucleotides, one or more of the consecutive guanines can be substituted with hypoxanthine. The substitution of hypoxanthine for one or more guanines in a string of three or more consecutive guanine bases can reduce aggregation of the antisense oligonucleotide, thereby facilitating purification.

The oligonucleotides provided herein are synthesized and do not include antisense compositions of biological origin. The molecules of the disclosure may also be mixed, encapsulated, conjugated or otherwise associated with other molecules, molecule structures or mixtures of compounds, as for example, liposomes, receptor targeted molecules, oral, rectal, topical or other formulations, for assisting in uptake, distribution, or absorption, or a combination thereof.

As used herein, a “nucleic acid analog” refers to a non-naturally occurring nucleic acid molecule. A nucleic acid is a polymer of nucleotide subunits linked together into a linear structure. Each nucleotide consists of a nitrogen-containing aromatic base attached to a pentose (five-carbon) sugar, which is in turn attached to a phosphate group. Successive phosphate groups are linked together through phosphodiester bonds to form the polymer.

The two common forms of naturally occurring nucleic acids are deoxyribonucleic acid (DNA) and ribonucleic acid (RNA). One end of the chain carries a free phosphate group attached to the 5-carbon atom of a sugar moiety; this is called the 5′ end of the molecule. The other end has a free hydroxyl (—OH) group at the 3′-carbon of a sugar moiety and is called the 3′ end of the molecule. A nucleic acid analog can include one or more non-naturally occurring nucleobases, sugars, and/or internucleotide linkages, for example, a phosphorodiamidate morpholino oligomer (PMO). As disclosed herein, in certain embodiments, a “nucleic acid analog” is a PMO, and in certain embodiments, a “nucleic acid analog” is a positively charged cationic PMO.

A “morpholino oligomer” or “PMO” refers to a polymeric molecule having a backbone that supports bases capable of hydrogen bonding to typical polynucleotides, wherein the polymer lacks a pentose sugar backbone moiety, and more specifically a ribose backbone linked by phosphodiester bonds which is typical of nucleotides and nucleosides, but instead contains a ring nitrogen with coupling through the ring nitrogen. An exemplary “morpholino” oligomer comprises morpholino subunit structures linked together by phosphoramidate or phosphorodiamidate linkages, joining the morpholino nitrogen of one subunit to the 5′ exocyclic carbon of an adjacent subunit, each subunit comprising a purine or pyrimidine base-pairing moiety effective to bind, by base-specific hydrogen bonding, to a base in a polynucleotide. Morpholino oligomers (including antisense oligomers) are detailed, for example, in U.S. Pat. Nos. 5,034,506; 5,142,047; 5,166,315; 5,185,444; 5,217,866; 5,506,337; 5,521,063; 5,698,685; 8,076,476; and 8,299,206; and PCT publication number WO 2009/064471, all of which are incorporated herein by reference in their entirety.

A preferred morpholino oligomer is a phosphorodiamidate-linked morpholino oligomer, referred to herein as a PMO. Such oligomers are composed of morpholino subunit structures such as those shown below:

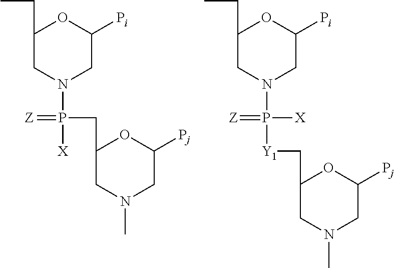

where X is NH₂, NHR, or NR₂ (where R is lower alkyl, preferably methyl), Y, is O, and Z is O, and Pᵢ and Pⱼ are purine or pyrimidine base-pairing moieties effective to bind, by base-specific hydrogen bonding, to a base in a polynucleotide. Also preferred are structures having an alternate phosphorodiamidate linkage, where X is lower alkoxy, such as methoxy or ethoxy, Y₁ is NH or NR, where R is lower alkyl, and Z is O.

Representative PMOs include PMOs wherein the intersubunit linkages are linkage (A1). See Table 1.

TABLE 1
Representative Intersubunit Linkages

| No. | Name | Structure |
|---|---|---|
| A1 | PMO | 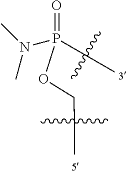 [SMILES: *COP(*)(=O)N(C)C] |
| A2 | PMO⁺ (unprotonated form depicted) | 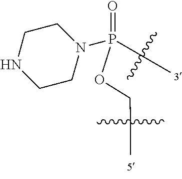 [SMILES: *COP(*)(=O)N1CCNCC1] |
| A3 | PMO⁺ (+) | 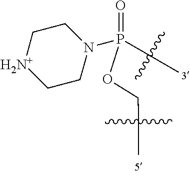 [SMILES: *COP(*)(=O)N1CC[NH2+]CC1] |

A “phosphoramidate” group comprises phosphorus having three attached oxygen atoms and one attached nitrogen atom, while a “phosphorodiamidate” group comprises phosphorus having two attached oxygen atoms and two attached nitrogen atoms. A representative phosphorodiamidate example is below:

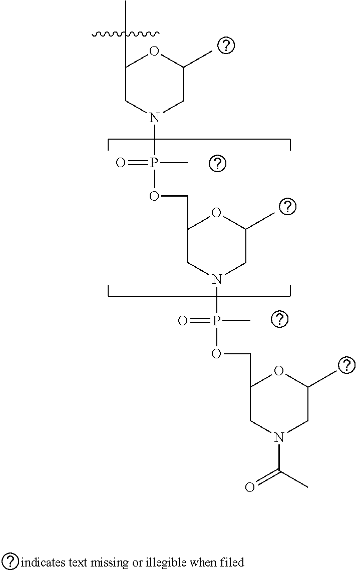

each Pᵢ is independently selected from H, a nucleobase, and a nucleobase functionalized with a chemical protecting-group, wherein the nucleobase independently at each occurrence comprises a C₃₋₆ heterocyclic ring selected from pyridine, pyrimidine, triazinane, purine, and deaza-purine; and n is an integer of 6-38.

In the uncharged or the modified intersubunit linkages of the oligomers described herein, one nitrogen is always pendant to the backbone chain. The second nitrogen, in a phosphorodiamidate linkage, is typically the ring nitrogen in a morpholino ring structure.

PMOs are water-soluble, uncharged or substantially uncharged antisense molecules that inhibit gene expression by preventing binding or progression of splicing or translational machinery components. PMOs have also been shown to inhibit or block viral replication (Stein, Skilling et al. 2001; McCaffrey, Meuse et al. 2003). They are highly resistant to enzymatic digestion (Hudziak, Barofsky et al. 1996). PMOs have demonstrated high antisense specificity and efficacy in vitro in cell-free and cell culture models (Stein, Foster et al. 1997; Summerton and Weller 1997), and in vivo in zebrafish, frog and sea urchin embryos (Heasman, Kofron et al. 2000; Nasevicius and Ekker 2000), as well as in adult animal models, such as rats, mice, rabbits, dogs, and pigs (see e.g. Arora and Iversen 2000; Qin, Taylor et al. 2000; Iversen 2001; Kipshidze, Keane et al. 2001; Devi 2002; Devi, Oldenkamp et al. 2002; Kipshidze, Kim et al. 2002; Ricker, Mata et al. 2002).

Antisense PMO oligomers have been shown to be taken up into cells and to be more consistently effective in vivo, with fewer nonspecific effects, than other widely used antisense oligonucleotides (see e.g. P. Iversen, “Phosphoramidite Morpholino Oligomers,” in Antisense Drug Technology, S. T. Crooke, ed., Marcel Dekker, Inc., New York, 2001). Conjugation of PMOs to arginine-rich peptides has been shown to increase their cellular uptake (see e.g., U.S. Pat. No. 7,468,418, incorporated herein by reference in its entirety).

“Charged,” “uncharged,” “cationic,” and “anionic” as used herein refer to the predominant state of a chemical moiety at near-neutral pH, e.g., about 6 to 8. For example, the term may refer to the predominant state of the chemical moiety at physiological pH, that is, about 7.4.

A “cationic PMO” or “PMO+” refers to a phosphorodiamidate morpholino oligomer comprising any number of (1-piperazino)phosphinylideneoxy, (1-(4-(ω-guanidino-alkanoyl))-piperazino)phosphinylideneoxy linkages (A2 and A3; see Table 1) that have been described previously (see e.g., PCT publication WO 2008/036127 which is incorporated herein by reference in its entirety).

The “backbone” of an oligonucleotide analog (e.g., an uncharged oligonucleotide analogue) refers to the structure supporting the base-pairing moieties; e.g., for a morpholino oligomer, as described herein, the “backbone” includes morpholino ring structures connected by intersubunit linkages (e.g., phosphorus-containing linkages). A “substantially uncharged backbone” refers to the backbone of an oligonucleotide analogue wherein less than 50% of the intersubunit linkages are charged at near-neutral pH. For example, a substantially uncharged backbone may comprise less than 50%, less than 40%, less than 30%, less than 20%, less than 10%, less than 5% or even 0% intersubunit linkages which are charged at near neutral pH. In some embodiments, the substantially uncharged backbone comprises at most one charged (at physiological pH) intersubunit linkage for every four uncharged (at physiological pH) linkages, at most one for every eight or at most one for every sixteen uncharged linkages. In some embodiments, the nucleic acid analogs described herein are fully uncharged.

The term “targeting base sequence” or simply “targeting sequence” is the sequence in the nucleic acid analog that is complementary (meaning, in addition, substantially complementary) to a target sequence, e.g., a target sequence in the RNA genome of human. The entire sequence, or only a portion, of the analog compound may be complementary to the target sequence. For example, in an analog having 20 bases, only 12-14 may be targeting sequences. Typically, the targeting sequence is formed of contiguous bases in the analog, but may alternatively be formed of non-contiguous sequences that when placed together, e.g., from opposite ends of the analog, constitute sequence that spans the target sequence.

As used herein, a “cell-penetrating peptide” (CPP) or “carrier peptide” is a relatively short peptide capable of promoting uptake of PMOs by cells, thereby delivering the PMOs to the interior (cytoplasm) of the cells. The CPP or carrier peptide typically is about 12 to about 40 amino acids long. The length of the carrier peptide is not particularly limited and varies in different embodiments. In some embodiments, the carrier peptide comprises from 4 to 40 amino acid subunits. In other embodiments, the carrier peptide comprises from 6 to 30, from 6 to 20, from 8 to 25 or from 10 to 20 amino acid subunits. In particular embodiments, the linking peptides of the conjugates provided herein may act as CPPs upon enzymatic cleavage. In another embodiment, the linking peptides of the conjugates provided herein act as CPPs even without enzymatic cleavage. In some embodiments, enzymatic cleavage preferentially occurs between the M and J moieties. In a further embodiment, the enzymatic cleavage preferentially occurs between two adjacent amino acids in the L-configuration.

In certain embodiments, the linking moiety is attached to an antisense oligonucleotide-peptide conjugate from the oligonucleotide conjugate. In certain embodiments, the carrier peptide, when conjugated to an antisense oligomer, is effective to enhance the binding of the antisense oligomer to its target sequence, relative to the antisense oligomer in unconjugated form, as evidenced by:
(i) a decrease in expression of an encoded protein, relative to that provided by the unconjugated oligomer, when binding of the antisense oligomer to its target sequence is effective to block a translation start codon for the encoded protein, or
(ii) an increase in expression of an encoded protein, relative to that provided by the unconjugated oligomer, when binding of the antisense oligomer to its target sequence is effective to block an aberrant splice site in a pre-mRNA which encodes said protein when correctly spliced. Assays suitable for measurement of these effects are described further below. In one embodiment, conjugation of the peptide provides this activity in a cell-free translation assay, as described herein. In some embodiments, activity is enhanced by a factor of at least two, a factor of at least five or a factor of at least ten.

Alternatively or in addition, the carrier peptide is effective to enhance the transport of the nucleic acid analog into a cell, relative to the analog in unconjugated form. In certain embodiments, transport is enhanced by a factor of at least two, a factor of at least two, a factor of at least five or a factor of at least ten.

As used herein, a “peptide-conjugated phosphorodiamidate-linked morpholino oligomer” or “PPMO” refers to a PMO covalently linked to a peptide, such as a cell-penetrating peptide (CPP) or carrier peptide. The cell-penetrating peptide promotes uptake of the PMO by cells, thereby delivering the PMO to the interior (cytoplasm) of the cells.

Depending on its amino acid sequence, a CPP can be generally effective or it can be specifically or selectively effective for PMO delivery to a particular type or particular types of cells. PMOs and CPPs are typically linked at their ends, e.g., the C-terminal end of the CPP can be linked to the 5′ end of the PMO, or the 3′ end of the PMO can be linked to the N-terminal end of the CPP. PPMOs can include uncharged PMOs, charged (e.g., cationic) PMOs, and mixtures thereof. In an embodiment, the linking moiety of the conjugates described herein may be cleaved to release a PPMO.

The carrier peptide may be linked to the nucleic acid analog either directly or via an optional linker, e.g., one or more additional naturally occurring amino acids, e.g., cysteine (C), glycine (G), or proline (P), or additional amino acid analogs, e.g., 6-aminohexanoic acid (X), beta-alanine (B), or XB.

An “amino acid subunit” is generally an α-amino acid residue (—CO—CHR—NH—); but may also be a β- or other amino acid residue (e.g., —CO—CH₂CHR—NH—), where R is an amino acid side chain.

The term “naturally occurring amino acid” refers to an amino acid present in proteins found in nature; examples include Alanine (A), Cysteine (C), Aspartic acid (D), Glutamic acid (E), Phenyalanine (F), Glycine (G), Histidine (H), Isoleucine (I), Lysine (K), Leucine (L). Methionine (M), Asparagine (N), Proline (P), Glutamine (Q), Arginine (R), Serine (S), Threonine (T), Valine (V), Tryptophan (W), and Tyrosine (Y). The term “non-natural amino acids” or “unnatural amino acid” refers to those amino acids not present in proteins found in nature; examples include beta-alanine (β-Ala) and 6-aminohexanoic acid (Ahx), γ-aminobutyric acid (Abu), homoleucine (Hle), norleucine (Nle), naphthylalanine (Nap), diphenylalanine (Dpa), diaminobutyric acid (Dab), aminopiperidine-carboxylic acid (Pip), aminomethylphenylalanine (Amf), and 2-amino-4-guanidinobutanoic acid (Gba). As used herein, an “effective amount” refers to any amount of a substance that is sufficient to achieve a desired biological result. A “therapeutically effective amount” refers to any amount of a substance that is sufficient to achieve a desired therapeutic result.

As used herein, a “subject” is a mammal, which can include a mouse, rat, hamster, guinea pig, rabbit, goat, sheep, cat, dog, pig, cow, horse, monkey, non-human primate, or human. In certain embodiments, a subject is a human.

“Treatment” of an individual (e.g., a mammal, such as a human) or a cell is any type of intervention used to alter the natural course of the individual or cell. Treatment includes, but is not limited to, administration of a pharmaceutical composition, and may be performed either prophylactically or subsequent to the initiation of a pathologic event or contact with an etiologic agent.

### II. Peptide-Oligonucleotide-Conjugates

In an embodiment, provided herein are conjugates comprising an antisense oligonucleotide covalently linked to a cell-penetrating peptide. In some embodiments, the antisense oligonucleotide is selected from one or more chemistries described herein.

In an embodiment, provided herein is an oligonucleotide conjugate comprising a compound of Formula I:

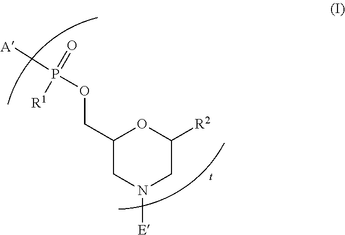

or a pharmaceutically acceptable salt thereof,
wherein:
A′ is selected from —N(H)CH₂C(O)NH₂, —N(C₁₋₆-alkyl)CH₂C(O)NH₂,

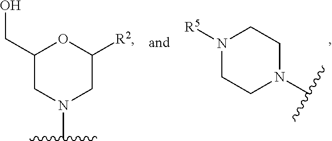

wherein
R⁵ is —C(O)(O-alkyl)ₓ-OH, wherein x is 3-10 and each alkyl group is, independently at each occurrence, C₂₋₆-alkyl,
or R⁵ is selected from H, —C(O)C₁₋₆-alkyl, trityl, monomethoxytrityl, —(C₁₋₆-alkyl)-R⁶, —(C₁₋₆-heteroalkyl)-R⁶, aryl-R⁶, heteroaryl-R⁶, —C(O)O—(C₁₋₆-alkyl)-R⁶, —C(O)O-aryl-R⁶, —C(O)O— heteroaryl-R⁶, and

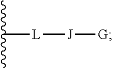

R⁶ is selected from OH, SH, and NH₂, or R⁶ is O, S, or NH, each of which is covalently linked to a solid support;
each R¹ is independently selected from OH and —N(R³)(R⁴), wherein each R³ and R⁴ are, independently at each occurrence, H or —C₁₋₆-alkyl;
each R² is independently, at each occurrence, selected from H, a nucleobase, and a nucleobase functionalized with a chemical protecting-group, wherein the nucleobase, independently at each occurrence, comprises a C₃₋₆-heterocyclic ring selected from pyridine, pyrimidine, purine, and deaza-purine;
t is 8-40;
E′ is selected from H, —C₁₋₆-alkyl, —C(O)C₁₋₆-alkyl, benzoyl, stearoyl, trityl, monomethoxytrityl, dimethoxytrityl, trimethoxytrityl,

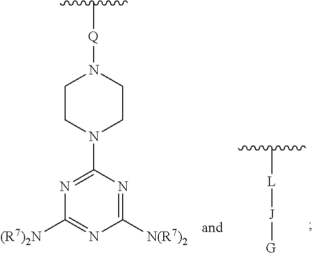

wherein
Q is —C(O)(CH₂)₆C(O)— or —C(O)(CH₂)₂S₂(CH₂)₂C(O)—;
R⁷ is —(CH₂)₂OC(O)N(R⁸)₂, wherein R⁸ is —(CH₂)₆NHC(═NH)NH₂;
L is —C(O)(CH₂)₁₋₈C(O)—C₇₋₁₈-heteroaromatic-((CH₂)₁₋₈C(O)—, —C(O)(CH₂)₁₋₈C(O)—C₇₋₁₈-heteroaromatic-(W)—; —C(O)(CH₂)₁₋₈C(O)—C₇₋₁₈-heteroaromatic-(W—W)—;
W is independently at each occurrence a linking amino acid;
J is a cell-penetrating peptide selected from d-R₃, d-BPEP, d-DPV7, d-DPV6, d-penetratin, d-Bac7, d-MPG, d-Hel11-7, d-TAT, d-TATp, d-WR₃, d-WBPEP, d-WDPV7, d-WDPV6, d-WTAT, or d-WTATp; and
G is selected from H, —C(O)C₁₋₆-alkyl, benzoyl, and stearoyl, wherein G is covalently linked to J;
provided that
   A′ is

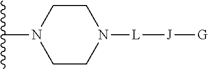

or E′ is

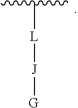

In another aspect, provided herein is an oligonucleotide conjugate comprising a compound of Formula II:

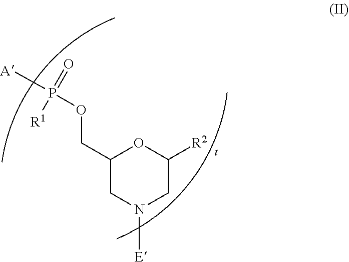

or a pharmaceutically acceptable salt thereof,
wherein:
A′ is selected from —N(H)CH₂C(O)NH₂, —N(C₁₋₆-alkyl)CH₂C(O)NH₂,

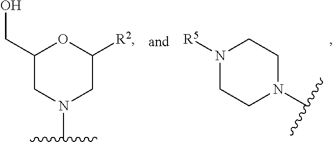

wherein
R⁵ is —C(O)(O-alkyl)ₓ-OH, wherein x is 3-10 and each alkyl group is, independently at each occurrence, C₂₋₆-alkyl,
or R⁵ is selected from H, —C(O)C₁₋₆-alkyl, trityl, monomethoxytrityl, —(C₁₋₆-alkyl)-R⁶, —(C₁₋₆-heteroalkyl)-R⁶, aryl-R⁶, heteroaryl-R⁶, —C(O)O—(C₁₋₆-alkyl)-R⁶, —C(O)O-aryl-R⁶, —C(O)O— heteroaryl-R⁶, and

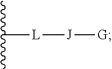

R⁶ is selected from OH, SH, and NH₂, or R⁶ is O, S, or NH, each of which is covalently linked to a solid support;
each R¹ is independently selected from OH and —N(R³)(R⁴), wherein each R³ and R⁴ are, independently at each occurrence, H or —C₁₋₆-alkyl;
each R² is independently, at each occurrence, selected from H, a nucleobase, and a nucleobase functionalized with a chemical protecting-group, wherein the nucleobase, independently at each occurrence, comprises a C₃₋₆-heterocyclic ring selected from pyridine, pyrimidine, purine, and deaza-purine;
t is 8-40;
E′ is selected from H, —C₁₋₆-alkyl, —C(O)C₁₋₆-alkyl, benzoyl, stearoyl, trityl, monomethoxytrityl, dimethoxytrityl, trimethoxytrityl,

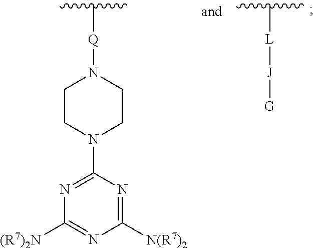

wherein
Q is —C(O)(CH₂)₆C(O)— or —C(O)(CH₂)₂S₂(CH₂)₂C(O)—;
R⁷ is —(CH₂)₂OC(O)N(R⁸)₂, wherein R⁸ is —(CH₂)₆NHC(═NH)NH₂;
L is —C(O)(CH₂)₁₋₈C(O)—C₇₋₁₈-heteroaromatic-((CH₂)₁₋₈C(O)—, —C(O)(CH₂)₁₋₈C(O)—C₇₋₁₈-heteroaromatic-(W)—; —C(O)(CH₂)₁₋₈C(O)—C₇₋₁₈-heteroaromatic-(W—W)—;
W is independently at each occurrence a linking amino acid;
J is a cell-penetrating peptide selected from WR₈, WTAT, WBPEP, WDPV7, WTATp, WDPV6, TATp, MPG, or Hel11-7, wherein all chiral amino acids of the peptide are in the L-configuration; and
G is selected from H, —C(O)C₁₋₆-alkyl, benzoyl, and stearoyl, wherein G is covalently linked to J;
provided that
   A′ is

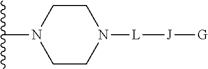

or E′ is

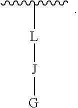

In yet another aspect, provided herein is an oligonucleotide conjugate comprising a compound of Formula III:

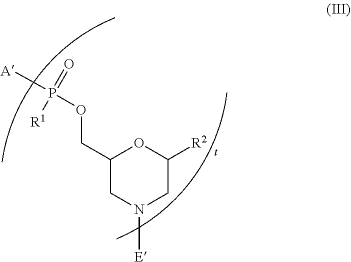

or a pharmaceutically acceptable salt thereof,
wherein:
A′ is selected from —N(H)CH₂C(O)NH₂, —N(C₁₋₆-alkyl)CH₂C(O)NH₂,

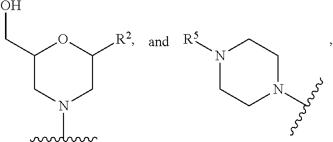

wherein
R⁵ is —C(O)(O-alkyl)ₓ-OH, wherein x is 3-10 and each alkyl group is, independently at each occurrence, C₂₋₆-alkyl,
or R⁵ is selected from H, —C(O)C₁₋₆-alkyl, trityl, monomethoxytrityl, —(C₁₋₆-alkyl)-R⁶, —(C₁₋₆-heteroalkyl)-R⁶, aryl-R⁶, heteroaryl-R⁶, —C(O)O—(C₁₋₆-alkyl)-R⁶, —C(O)O-aryl-R⁶, —C(O)O— heteroaryl-R⁶, and

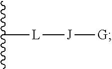

R⁶ is selected from OH, SH, and NH₂, or R⁶ is O, S, or NH, each of which is covalently linked to a solid support;
each R¹ is independently selected from OH and —N(R³)(R⁴), wherein each R³ and R⁴ are, independently at each occurrence, H or —C₁₋₆-alkyl;
each R² is independently, at each occurrence, selected from H, a nucleobase, and a nucleobase functionalized with a chemical protecting-group, wherein the nucleobase, independently at each occurrence, comprises a C₃₋₆-heterocyclic ring selected from pyridine, pyrimidine, purine, and deaza-purine;
t is 8-40;
E′ is selected from H, —C₁₋₆-alkyl, —C(O)C₁₋₆-alkyl, benzoyl, stearoyl, trityl, monomethoxytrityl, dimethoxytrityl, trimethoxytrityl,

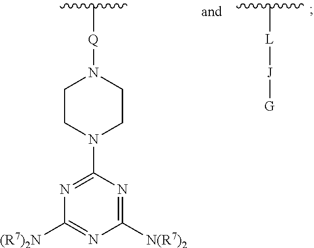

wherein
Q is —C(O)(CH₂)₆C(O)— or —C(O)(CH₂)₂S₂(CH₂)₂C(O)—;
R⁷ is —(CH₂)₂OC(O)N(R⁸)₂, wherein R⁸ is —(CH₂)₆NHC(═NH)NH₂;
L is —C(O)(CH₂)₁₋₈C(O)—C₇₋₁₈-heteroaromatic-((CH₂)₁₋₈C(O)—, —C(O)(CH₂)₁₋₈C(O)—C₇₋₁₈-heteroaromatic-(W)—; —C(O)(CH₂)₁₋₈C(O)—C₇₋₁₈-heteroaromatic-(W—W)—;
W is independently at each occurrence a linking amino acid;
J is a cell-penetrating peptide comprising 3 to 15 amino acids selected from unnatural amino acids or D-amino acids, further comprising a C-terminal sequence having a KWKK, KKWK, KWWKK, WWKK, WKK, KKKK, or KK motif; and
G is selected from H, —C(O)C₁₋₆-alkyl, benzoyl, and stearoyl, wherein G is covalently linked to J;
provided that
A′ is

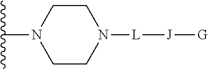

or E′ is

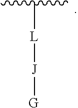

In an embodiment, E′ is selected from H, —C₁₋₆-alkyl, —C(O)C₁₋₆-alkyl, benzoyl, stearoyl, trityl, monomethoxytrityl, dimethoxytrityl, trimethoxytrityl, and

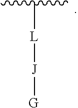

In an embodiment, A′ is selected from —N(C₁₋₆-alkyl)CH₂C(O)NH₂,

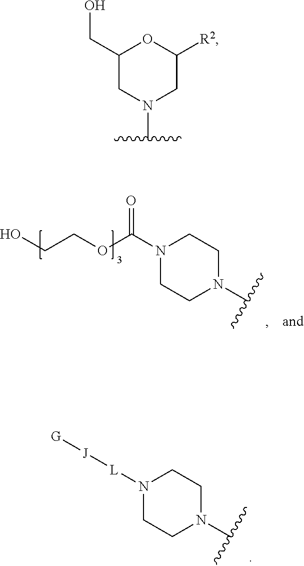

In an embodiment, E′ is selected from H, —C(O)CH₃, benzoyl, stearoyl, trityl, 4-methoxytrityl, and

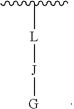

In another embodiment, A′ is selected from —N(C₁₋₆-alkyl)CH₂C(O)NH₂,

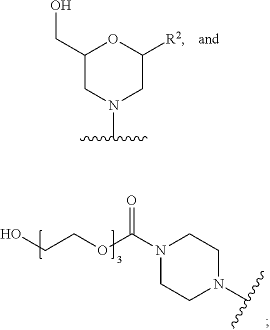

and
E′ is G

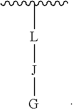

In another embodiment, A′ is

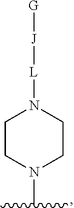

and
E′ is selected from H, —C(O)CH₃, trityl, 4-methoxytrityl, benzoyl, and stearoyl.

In an embodiment, the peptide-oligonucleotide conjugate of Formula I or Formula II is a peptide-oligonucleotide conjugate selected from:

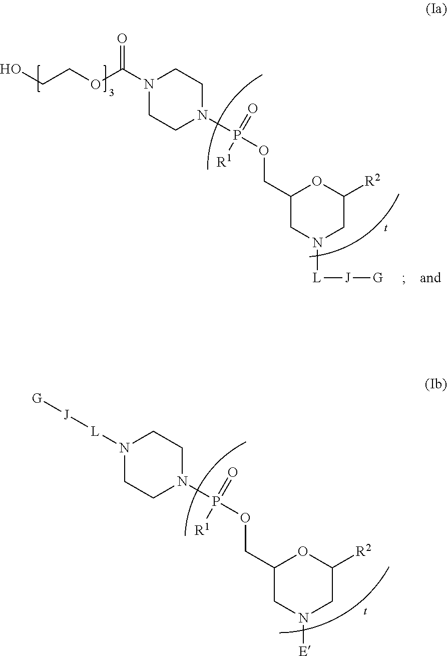

wherein E′ is selected from H, C₁₋₆-alkyl, —C(O)CH₃, benzoyl, and stearoyl.

In another embodiment, L is optionally further bound to biotin.

In another embodiment, J is optionally bound to L via a trypsin cleavable linker.

In another embodiment, the peptide-oligonucleotide conjugate is of the formula (Ia).

In another embodiment, the peptide-oligonucleotide conjugate is of the formula (Ib).

In another embodiment, each R¹ is N(CH₃)₂.

In another embodiment, each R² is a nucleobase, independently at each occurrence, selected from adenine, guanine, cytosine, 5-methyl-cytosine, thymine, uracil, and hypoxanthine.

In another embodiment, L is —C(O)(CH₂)₁₋₈C(O)-DBCO—(CH₂)₁₋₈C(O)—.

In another embodiment, L is

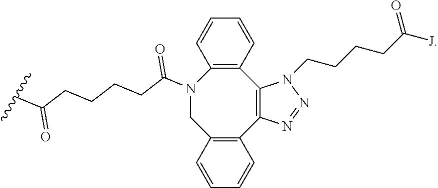

In an embodiment, L is —C(O)(CH₂)₁₋₈C(O)-DBCO—(W)—.

In an embodiment, L is —C(O)(CH₂)₁₋₈C(O)-DBCO—(W—W)—.

In an embodiment, W is independently selected from glycine, proline, β-alanine, 6-aminohexanoic acid, or lysine. In another embodiment, W is lysine.

In an embodiment, W—W is lysine-(6-amino hexanoic acid).

In an embodiment, L is optionally further bound to biotin at the N-terminus of the linking amino acid.

In an embodiment, L is

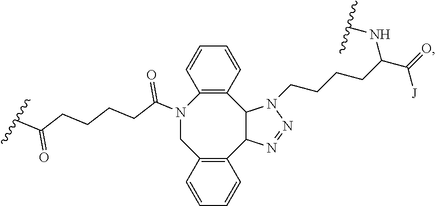

wherein N(H) is bound to biotin.

In an embodiment, L is

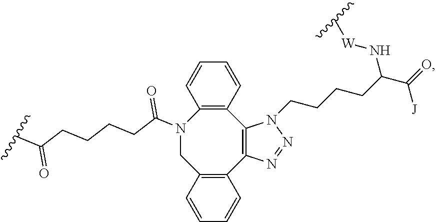

wherein W is bound to biotin.

In an embodiment, biotin has the structure:

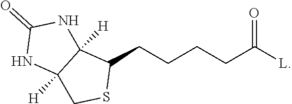

In a further embodiment, biotin is bound to L via 6-aminohexanoic acid or lysine.

In an embodiment, provided herein is a biotin-sulfoCy5-labeled PMO-CPP having the following structure:

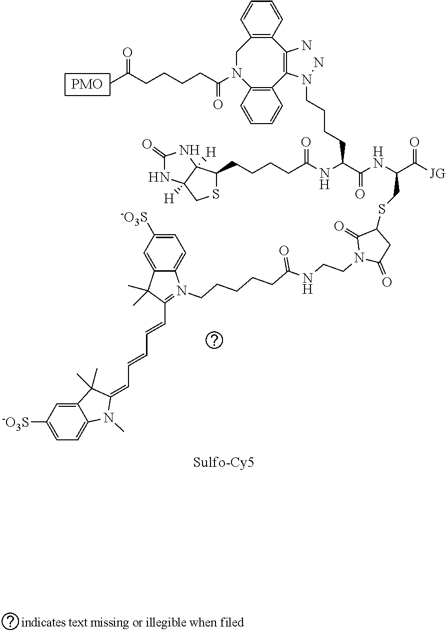

wherein J is a cell penetrating peptide comprising a cell penetrating peptide can be 3 to 15 amino acids selected from unnatural amino acids or D-amino acids excluding the C-terminal sequence. In an embodiment, the cell penetrating peptide further comprises a C-terminal sequence having a KWKK, KKWK, KWWKK, WWKK, WKK, KKKK, or KK motif. In another embodiment, the cell penetrating peptide further comprises a C-terminal sequence having a (d-K)(d-W)(d-K)(d-K), (d-K)(d-K)(d-W)(d-K), (d-K)(d-W)(d-W)(d-K)(d-K), (d-W)(d-W)(d-K)(d-K), (d-W)(d-K)(d-K), (d-K)(d-K)(d-K)(d-K), or (d-K)(d-K) motif.

In an embodiment, J is selected from d-R₃, d-BPEP, d-DPV7, d-DPV6, d-penetratin, d-Bac7, d-MPG, d-Hel11-7, d-TAT, d-TATp, d-WR₃, d-WBPEP, d-WDPV7, d-WDPV6, d-WTAT, or d-WTATp. In a further embodiment, J is selected from d-DPV7, d-DPV6, d-penetratin, d-Bac7, d-MPG, d-Hel11-7, d-WR₃, d-WBPEP, d-WDPV7, d-WDPV6, d-WTAT, or d-WTATp.

In an embodiment, the unnatural amino acids are selected from Abu (γ-aminobutyric acid), B (β-alanine), HIe (homoleucine), Ne (norleucine), Nap (naphthylalanine), Dpa (diphenylalanine), Dab (diaminobutyric acid), Pip (aminopiperidine-carboxylic acid), Amf (aminomethylphenylalanine), and Gba (2-amino-4-guanidinobutanoic acid).

In an embodiment, the cell penetrating peptide is selected from SEQ ID NOS: 33-669.

In an embodiment, the cell penetrating peptide is selected from:

SEQ ID NO.: 657
(B)(d-Arg)(d-Arg)(Abu)(Dab)(d-His);
SEQ ID NO.: 664
(Abu)(Gly)(d-Asn)(Nle)(d-Asn)(d-His);
SEQ ID NO.: 644
(Nle)(d-Pro)(d-Asp)(d-Glu)(d-Thr);
and
SEQ ID NO.: 646
(B)(Abu)(d-Ser)(Abu)(Hle).

In an embodiment, the C-terminal sequence is a KWKK motif. In another embodiment, the C-terminal sequence is a (d-K)(d-W)(d-K)(d-K) motif.

In an embodiment, J is bound to L via a trypsin cleavable linker.

In an embodiment, the trypsin cleavable linker is GGKGG.

In an embodiment, G is selected from H, C(O)CH₃, benzoyl, and stearoyl. In another embodiment, G is H or —C(O)CH₃. In a further embodiment, G is H. In another embodiment, G is —C(O)CH₃.

### III. Oligomer Chemistry Features

Also provided herein are oligonucleotide conjugates, wherein the oligonucleotide is a modified antisense oligomer. Examples of modified antisense oligomers include, without limitation, morpholino oligomers. In some embodiments, the nucleobases of the modified antisense oligomer are linked to morpholino ring structures, wherein the morpholino ring structures are joined by phosphorous-containing intersubunit linkages joining a morpholino nitrogen of one ring structure to a 5′ exocyclic carbon of an adjacent ring structure.

### IV. Target Sequences

In some embodiments for antisense applications, the oligomer can be 100% complementary to the nucleic acid target sequence, or it may include mismatches, e.g., to accommodate variants, as long as a heteroduplex formed between the oligomer and nucleic acid target sequence is sufficiently stable to withstand the action of cellular nucleases and other modes of degradation which may occur in vivo. Mismatches, if present, are less destabilizing toward the end regions of the hybrid duplex than in the middle. The number of mismatches allowed will depend on the length of the oligomer, the percentage of G:C base pairs in the duplex, and the position of the mismatch(es) in the duplex, according to well understood principles of duplex stability. Although such an antisense oligomer is not necessarily 100% complementary to the nucleic acid target sequence, it is effective to stably and specifically bind to the target sequence, such that a biological activity of the nucleic acid target, e.g., expression of encoded protein(s), is modulated.

The stability of the duplex formed between an oligomer and the target sequence is a function of the binding Tₘ and the susceptibility of the duplex to cellular enzymatic cleavage. The Tₘ of an antisense compound with respect to complementary-sequence RNA may be measured by conventional methods, such as those described by Hames et al., Nucleic Acid Hybridization, IRL Press, 1985, pp. 107-108 or as described in Miyada C G. and Wallace R B (1987) Oligonucleotide hybridization techniques, Methods Enzymol. Vol. 154 pp. 94-107.

In some embodiments, each antisense oligomer has a binding Tₘ, with respect to a complementary-sequence RNA, of greater than body temperature or in other embodiments greater than 50° C. In other embodiments Tₘ's are in the range 60-80° C. or greater. According to well known principles, the Tₘ of an oligomer compound, with respect to a complementary-based RNA hybrid, can be increased by increasing the ratio of C:G paired bases in the duplex, and/or by increasing the length (in base pairs) of the heteroduplex. At the same time, for purposes of optimizing cellular uptake, it may be advantageous to limit the size of the oligomer. For this reason, compounds that show high Tₘ (50° C. or greater) at a length of 20 bases or less are generally preferred over those requiring greater than 20 bases for high Tₘ values. For some applications, longer oligomers, for example longer than 20 bases, may have certain advantages.

The targeting sequence bases may be normal DNA bases or analogues thereof, e.g., uracil and inosine that are capable of Watson-Crick base pairing to target-sequence RNA bases.

An antisense oligomer can be designed to block or inhibit or modulate translation of mRNA or to inhibit or modulate pre-mRNA splice processing, or induce degradation of targeted mRNAs, and may be said to be “directed to” or “targeted against” a target sequence with which it hybridizes. In certain embodiments, the target sequence includes a region including a 3′ or 5′ splice site of a pre-processed mRNA, a branch point, or other sequence involved in the regulation of splicing. The target sequence may be within an exon or within an intron or spanning an intron/exon junction.

An antisense oligomer having a sufficient sequence complementarity to a target RNA sequence to modulate splicing of the target RNA means that the antisense agent has a sequence sufficient to trigger the masking of a binding site for a native protein that would otherwise modulate splicing and/or alters the three-dimensional structure of the targeted RNA. Likewise, an oligomer reagent having a sufficient sequence complementary to a target RNA sequence to modulate splicing of the target RNA means that the oligomer reagent has a sequence sufficient to trigger the masking of a binding site for a native protein that would otherwise modulate splicing and/or alters the three-dimensional structure of the targeted RNA.

In certain embodiments, the degree of complementarity between the target sequence and antisense targeting sequence is sufficient to form a stable duplex. The region of complementarity of the antisense oligomers with the target RNA sequence may be as short as 8-11 bases, but can be 12-15 bases or more, e.g., 10-40 bases, 12-30 bases, 12-25 bases, 15-25 bases, 12-20 bases, or 15-20 bases, including all integers in between these ranges. An antisense oligomer of about 14-15 bases is generally long enough to have a unique complementary sequence. In certain embodiments, a minimum length of complementary bases may be required to achieve the requisite binding Tm, as discussed herein.

In certain embodiments, oligomers as long as 40 bases may be suitable, where at least a minimum number of bases, e.g., 10-12 bases, are complementary to the target sequence. In some embodiments, facilitated or active uptake in cells is optimized at oligomer lengths of less than about 30 bases. For PMO oligomers, described further herein, an optimum balance of binding stability and uptake generally occurs at lengths of 18-25 bases. Included in the disclosure are antisense oligomers (e.g., PMOs) that consist of about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 bases, in which at least about 6, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or40 contiguous or non-contiguous bases are complementary to the desired target sequences.

In certain embodiments, antisense oligomers may be 100% complementary to the target sequence, or may include mismatches, e.g., to accommodate variants, as long as a heteroduplex formed between the oligomer and target sequence is sufficiently stable to withstand the action of cellular nucleases and other modes of degradation which may occur in vivo. Hence, certain oligomers may have substantial complementarity, meaning, about or at least about 70% sequence complementarity, e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence complementarity, between the oligomer and the target sequence. Oligomer backbones that are less susceptible to cleavage by nucleases are discussed herein. Mismatches, if present, are typically less destabilizing toward the end regions of the hybrid duplex than in the middle. The number of mismatches allowed will depend on the length of the oligomer, the percentage of G:C base pairs in the duplex, and the position of the mismatch(es) in the duplex, according to well understood principles of duplex stability. Although such an antisense oligomer is not necessarily 100% complementary to the target sequence, it is effective to stably and specifically bind to the target sequence, such that splicing of the target pre-RNA is modulated.

The stability of the duplex formed between an oligomer and a target sequence is a function of the binding Tₘ and the susceptibility of the duplex to cellular enzymatic cleavage. The Tₘ of an oligomer with respect to complementary-sequence RNA may be measured by conventional methods, such as those described by Hames et al., Nucleic Acid Hybridization, IRL Press, 1985, pp. 107-108 or as described in Miyada C. G. and Wallace R. B., 1987, Oligomer Hybridization Techniques, Methods Enzymol. Vol. 154 pp. 94-107. In certain embodiments, antisense oligomers may have a binding Tₘ, with respect to a complementary-sequence RNA, of greater than body temperature and preferably greater than about 45° C. or 50° C. Tₘ's in the range 60-80° C. or greater are also included. According to well-known principles, the Tₘ of an oligomer, with respect to a complementary-based RNA hybrid, can be increased by increasing the ratio of C:G paired bases in the duplex, and/or by increasing the length (in base pairs) of the heteroduplex. At the same time, for purposes of optimizing cellular uptake, it may be advantageous to limit the size of the oligomer. For this reason, compounds that show high Tₘ (45-50° C. or greater) at a length of 25 bases or less are generally preferred over those requiring greater than 25 bases for high Tₘ values.

In one aspect, the disclosure provides an antisense oligomer conjugate, or a pharmaceutically acceptable salt thereof, capable of binding a selected target to induce exon skipping in the human dystrophin gene, wherein the antisense oligomer conjugate, or a pharmaceutically acceptable salt thereof, comprises a sequence of bases that is complementary to an exon target region of the dystrophin pre-mRNA designated as an annealing site, wherein each nucleobase R², as recited in Formula (I), Formula (II), Formula (III) and described throughout the specification, from 1 to t and 5′ to 3′ can be selected from:

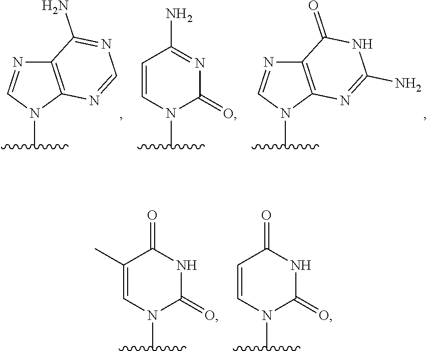

methylated guanine, methylated adenine, and

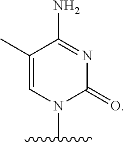 [SMILES: *n1cc(C)c(N)nc1=O]

In a particular embodiment, the sequence listing for the oligonucleotide is GCTATTACCTTAACCCAG.

In an embodiment, provided herein is a compound having the following structure:

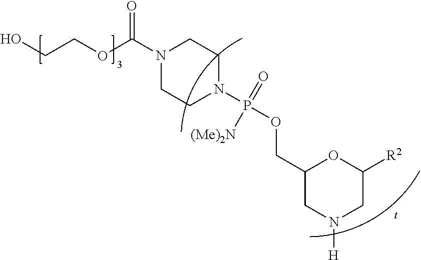

wherein z is 18 and R² is a sequence of nucleobases having the sequence of GCTATTACCTTAACCCAG. This compound is also referred to herein as “PMO IVS2-654.”

### V. Cell Penetrating Peptides (CPPs)

As described, cell-penetrating peptides (CPP) within the scope of substituent J have been shown to be effective in enhancing penetration of antisense oligomers into a cell and to cause exon skipping in different muscle groups in animal models.

Exemplary peptides are given below in Table 2.

TABLE 2
Cell-Penetrating Peptides

| Name | | |
|---|---|---|
| (Designation) | Sequence | SEQ ID NO. |
| R₈ | RRRRRRRR | 1 |
| BPEP | RXRRBRRXRRBR | 2 |
| DPV7 | KRKKKGKLGKKRDP | 3 |
| DPV6 | GRPRESGKKRKRKRLKP | 4 |
| Penetratin | RQIKIWFQNRRMKWKK | 5 |
| Bac7 | RRIRPRPPRLPRPRPRPLPFPRPG | 6 |
| MPG | LAFLGFLGAAGSTMGAWSQPKKKRKV | 7 |
| Hel11-7 | LLKLLLKLWKKLLKLLK | 8 |
| TAT | RKKRRQRRR | 9 |
| TATp | GRKKRRQRRRPPQ | 10 |
| WR₈ | WRRRRRRRR | 11 |
| WBPEP | WRXRRBRRXRRBR | 12 |
| WDPV7 | WKRKKKGKLGKKRDP | 13 |
| WDPV6 | WGRPRESGKKRKRKRLKP | 14 |
| WTAT | WRKKRRQRRR | 15 |
| WTATp | WGRKKRRQRRRPPQ | 16 |
| d-R₈ | dRdRdRdRdRdRdRdR | 17 |
| d-BPEP | dRXdRdRBdRdRXdRdRBdR | 18 |
| d-DPV7 | dKdRdKdKdKGdKdLGdKdKdRdDdP | 19 |
| d-DPV6 | GdRdPdRdEdSdGdKdKdRdKdRdKdRdLdKdP | 20 |
| d-Penetratin | dRdQdldKdldWdFdQdNdRdRdMdKdWdKdK | 21 |
| d-Bac7 | dRdRdldRdPdRdPdPdRdLdPdRdPdRdPdRdPdLdPdFdPdRdPG | 22 |
| d-MPG | dLdAdFdLGdFdLdGdAdAGdSdTdMGdAdWdSdQdPdKdKdKdRdKdV | 23 |
| d-Hel11-7 | dLdLdKdLdLdLdKdLdWdKdKdLdLdKdLdLdK | 24 |
| d-Tat | dRdKdKdRdRdQdRdRdR | 25 |
| d-TATp | GdRdKdKdRdRdQdRdRdRdPdPdQ | 26 |
| d-WR₈ | dWdRdRdRdRdRdRdRdR | 27 |
| d-WBPEP | dWdRXdRdRBdRdRXdRdRBdR | 28 |
| d-WDPV7 | dWdKdRdKdKdKGdKdLGdKdKdRdDdP | 29 |
| d-WDPV6 | dWGdRdPdRdEdSdGdKdKdRdKdRdKdRdLdKdP | 30 |
| d-WTat | dWdRdKdKdRdRdQdRdRdR | 31 |
| d-WTATp | dWGdRdKdKdRdRdQdRdRdRdPdPdQ | 32 |

ªSequences assigned to SEQ ID NOs do not include the linkage portion (e.g.,
proline, beta-alanine, and glycine). X and B refer to 6-aminohexanoic acid and
beta-alanine, respectively.

In an embodiment, the cell penetrating peptides provided above are bound to L via a trypsin cleavable linker. In an embodiment, the trypsin cleavable linker is GGKGG.

In another embodiment, provided herein is a biotin/trypsin-cleavable linker/cell penetrating peptide conjugate that can be used in the compositions provided herein, having the formula: Biotin-GGKGG-D-Arg₄, Biotin-GGKGG-D-Arge, Biotin-GGKGG-D-Arg₈, and Biotin-GGKGG-D-Arg₁₀.

In still another embodiment, provided herein is a trypsin-cleavable linker/cell penetrating peptide conjugate that can be used in the compositions provided herein, having the formula: GGKGG-D-Arg₄, GGKGG-D-Arg₆, GGKGG-D-Arg₈, and GGKGG-D-Arg.

Additional exemplary peptides are given below in Table 3.

TABLE 3
Cell-Penetrating Peptides

| Sequence | SEQ ID NO. |
|---|---|
| 33 | (Hle)(Pip)(d-Asp)(Nap)(d-Asp)(Dpa) |
| 34 | (Dpa)(d-Phe)(d-Lys)(d-Phe)(d-Asn)(d-Thr) |
| 35 | (Nap)(d-Nle)(Abu)(d-Pro)(d-Nle)(Amf) |
| 36 | (Dpa)(d-Asn)(d-Thr)(d-Arg)(Dpa)(d-Asn) |
| 37 | (Nap)(d-Thr)(d-Phe)(d-Nle)(Gly)(d-Glu) |
| 38 | (Dpa)(d-Phe)(d-Lys)(Nap)(d-Ser)(d-Asp) |
| 39 | (Gly)(Dpa)(B)(Dpa)(d-Phe)(d-Gln) |
| 40 | (d-Asp)(d-Thr)(B)(d-Nle)(Dpa)(Dpa) |
| 41 | (Nap)(d-Pro)(d-Nle)(d-Glu)(Nap)(Hle) |
| 42 | (Hle)(d-Phe)(Hle)(d-Asn)(d-Thr)(d-Gln) |
| 43 | (d-Asn)(d-Nle)(Abu)(d-Nle)(Hle)(d-Gln) |
| 44 | (d-Asp)(Dpa)(Amf)(d-Asp)(Dpa)(d-Lys) |
| 45 | (d-Ser)(Dab)(Nap)(d-Asp)(Dpa)(d-Gln) |
| 46 | (d-Phe)(d-Ser)(Dpa)(Gly)(Hle)(d-Arg) |
| 47 | (Gly)(Dpa)(d-Nle)(d-His)(Dpa)(Gly) |
| 48 | (d-Ser)(d-Glu)(Dpa)(d-Thr)(Nap)(Gly) |
| 49 | (Hle)(d-Thr)(d-Glu)(Hle)(Dpa)(d-Pro) |
| 50 | (Gly)(d-Phe)(d-Pro)(d-Nle)(d-Glu)(Dpa) |
| 51 | (Nap)(Hle)(d-Thr)(d-Gln)(Nap)(d-Glu) |
| 52 | (Dpa)(Nap)(Hle)(d-Gln)(Pip)(d-Phe) |
| 53 | (d-Pro)(d-Lys)(d-Nle)(d-Asp)(d-Phe) |
| | (d-Nle) |
| 54 | (Hle) (Dab)(d-Phe)(d-Gln)(d-Arg)(Dpa) |
| 55 | (Dpa)(Dpa)(d-Lys)(d-His)(Hle)(d-Thr) |
| 56 | (d-Ser)(d-Asn)(Dpa)(d-Asp)(Dpa)(Pip) |
| 57 | (d-Thr)(d-Asn)(d-Phe)(d-Nle)(Gly)(Dpa) |
| 58 | (d-Phe)(Hle)(Hle)(d-Asn)(d-Thr)(d-Gln) |
| 59 | (d-Lys)(d-Phe)(d-Ser)(d-Nle)(d-Glu)(Dab) |
| 60 | (B)(d-Lys)(d-Gln)(Dpa)(d-Phe)(d-Asn) |
| 61 | (Pip)(Hle)(d-Arg)(Amf)(Gly)(Dpa) |
| 62 | (Dpa)(Gly)(d-Glu)(d-Asn)(d-Asp)(d-Nle) |
| 63 | (d-Phe)(Dpa)(d-Gln)(d-Glu)(Abu)(d-Nle) |
| 64 | (Nap)(B)(d-Phe)(Dpa)(Amf)(d-Glu) |
| 65 | (d-Asp)(d-Thr)(B)(d-Nle)(Nap)(Dpa) |
| 66 | (d-Phe)(Gly)(d-Pro)(d-Nle)(d-Glu)(Dpa) |
| 67 | (Dpa)(Gly)(B)(Dpa)(d-Phe)(d-Gln) |
| 68 | (d-Phe)(d-Lys)(Nap)(d-Asn)(d-Phe)(d-Lys) |
| 69 | (d-Asn)(d-Phe)(d-Asp)(d-Ser)(Dpa)(Amf) |
| 70 | (d-Pro)(d-Ser)(d-Phe)(Dpa)(d-Glu)(B) |
| 71 | (d-Pro)(d-Nle)(d-Nle)(d-Glu)(Nap)(Hle) |
| 72 | (Abu)(Dpa)(d-Phe)(Hle)(d-Ser)(Hle) |
| 73 | (Gly)(Dpa)(Nle)(Dpa)(Gly) |
| 74 | (B)(Hle)(d-Thr)(d-Nle)(Gly)(d-Asn) |
| 75 | (d-Lys)(Dpa)(Gly)(d-Gln)(Amf)(d-Nle) |
| 76 | (Dpa)(Abu)(d-Asn)(d-Arg)(d-Nle)(d-Thr) |
| 77 | (d-Glu)(B)(Dpa)(Dab)(d-Nle)(Gly) |
| 78 | (d-Thr)(d-Arg)(Dpa)(d-Gln)(B)(d-Phe) |
| 79 | (d-Thr)(d-Lys)(Dpa)(Abu)(d-Lys)(Dpa) |
| 80 | (Dab)(Dpa)(d-Asn)(Dpa)(Hle)(B) |
| 81 | (d-Glu)(Hle)(d-Thr)(Nap)(d-Gln)(Nap) |
| 82 | (Abu)(d-Asp)(Dpa)(Dab)(d-Nle)(Gly) |
| 83 | (Hle)(d-Lys)(Dpa)(Gly)(d-Thr)(d-Pro) |
| 84 | (d-Gln)(Nap)(d-Nle)(Pip)(Amf)(Hle) |
| 85 | (d-Asn)(Gly)(Dpa)(d-Thr)(Nap)(Gly) |
| 86 | (Gly)(d-Asn)(Dpa)(d-Thr)(Nap)(Gly) |
| 87 | (d-Ser)(d-Phe)(d-Glu)(d-Asp)(Gly)(Gly) |
| 88 | (d-Phe)(d-Phe)(d-Thr)(d-Phe)(d-Thr)(Abu) |
| 89 | (d-Pro)(Dpa)(Hle)(Hle)(Abu)(d-Pro) |
| 90 | (d-Asn)(Abu)(d-Glu)(d-Lys)(d-Nle)(Nap) |
| 91 | (d-Phe)(Dpa)(Dpa)(Pip)(d-Phe)(Dpa) |
| 92 | (d-Lys)(Hle)(Dpa)(Gly)(d-Thr)(d-Pro) |
| 93 | (Hle)(d-Thr)(d-Glu)(Hle)(d-Pro)(Dpa) |
| 94 | (d-Thr)(Dab)(d-Asp)(d-Nle)(d-Lys)(Nap) |
| 95 | (d-Glu)(d-Phe)(Nap)(d-Asn)(Nap)(Amf) |
| 96 | (B)(d-Pro)(d-Pro)(Nap)(Nap)(d-Asn) |
| 97 | (d-Lys)(d-Thr)(Dpa)(Abu)(d-Lys)(Dpa) |
| 98 | (Gly)(d-Lys)(d-Glu)(Hle)(d-Phe)(Amf) |
| 99 | (d-Lys)(Dab)(d-Thr)(Dpa)(d-Gln)(d-Nle) |
| 100 | (d-Phe)(d-Nle)(B)(Abu)(Dab)(Nap) |
| 101 | (Dpa)(Hle)(B)(Gly)(Pip)(d-Phe) |
| 102 | (Dpa)(d-Gln)(Abu)(Abu)(Pip)(Dpa) |
| 103 | (d-Lys)(Gly)(d-Glu)(Hle)(d-Phe)(Amf) |
| 104 | (d-Phe)(d-Phe)(d-Nle)(d-Glu)(Nap)(Hle) |
| 105 | (d-Ser)(d-Asn)(d-Phe)(d-Nle)(Gly)(Dpa) |
| 106 | (d-Phe)(d-Phe)(d-Asn)(Nap)(B)(Nap) |
| 107 | (d-Asp)(Abu)(Dpa)(Dab)(d-Nle)(Gly) |
| 108 | (d-Pro)(Nap)(d-Nle)(d-Glu)(Nap)(Hle) |
| 109 | (B)(B)(d-Lys)(d-Asp)(d-Nle)(d-Phe) |
| 110 | (d-Phe)(Amf)(d-Asp)(d-Asp)(Dpa)(d-Phe) |
| 111 | (d-Nle)(d-Asn)(d-Thr)(d-Gln)(Nap)(d-Glu) |
| 112 | (d-Phe)(d-Ser)(d-Glu)(d-Asp)(Gly)(Gly) |
| 113 | (Amf)(B)(d-Gln)(Hle)(Dpa)(Gly) |
| 114 | (Gly)(Dpa)(Dpa)(Gly)(Gly)(Dab) |
| 115 | (Abu)(Dpa)(d-Phe)(Hle)(d-Ser)(Pip) |
| 70 | (d-Pro)(d-Ser)(d-Phe)(Dpa)(d-Glu)(B) |
| 116 | (d-Thr)(Pip)(d-Thr)(d-Gln)(Nap)(d-Glu) |
| 117 | (d-Arg)(d-Lys)(d-Nle)(Hle)(d-Lys)(d-Nle) |
| 118 | (Gly)(Gly)(Dab)(d-Nle)(Hle)(d-Nle) |
| 119 | (d-Gln)(d-His)(d-Asn)(Dpa)(Hle)(B) |
| 120 | (Dpa)(B)(d-Nle)(d-Glu)(Nap)(Hle) |
| 121 | (d-Glu)(d-Phe)(Gly)(Gly)(Nap)(Amf) |
| 122 | (d-Phe)(d-Phe)(d-Asn)(d-Arg)(Abu)(Dpa) |
| 123 | (d-Asn)(d-Glu)(d-His)(d-Thr)(Nap)(Gly) |
| 124 | (d-Arg)(d-Thr)(Dpa)(d-Gln)(B)(d-Phe) |
| 125 | (d-His)(d-Gln)(d-Asn)(Dpa)(Hle)(B) |
| 126 | (Abu)(d-Phe)(d-Glu)(d-Glu)(B)(d-Asp) |
| 127 | (B)(Abu)(B)(Dpa)(Hle)(Gly) |
| 128 | (d-Asn)(d-His)(d-Asn)(Dpa)(Hle)(B) |
| 129 | (Abu)(d-Pro)(d-Pro)(B)(d-Ser)(d-Glu) |
| 130 | (d-Thr)(d-Arg)(Dpa)(Abu)(d-Asn)(d-Phe) |
| 131 | (d-Pro)(Abu)(d-Pro)(Gly)(Gly)(d-His) |
| 132 | (Pip)(Nap)(Hle)(d-Asp)(d-Thr)(Pip) |
| 133 | (B)(Gly)(d-Pro)(Abu)(Dpa)(Nap) |
| 134 | (d-Asn)(d-His)(d-Glu)(d-Thr)(Nap)(Gly) |
| 135 | (d-Nle)(B)(Abu)(d-Ser)(d-Asn)(d-Asp) |
| 136 | (Hle)(d-Lys)(d-Nle)(d-Glu)(d-Ser)(d-Glu) |
| 137 | (Amf)(d-Thr)(d-Lys)(d-Nle)(d-Gln)(Dpa) |
| 138 | (Dpa)(B)(Dpa)(d-Phe)(B)(Gly) |
| 139 | (B)(d-Ser)(d-Ser)(d-Ser)(Hle)(Hle) |
| 140 | (d-Glu)(d-Ser)(ox-His)(d-Ser)(Dpa)(Gly) |
| 141 | (Dab)(Gly)(ox-His)(d-Glu)(Nap)(Hle) |
| 142 | (B)(Abu)(d-Nle)(d-Ser)(d-Gln)(d-Gln) |
| 143 | (Abu)(d-Nle)(B)(d-Ser)(d-Asn)(d-Asp) |
| 144 | (Dpa)(d-Glu)(d-Pro)(B)(d-His)(Gly) |
| 145 | (Gly)(Gly)(Hle)(d-Arg)(d-Nle)(d-Phe) |
| 146 | (Dpa)(d-Phe)(d-Lys)(d-Phe)(d-Asp)(Dab) |
| 147 | (d-Glu)(d-Asn)(d-His)(d-Thr)(Nap)(Gly) |
| 148 | (d-Nle)(d-Gln)(d-Arg)(d-Glu)(d-Gln)(Nap) |
| 149 | (d-Nle)(d-Glu)(d-Phe)(d-Thr)(d-Asp) |
| | (d-Thr) |
| 150 | (d-Glu)(Hle)(d-Thr)(Gly)(B)(Nap) |
| 151 | (Pip)(d-Ser)(d-Pro)(d-Glu)(Nap)(Hle) |
| 152 | (d-Gln)(d-Pro)(Abu)(d-Glu)(Nap)(Hle) |
| 153 | (d-Ser)(d-Arg)(d-Asn)(Dpa)(Abu)(Gly) |
| 154 | (Dpa)(d-Thr)(d-Asn)(d-Glu)(d-Thr)(d-Ser) |
| 155 | (d-Asn)(Hle)(d-Pro)(Nap)(B)(d-Ser) |
| 156 | (Pip)(Gly)(Gly)(d-Phe)(Hle)(Dpa) |
| 157 | (Abu)(d-Pro)(B)(Gly)(Dpa)(Nap) |
| 158 | (Gly)(Dab)(d-Ser)(d-Nle)(d-Glu)(Dpa) |
| 159 | (Abu)(d-Gln)(Abu)(d-Nle)(Hle)(d-Gln) |
| 160 | (Amf)(d-Asn)(Abu)(Hle)(Dpa)(Gly) |
| 161 | (d-Glu)(ox-His)(d-Ser)(d-Ser)(Dpa)(Gly) |
| 162 | (d-Arg)(d-Asn)(d-Glu)(d-Ser)(d-His) |
| | (d-Phe) |
| 163 | (d-Gln)(d-Gln)(d-Ser)(d-Pro)(d-Nle)(Gly) |
| 164 | (d-Asn)(Gly)(d-His)(d-Asn)(B)(Dpa) |
| 165 | (B)(Dpa)(d-Nle)(d-Glu)(Nap)(Hle) |
| 166 | (B)(Gly)(B)(Hle)(Dpa)(Gly) |
| 167 | (Pip)(d-His)(d-Asp)(d-Phe)(d-Arg)(d-Asp) |
| 168 | (Dpa)(d-Gln)(d-Nle)(Gly)(Pip)(Dpa) |
| 169 | (Dab)(Dpa)(d-Asn)(Dpa)(d-Nle)(Abu) |
| 170 | (d-Pro)(Gly)(d-Asn)(Hle)(Gly)(d-Asn) |
| 171 | (Pip)(Gly)(d-His)(Nap)(Amf) (Dab) |
| 172 | (Gly)(Gly)(ox-His)(Nap)(d-Asp)(Dpa) |
| 173 | (d-Thr)(B)(Pip)(d-Nle)(Hle)(d-Gln) |
| 174 | (d-Asn)(d-His)(Gly)(B)(d-Asn)(Dpa) |
| 175 | (d-Thr)(d-Phe)(d-Asp)(Dpa)(Hle)(B) |
| 176 | (d-Glu)(d-His)(d-Pro)(Dpa)(Hle)(B) |
| 177 | (Abu)(B)(B)(Dpa)(Hle)(Gly) |
| 178 | (d-Lys)(d-Pro)(d-Nle)(d-Asp)(d-Phe) |
| | (d-Nle) |
| 179 | (Hle)(d-Pro)(d-Nle)(d-Ser)(Abu)(B) |
| 180 | (Dpa)(d-Thr)(B)(Abu)(Dpa)(d-Asn) |
| 181 | (Abu)(Dpa)(d-Phe)(Hle)(d-Ser)(Dpa) |
| 182 | (d-His)(d-Pro)(d-Glu)(Dpa)(Hle)(B) |
| 183 | (d-Nle)(Abu)(d-Thr)(d-Nle)(Gly)(d-Asn) |
| 184 | (B)(Nap)(d-Phe)(Dpa)(Amf)(d-Glu) |
| 185 | (Dpa)(Gly)(d-Nle)(d-His)(Dpa)(Gly) |
| 186 | (Pip)(d-Ser)(Abu)(d-Nle)(Hle)(d-Gln) |
| 187 | (d-Asp)(Abu)(d-Ser)(d-Nle)(Dpa)(Dpa) |
| 188 | (d-Gln)(Abu)(d-Pro)(d-Glu)(Nap)(Hle) |
| 189 | (Amf)(Abu)(B)(Abu)(Hle)(Dpa) |
| 190 | (d-Nle)(Abu)(d-Nle)(Hle)(B)(Gly) |
| 191 | (d-His)(Gly)(B)(d-Nle)(Dpa)(Gly) |
| 192 | (Gly)(B)(d-His)(d-Nle)(Dpa)(Gly) |
| 193 | (Pip)(d-His)(Gly)(Nap)(Amf)(Dab) |
| 194 | (Dpa)(Hle)(Gly)(B)(Pip)(d-Phe) |
| 195 | (d-Nle)(d-Pro)(d-Nle)(d-Glu)(Nap)(Hle) |
| 196 | (Pip)(B)(d-Nle)(Gly)(Hle)(d-His) |
| 197 | (Amf)(d-Ser)(Gly)(Nap)(Amf)(Dab) |
| 198 | (Hle)(d-Pro)(B)(Gly)(Gly)(Nap) |
| 199 | (d-Arg)(d-Lys)(d-Nle)(Hle)(d-Nle)(d-Lys) |
| 200 | (d-Nle)(Gly)(B)(d-Arg)(d-Nle)(d-Phe) |
| 201 | (Gly)(Gly)(Abu)(Hle)(Dpa)(Gly) |
| 202 | (Gly)(Gly)(d-His)(d-Phe)(Hle)(d-Ser) |
| 203 | (d-Asn)(d-Phe)(Gly)(d-Glu)(d-Pro)(Dpa) |
| 204 | (d-Asp)(Nap)(Gly)(d-Ser)(Dpa)(Gly) |
| 205 | (d-Thr)(ox-His)(d-Asp)(d-Gln)(d-Pro)(Dpa) |
| 206 | (d-Asn)(Nap)(Gly)(d-Glu)(d-Pro)(Dpa) |
| 207 | (d-Nle)(Abu)(B)(d-Ser)(d-Asn)(d-Asp) |
| 208 | (d-Thr)(Gba)(d-Asn)(Dpa)(Abu)(Gly) |
| 209 | (d-Thr)(Pip)(B)(d-Nle)(Hle)(d-Gln) |
| 210 | (d-Asp)(Abu)(d-Ser)(d-Nle)(Nap)(Dpa) |
| 211 | (B)(Gly)(d-His)(d-Nle)(Dpa)(Gly) |
| 212 | (Nap)(Gly)(d-Asp)(d-Gln)(d-Pro)(Dpa) |
| 213 | (d-Lys)(B)(d-Phe)(B)(Hle)(Dpa) |
| 214 | (Gly)(d-Phe)(d-Ser)(d-Nle)(d-Nle)(d-Phe) |
| 215 | (d-Lys)(B)(ox-His)(d-Gln)(Dpa)(d-Asn) |
| 216 | (Gly)(Gly)(d-Asn)(d-Phe)(d-Phe)(d-Gln) |
| 217 | (d-Asp)(Pip)(d-His)(d-Phe)(d-Arg)(d-Asp) |
| 218 | (ox-His)(d-Gln)(d-Pro)(d-Phe)(d-Arg) |
| | (d-Asp) |
| 219 | (Gly)(d-Ser)(Amf)(Nap)(Amf)(Dab) |
| 220 | (Pip)(d-Asp)(d-His)(d-Phe)(d-Arg)(d-Asp) |
| 221 | (Gly)(ox-His)(Gly)(Nap)(d-Asp)(Dpa) |
| 222 | (B)(Dab)(d-His)(d-Phe)(Hle)(d-Ser) |
| 223 | (Nap)(d-Glu)(d-Gln)(d-Pro)(Pip)(d-Phe) |
| 224 | (Abu)(B)(d-Nle)(d-Ser)(d-Gln)(d-Gln) |
| 225 | (Abu)(Dpa)(d-Asn)(d-Arg)(d-Nle)(d-Thr) |
| 226 | (Dpa)(d-Thr)(Abu)(B)(Dpa)(d-Asn) |
| 227 | (d-His)(Gly)(Gly)(d-Phe)(Hle)(d-Ser) |
| 228 | (d-Pro)(d-Nle)(d-Asn)(d-Asp)(d-Pro)(Hle) |
| 229 | (Gly)(d-His)(Gly)(Abu)(d-Lys)(Dpa) |
| 230 | (Nap)(d-Glu)(ox-His)(B)(Hle)(d-Phe) |
| 231 | (d-Asp)(d-Pro)(d-Asp)(d-His)(d-Asp)(Dpa) |
| 232 | (Pip)(Pip)(d-Glu)(d-Pro)(d-Thr)(Pip) |
| 233 | (Dpa)(d-Glu)(d-Asn)(d-Gln)(d-Pro)(Dpa) |
| 234 | (d-Asn)(Nap)(d-Asp)(B)(d-Pro)(Dpa) |
| 235 | (Abu)(B)(d-Asn)(Hle)(d-His)(d-Asn) |
| 236 | (B)(d-Ser)(d-Thr)(Dab)(Dpa)(d-Asn) |
| 237 | (d-Pro)(d-Nle)(d-Glu)(d-Pro)(d-Thr)(Pip) |
| 238 | (d-Asn)(d-His)(B)(Dpa)(d-Phe)(d-Gln) |
| 239 | (Dpa)(Abu)(d-Gln)(Abu)(Pip)(Dpa) |
| 240 | (Abu)(d-Pro)(Gly)(B)(Dpa)(Nap) |
| 241 | (d-Ser)(ox-His)(d-Glu)(d-Ser)(Dpa)(Gly) |
| 242 | (d-Gln)(d-Gln)(d-Nle)(d-Ser)(d-Pro)(Gly) |
| 243 | (d-Ser)(Amf)(d-Ser)(Gly)(Pip)(Dab) |
| 244 | (d-Asn)(d-Pro)(B)(Gly)(Gly)(Dpa) |
| 245 | (Abu)(B)(d-Nle)(d-Ser)(d-Asn)(d-Asp) |
| 246 | (d-Asn)(Hle)(d-Pro)(B)(Nap)(d-Ser) |
| 247 | (ox-His)(Gly)(Gly)(Nap)(d-Asp)(Dpa) |
| 248 | (d-Nle)(Abu)(d-Ser)(B)(d-Asn)(d-Asp) |
| 249 | (Pip)(Gly)(d-Ser)(Hle)(d-His)(d-Asn) |
| 250 | (Dpa)(d-Lys)(B)(Gly)(Gly)(d-Pro) |
| 251 | (B)(d-Nle)(d-Asp)(Gly)(Pip)(Pip) |
| 252 | (d-Lys)(B)(Amf)(d-Thr)(d-Thr)(Dab) |
| 253 | (d-Pro)(d-Glu)(Amf)(Gly)(d-Ser)(B) |
| 254 | (Dpa)(B)(Gly)(Dpa)(d-Phe)(d-Gln) |
| 255 | (Abu)(d-Glu)(d-His)(Dpa)(d-Phe)(d-Gln) |
| 256 | (Abu)(B)(d-Ser)(d-Phe)(d-Phe)(d-Gln) |
| 257 | (Gly)(Gly)(Dab)(ox-His)(Nap)(Hle) |
| 258 | (d-Phe)(d-Asp)(Gly)(d-Gln)(d-Pro)(Dpa) |
| 259 | (Gly)(Pip)(d-Ser)(Hle)(d-His)(d-Asn) |
| 260 | (d-Lys)(Abu)(Amf)(d-Ser)(d-Thr)(Dab) |
| 261 | (Abu)(Gly)(Gly)(Hle)(Dpa)(Gly) |
| 262 | (d-Glu)(d-Ser)(B)(d-Nle)(Dpa)(Dpa) |
| 263 | (Abu)(d-Pro)(d-Gln)(d-Glu)(Nap)(Hle) |
| 264 | (d-Ser)(B)(Gly)(Abu)(Gly)(Hle) |
| 265 | (d-Phe)(d-Pro)(B)(Hle)(d-Arg)(d-Asp) |
| 266 | (B)(Dab)(ox-His)(Amf)(Gly)(Nap) |
| 267 | (Pip)(d-Nle)(d-Glu)(d-Gln)(d-His)(d-Asn) |
| 268 | (d-Asp)(Abu)(d-His)(Dpa)(Hle)(B) |
| 269 | (Gly)(Gly)(d-Nle)(B)(d-His)(Dpa) |
| 270 | (B)(d-Nle)(d-Asp)(Pip)(Gly)(Pip) |
| 271 | (Gly)(Gly)(d-His)(Abu)(d-Lys)(Dpa) |
| 272 | (Nap)(d-Glu)(Pip)(d-Pro)(d-Gln)(d-Phe) |
| 273 | (d-Gln)(B)(d-Glu)(d-Lys)(d-Nle)(Nap) |
| 274 | (d-Asn)(B)(d-Asp)(Nap)(d-Pro)(Dpa) |
| 275 | (d-Nle)(d-Lys)(Abu)(d-Asn)(d-Glu)(Amf) |
| 276 | (B)(ox-His)(Dab)(d-Glu)(d-Thr)(d-Ser) |
| 277 | (d-Asn)(Pip)(d-Gln)(d-Ser)(Hle)(d-Ser) |
| 278 | (ox-His)(d-Pro)(d-Ser)(Dpa)(Hle)(B) |
| 279 | (d-Thr)(Gly)(d-Phe)(d-Pro)(Gly)(Dpa) |
| 280 | (d-Asn)(Gly)(d-Glu)(Nap)(d-Pro)(Dpa) |
| 281 | (d-Asn)(d-Asp)(Nap)(B)(d-Pro)(Dpa) |
| 282 | (d-Nle)(B)(Gly)(d-Arg)(d-Nle)(d-Phe) |
| 283 | (d-Thr)(d-Pro)(Dab)(d-Nle)(Hle)(d-Gln) |
| 284 | (d-Asn)(Nap)(d-Glu)(Gly)(d-Pro)(Dpa) |
| 285 | (d-Asn)(Gly)(Nap)(d-Glu)(d-Pro)(Dpa) |
| 286 | (Abu)(Dpa)(d-Pro)(Gly)(Abu)(B) |
| 287 | (d-Lys)(Dpa)(Gly)(B)(Amf)(d-Nle) |
| 288 | (d-His)(Gly)(Amf)(d-Lys)(d-Ser)(d-Asp) |
| 289 | (d-Gln)(Dpa)(d-Asn)(d-Nle)(d-His)(Abu) |
| 290 | (Dpa)(d-Gln)(Pip)(d-Pro)(Hle)(d-Thr) |
| 291 | (ox-His)(Gly)(Gly)(Gly)(Nap)(Hle) |
| 292 | (d-Gln)(Gba)(d-Glu)(d-Ser)(d-His)(d-Phe) |
| 293 | (Dpa)(Pip)(d-Ser)(Abu)(Pip)(Dpa) |
| 294 | (d-Asp)(d-His)(Pip)(d-Phe)(d-Arg)(d-Asp) |
| 295 | (Dpa)(B)(Dpa)(d-Phe)(Gly)(B) |
| 296 | (B)(d-Gln)(d-Glu)(d-Lys)(d-Nle)(Nap) |
| 297 | (Pip)(Gly)(Gly)(Hle)(Hle)(d-His) |
| 298 | (Abu)(B)(Amf)(Abu)(Hle)(Dpa) |
| 299 | (d-Asn)(d-Pro)(d-Pro)(d-Phe)(Hle)(d-Ser) |
| 300 | (d-His)(B)(Dab)(d-Phe)(Hle)(d-Ser) |
| 301 | (d-Glu)(d-His)(Abu)(Dpa)(d-Phe)(d-Gln) |
| 302 | (d-Glu)(Hle)(d-Thr)(d-Gln)(Nap)(Nap) |
| 303 | (Hle)(d-Pro)(d-Glu)(d-Pro)(d-Thr)(Pip) |
| 304 | (Nap)(d-Asp)(Gly)(d-Ser)(Dpa)(Gly) |
| 305 | (d-Glu)(Hle)(d-Thr)(Nap)(B)(Gly) |
| 306 | (d-Asn)(Abu)(B)(Hle)(d-His)(d-Asn) |
| 307 | (d-Nle)(Hle)(d-His)(d-Asp)(d-His)(Amf) |
| 308 | (Dpa)(Amf)(d-Ser)(d-Phe)(Gly)(B) |
| 309 | (d-Thr)(Dpa)(Abu)(B)(Dpa)(d-Asn) |
| 310 | (Hle)(Gly)(Dpa)(d-Phe)(d-Ser)(B) |
| 311 | (Nap)(Dab)(B)(d-Ser)(Hle)(d-Ser) |
| 312 | (Abu)(Dpa)(d-Pro)(Gly)(B)(Abu) |
| 313 | (Dpa)(d-Ser)(Pip)(Abu)(Pip)(Dpa) |
| 314 | (d-Ser)(d-Glu)(d-Nle)(Gly)(Gly)(d-Gln) |
| 315 | (d-Lys)(d-Arg)(d-Asp)(Gly)(d-Pro)(Gly) |
| 316 | (d-Gln)(d-Pro)(Pip)(B)(d-Asn)(d-Pro) |
| 317 | (Dpa)(Pip)(Gly)(Gly)(Hle)(Dab) |
| 318 | (Abu)(d-Pro)(Gly)(d-Pro)(Gly)(d-His) |
| 319 | (B)(Abu)(ox-His)(Abu)(d-Nle)(d-Phe) |
| 320 | (Dpa)(Pip)(B)(d-Thr)(Pip)(Dpa) |
| 321 | (d-Pro)(Abu)(Gly)(d-Pro)(Gly)(d-His) |
| 322 | (d-Arg)(d-Gln)(d-Asp)(d-Ser)(d-Phe) |
| | (d-Gln) |
| 323 | (Dpa)(Abu)(ox-His)(d-Nle)(Hle)(d-Thr) |
| 324 | (d-Pro)(Hle)(d-Nle)(d-Ser)(Abu)(B) |
| 325 | (B)(d-Nle)(Pip)(d-Asp)(Gly)(Pip) |
| 326 | (B)(d-His)(Dab)(d-Phe)(Hle)(d-Ser) |
| 327 | (d-Pro)(B)(d-Pro)(Nap)(Nap)(d-Asn) |
| 328 | (Gly)(Dab)(Gly)(ox-His)(Nap)(Hle) |
| 329 | (d-Pro)(d-Nle)(d-Asp)(B)(Gly)(Hle) |
| 330 | (d-Lys)(d-Ser)(d-Thr)(Dab)(Dab)(Nap) |
| 331 | (d-Phe)(Abu)(Gly)(d-Ser)(d-Phe)(Nap) |
| 332 | (Abu)(B)(d-Pro)(d-Pro)(Abu)(Gly) |
| 333 | (d-Asp)(Gly)(d-Phe)(d-Gln)(d-Pro)(Dpa) |
| 334 | (Abu)(Hle)(Gly)(d-Ser)(d-Asn)(d-Asp) |
| 335 | (d-Ser)(d-Phe)(Gly)(d-Nle)(d-Nle)(d-Phe) |
| 336 | (Hle)(Gly)(Dpa)(d-Phe)(d-Thr)(Gly) |
| 337 | (Abu)(d-Pro)(B)(d-Pro)(d-Ser)(d-Glu) |
| 338 | (d-Asp)(d-Phe)(Gly)(d-Gln)(d-Pro)(Dpa) |
| 339 | (Gly)(Gly)(Gly)(Gly)(d-Nle)(d-Asp) |
| 340 | (d-Pro)(d-Glu)(Amf)(d-Thr)(Gly)(Gly) |
| 341 | (Hle)(Dpa)(Gly)(B)(Pip)(d-Phe) |
| 342 | (B)(Nap)(d-Phe)(Abu)(Hle)(Hle) |
| 343 | (Hle)(d-Pro)(d-Glu)(Pip)(d-Pro)(d-Thr) |
| 344 | (d-Pro)(Hle)(d-Nle)(d-Ser)(B)(Abu) |
| 345 | (Nap)(d-Pro)(Abu)(d-Nle)(d-Nle)(Amf) |
| 346 | (Pip)(B)(d-Thr)(d-Nle)(Hle)(d-Gln) |
| 347 | (d-Phe)(Dpa)(Dpa)(d-Phe)(Pip)(Dpa) |
| 348 | (d-Lys)(d-Thr)(Amf)(d-Ser)(d-His)(Amf) |
| 349 | (Pip)(d-Pro)(d-Ser)(Pip)(Gly)(Gly) |
| 350 | (d-Lys)(d-Lys)(B)(Gba)(d-Thr)(Nap) |
| 351 | (d-Arg)(d-Thr)(Dpa)(Abu)(d-Asn)(d-Phe) |
| 352 | (d-Nle)(Abu)(B)(Gly)(Dpa)(Gly) |
| 353 | (d-Pro)(B)(d-Phe)(Abu)(Dpa)(Dpa) |
| 354 | (d-Asp)(d-Asp)(d-Lys)(d-Nle)(Dpa)(Dpa) |
| 355 | (d-Nle)(d-Thr)(d-Ser)(d-His)(d-Nle)(Nap) |
| 356 | (d-Asp)(d-Phe)(d-Thr)(d-Asp)(Gly)(Gly) |
| 357 | (Pip)(Dpa)(Gly)(Gly)(Hle)(Dab) |
| 358 | (d-Phe)(Gly)(d-Ser)(d-Nle)(d-Nle)(d-Phe) |
| 359 | (Hle)(d-Lys)(Dpa)(Gly)(d-Pro)(d-Thr) |
| 360 | (Gly)(Gly)(d-Gln)(B)(Amf)(d-Asp) |
| 361 | (B)(Gly)(Abu)(d-Glu)(Gly)(d-Phe) |
| 362 | (B)(d-Nle)(d-Thr)(d-Thr)(Abu)(d-His) |
| 363 | (Gly)(Dab)(d-Lys)(d-Glu)(d-Glu)(Dpa) |
| 364 | (Gly)(Dpa)(d-Pro)(Abu)(d-Pro)(d-Asn) |
| 365 | (d-Nle)(Abu)(B)(d-Ser)(d-Gln)(d-Gln) |
| 366 | (Dab)(B)(ox-His)(Amf)(Gly)(Nap) |
| 367 | (Hle)(d-Glu)(d-Thr)(Nap)(d-Gln)(Nap) |
| 368 | (d-Ser)(d-Glu)(d-Nle)(d-Asn)(B)(Gly) |
| 369 | (d-Lys)(d-Phe)(Gly)(Abu)(Hle)(Dpa) |
| 370 | (d-Nle)(Gly)(d-Phe)(Dab)(Hle)(Dpa) |
| 371 | (ox-His)(B)(Hle)(Dpa)(Hle)(d-Thr) |
| 372 | (d-Pro)(Pip)(d-Gln)(Dpa)(Hle)(d-Thr) |
| 373 | (Gly)(B)(Dpa)(d-Phe)(Gly)(B) |
| 374 | (Gly)(Gly)(B)(d-Gln)(Amf)(d-Asp) |
| 375 | (Amf)(B)(Abu)(Abu)(Hle)(Dpa) |
| 376 | (d-Ser)(B)(d-Ser)(d-Ser)(Hle)(Hle) |
| 377 | (B)(d-Lys)(Dab)(ox-His)(Gly)(d-Pro) |
| 378 | (d-Lys)(Abu)(d-Ser)(Amf)(d-Thr)(Dab) |
| 379 | (Abu)(B)(d-Ser)(Dpa)(Gly)(d-Ser) |
| 380 | (d-Ser)(Amf)(Gly)(d-Ser)(Pip)(Dab) |
| 381 | (Hle)(Nle)(d-Glu)(d-Ser)(d-Thr)(d-Asn) |
| 382 | (B)(B)(d-Asp)(Gly)(Dpa)(d-Asn) |
| 383 | (Gly)(d-Ser)(Hle)(d-Phe)(d-Glu)(d-Gln) |
| 384 | (d-Pro)(d-Thr)(d-Asp)(Gly)(d-Thr)(Gly) |
| 385 | (d-Pro)(d-Thr)(Hle)(d-Ser)(d-Asp)(Nap) |
| 386 | (d-Asn)(d-Ser)(d-Ser)(Pip)(Hle)(Nle) |
| 387 | (Gly)(d-Pro)(d-Glu)(d-Glu)(Gly)(Nap) |
| 388 | (B)(d-Asn)(Dpa)(d-Ser)(Hle)(Dab) |
| 389 | (Gly)(d-Asp)(d-Asp)(Dpa)(d-Asn)(d-Ser) |
| 390 | (d-Asp)(d-Thr)(Hle)(d-Pro)(d-Gln)(Hle) |
| 391 | (d-Pro)(d-Thr)(d-Asp)(d-Asp)(d-Asn)(Hle) |
| 392 | (d-Thr)(d-Pro)(Hle)(d-Ser)(d-Asp)(Nap) |
| 393 | (Gly)(d-Arg)(d-Glu)(d-Thr)(Hle)(d-Asn) |
| 394 | (B)(Hle)(d-Glu)(d-Gln)(d-Glu)(Amf) |
| 395 | (Gly)(d-Phe)(d-Pro)(Pip)(Nie)(Gly) |
| 396 | (B)(d-Thr)(d-Gln)(Hle)(d-Glu)(d-Asp) |
| 397 | (d-Phe)(Hle)(d-Glu)(d-Ser)(B)(d-Thr) |
| 398 | (d-Asn)(Gly)(Hle)(B)(Ne)(d-Asp) |
| 399 | (B)(d-Arg)(Nle)(d-Arg)(d-Glu)(d-Gln) |
| 400 | (Gly)(Gly)(B)(d-Phe)(d-Asn)(d-Thr) |
| 401 | (Nle)(d-Arg)(Nle)(d-Gln)(B)(d-Asp) |
| 402 | (d-Asp)(d-Asn)(d-Gln)(Abu)(d-Thr)(d-Phe) |
| 403 | (Nle)(Hle)(d-Glu)(d-Ser)(d-Thr)(d-Asn) |
| 404 | (d-Asn)(d-Ser)(Amf)(Dab)(d-Phe)(Gly) |
| 405 | (d-Gln)(B)(d-Phe)(d-Arg)(d-Asn)(Amf) |
| 406 | (d-Asn)(d-Lys)(d-Thr)(Gly)(Hle)(Abu) |
| 407 | (B)(B)(B)(Hle)(d-His)(Hle) |
| 408 | (Gly)(B)(B)(d-Glu)(Nle)(Dpa) |
| 409 | (d-Pro)(d-Thr)(Dpa)(Abu)(B)(d-Lys) |
| 410 | (B)(Nie)(Dab)(d-Phe)(Gly)(d-Thr) |
| 411 | (B)(Nie)(Dab)(d-Lys)(B)(d-Phe) |
| 412 | (d-Gln)(d-Ser)(d-Lys)(Hle)(d-Phe)(Dab) |
| 413 | (d-Arg)(d-Phe)(d-Lys)(d-Asp)(Hle)(Gly) |
| 414 | (Abu)(Gly)(d-Asp)(Gly)(Dpa)(d-Asn) |
| 415 | (B)(d-Thr)(Dpa)(d-Asn)(d-Gln)(Nap) |
| 416 | (Abu)(B)(d-Glu)(Pip)(d-Arg)(Nle) |
| 417 | (Gly)(d-Asp)(d-Arg)(d-Ser)(Dab)(d-Phe) |
| 418 | (B)(d-Arg)(Hle)(d-Phe)(Gly)(d-Arg) |
| 419 | (d-Arg)(d-Asp)(Hle)(d-Ser)(Pip)(d-Gln) |
| 420 | (Gly)(Gly)(d-Ser)(Pip)(Hle)(Nle) |
| 421 | (d-Asn)(d-Asp)(d-Gln)(Abu)(d-Thr)(d-Phe) |
| 422 | (B)(Nie)(B)(B)(d-Thr)(Hle) |
| 423 | (Gly)(Dpa)(Gly)(d-Asp)(Gly)(B) |
| 424 | (Gly)(Gly)(d-Asn)(d-Pro)(Hle)(Dab) |
| 425 | (d-Phe)(d-Thr)(d-Lys)(d-Asn)(d-Thr) |
| | (d-Phe) |
| 426 | (d-Asn)(d-Asp)(B)(d-Thr)(Gly)(d-Phe) |
| 427 | (d-Asn)(Gly)(d-Gln)(d-Ser)(Nap)(d-Asp) |
| 428 | (Gly)(Abu)(d-Asp)(Gly)(Dpa)(d-Asn) |
| 429 | (B)(Abu)(d-Asp)(Hle)(Amf)(Nle) |
| 430 | (d-Asn)(Gly)(d-Glu)(Nap)(d-Asn)(d-Asp) |
| 431 | (B)(Gly)(Gly)(d-Gln)(Dpa)(d-Phe) |
| 432 | (B)(Abu)(d-Glu)(Pip)(d-Arg)(Nle) |
| 433 | (B)(Abu)(Nle)(d-Arg)(d-Pro)(d-Pro) |
| 434 | (Gly)(Gly)(Nle)(d-Thr)(d-Asp)(Hle) |
| 435 | (B)(d-Asn)(Gly)(B)(Dpa)(d-Phe) |
| 436 | (Gly)(Gly)(Abu)(d-Glu)(Nle)(Dpa) |
| 437 | (d-Asn)(d-Gln)(d-Glu)(d-Gln)(d-Ser) |
| | (d-Phe) |
| 438 | (Gly)(Dpa)(Gly)(B)(d-His)(B) |
| 439 | (Gly)(Gly)(d-Thr)(Abu)(d-Gln)(Nap) |
| 440 | (Dab)(d-Thr)(d-Asp)(Gly)(d-Thr)(Gly) |
| 441 | (d-Asn)(Gly)(d-Asp)(d-Asp)(d-Pro)(Nap) |
| 442 | (d-Pro)(d-Thr)(d-Phe)(d-Glu)(Gly)(B) |
| 443 | (d-Lys)(d-Asn)(d-Thr)(Gly)(Hle)(Abu) |
| 444 | (d-Thr)(d-Pro)(Dpa)(Abu)(B)(d-Lys) |
| 445 | (B)(Ne)(Amf)(B)(Gly)(d-Thr) |
| 446 | (Nle)(Nle)(d-Arg)(d-Glu)(Gly)(B) |
| 447 | (d-Pro)(d-Ser)(d-Pro)(d-Lys)(Pip)(d-Gln) |
| 448 | (d-Asn)(d-Lys)(B)(d-Phe)(d-Asp)(d-Asp) |
| 449 | (Gly)(Gly)(Dpa)(Abu)(d-Lys)(d-Ser) |
| 450 | (B)(Abu)(Nle)(d-Pro)(d-Pro)(d-Arg) |
| 451 | (Gly)(d-Gln)(Gba)(d-Ser)(d-Ser)(d-Asn) |
| 452 | (d-Ser)(d-Pro)(d-Pro)(d-Glu)(d-Glu)(Dab) |
| 453 | (Gly)(Gly)(Nle)(d-Asn)(B)(Hle) |
| 454 | (Gly)(Gly)(d-Gln)(d-Ser)(Nap)(d-Asp) |
| 455 | (B)(Gly)(B)(Hle)(d-Phe)(d-Ser) |
| 456 | (d-Asn)(Nle)(d-Pro)(d-Asn)(d-Ser)(Nap) |
| 457 | (Gly)(d-Ser)(Hle)(d-Asp)(Gly)(Gly) |
| 458 | (Abu)(d-Pro)(Nle)(d-Pro)(d-Pro)(Gly) |
| 459 | (Abu)(Gly)(Gly)(Gly)(d-Arg)(Dpa) |
| 460 | (d-Gln)(Dab)(d-Asp)(Pip)(d-Gln)(Gly) |
| 461 | (Abu)(B)(d-Lys)(B)(d-Thr)(d-Ser) |
| 462 | (Gly)(Nle)(d-Glu)(d-His)(B)(Dab) |
| 463 | (Abu)(d-Ser)(B)(d-Asn)(Amf)(d-Phe) |
| 464 | (d-Asp)(Hle)(B)(Gly)(Gly)(d-Glu) |
| 465 | (d-Asn)(Gly)(Dpa)(Gly)(B)(Gly) |
| 466 | (d-Thr)(d-Pro)(d-Asp)(Gly)(d-Thr)(Gly) |
| 467 | (d-Asn)(d-Asp)(Gly)(d-Asp)(d-Pro)(Nap) |
| 468 | (d-Pro)(d-Thr)(d-Phe)(d-Glu)(B)(Gly) |
| 469 | (d-Lys)(B)(d-Asn)(d-Phe)(d-Asp)(d-Asp) |
| 470 | (d-Gln)(d-Glu)(d-Lys)(d-Asp)(Hle)(Gly) |
| 471 | (Gly)(Dpa)(B)(Gly)(d-His)(B) |
| 472 | (B)(Nle)(d-Arg)(Hle)(Gly)(B) |
| 473 | (B)(Abu)(Gly)(Hle)(d-His)(Hle) |
| 474 | (d-Arg)(B)(d-Ser)(d-Pro)(d-Phe)(d-Glu) |
| 475 | (ox-His)(d-Asp)(d-Asp)(d-Glu)(Abu)(d-Ser) |
| 476 | (Abu)(B)(d-Asp)(Pip)(d-Glu)(d-His) |
| 477 | (d-Asp)(d-Gln)(Hle)(Gly)(Gly)(Nle) |
| 478 | (d-Pro)(d-Gln)(Gly)(Nap)(d-Glu)(d-Asp) |
| 479 | (d-Pro)(d-Ser)(d-Asn)(d-Asp)(d-Glu)(Pip) |
| 480 | (Gly)(d-Asn)(d-Phe)(Gly)(d-Glu)(Nle) |
| 481 | (Gly)(d-Asn)(Dpa)(Gly)(B)(Gly) |
| 482 | (d-Asn)(Dab)(Dab)(d-Pro)(d-Phe)(d-Glu) |
| 483 | (d-Asn)(B)(Gly)(d-Asn)(Nle)(d-Arg) |
| 484 | (Gly)(B)(d-Gln)(Amf)(Nap)(d-Gln) |
| 485 | (d-Phe)(d-Asp)(d-Ser)(B)(Hle)(Pip) |
| 486 | (Gba)(d-Glu)(Gly)(d-Ser)(Dab)(d-Phe) |
| 487 | (Gly)(Gly)(Gly)(Nle)(d-Pro)(d-Asp) |
| 488 | (Gly)(Gly)(Gly)(Dpa)(d-Gln)(Gly) |
| 489 | (Pip)(d-Thr)(d-Pro)(d-Asn)(d-Ser)(Nap) |
| 490 | (Nle)(Abu)(d-Glu)(d-Gln)(d-Glu)(Amf) |
| 491 | (d-Phe)(Hle)(d-Glu)(B)(d-Ser)(d-Thr) |
| 492 | (d-Asp)(Dpa)(d-Ser)(B)(Gly)(B) |
| 493 | (d-Asp)(d-Ser)(d-Phe)(d-Asn)(d-Lys) |
| | (d-Phe) |
| 494 | (B)(Gly)(B)(Hle)(B)(d-Arg) |
| 495 | (Abu)(Pip)(d-Asn)(d-Arg)(d-Ser)(Nap) |
| 496 | (Gly)(Gly)(d-Asn)(Abu)(d-Phe)(d-Gln) |
| 497 | (Gly)(d-Asn)(d-Asn)(Gly)(Dpa)(d-Asn) |
| 498 | (Gly)(Pip)(d-Asp)(Nle)(B)(d-Asn) |
| 499 | (d-Lys)(d-Pro)(d-Pro)(d-Ser)(Pip)(d-Gln) |
| 500 | (Gly)(B)(d-Phe)(Gly)(Gly)(d-Thr) |
| 501 | (d-Asp)(B)(Gly)(Abu)(d-Thr)(d-Phe) |
| 502 | (Gly)(Gly)(Gly)(Nap)(d-Asp)(Dab) |
| 503 | (Gly)(d-Asn)(d-Glu)(Nap)(d-Asn)(d-Asp) |
| 504 | (B)(d-Arg)(Nle)(d-Pro)(Gly)(Gly) |
| 505 | (Gly)(Gba)(d-Glu)(d-Ser)(Dab)(d-Phe) |
| 506 | (Gly)(B)(Gly)(d-Phe)(d-Asn)(d-Thr) |
| 507 | (d-Glu)(d-Ser)(d-Ser)(d-Thr)(Gly)(d-Phe) |
| 508 | (B)(d-Phe)(Nle)(d-Asp)(Abu)(B) |
| 509 | (Nle)(B)(d-Thr)(Nap)(Gly)(Gly) |
| 510 | (B)(Dab)(Nap)(ox-His)(d-Asp)(Nap) |
| 511 | (B)(d-Asn)(d-Pro)(Nle)(Pip)(d-Gln) |
| 512 | (Abu)(B)(d-Arg)(d-Pro)(Pip)(d-Gln) |
| 513 | (Dab)(d-Thr)(Gly)(d-Asp)(d-Thr)(Gly) |
| 514 | (Nle)(d-Asp)(d-His)(d-Asp)(Nle)(d-Phe) |
| 515 | (Gly)(d-Asn)(d-Asp)(d-Asp)(d-Pro)(Nap) |
| 516 | (Gly)(Gly)(Nle)(Gly)(d-Phe)(d-Asn) |
| 517 | (d-Lys)(B)(ox-Amf)(d-Asp)(B)(d-Lys) |
| 518 | (Nle)(B)(Dab)(d-Phe)(Gly)(d-Thr) |
| 519 | (Abu)(B)(d-Ser)(d-Asn)(Amf)(d-Phe) |
| 520 | (Dab)(d-Ser)(d-Asn)(Pip)(Nle)(Gly) |
| 521 | (d-Phe)(Dab)(d-Asp)(Abu)(Abu)(Dpa) |
| 522 | (ox-Nap)(Dab)(Gly)(Abu)(d-Phe)(Gly) |
| 523 | (Gly)(Pip)(Gba)(d-Ser)(d-Thr)(d-Gln) |
| 524 | (d-Lys)(d-Asp)(Gly)(Gly)(Amf)(d-Phe) |
| 525 | (B)(Abu)(d-Asn)(d-Arg)(d-Asn)(Gly) |
| 526 | (d-Asn)(Nle)(Gly)(Gly)(B)(Hle) |
| 527 | (d-Pro)(Gly)(d-Glu)(d-Glu)(Gly)(Nap) |
| 528 | (Abu)(Amf)(d-His)(Gly)(d-Phe)(Gly) |
| 529 | (Abu)(Dab)(Hle)(d-Thr)(Gly)(Abu) |
| 530 | (Nle)(d-Arg)(Nle)(Abu)(d-Asn)(d-Asp) |
| 531 | (d-Pro)(Gly)(B)(Gly)(Abu)(Hle) |
| 532 | (d-Asp)(Dpa)(d-Ser)(Gly)(Gly)(Abu) |
| 533 | (Abu)(Gba)(d-Ser)(d-Pro)(d-Phe)(d-Glu) |
| 534 | (B)(Gly)(B)(d-Glu)(Nle)(Dpa) |
| 535 | (d-Lys)(ox-His)(ox-His)(d-Ser)(d-Asp) |
| | (Nap) |
| 536 | (Nle)(d-Asn)(Abu)(d-Pro)(Pip)(d-Gln) |
| 537 | (Amf)(d-Thr)(d-Pro)(d-Pro)(B)(d-Asp) |
| 538 | (Gly)(d-Ser)(ox-Amf)(B)(Gly)(Amf) |
| 539 | (Gly)(Gly)(B)(d-Gln)(Dpa)(d-Phe) |
| 540 | (d-Arg)(d-Asp)(Gly)(d-Ser)(Dab)(d-Phe) |
| 541 | (Abu)(d-Gln)(Dab)(d-Phe)(d-Asp)(d-Asp) |
| 542 | (d-Lys)(d-Asn)(B)(d-Phe)(d-Asp)(d-Asp) |
| 543 | (Gly)(Gly)(d-Phe)(d-Asn)(d-Lys)(d-Phe) |
| 544 | (Nle)(d-Glu)(B)(Gly)(Gly)(d-Glu) |
| 545 | (d-Asn)(B)(Gly)(d-Ser)(Nap)(d-Asp) |
| 546 | (d-Asp)(B)(Abu)(Hle)(Amf)(Nle) |
| 547 | (B)(d-Asn)(d-Pro)(Nap)(d-Glu)(d-Asp) |
| 548 | (Gly)(Nle)(d-Glu)(B)(d-His)(Dab) |
| 549 | (d-Arg)(d-Pro)(d-Asp)(ox-His)(d-Asp)(Nap) |
| 550 | (Abu)(B)(d-Thr)(Amf)(d-Phe)(d-His) |
| 551 | (Abu)(B)(B)(d-Asn)(d-Phe)(Pip) |
| 552 | (Gly)(Gly)(d-Pro)(B)(d-Glu)(Nle) |
| 553 | (Gly)(Gly)(d-Ser)(Nle)(Abu)(Nap) |
| 554 | 0(d-Ser)(Hle)(B)(Gly)(Gly) |
| 555 | (B)(d-Asn)(d-Asp)(Nap)(d-Asn)(d-Asp) |
| 556 | (B)(d-Asp)(Amf)(Gly)(d-Ser)(d-Thr) |
| 557 | (B)(Nle)(Dab)(B)(ox-His)(Nle) |
| 558 | (d-Lys)(B)(B)(B)(d-Thr)(ox-His) |
| 559 | (d-Lys)(B)(Gly)(ox-His)(d-Thr)(d-Gln) |
| 560 | (Gly)(d-Ser)(Amf)(Gly)(d-Ser)(Amf) |
| 561 | (Abu)(B)(Gba)(d-Ser)(d-Asp)(d-Phe) |
| 562 | (d-Thr)(d-Phe)(d-Lys)(d-Asn)(d-Thr) |
| | (d-Phe) |
| 563 | (Abu)(B)(Gly)(d-Ser)(Dab)(d-Thr) |
| 564 | (B)(Gly)(Abu)(Hle)(d-His)(Hle) |
| 565 | (Abu)(Gly)(d-Glu)(Hle)(Amf)(Nle) |
| 566 | (d-Asp)(B)(Dab)(d-Pro)(ox-His)(d-Glu) |
| 567 | (d-Pro)(d-Glu)(Gly)(d-Glu)(Gly)(Nap) |
| 568 | (Nle)(Nle)(Abu)(B)(d-Glu)(d-Gln) |
| 569 | (d-Asn)(Abu)(Hle)(B)(Abu)(B) |
| 570 | (Gly)(Gly)(Gly)(d-Asp)(Nap)(Dab) |
| 571 | (Gly)(d-Asn)(d-Gln)(d-Ser)(Nap)(d-Asp) |
| 572 | (ox-Nap)(d-Pro)(d-Asp)(Gly)(Hle)(d-Pro) |
| 573 | (B)(Gly)(B)(B)(Hle)(d-Arg) |
| 574 | (Nle)(d-Thr)(Nle)(Gly)(Gly)(d-Thr) |
| 575 | (d-Asn)(d-Asp)(Pip)(d-Ser)(d-Thr)(d-Phe) |
| 576 | (d-His)(d-His)(Gly)(B)(Gly)(Gly) |
| 577 | (Abu)(Nle)(d-Pro)(d-Pro)(d-Pro)(Gly) |
| 578 | (Dab)(Hle)(Hle)(Abu)(B)(Gly) |
| 579 | (Gly)(d-Glu)(Nle)(Pip)(d-Asn)(Gly) |
| 580 | (d-Ser)(d-Pro)(d-Pro)(d-Lys)(Pip)(d-Gln) |
| 581 | (Gly)(d-Ser)(Nle)(d-Glu)(Gly)(Gly) |
| 582 | (B)(Nle)(B)(Abu)(Hle)(d-Gln) |
| 583 | (Gly)(Gly)(d-Glu)(Hle)(d-Asn)(Nle) |
| 584 | (B)(d-Asn)(Dpa)(Nie)(d-Thr)(Dab) |
| 585 | (B)(Nle)(d-Arg)(Hle)(B)(Gly) |
| 586 | (B)(Nle)(Abu)(B)(Hle)(d-Gln) |
| 587 | (Gly)(Gly)(Abu)(d-Thr)(d-Gln)(Nap) |
| 588 | (d-Asp)(d-Thr)(d-Pro)(Hle)(d-Gln)(Hle) |
| 589 | (Nap)(Hle)(Gly)(Gly)(d-Ser)(Nap) |
| 590 | (B)(Nle)(Dab)(d-Pro)(B)(ox-His) |
| 591 | (B)(d-His)(Dab)(d-Phe)(d-Phe)(Gly) |
| 592 | (Abu)(Gly)(Hle)(B)(Amf)(d-Glu) |
| 593 | (Gly)(d-Gln)(Gba)(d-Ser)(d-Asn)(d-Ser) |
| 594 | (d-Ser)(d-Ser)(Dpa)(B)(Gly)(Gly) |
| 595 | (Nle)(d-Asp)(d-Asp)(Pip)(d-Gln)(Gly) |
| 596 | (d-Pro)(B)(d-Asp)(d-Thr)(Hle)(d-Asp) |
| 597 | (d-Asp)(d-Phe)(d-Ser)(B)(Hle)(Pip) |
| 598 | (d-Pro)(Pip)(d-Thr)(d-Asn)(d-Ser)(Nap) |
| 599 | (d-Gln)(ox-His)(d-Asn)(Pip)(d-Asp)(Nap) |
| 600 | (d-Gln)(Pip)(ox-His)(d-Asn)(d-Asp)(Nap) |
| 601 | (Abu)(Gly)(d-Asn)(Abu)(B)(Dpa) |
| 602 | (B)(B)(Gly)(Gly)(d-Arg)(Dpa) |
| 603 | (Gly)(d-Ser)(Gly)(d-Ser)(Amf)(Amf) |
| 604 | (d-Asp)(d-Ser)(d-Phe)(B)(Hle)(Pip) |
| 605 | (Gly)(Hle)(d-Thr)(Nap)(Gly)(Gly) |
| 606 | (B)(Nap)(Dab)(d-Ser)(d-Phe)(Gly) |
| 607 | (Dab)(d-Ser)(d-His)(Nap)(d-Asp)(Nap) |
| 608 | (Nle)(d-Phe)(d-Asn)(B)(d-Ser)(d-Ser) |
| 609 | (d-Asn)(B)(d-Pro)(d-Pro)(Nle)(d-His) |
| 610 | (d-Gln)(d-Asn)(d-Glu)(d-Gln)(d-Ser) |
| | (d-Phe) |
| 611 | (Abu)(B)(d-Lys)(Abu)(d-Ser)(d-Ser) |
| 612 | (B)(d-Lys)(Gly)(ox-His)(d-Thr)(d-Gln) |
| 613 | (B)(d-Asp)(d-Asn)(Pip)(Nle)(Gly) |
| 614 | (Gly)(d-Ser)(Abu)(B)(d-Gln)(Nap) |
| 615 | (Gly)(d-Ser)(Abu)(d-Arg)(d-Asp)(d-Phe) |
| 616 | (Nle)(d-Lys)(Gly)(d-Glu)(ox-His)(d-Lys) |
| 617 | (B)(d-Ser)(d-Ser)(d-Thr)(Gba)(Pip) |
| 618 | (B)(Gly)(d-Phe)(Gly)(Gly)(d-Thr) |
| 619 | (Abu)(Gly)(Gly)(Gly)(Gly)(d-Phe) |
| 620 | (d-Pro)(d-Pro)(d-Ser)(d-Glu)(d-Glu)(Dab) |
| 621 | (Gly)(Abu)(Gly)(Gly)(d-Arg)(Dpa) |
| 622 | (d-Asp)(Abu)(B)(Hle)(Amf)(Nle) |
| 623 | (d-Asn)(d-Pro)(d-Ser)(Nle)(d-Phe)(d-Glu) |
| 624 | (d-Lys)(B)(B)(d-Asp)(ox-Amf)(d-Lys) |
| 625 | (d-Gln)(B)(d-Pro)(Nie)(Pip)(d-Gln) |
| 626 | (d-Gln)(d-Phe)(d-Asp)(d-His)(Gly)(Gly) |
| 627 | (d-Pro)(d-Asn)(d-Ser)(Nie)(d-Phe)(d-Glu) |
| 628 | (d-Asp)(d-Thr)(d-Gln)(Hle)(d-Pro)(Hle) |
| 629 | (B)(d-Glu)(Gly)(d-His)(Dab)(Hle) |
| 630 | (Nle)(d-His)(d-His)(B)(d-Glu)(Amf) |
| 631 | (d-Thr)(Dab)(d-Asp)(Gly)(d-Thr)(Gly) |
| 632 | (B)(Abu)(d-Phe)(Gba)(d-Ser)(d-Ser) |
| 633 | (d-Phe)(Dab)(d-Glu)(B)(Abu)(Dpa) |
| 634 | (Gly)(Gly)(d-Glu)(Hle)(d-His)(d-Pro) |
| 635 | (Gly)(Dpa)(B)(Gly)(B)(d-His) |
| 636 | (B)(Abu)(Nle)(d-Lys)(d-His)(Abu) |
| 637 | (Gly)(d-Ser)(B)(Gly)(ox-Amf)(Amf) |
| 638 | (Gly)(Abu)(ox-His)(d-His)(d-Ser)(d-Asn) |
| 639 | (B)(d-Pro)(d-Asp)(Hle)(d-Thr)(d-Asp) |
| 640 | (Gly)(Gly)(Gly)(d-Pro)(Nle)(d-Asp) |
| 641 | (Abu)(d-Pro)(d-Pro) |
| 642 | (d-Pro)(d-Pro)(Abu) |
| 643 | (Nle)(d-Thr)(d-Glu)(d-Arg) |
| 644 | (Nle)(d-Pro)(d-Asp)(d-Glu)(d-Thr) |
| 645 | (Nle)(d-Pro)(d-Asp)(d-Arg)(ox-Trp) |
| 646 | (B)(Abu)(d-Ser)(Abu)(Hle) |
| 647 | (d-Lys)(Nap)(d-Glu)(d-Thr) |
| 643 | (Nle)(d-Thr)(d-Glu)(d-Arg) |
| 648 | (Nle)(d-Pro)(d-Asp)(d-Arg)W(ox-Trp) |
| 649 | (Nle)(d-Asp)(Dab)(Hle) |
| 650 | (d-Lys)(d-Asp)(Nle)(d-Gln)(d-Gln) |
| 651 | (Nle)(d-Thr)(d-Glu)(B)(Abu) |
| 652 | (d-Ser)(Pip)(d-Ser)(Gly)(Hle) |
| 653 | (Nle)(d-Thr)(d-Pro)(d-Thr)(d-Ser) |
| 654 | (B)(Abu)(B)(d-Thr)(Hle) |
| 655 | (d-Lys)(d-Asp)(d-Asn)(Hle) |
| 656 | (B)(d-Glu)(Dab)(Gly)(Hle) |
| 657 | (B)(d-Arg)(d-Arg)(Abu)(Dab)(d-His) |
| 658 | (B)(d-Arg)(Abu)(d-Arg)(Dab)(d-His) |
| 659 | (Nle)(d-Asn)(Hle)(Nle)(d-Thr)(d-His) |
| 660 | (Gba)(d-Asn)(Nle)(d-Asn)(d-His) |
| 661 | (ox-His)(d-His)(Hle) |
| 662 | (d-Arg)(ox-Nap)(d-Asn)(d-His) |
| 663 | (d-Lys)(B)(Abu)(d-Arg)(d-His) |
| 664 | (Abu)(Gly)(d-Asn)(Nle)(d-Asn)(d-His) |
| 665 | (d-Lys)(d-Arg)(d-Arg)(d-His) |
| 666 | (B)(d-Arg)(Nle)(Gba)(Dpa) |
| 667 | (Nle)(d-Asn)(d-Lys)(B)(Gba)(d-His) |
| 668 | (Nle)(Hle)(ox-Amf)(d-Pro)(d-Phe) |
| 669 | (B)(d-Arg)(Gba)(Dpa)(Nle) |

In some embodiments, the cell penetrating peptide can be 3 to 15 amino acids selected from unnatural amino acids or D-amino acids excluding the C-terminal sequence. In some embodiments, the cell penetrating peptide can be 3 to 15 amino acids selected from unnatural amino acids or D-amino acids including the C-terminal sequence. Included in the disclosure are cell penetrating peptides that can be 3 to 15, 3 to 12, 3 to 10, 3 to 8, 3 to 6, 4 to 15, 4 to 12, 4 to 10, 4 to 8, 4 to 6, 5 to 15, 5 to 12, 5 to 10, 5 to 8, 5 to 6, 6 to 15, 6 to 12, 6 to 10, 6 to 8, 4, 6, or 8 amino acids selected from unnatural amino acids or D-amino acids excluding the C-terminal sequence. In an embodiment, the cell penetrating peptide further comprises a C-terminal sequence having a KWKK, KKWK, KWWKK, WWKK, WKK, KKKK, or KK motif. In another embodiment, the cell penetrating peptide further comprises a C-terminal sequence having a (d-K)(d-W)(d-K)(d-K), (d-K)(d-K)(d-W)(d-K), (d-K)(d-W)(d-W)(d-K)(d-K), (d-W)(d-W)(d-K)(d-K), (d-W)(d-K)(d-K), (d-K)(d-K)(d-K)(d-K), or (d-K)(d-K) motif.

In an embodiment, the cell penetrating peptides provided above are bound to L via a trypsin cleavable linker. In an embodiment, the trypsin cleavable linker is GGKGG.

In another embodiment, provided herein is a trypsin-cleavable linker/cell penetrating peptide conjugate that can be used in the compositions provided herein, having the formula: GGKGG-(cell penetrating peptide)-(KWKK), GGKGG-(cell penetrating peptide)-(KKWK), GGKGG-(cell penetrating peptide)-(KWWKK), GGKGG-(cell penetrating peptide)-(WWKK), GGKGG-(cell penetrating peptide)-(WKK), GGKGG-(cell penetrating peptide)-(KKKK), or GGKGG-(cell penetrating peptide)-(KK).

In still another embodiment, provided herein is a biotin/trypsin-cleavable linker/cell penetrating peptide conjugate that can be used in the compositions provided herein, having the formula: Biotin-GGKGG-(cell penetrating peptide)-(KWKK), Biotin-GGKGG-(cell penetrating peptide)-(KKWK), Biotin-GGKGG-(cell penetrating peptide)-(KWWKK), Biotin-GGKGG-(cell penetrating peptide)-(WWKK), Biotin-GGKGG-(cell penetrating peptide)-(WKK), Biotin-GGKGG-(cell penetrating peptide)-(KKKK), or Biotin-GGKGG-(cell penetrating peptide)-(KK).

In yet another embodiment, provided herein is a trypsin-cleavable linker/isoseramox or d-Ser cleavable linker (s)/cell penetrating peptide conjugate that can be used in the compositions provided herein, having the formula: GGKGG-(s)-(cell penetrating peptide)-(KWKK), GGKGG-(s)-(cell penetrating peptide)-(KKWK), GGKGG-(s)-(cell penetrating peptide)-(KWWKK), GGKGG-(s)-(cell penetrating peptide)-(WWKK), GGKGG-(s)-(cell penetrating peptide)-(WKK), GGKGG-(s)-(cell penetrating peptide)-(KKKK), or GGKGG-(s)-(cell penetrating peptide)-(KK).

In another embodiment, provided herein is a trypsin-cleavable linker/cell penetrating peptide conjugate that can be used in the compositions provided herein, having the formula: GGKGG-(cell penetrating peptide)-((d-K)(d-W)(d-K)(d-K)), GGKGG-(cell penetrating peptide)-((d-K)(d-K)(d-W)(d-K), GGKGG-(cell penetrating peptide)-((d-K)(d-W)(d-W)(d-K)(d-K), GGKGG-(cell penetrating peptide)-((d-W)(d-W)(d-K)(d-K)), GGKGG-(cell penetrating peptide)-((d-W)(d-K)(d-K)), GGKGG-(cell penetrating peptide)-((d-K)(d-K)(d-K)(d-K)), or GGKGG-(cell penetrating peptide)-((d-K)(d-K)).

In still another embodiment, provided herein is a biotin/trypsin-cleavable linker/cell penetrating peptide conjugate that can be used in the compositions provided herein, having the formula: Biotin-GGKGG-(cell penetrating peptide)-((d-K)(d-W)(d-K)(d-K)), Biotin-GGKGG-(cell penetrating peptide)-((d-K)(d-K)(d-W)(d-K), Biotin-GGKGG-(cell penetrating peptide)-((d-K)(d-W)(d-W)(d-K)(d-K), Biotin-GGKGG-(cell penetrating peptide)-((d-W)(d-W)(d-K)(d-K)), Biotin-GGKGG-(cell penetrating peptide)-((d-W)(d-K)(d-K)), Biotin-GGKGG-(cell penetrating peptide)-((d-K)(d-K)(d-K)(d-K)), or Biotin-GGKGG-(cell penetrating peptide)-((d-K)(d-K)).

In yet another embodiment, provided herein is a trypsin-cleavable linker/isoseramox or d-Ser cleavable linker (s)/cell penetrating peptide conjugate that can be used in the compositions provided herein, having the formula: GGKGG-(s)-(cell penetrating peptide)-((d-K)(d-W)(d-K)(d-K)), GGKGG-(s)-(cell penetrating peptide)-((d-K)(d-K)(d-W)(d-K), GGKGG-(s)-(cell penetrating peptide)-((d-K)(d-W)(d-W)(d-K)(d-K), GGKGG-(s)-(cell penetrating peptide)-((d-W)(d-W)(d-K)(d-K)), GGKGG-(s)-(cell penetrating peptide)-((d-W)(d-K)(d-K)), GGKGG-(s)-(cell penetrating peptide)-((d-K)(d-K)(d-K)(d-K)), or GGKGG-(s)-(cell penetrating peptide)-((d-K)(d-K)).

### VI. Pharmaceutical Compositions

The present disclosure also provides for formulation and delivery of the disclosed conjugates. Accordingly, an aspect of the present disclosure is a pharmaceutical composition comprising conjugates as disclosed herein and a pharmaceutically acceptable carrier.

Effective delivery of the conjugates to the target nucleic acid is an important aspect of treatment. Routes of conjugate delivery include, but are not limited to, various systemic routes, including oral and parenteral routes, e.g., intravenous, subcutaneous, intraperitoneal, and intramuscular, as well as inhalation, transdermal and topical delivery. The appropriate route may be determined by one of skill in the art, as appropriate to the condition of the subject under treatment.

The conjugate can be administered in any convenient vehicle which is physiologically and/or pharmaceutically acceptable. Such a composition can include any of a variety of standard pharmaceutically acceptable carriers employed by those of ordinary skill in the art. Examples include, but are not limited to, saline, phosphate buffered saline (PBS), water (e.g., sterile water for injection), aqueous ethanol, emulsions such as oil/water emulsions or triglyceride emulsions, tablets and capsules. The choice of suitable physiologically acceptable carrier will vary dependent upon the chosen mode of administration.

The instant compounds (e.g., a conjugate) can generally be utilized as the free acid or free base. Alternatively, the instant compounds may be used in the form of acid or base addition salts. Acid addition salts of the free amino compounds may be prepared by methods well known in the art, and may be formed from organic and inorganic acids. Suitable organic acids include maleic, fumaric, benzoic, ascorbic, succinic, methanesulfonic, acetic, trifluoroacetic, oxalic, propionic, tartaric, salicylic, citric, gluconic, lactic, mandelic, cinnamic, aspartic, stearic, palmitic, glycolic, glutamic, and benzenesulfonic acids. Suitable inorganic acids include hydrochloric, hydrobromic, sulfuric, phosphoric, and nitric acids. Base addition salts included those salts that form with the carboxylate anion and include salts formed with organic and inorganic cations such as those chosen from the alkali and alkaline earth metals (for example, lithium, sodium, potassium, magnesium, barium and calcium), as well as the ammonium ion and substituted derivatives thereof (for example, dibenzylammonium, benzylammonium, 2-hydroxyethylammonium, and the like). Thus, the term “pharmaceutically acceptable salt” of Formula (I) and Formula (II) are intended to encompass any and all acceptable salt forms.

In addition, prodrugs are also included within the context of this invention. Prodrugs are any covalently bonded carriers that release a compound of Formula (I) or Formula (II) in vivo when such prodrug is administered to a patient. Prodrugs are generally prepared by modifying functional groups in a way such that the modification is cleaved, either by routine manipulation or in vivo, yielding the parent compound. Prodrugs include, for example, compounds of this invention wherein hydroxy, amine or sulfhydryl groups are bonded to any group that, when administered to a patient, cleaves to form the hydroxy, amine or sulfhydryl groups. Thus, representative examples of prodrugs include (but are not limited to) acetate, formate and benzoate derivatives of alcohol and amine functional groups of the compounds of Formula (I) and Formula (II). Further, in the case of a carboxylic acid (—COOH), esters may be employed, such as methyl esters, ethyl esters, and the like.

### VII. Methods of Treatment

Provided herein is a method of treating a neuromuscular disease. The method comprises administering to a patient in need thereof a therapeutically effective amount of an oligonucleotide conjugate disclosed herein, or a pharmaceutical composition thereof. In an embodiment, the neuromuscular disease is Duchenne muscular dystrophy.

In certain embodiments, the method is an in vitro method. In certain other embodiments, the method is an in vivo method.

In certain embodiments, the host cell is a mammalian cell. In certain embodiments, the host cell is a non-human primate cell. In certain embodiments, the host cell is a human cell.

In certain embodiments, the host cell is a naturally occurring cell. In certain other embodiments, the host cell is an engineered cell.

In certain embodiments, the conjugate is administered to a mammalian subject, e.g., a human or a laboratory or domestic animal, in a suitable pharmaceutical carrier.

In certain embodiments, the conjugate is administered to a mammalian subject, e.g., a human or laboratory or domestic animal, together with an additional agent. The conjugate and the additional agent can be administered simultaneously or sequentially, via the same or different routes and/or sites of administration. In certain embodiments, the conjugate and the additional agent can be co-formulated and administered together. In certain embodiments, the conjugate and the additional agent can be provided together in a kit.

In one embodiment, the conjugate, contained in a pharmaceutically acceptable carrier, is delivered orally.

In one embodiment, the conjugate, contained in a pharmaceutically acceptable carrier, is delivered intravenously (i.v.).

Additional routes of administration, e.g., subcutaneous, intraperitoneal, and pulmonary, are also contemplated by the instant disclosure.

In another application of the method, the subject is a livestock animal, e.g., a pig, cow, or goat, etc., and the treatment is either prophylactic or therapeutic. Also contemplated is, in a method of feeding livestock with a food substance, an improvement in which the food substance is supplemented with an effective amount of a conjugate composition as described above.

In an embodiment, the conjugate is administered in an amount and manner effective to result in a peak blood concentration of at least 200 nM conjugate. In one embodiment, the conjugate is administered in an amount and manner effective to result in a peak plasma concentration of at least 200 nM conjugate. In one embodiment, the conjugate is administered in an amount and manner effective to result in a peak serum concentration of at least 200 nM conjugate.

In an embodiment, the conjugate is administered in an amount and manner effective to result in a peak blood concentration of at least 400 nM conjugate. In one embodiment, the conjugate is administered in an amount and manner effective to result in a peak plasma concentration of at least 400 nM conjugate. In one embodiment, the conjugate is administered in an amount and manner effective to result in a peak serum concentration of at least 400 nM conjugate.

Typically, one or more doses of conjugate are administered, generally at regular intervals, for a period of about one to two weeks. Preferred doses for oral administration are from about 0.01-15 mg conjugate per kg body weight. In some cases, doses of greater than 15 mg conjugate/kg may be necessary. For i.v. administration, preferred doses are from about 0.005 mg to 15 mg conjugate per kg body weight. The conjugate may be administered at regular intervals for a short time period, e.g., daily for two weeks or less. However, in some cases the conjugate is administered intermittently over a longer period of time. Administration may be followed by, or accompanied by, administration of an antibiotic or other therapeutic treatment. The treatment regimen may be adjusted (dose, frequency, route, etc.) as indicated, based on the results of immunoassays, other biochemical tests, and physiological examination of the subject under treatment.

An effective in vivo treatment regimen using the conjugates may vary according to the duration, dose, frequency and route of administration, as well as the condition of the subject under treatment (i.e., prophylactic administration versus administration in response to localized or systemic infection). Accordingly, such in vivo therapy will often require monitoring by tests under treatment, and corresponding adjustments in the dose or treatment regimen, in order to achieve an optimal therapeutic outcome.

In some embodiments, the conjugate is actively taken up by mammalian cells. In further embodiments, the conjugate can be conjugated to a transport moiety (e.g., transport peptide) as described herein to facilitate such uptake.

In some embodiments, the oligonucleotide conjugates of the instant application have about 20% less kidney accumulation as compared to the unconjugated oligomers. In a further embodiment, the oligonucleotide conjugates of the instant application have about a 5-fold concentration increase in muscle cells as compared to the unconjugated oligomer. In a further embodiment, the oligonucleotide conjugates of the instant application have about a 50-fold concentration increase in muscle cells as compared to the unconjugated oligomer.

### VIII. Methods of Identifying a Peptide

Provided herein is a method of identifying a peptide capable of delivering a peptide or peptide-conjugated phosphorodiamidate morpholino oligomer (PPMO) into a cell. In particular, provided herein is a method for identifying peptides capable of delivering macromolecule cargo to the cytosol.

The method comprises:
(a) treating a cell with a peptide or a peptide-conjugated phosphorodiamidate morpholino oligomer (PPMO);
(b) treating the cell with a digestive enzyme;
(c) lysing the cell for whole cell extraction forming a whole cell lysate or lysing the cell for cytosolic extraction forming a cytosolic fraction; and
(d) analyzing the whole cell lysate or cytosolic fraction with mass spectrometry to identify the peptide.

In an embodiment, the method further comprises washing the cell. In another embodiment, the cell is washed with PBS, TRIS, or HEpes. In yet another embodiment, the cell is washed with PBS.

In an embodiment, the method further comprises analyzing the whole cell lysate or cytosolic fraction via Western blot for the presence of a cytosolic marker (Erk 1/2) or a late-endosomal marker (Rab5). In a further embodiment, the absence of Rab5 in the Western blot of the cytosolic fraction indicates exclusion of endosomes.

In an embodiment, the digestive enzyme is chymotrypsin or trypsin. In another embodiment, the digestive enzyme is trypsin.

In an embodiment, the method comprises lysing the whole cell with RIPA buffer. In another embodiment, the method comprises lysing the cytosol with digitonin buffer.

In an embodiment, the method comprises analyzing the peptide sequence with mass spectrometry with a mixed fragmentation method optimized for cationic peptides, consisting of electron-transfer dissociation (ETD), higher-energy ETD, and higher-energy collisional dissociation (HCD).

In an embodiment, the cell is a HeLa cell, a C2C12 mouse myoblast, or a CHOK1 cell. In another embodiment, the cell is a HeLa cell or a C2C12 mouse myoblast.

In an embodiment, the peptide comprises 4 to 15 amino acids selected from unnatural amino acids or D-amino acids. In another embodiment, the peptide further comprises a C-terminal sequence having a KWKK, KKWK, KWWKK, WWKK, WKK, KKKK, or KK motif. In yet another embodiment, the peptide further comprises a C-terminal sequence having a KWKK motif. In yet another embodiment, the peptide further comprises a C-terminal sequence having a (d-K)(d-W)(d-K)(d-K), (d-K)(d-K)(d-W)(d-K), (d-K)(d-W)(d-W)(d-K)(d-K), (d-W)(d-W)(d-K)(d-K), (d-W)(d-K)(d-K), (d-K)(d-K)(d-K)(d-K), or (d-K)(d-K) motif.

In an embodiment, the unnatural amino acids are selected from Abu (γ-aminobutyric acid), B (μ-alanine), Hle (homoleucine), Ne (norleucine), Nap (naphthylalanine), Dpa (diphenylalanine), Dab (diaminobutyric acid), Pip (aminopiperidine-carboxylic acid), Amf (aminomethylphenylalanine), and Gba (2-amino-4-guanidinobutanoic acid).

In an embodiment, the treating of the cell comprises treating the cell with a peptide-library or a PPMO-library.

Additionally provided herein is a method of identifying a peptide capable of delivering a phosphorodiamidate morpholino oligomer (PMO) into a cell. The method comprises:
a) treating the cell with a PPMO-library or peptide-library;
b) washing the cell with PBS and heparin;
c) lysing the cell with RIPA (whole cell extract) or digitonin (cytosolic extract);
d) incubating the whole cell extract or digitonin extract with magnetic streptavidin beads;
e) cleaving the peptides from the streptavidin beads under oxidative conditions;
f) desalting the peptides through solid-phase extraction resulting in PPMO-library fractions or peptide-library fraction;
g) sequencing the peptides by nLC-MS/MS; and
h) identifying peptide sequences found only in the PPMO-library fractions that do not overlap with peptides found in the cell only control or the peptide-library fractions.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### EXAMPLES

Examples have been set forth below for the purpose of illustration and to describe certain specific embodiments of the disclosure. However, the scope of the claims is not to be in any way limited by the examples set forth herein. Various changes and modifications to the disclosed embodiments will be apparent to those skilled in the art and such changes and modifications including, without limitation, those relating to the chemical structures, substituents, derivatives, formulations or methods of the disclosure may be made without departing from the spirit of the disclosure and the scope of the appended claims. Definitions of the variables in the structures in the schemes herein are commensurate with those of corresponding positions in the formulae presented herein.

### Example 1—General Method for Peptide Preparation and Purification

To synthesize the constructs, L-peptides were synthesized via automated fast-flow peptide synthesis, and D-peptides were synthesized using semi-automated fast flow peptide synthesis. Azido-lysine and biotin moieties were added to the N-terminus of the peptides manually, and the peptides were simultaneously cleaved and deprotected before purification via RP-HPLC.

Fast-flow Peptide Synthesis: Peptides were synthesized on a 0.1 mmol scale using an automated fast-flow peptide synthesizer for L-peptides and a semi-automated fast-flow peptide synthesizer for D-peptides. Automated synthesis conditions were used as previously reported. Briefly, a 100 mg portion of ChemMatrix Rink Amide HYR resin was loaded into a reactor maintained at 90° C. All reagents were flowed at 40 mL/min with HPLC pumps through a stainless-steel loop maintained at 90° C. before introduction into the reactor. For each coupling, 10 mL of a solution containing 0.4 M amino acid and 0.38 M HATU in DMF were mixed with 600 μL diisopropylethylamine and delivered to the reactor. Fmoc removal was accomplished using 10.4 mL of 20% (v/v) piperidine. Between each step, DMF (15 mL) was used to wash out the reactor. To couple unnatural amino acids or to cap the peptide (e.g. with 4-pentynoic acid), the resin was incubated for 30 min at room temperature with amino acid (1 mmol) dissolved in 2.5 mL 0.4 M HATU in DMF with 500 μL diisopropylethylamine. After completion of the synthesis, the resin was washed 3 times with dichloromethane and dried under vacuum.

Semi-automated synthesis was carried out as previously described (Simon, M. D.; Heider, P. L.; Adamo, A.; Vinogradov, A. A.; Mong, S. K.; Li, X.; Berger, T.; Policarpo, R. L.; Zhang, C.; Zou, Y.; Liao, X.; Spokoyny, A. M.; Jensen, K. F.; Pentelute, B. L. 2014, 15 (5), 713-720.). 1 mmol of amino acid was combined with 2.5 mL 0.4 M HATU and 500 μL DIEA and mixed before being delivered to the reactor containing resin via syringe pump at 6 mL/min. The reactor was submerged in a water bath heated to 70° C. An HPLC pump delivered either DMF (20 mL) for washing or 20% piperidine/DMF (6.7 mL) for Fmoc deprotection, at 20 mL/min.

Peptide Cleavage and Deprotection: Each peptide was subjected to simultaneous global side-chain deprotection and cleavage from resin by treatment with 5 mL of 94% trifluoroacetic acid (TFA), 2.5% thioanisole, 2.5% water, and 1% triisopropylsilane (TIPS) (v/v) at room temperature for 2 to 4 hours. The cleavage cocktail was first concentrated by bubbling N₂ through the mixture, and cleaved peptide was precipitated and triturated with 40 mL of cold ether (chilled in dry ice). The crude product was pelleted by centrifugation for three minutes at 4,000 rpm and the ether was decanted. This wash step was repeated two more times. After the third wash, the pellet was dissolved in 50% water and 50% acetonitrile containing 0.1% TFA, filtered through a fritted syringe to remove the resin and lyophilized.

Peptide Purification: The peptides were dissolved in water and acetonitrile containing 0.1% TFA, filtered through a 0.22 μm nylon filter and purified by mass-directed semi-preparative reversed-phase HPLC. Solvent A was water with 0.1% TFA additive and Solvent B was acetonitrile with 0.1% TFA additive. A linear gradient that changed at a rate of 0.5% B/min was used. Most of the peptides were purified on an Agilent Zorbax SB C18 column: 9.4×250 mm, 5 μm. Using mass data about each fraction from the instrument, only pure fractions were pooled and lyophilized. The purity of the fraction pool was confirmed by LC-MS.

### Example 2—Peptide Conjugation

PMO was modified with a dibenzocyclooctyne (DBCO) moiety and purified before attachment to the azido-peptides. PMO IVS-654 (50 mg, 8 μmol) was dissolved in 150 μL DMSO. To the solution was added a solution containing 2 equivalents of dibenzocyclooctyne acid (5.3 mg, 16 μmol) activated with HBTU (37.5 μL of 0.4 M HBTU in DMF, 15 μmol) and DIEA (2.8 μL, 16 μmol) in 40 μL DMF (Final reaction volume=0.23 mL). The reaction proceeded for 25 min before being quenched with 1 mL of water and 2 mL of ammonium hydroxide. The ammonium hydroxide hydrolyzed any ester formed during the course of the reaction. After 1 hour, the solution was diluted to 40 mL in water/acetonitrile and purified using reverse-phase HPLC (Agilent Zorbax SB C3 column: 21.2×100 mm, 5 μm) and a linear gradient from 2 to 60% B (solvent A: water; solvent B: acetonitrile) over 58 min (1% B/min). Using mass data about each fraction from the instrument, only pure fractions were pooled and lyophilized. The purity of the fraction pool was confirmed by LC-MS.

Coniugation to peptides: PMO-DBCO (1 eq, 5 mM, water) was conjugated to azido-peptides (1.5 eq, 5 mM, water) at room temperature for 2 h. Reaction progress was monitored by liquid-chromatography mass-spectrometry (LC-MS) and purified when PMO-DBCO was consumed. Purification was conducted using mass-directed HPLC (Solvent A: 100 mM ammonium acetate in water, Solvent B: acetonitrile) with a linear gradient that changed at a rate of 0.5% B/min, on an Agilent Zorbax SB C13 column: 9.4×250 mm, 5 μm. Using mass data about each fraction from the instrument, only pure fractions were pooled and lyophilized. The purity of the fraction pool was confirmed by LC-MS.

### Example 3—Enhanced Green Fluorescent Protein Assay

Purified constructs were then tested using an activity-based readout in which nuclear delivery results in fluorescence. Briefly, HeLa cells stably transfected with an Enhanced Green Fluorescent Protein (EGFP) gene interrupted by a mutated intron of ß-globin (IVS2-654) produce a non-fluorescent EGFP protein. Successful delivery of PMO IVS2-654 to the nucleus results in corrective splicing and EGFP synthesis.

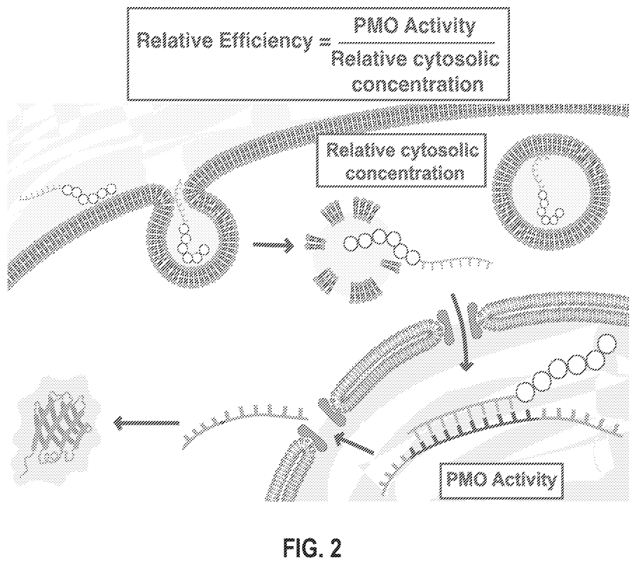

The amount of PMO delivered to the nucleus is therefore correlated with EGFP fluorescence, quantified by flow cytometry. Activity is reported as mean fluorescence intensity (MFI) relative to PMO alone. This activity assay provides indirect information on how much active PMO is delivered to the nucleus. Relative efficiency of a PMO-CPP could be characterized by comparing activity to internal concentration, as discussed later (FIG. 2).

HeLa 654 cells obtained from the University of North Carolina Tissue Culture Core facility were maintained in MEM supplemented with 10% (v/v) fetal bovine serum (FBS) and 1% (v/v) penicillin-streptomycin at 37° C. and 5% CO2. 18 h prior to treatment, the cells were plated at a density of 5,000 cells per well in a 96-well plate in MEM supplemented with 10% FBS and 1% penicillin-streptomycin.

For individual peptide testing, PMO-peptides were dissolved in PBS without Ca2+ or Mg2+ at a concentration of 1 mM (determined by UV) before being diluted in MEM. Cells were incubated at the designated concentrations for 22 h at 37° C. and 5% CO2. Next, the treatment media was removed, and the cells were washed once before being incubated with 0.25% Trypsin-EDTA for 15 min at 37° C. and 5% CO₂. Lifted cells were transferred to a V-bottom 96-well plate and washed once with PBS, before being resuspended in PBS containing 2% FBS and 2 μg/mL propidium iodide (PI). Flow cytometry analysis was carried out on a BD LSRII flow cytometer at the Koch Institute. Gates were applied to the data to ensure that cells that were positive for propidium iodide or had forward/side scatter readings that were sufficiently different from the main cell population were excluded. Each sample was capped at 5,000 gated events.

Analysis was conducted using Graphpad Prism 7 and FlowJo. For each sample, the mean fluorescence intensity (MFI) and the number of gated cells was measured. To report activity, triplicate MFI values were averaged and normalized to the PMO alone condition.

### Example 4—Endocytosis Inhibition Assay

Chemical endocytosis inhibitors were used to probe the mechanism of delivery of PMO by these peptides in a pulse-chase format. Such analysis has been previously conducted on similar PMO-peptide constructs previously with comparable outcomes (Fadzen, C. M.; Holden, R. L.; Wolfe, J. M.; Choo, Z.-N.; Schissel, C. K.; Yao, M.; Hanson, G. J.; Pentelute, B. L. Biochemistry 2019). For the PMO constructs, HeLa 654 cells were preincubated with various chemical inhibitors for 30 minutes before treatment with PMO-CPP constructs for three hours. The panel included: a panel of endocytosis inhibitors including: chlorpromazine (CPZ), which is demonstrated to interfere with clathrin-mediated endocytosis; cytochalasin D (CyD), which inhibits phagocytosis and micropinocytosis; wortmannin (Wrt), which alters various endocytosis pathways by inhibiting phosphatidylinositol kinases; EIPA (5-(N-ethyl-Nisopropyl) amiloride), which inhibits micropinocytosis; and Dynasore (Dyn), which also inhibits clathrin-mediated endocytosis (Kjeken, R.; Mousavi, S. A.; Brech, A.; Griffiths, G.; Berg, T. Biochem. J. 2001, 357 (Pt 2), 497-503.; Dutta, D.; Donaldson, J. G. Cell. Logist. 2012, 2 (4), 203-208.). Treatment media was then replaced with fresh media and the cells were incubated for 22 hours at 37° C. and 5% CO₂. Cells were then lifted as previously described and EGFP synthesis was measured by flow cytometry.

### Example 5—Lactate Dehydrogenase (LDH) Release Assay

Cytotoxicity assays were performed in HeLa 654 cells. Cell supernatant following treatment for flow cytometry was transferred to a new 96-well plate for analysis of LDH release. To each well of the 96-well plate containing supernatant was added CytoTox 96 Reagent (Promega). The plate was shielded from light and incubated at room temperature for 30 minutes. Equal volume of Stop Solution was added to each well, mixed, and the absorbance of each well was measured at 490 nm. The blank measurement was subtracted from each measurement, and % LDH release was calculated as % cytotoxicity=100×Experimental LDH Release (OD490)/Maximum LDH Release (OD490).

### Example 6—Serum Stability Assay

Each PMO-peptide was dissolved in PBS to a concentration of 1 mM, as confirmed by UV-Vis. PMO-peptide was then added to a solution of either PBS or PBS containing 25% human serum to a final concentration of 50 μM and incubated at 37° C. 10 μL aliquots were removed at varying timepoints (t=0, 1 h, 6 h, 24 h), and quenched with 20 μL 1M guanidinium hydrochloride and 50 mM EDTA. 50 μL ice-cold acetonitrile was then added and the aliquots were flash frozen until LCMS analysis. Samples were thawed, and a portion of the aqueous layer was diluted before analysis by LC-qTOF. The mass spectrum for the PMO-peptides was analyzed by deconvolution, to best demonstrate whether the analyte had stayed intact or degraded.

### Example 7—Uptake Assay

Cell treatment: Cells were plated either in 6-well or 12-well plates at a density such that they reached 80% confluency the following day. CPP or PMO-CPP stock solutions were made fresh to 1 mM in cation-free PBS, as determined by UV-Vis. Treatment solution was then prepared by adding the stock solution to cell media at the concentrations described. Two wells were left untreated as controls. The plates were then incubated at 37° C. and 5% CO₂ for the designated time. For the experiment to arrest energy-dependent uptake, the plate was incubated at 4° C. Following incubation, the cells were washed three times with media, followed by 0.1 mg/mL Heparin in PBS for 5 min. Supernatant was aspirated and cells were lifted by incubating in trypsin-EDTA for 10 min at 37° C. Trypsin was quenched by adding cell media, and cells were transferred to Eppendorf tubes and pelleted at 500 rcf for 3 min. Pellets were washed by mixing with PBS, repeated twice.

Lysis: To acquire whole cell lysate, 50 μL RIPA (1× RIPA, protease inhibitor cocktail, water) was added to the cell pellet, mixed gently, and placed on ice for 1 h. To extract the cytosol, 50 μL digitonin buffer (0.05 mg/mL digitonin, 250 mM sucrose, PBS) was added to a cell pellet, mixed very gently, and placed on ice for 10 min. Samples were then pelleted by centrifugation at 16,000 rcf for 5 min. Supernatants were transferred to new Eppendorf tubes and kept on ice. Extracted protein was quantified using Pierce Rapid Gold BCA Protein Assay Kit (Thermo Fisher). 10 μg protein from each sample was then analyzed by SDS-PAGE gel for 35 min at 165 V and then transferred to a nitrocellulose membrane soaked in 48 mM Tris, 39 mM glycine, 0.0375% SDS, 20% methanol using a TransBlot Turbo Semi-Dry Transfer Unit (BioRad) for 7 mins. The membrane was blocked at 4° C. overnight in LI-Cor Odyssey blocking buffer (PBS). The membrane was then immunostained for 1 h with anti-Erk1/2 and anti-Rab5 in TBST at room temperature. After incubation, the membrane was washed three times with TBST and incubated with the appropriate secondary antibody in TBST for 1 h at room temperature, then washed with TBST. Finally, the membrane was incubated with streptavidin-HRP for 1 h and washed with TBST. To visualize HRP, the membrane was treated with SuperSignal West Pico PLUS chemiluminescent substrate (Thermo Fisher) immediately before imaging on a ChemiDoc MP Imaging System (Bio-Rad).

MALDI-TOF: The remaining cell extracts were then used for affinity capture and MALDI-TOF analysis, following an adapted protocol.⁶ 10 μL Dynabeads™ MyOne™ Streptavidin T1 (Thermo Fisher) were transferred to tubes in a magnet stand and washed with PBS. Cell extracts were added to the corresponding bead-containing tube and rotated at 4° C. overnight. One tube contained beads that were added to an equimolar solution of peptide conjugates used in the experiment. To insure the same equivalency are in the control tube as used in the experiment, this control solution was taken directly from the combined stock solution used in the initial cell treatment. The following day, the beads slurries were washed with a series of buffers: 2×100 μL Buffer A (50 mM Tris-HCl (pH 7.4) and 0.1 mg/mL BSA), 2×100 μL Buffer B (50 mM Tris-HCl (pH 7.4), 0.1 mg/mL BSA, and 0.1% SDS), 2×100 μL Buffer C (50 mM Tris-HCl (pH 7.4), 0.1 mg/mL BSA, and 1 M NaCl), and 2×100 μL water. Beads were then incubated with 100 μL of 1 mM biotin for 2 min, before washed with 5×50 μL water. Supernatant was removed and the beads were brought up in 3 μL MALDI matrix (saturated alpha-Cyano-4-hydroxycinnamic acid, CHCA) and transferred to the MALDI plate to dry. Beads were analyzed by MALDI-ToF on a high-resolution Bruker Autoflex LRF Speed mass spectrometer in linear positive mode.

Relative concentrations of peptides in the mixture were determined as follows. Analytes in a mixture ionize according to their response factor (F). F was determined by normalizing the intensities of each analyte to one analyte in the control sample, where the concentration of each analyte is arbitrarily set to 1. The values of F is then used in the experimental spectra containing the same mixture of analytes to determine their relative concentrations.

$$\frac{{I}_{x}}{[ X ]} = F ( \frac{{I}_{s}}{[ S ]} )$$

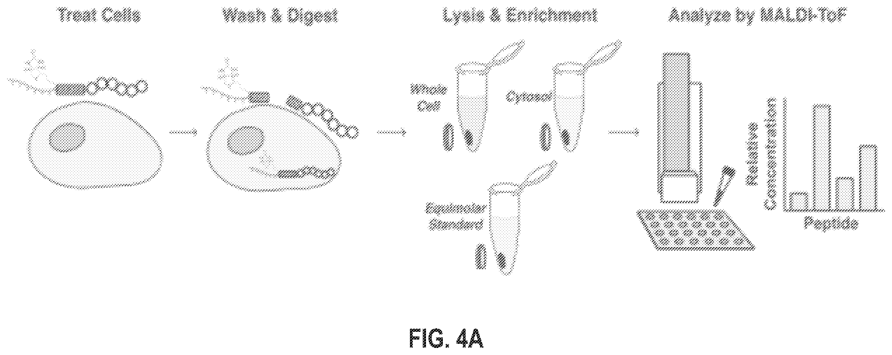

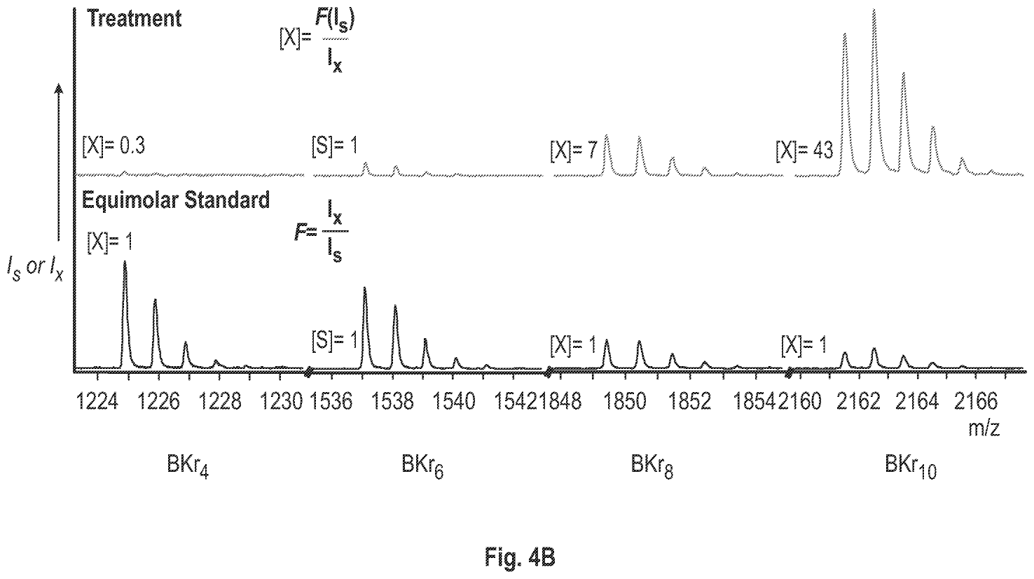

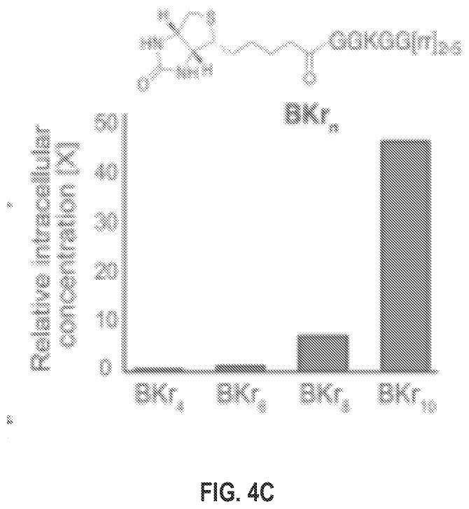

The proteostability of D-peptides allows for the recovery of mixtures of intact constructs after being internalized into cells. While MALDI-ToF has been used previously to analyze quantity of L-peptides recovered from inside cells, it has not yet been used to profile drug-peptide conjugates, D-peptides, or mixtures of more than three conjugates at a time. The use of D-peptides would allow for a mixture of conjugates to be analyzed, because without degradation, only the parent peak would be observed. Moreover, this platform has not yet been used to study peptides recovered from cell fractions. Through a series of experiments, the results demonstrate that mixtures of intact PMO-D-CPPs can be recovered from the cytosol of cells and analyzed by MALDI-ToF to estimate their relative abundances without the need for isotope labeling or standard curves (FIG. 4A).

The first step was to recapitulate a known empirical trend that more Arg residues leads toward greater uptake. A simple model system of four polyarginine peptides with a trypsin cleavable linker (GGKGG referenced herein as K) and a single biotin label was used. Biotin-K-D-Arg₄, D-Arg₆, D.Arg₈, and D-Arg₁₀ were incubated with HeLa cells for 1 h. The cells were then washed extensively with PBS and heparin and trypsinized, and the whole cell lysate was extracted. Fully intact biotinylated peptides were captured with magnetic streptavidin dynabeads, washed, and plated directly for MALDI analysis. Also plated were Dynabeads incubated in an equimolar mixture of the same constructs as determined by UV-Vis (FIG. 4B).

The relative concentration of peptides on the beads can be estimated by determining the analyte's response factor (F) from the equimolar standard (FIG. 4C). In the standard, each analyte's concentration equals 1 mM, and each analyte's response factor (F) is determined by normalizing their intensities to an internal control (S). Here, BKr₆ was selected as the arbitrary standard, where F=1. The response factor of each analyte should remain consistent across samples that contain the same analytes (Duncan, M. W. Practical Quantitative Biomedical Applications of MALDI-TOF Mass Spectrometry.; Ho, H.-P.; Rathod, P.; Louis, M.; Tada, C. K.; Rahaman, S.; Mark, K. J.; Leng, J.; Dana, D.; Kumar, S.; Lichterfeld, M.; Chang, E. J. Rapid Commun. Mass Spectrom. RCM 2014, 28 (24), 2681-2689.), and was used to calculate the fold change in concentration in the experimental samples. The relative concentrations [X] of the analytes, normalized to the ‘internal standard’ BKr₆ are shown as bar graph (FIG. 4C). There is a clear increase in concentration of the constructs with more Arg residues, with BKr₁₀ having 40-fold greater concentration than BKr₆. This trend of greater number of Arg residues leading to greater uptake is already well documented in the literature (Brock, R. Bioconjug. Chem. 2014, 25 (5), 863-868.; Fuchs, S. M.; Protein Sci. 2005, 14 (6), 1538-1544.).

The study confirmed by orthogonal means that the PMO-peptide conjugates entered via energy-dependent uptake and that outer membrane-bound conjugates do not contaminate lysate samples. First, an EGFP assay determined that for both PMO-D- and L-DPV7, incubation at reduced temperature negatively impacted PMO delivery. Then, HeLa cells were incubated with three PMO-D-CPPs at 37° C. or 4° C. before washing and lysis as before. Analysis by Western blot shows presence of both cytosolic and endosomal markers in both whole cell lysates, but shows a marked absence of biotinylated construct in the 4C condition by Streptavidin labeling. Analysis of these samples by MALDI-TOF also shows significantly reduced signal in the 4° C. condition compared to 37C, where only PMO-D-DPV7 is detected at reduced temperature. At the same time, no construct was detected in the 4° C. cytosolic condition. By using the constructs response factor (F) from the equimolar condition, it was evident that PMO-D-DPV7 had the highest relative intracellular concentration, although the three constructs had very similar concentrations.

### Example 8—Supplementary Uptake Data (Western Blot)

The orthogonal means that the PMO-peptide conjugates entered via energy-dependent uptake and that outer membrane-bound conjugates do not contaminate lysate samples were confirmed. First, an EGFP assay determined that for both PMO-D- and L-DPV7, incubation at reduced temperature negatively impacted PMO delivery. Then, HeLa cells were incubated with three PMO-D-CPPs at 37° C. or 4° C. before washing and lysis as before. Analysis by Western blot shows the presence of both cytosolic and endosomal markers in both whole cell lysates, but shows a marked absence of biotinylated construct in the 4° C. condition by Streptavidin labeling. Analysis of these samples by MALDI-TOF also shows significantly reduced signal in the 4° C. condition compared to 37° C., where only PMO-D-DPV7 is detected at reduced temperature. At the same time, no construct was detected in the 4° C. cytosolic condition. By using the constructs response factor (F) from the equimolar condition, it was evident that PMO-D-DPV7 had the highest relative intracellular concentration, although the three constructs had very similar concentrations.

Interestingly, the reduced temperature did not inhibit the uptake of the biotinylated-CPPs without the oligonucleotide cargo but rather equalized the relative concentration of the whole cell and cytosolic fractions. In the same manner, as the experiment with PMO-D-CPPs, biotin-D-DPV7, TATp, and DPV6 were incubated in HeLa cells at 37° C. and 4° C. Cytosol and whole-cell lysate were extracted, confirmed by Western blot. Samples were then analyzed by MALDI and the relative concentrations were determined, normalized to TATp. At 37° C., the determined relative calculations vary between the whole cell and cytosolic samples; DPV7 appears to have the highest concentration in the cytosol. However, at reduced temperature, the relative concentrations are nearly identical between the lysates. These experiments demonstrate that this method, in addition to determining relative internal concentration, is useful for investigating mechanisms of uptake.

### Example 9—Dose-Response Studies

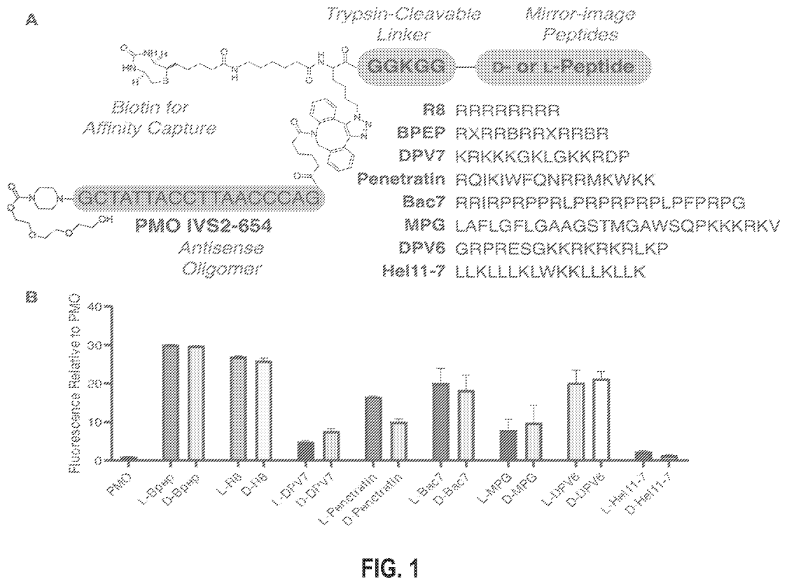

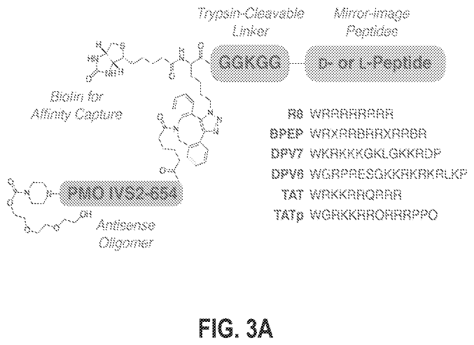

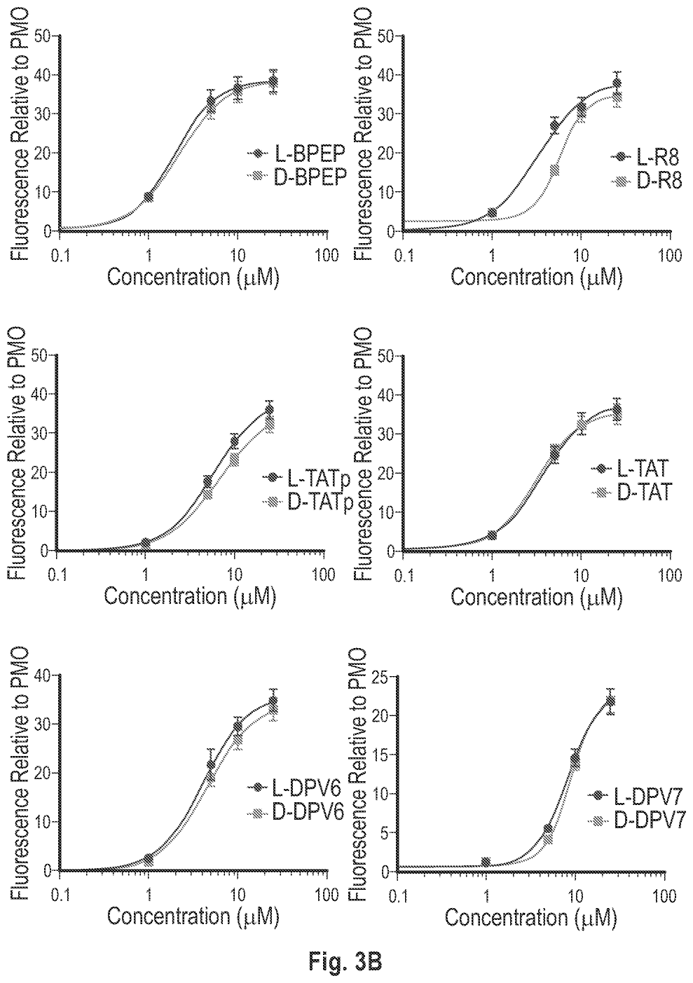

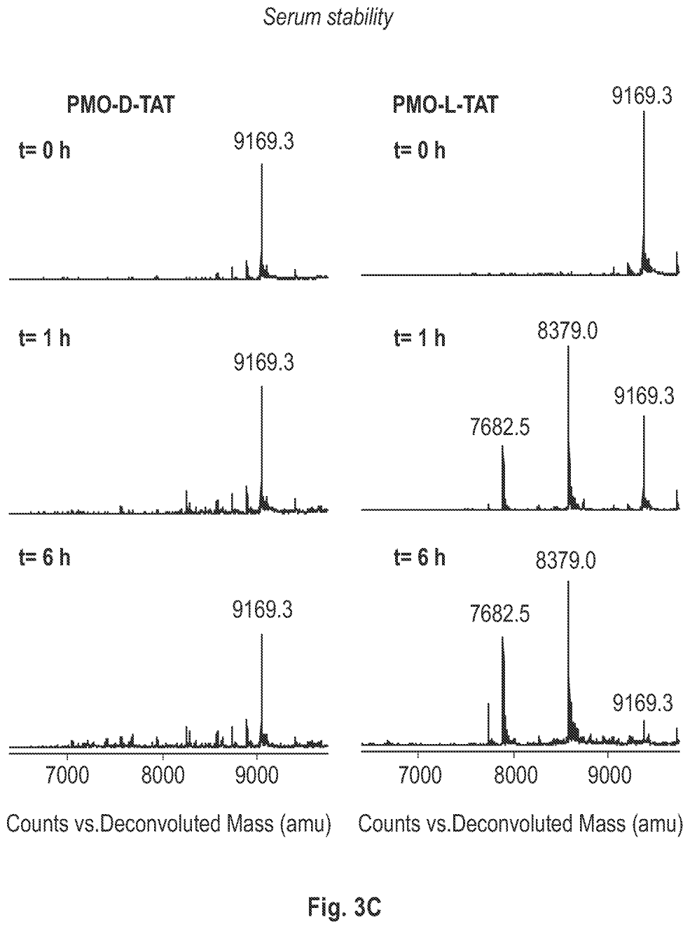

Dose-response studies with sequences in the D- and L-form confirmed similar activities between mirror image CPPs. From the initial proof-of-concept experiment, involving eight sequences in L- and D-form tested at a single concentration (5 μM), there was not a significant difference between the activities of the mirror image peptides (FIG. 1B). These constructs were tested at varying concentrations in the EGFP 654 assay, and the results further suggested that mirror image peptides shared nearly identical PMO delivery activities (FIG. 3A and Example 4).

Furthermore, the supernatant from these assays was tested for lactate dehydrogenase (LDH) release, indicative of membrane toxicity, and it was confirmed that the constructs did not elicit membrane toxicity at the doses tested (Example 5).

D-peptides are stable against degradation, illustrated by a time-course study in which both forms of PMO-CPPs were incubated in 25% human serum. While the studied PMO-D-CPPs remained intact 24 h later, the L-forms rapidly degraded into multiple fragments, leaving the parent construct as a minor product after only one hour of incubation (FIG. 3B). This observation furthers the notion that L-peptides are not suitable for investigation using mass spectrometry after recovery from a biological setting. However, D-peptide conjugates can be recovered from a biological environment such as serum without suffering degradation, allowing for their characterization via mass-spectrometry.

### Example 10—Cytosol Extraction and Evaluation of CPP Delivery Efficiency

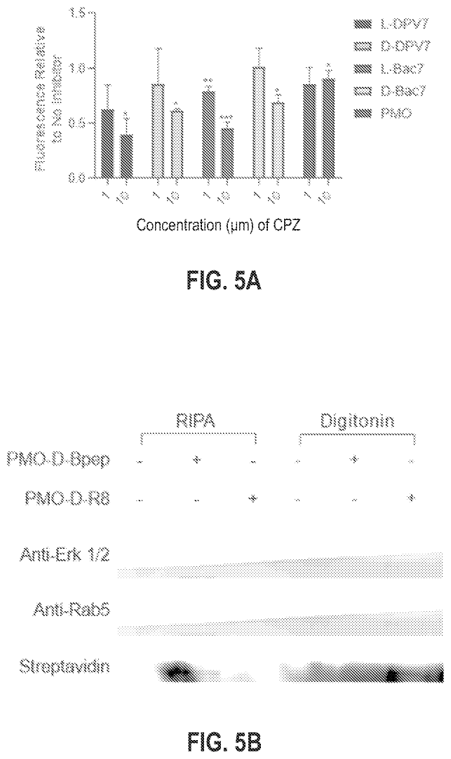

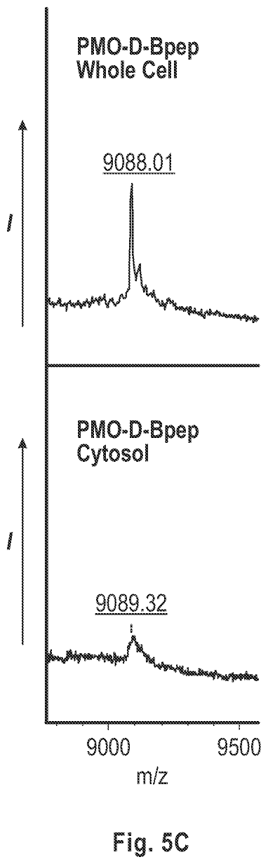

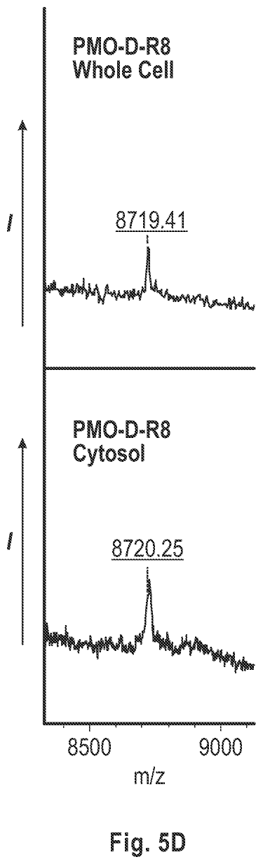

To evaluate CPP delivery efficiency using a panel of chemical endocytosis inhibitors, a pulse-chase EGFP 654 assay format was performed in which cells were pre-incubated with inhibitors before treatment with the L- and D-forms of PMO-DPV7 and PMO-Bac7 (Example 5). Analysis by flow cytometry revealed that chlorpromazine reduced activity in a dose-dependent manner (FIG. 5A). Chlorpromazine is an inhibitor of clathrin-mediated endocytosis and has been observed to inhibit activity of similar PMO constructs previously. While it is possible that multiple uptake mechanisms are occurring, these PMO-CPP conjugates are likely taken up by active transport.

Knowing that PMO-CPPs enter via endocytosis, extracting the cytosol and comparing to the whole-cell lysate is critical when evaluating relative concentrations of constructs internalized into cells. Not all endocytosed compounds are able to escape the endosome, and endosomal entrapment would lead to less active PMO delivered into the cytosol, measured by a lower cytosolic concentration relative to whole-cell lysate. Therefore, the biotinylated PMO-CPPs were extracted from the cytosol as well as the whole cell lysate following internalization and detected them by Western blot and MALDI. Individually, PMO-D-R₃ or PMO-D-Bpep were incubated at 5 μM with HeLa cells in a 12-well plate for 1 h before washing and digesting with trypsin. The cytosol was extracted using Digitonin buffer, which selectively permeabilizes the outer membrane. RIPA buffer was used to prepare whole cell lysates. To confirm cytosolic extraction, a portion of each sample was analyzed via Western blot using a cytosolic marker (Erk 1/2) and a late-endosomal marker (Rab5). Samples of cytosolic extract have markedly reduced Rab5 while all samples contain Erk 1/2 (FIG. 5B). Finally, as with the biotinylated peptides, the PMO-CPPs were then extracted from the samples with Streptavidin coated magnetic Dynabeads, washed extensively, and analyzed via MALDI-TOF. PMO-D-R₈ and PMO-D-Bpep were detected in their respective samples, presenting the first instance of an intact peptide-oligonucleotide conjugate being extracted from cells and analyzed by mass spectrometry (FIG. 5C-D). Moreover, incubation at lower temperature appeared to inhibit cytosolic localization of PMO-D-CPPs, but resulted in equal relative concentrations between whole cell and cytosolic fractions for biotin-D-CPPs (Example 7).

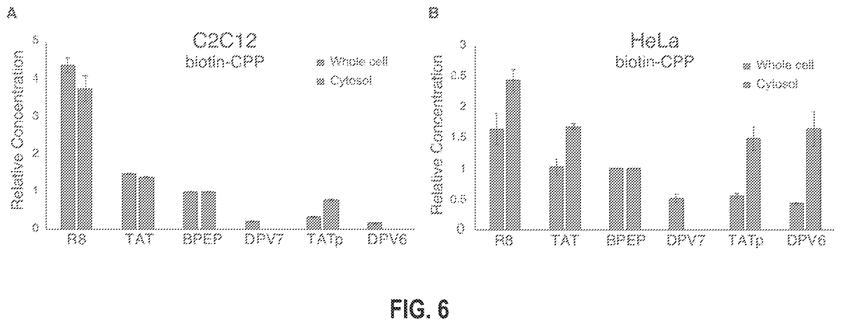

Next, the analytes tested were expanded and the uptake of six biotin-D-CPPs were profiled in the cytosol of distinct cell lines. Biotin-D-R₈, TAT, Bpep, DPV7, TATp, and DPV6 were profiled by MALDI in both HeLa (FIG. 6A) and C2C12 mouse myoblast (FIG. 6B) cell lines. Different uptake patterns were observed between the two cell lines; polyarginine was significantly more abundant in the C2C12 cells compared to the other peptides, and DPV6 and DPV7 were not detected in the cytosol. In HeLa, polyarginine had the highest relative concentration in both cytosol and whole cell, and DPV7 was again not detected in the cytosol. However, TAT, TATp, and DPV6 all had similar relative cytosolic concentrations. With these experiments it is demonstrated that the profiling platform could determine the relative concentration of six intact peptides extracted from whole cell and cytosol of two distinct cell lines.

Finally, the uptake of six PMO-D-CPPs. PMO-D-R₈, TAT, BPEP, DPV7, TATp, and DPV6 were profiled in HeLa cells as usual. Extraction of cytosol was confirmed by Western blot and the samples were analyzed by MALDI. Relative concentrations were normalized to BPEP. In the whole-cell extract, the relative concentrations were generally consistent across peptides, with DPV6 having a 1.5-fold higher relative concentration. On the other hand, relative concentrations in the cytosol were lower for the polyarginine peptides R₈ and TAT, and highest for BPEP and DPV6 (FIG. 7A). By comparing the relative concentrations in cytosolic and whole-cell lysates, the relative efficiencies demonstrate an effective metric for determining which of the six CPPs can effectively deliver PMO into the cytosol (FIG. 7B).

By combining relative internal concentrations with PMO delivery activity, relative delivery efficiency can be determined. Delivery efficiency would be a useful metric for comparing CPPs delivering active therapeutic cargo as it takes into account both the activity of the cargo as well as the internal concentration. A highly efficient peptide would have high activity with low internal concentration, and thus high relative efficiency.

### Example 11—Preparation of Peptide Libraries

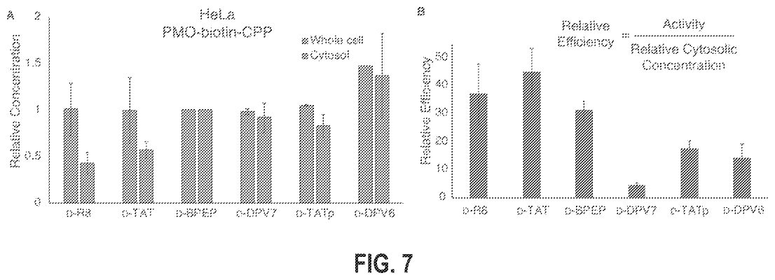

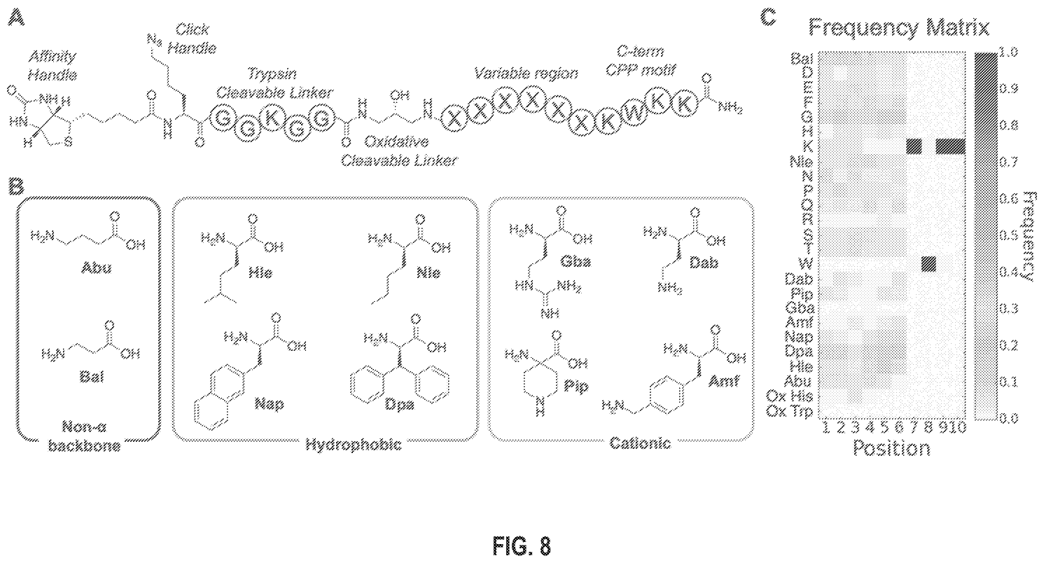

The library was prepared with a “CPP-like” C-terminal sequence and six variable positions containing D- and unnatural amino acids (FIG. 8A). Split-and-pool synthesis afforded 0.016 μg of peptide per bead for a low-redundancy, 95,000-member library with a theoretical diversity greater than 108. A KWKK motif, derived from the established cell-penetrating peptide penetratin, was installed at the C-terminus. Unnatural amino acids were chosen to expand the chemical diversity and potentially enhance cell penetration of the library peptides. The library includes unnatural residues with non-α backbones (γ-aminobutyric acid and β-alanine), 8 residues with hydrophobic and aromatic functionality (homoleucine, norleucine, naphthylalanine, and diphenylalanine), and additional charged residues and arginine analogues (diaminobutyric acid, aminopiperidine-carboxylic acid, aminomethylphenylalanine, and 2-amino-4-guanidinobutanoic acid) (FIG. 8B). The oxidative cleavable linker isoseramox was installed by reductive amination immediately following the variable region, followed by a trypsin cleavage site. Finally, azidolysine and biotin capped the N-terminus of the sequences to allow for PMO conjugation and affinity capture, respectively. Following cleavage from the resin, a portion of the library was conjugated by azide-alkyne cycloaddition to a model PMO derivatized with dibenzocyclooctyne (DECO), monitored by LC-MS. Quality control analysis of the library by Orbitrap nano-liquid chromatography-tandem mass spectrometry (nLC-MS/MS) confirmed successful synthesis and exhibited a range of incorporated residues (FIG. 80). This library design ensured the isolation of 10-mer peptides with a native N-terminus, suitable for sequencing via tandem mass spectrometry.

TABLE 4
Peptide Library

| SEQ | | Terminal Motif |
|---|---|---|
| ID NO. | Peptide Sequences | (d-amino acids) |
| 33 | (Hle)(Pip)(d-Asp)(Nap)(d-Asp)(Dpa) | KWKK |
| 34 | (Dpa)(d-Phe)(d-Lys)(d-Phe)(d-Asn)(d-Thr) | KWKK |
| 35 | (Nap)(d-Nle)(Abu)(d-Pro)(d-Nle)(Amf) | KWKK |
| 36 | (Dpa)(d-Asn)(d-Thr)(d-Arg)(Dpa)(d-Asn) | KWKK |
| 37 | (Nap)(d-Thr)(d-Phe)(d-Nle)(Gly)(d-Glu) | KWKK |
| 38 | (Dpa)(d-Phe)(d-Lys)(Nap)(d-Ser)(d-Asp) | KWKK |
| 39 | (Gly)(Dpa)(B)(Dpa)(d-Phe)(d-Gln) | KWKK |
| 40 | (d-Asp)(d-Thr)(B)(d-Nle)(Dpa)(Dpa) | KWKK |
| 41 | (Nap)(d-Pro)(d-Nle)(d-Glu)(Nap)(Hle) | KWKK |
| 42 | (Hle)(d-Phe)(Hle)(d-Asn)(d-Thr)(d-Gln) | KWKK |
| 43 | (d-Asn)(d-Nle)(Abu)(d-Nle)(Hle)(d-Gln) | KWKK |
| 44 | (d-Asp)(Dpa)(Amf)(d-Asp)(Dpa)(d-Lys) | KWKK |
| 45 | (d-Ser)(Dab)(Nap)(d-Asp)(Dpa)(d-Gln) | KWKK |
| 46 | (d-Phe)(d-Ser)(Dpa)(Gly)(Hle)(d-Arg) | KWKK |
| 47 | (Gly)(Dpa)(d-Nle)(d-His)(Dpa)(Gly) | KWKK |
| 48 | (d-Ser)(d-Glu)(Dpa)(d-Thr)(Nap)(Gly) | KWKK |
| 49 | (Hle)(d-Thr)(d-Glu)(Hle)(Dpa)(d-Pro) | KWKK |
| 50 | (Gly)(d-Phe)(d-Pro)(d-Nle)(d-Glu)(Dpa) | KWKK |
| 51 | (Nap)(Hle)(d-Thr)(d-Gln)(Nap)(d-Glu) | KWKK |
| 52 | (Dpa)(Nap)(Hle)(d-Gln)(Pip)(d-Phe) | KWKK |
| 53 | (d-Pro)(d-Lys)(d-Nle)(d-Asp)(d-Phe)(d-Nle) | KWKK |
| 54 | (Hle)(Dab)(d-Phe)(d-Gln)(d-Arg)(Dpa) | KWKK |
| 55 | (Dpa)(Dpa)(d-Lys)(d-His)(Hle)(d-Thr) | KWKK |
| 56 | (d-Ser)(d-Asn)(Dpa)(d-Asp)(Dpa)(Pip) | KWKK |
| 57 | (d-Thr)(d-Asn)(d-Phe)(d-Nle)(Gly)(Dpa) | KWKK |
| 58 | (d-Phe)(Hle)(Hle)(d-Asn)(d-Thr)(d-Gln) | KWKK |
| 59 | (d-Lys)(d-Phe)(d-Ser)(d-Nle)(d-Glu)(Dab) | KWKK |
| 60 | (B)(d-Lys)(d-Gln)(Dpa)(d-Phe)(d-Asn) | KWKK |
| 61 | (Pip)(Hle)(d-Arg)(Amf)(Gly)(Dpa) | KWKK |
| 62 | (Dpa)(Gly)(d-Glu)(d-Asn)(d-Asp)(d-Nle) | KWKK |
| 63 | (d-Phe)(Dpa)(d-Gln)(d-Glu)(Abu)(d-Nle) | KWKK |
| 64 | (Nap)(B)(d-Phe)(Dpa)(Amf)(d-Glu) | KWKK |
| 65 | (d-Asp)(d-Thr)(B)(d-Nle)(Nap)(Dpa) | KWKK |
| 66 | (d-Phe)(Gly)(d-Pro)(d-Nle)(d-Glu)(Dpa) | KWKK |
| 67 | (Dpa)(Gly)(B)(Dpa)(d-Phe)(d-Gln) | KWKK |
| 68 | (d-Phe)(d-Lys)(Nap)(d-Asn)(d-Phe)(d-Lys) | KWKK |
| 69 | (d-Asn)(d-Phe)(d-Asp)(d-Ser)(Dpa)(Amf) | KWKK |
| 70 | (d-Pro)(d-Ser)(d-Phe)(Dpa)(d-Glu)(B) | KWKK |
| 71 | (d-Pro)(d-Nle)(d-Nle)(d-Glu)(Nap)(Hle) | KWKK |
| 72 | (Abu)(Dpa)(d-Phe)(Hle)(d-Ser)(Hle) | KWKK |
| 73 | (Gly)(Dpa)(Nle)(Dpa)(Gly) | KWWKK |
| 74 | (B)(Hle)(d-Thr)(d-Nle)(Gly)(d-Asn) | KWKK |
| 75 | (d-Lys)(Dpa)(Gly)(d-Gln)(Amf)(d-Nle) | KWKK |
| 76 | (Dpa)(Abu)(d-Asn)(d-Arg)(d-Nle)(d-Thr) | KWKK |
| 77 | (d-Glu)(B)(Dpa)(Dab)(d-Nle)(Gly) | KWKK |
| 78 | (d-Thr)(d-Arg)(Dpa)(d-Gln)(B)(d-Phe) | KWKK |
| 79 | (d-Thr)(d-Lys)(Dpa)(Abu)(d-Lys)(Dpa) | KWKK |
| 80 | (Dab)(Dpa)(d-Asn)(Dpa)(Hle)(B) | KWKK |
| 81 | (d-Glu)(Hle)(d-Thr)(Nap)(d-Gln)(Nap) | KWKK |
| 82 | (Abu)(d-Asp)(Dpa)(Dab)(d-Nle)(Gly) | KWKK |
| 83 | (Hle)(d-Lys)(Dpa)(Gly)(d-Thr)(d-Pro) | KWKK |
| 84 | (d-Gln)(Nap)(d-Nle)(Pip)(Amf)(Hle) | KWKK |
| 85 | (d-Asn)(Gly)(Dpa)(d-Thr)(Nap)(Gly) | KWKK |
| 86 | (Gly)(d-Asn)(Dpa)(d-Thr)(Nap)(Gly) | KWKK |
| 87 | (d-Ser)(d-Phe)(d-Glu)(d-Asp)(Gly)(Gly) | KWKK |
| 88 | (d-Phe)(d-Phe)(d-Thr)(d-Phe)(d-Thr)(Abu) | KWKK |
| 89 | (d-Pro)(Dpa)(Hle)(Hle)(Abu)(d-Pro) | KWKK |
| 90 | (d-Asn)(Abu)(d-Glu)(d-Lys)(d-Nle)(Nap) | KWKK |
| 91 | (d-Phe)(Dpa)(Dpa)(Pip)(d-Phe)(Dpa) | KWKK |
| 92 | (d-Lys)(Hle)(Dpa)(Gly)(d-Thr)(d-Pro) | KWKK |
| 93 | (Hle)(d-Thr)(d-Glu)(Hle)(d-Pro)(Dpa) | KWKK |
| 94 | (d-Thr)(Dab)(d-Asp)(d-Nle)(d-Lys)(Nap) | KWKK |
| 95 | (d-Glu)(d-Phe)(Nap)(d-Asn)(Nap)(Amf) | KWKK |
| 96 | (B)(d-Pro)(d-Pro)(Nap)(Nap)(d-Asn) | KWKK |
| 97 | (d-Lys)(d-Thr)(Dpa)(Abu)(d-Lys)(Dpa) | KWKK |
| 98 | (Gly)(d-Lys)(d-Glu)(Hle)(d-Phe)(Amf) | KWKK |
| 99 | (d-Lys)(Dab)(d-Thr)(Dpa)(d-Gln)(d-Nle) | KWKK |
| 100 | (d-Phe)(d-Nle)(B)(Abu)(Dab)(Nap) | KWKK |
| 101 | (Dpa)(Hle)(B)(Gly)(Pip)(d-Phe) | KWKK |
| 102 | (Dpa)(d-Gln)(Abu)(Abu)(Pip)(Dpa) | KWKK |
| 103 | (d-Lys)(Gly)(d-Glu)(Hle)(d-Phe)(Amf) | KWKK |
| 104 | (d-Phe)(d-Phe)(d-Nle)(d-Glu)(Nap)(Hle) | KWKK |
| 105 | (d-Ser)(d-Asn)(d-Phe)(d-Nle)(Gly)(Dpa) | KWKK |
| 106 | (d-Phe)(d-Phe)(d-Asn)(Nap)(B)(Nap) | KWKK |
| 107 | (d-Asp)(Abu)(Dpa)(Dab)(d-Nle)(Gly) | KWKK |
| 108 | (d-Pro)(Nap)(d-Nle)(d-Glu)(Nap)(Hle) | KWKK |
| 109 | (B)(B)(d-Lys)(d-Asp)(d-Nle)(d-Phe) | KWKK |
| 110 | (d-Phe)(Amf)(d-Asp)(d-Asp)(Dpa)(d-Phe) | KWKK |
| 111 | (d-Nle)(d-Asn)(d-Thr)(d-Gln)(Nap)(d-Glu) | KWKK |
| 112 | (d-Phe)(d-Ser)(d-Glu)(d-Asp)(Gly)(Gly) | KWKK |
| 113 | (Amf)(B)(d-Gln)(Hle)(Dpa)(Gly) | KWKK |
| 114 | (Gly)(Dpa)(Dpa)(Gly)(Gly)(Dab) | KWKK |
| 115 | (Abu)(Dpa)(d-Phe)(Hle)(d-Ser)(Pip) | KWKK |
| 70 | (d-Pro)(d-Ser)(d-Phe)(Dpa)(d-Glu)(B) | KKWK |
| 116 | (d-Thr)(Pip)(d-Thr)(d-Gln)(Nap)(d-Glu) | KWKK |
| 117 | (d-Arg)(d-Lys)(d-Nle)(Hle)(d-Lys)(d-Nle) | KWKK |
| 118 | (Gly)(Gly)(Dab)(d-Nle)(Hle)(d-Nle) | KWKK |
| 119 | (d-Gln)(d-His)(d-Asn)(Dpa)(Hle)(B) | KWKK |
| 120 | (Dpa)(B)(d-Nle)(d-Glu)(Nap)(Hle) | KWKK |
| 121 | (d-Glu)(d-Phe)(Gly)(Gly)(Nap)(Amf) | KWKK |
| 122 | (d-Phe)(d-Phe)(d-Asn)(d-Arg)(Abu)(Dpa) | KWKK |
| 123 | (d-Asn)(d-Glu)(d-His)(d-Thr)(Nap)(Gly) | KWKK |
| 124 | (d-Arg)(d-Thr)(Dpa)(d-Gln)(B)(d-Phe) | KWKK |
| 125 | (d-His)(d-Gln)(d-Asn)(Dpa)(Hle)(B) | KWKK |
| 126 | (Abu)(d-Phe)(d-Glu)(d-Glu)(B)(d-Asp) | KWKK |
| 127 | (B)(Abu)(B)(Dpa)(Hle)(Gly) | KWKK |
| 128 | (d-Asn)(d-His)(d-Asn)(Dpa)(Hle)(B) | KWKK |
| 129 | (Abu)(d-Pro)(d-Pro)(B)(d-Ser)(d-Glu) | KWKK |
| 130 | (d-Thr)(d-Arg)(Dpa)(Abu)(d-Asn)(d-Phe) | KWKK |
| 131 | (d-Pro)(Abu)(d-Pro)(Gly)(Gly)(d-His) | KWKK |
| 132 | (Pip)(Nap)(Hle)(d-Asp)(d-Thr)(Pip) | KWKK |
| 133 | (B)(Gly)(d-Pro)(Abu)(Dpa)(Nap) | KWKK |
| 134 | (d-Asn)(d-His)(d-Glu)(d-Thr)(Nap)(Gly) | KWKK |
| 135 | (d-Nle)(B)(Abu)(d-Ser)(d-Asn)(d-Asp) | KWKK |
| 136 | (Hle)(d-Lys)(d-Nle)(d-Glu)(d-Ser)(d-Glu) | KWKK |
| 137 | (Amf)(d-Thr)(d-Lys)(d-Nle)(d-Gln)(Dpa) | KWKK |
| 138 | (Dpa)(B)(Dpa)(d-Phe)(B)(Gly) | KWKK |
| 139 | (B)(d-Ser)(d-Ser)(d-Ser)(Hle)(Hle) | KWKK |
| 140 | (d-Glu)(d-Ser)(ox-His)(d-Ser)(Dpa)(Gly) | KKKK |
| 141 | (Dab)(Gly)(ox-His)(d-Glu)(Nap)(Hle) | KWKK |
| 142 | (B)(Abu)(d-Nle)(d-Ser)(d-Gln)(d-Gln) | KWKK |
| 143 | (Abu)(d-Nle)(B)(d-Ser)(d-Asn)(d-Asp) | KWKK |
| 144 | (Dpa)(d-Glu)(d-Pro)(B)(d-His)(Gly) | KWKK |
| 145 | (Gly)(Gly)(Hle)(d-Arg)(d-Nle)(d-Phe) | KWKK |
| 146 | (Dpa)(d-Phe)(d-Lys)(d-Phe)(d-Asp)(Dab) | KWKK |
| 147 | (d-Glu)(d-Asn)(d-His)(d-Thr)(Nap)(Gly) | KWKK |
| 148 | (d-Nle)(d-Gln)(d-Arg)(d-Glu)(d-Gln)(Nap) | KWKK |
| 149 | (d-Nle)(d-Glu)(d-Phe)(d-Thr)(d-Asp)(d-Thr) | KWKK |
| 150 | (d-Glu)(Hle)(d-Thr)(Gly)(B)(Nap) | KWKK |
| 151 | (Pip)(d-Ser)(d-Pro)(d-Glu)(Nap)(Hle) | KWKK |
| 152 | (d-Gln)(d-Pro)(Abu)(d-Glu)(Nap)(Hle) | KWKK |
| 153 | (d-Ser)(d-Arg)(d-Asn)(Dpa)(Abu)(Gly) | KWKK |
| 154 | (Dpa)(d-Thr)(d-Asn)(d-Glu)(d-Thr)(d-Ser) | KWKK |
| 155 | (d-Asn)(Hle)(d-Pro)(Nap)(B)(d-Ser) | KWKK |
| 156 | (Pip)(Gly)(Gly)(d-Phe)(Hle)(Dpa) | KWKK |
| 157 | (Abu)(d-Pro)(B)(Gly)(Dpa)(Nap) | KWKK |
| 158 | (Gly)(Dab)(d-Ser)(d-Nle)(d-Glu)(Dpa) | KWKK |
| 159 | (Abu)(d-Gln)(Abu)(d-Nle)(Hle)(d-Gln) | KWKK |
| 160 | (Amf)(d-Asn)(Abu)(Hle)(Dpa)(Gly) | KWKK |
| 161 | (d-Glu)(ox-His)(d-Ser)(d-Ser)(Dpa)(Gly) | KKKK |
| 162 | (d-Arg)(d-Asn)(d-Glu)(d-Ser)(d-His)(d-Phe) | KWKK |
| 163 | (d-Gln)(d-Gln)(d-Ser)(d-Pro)(d-Nle)(Gly) | KWKK |
| 164 | (d-Asn)(Gly)(d-His)(d-Asn)(B)(Dpa) | KWKK |
| 165 | (B)(Dpa)(d-Nle)(d-Glu)(Nap)(Hle) | KWKK |
| 166 | (B)(Gly)(B)(Hle)(Dpa)(Gly) | KWKK |
| 167 | (Pip)(d-His)(d-Asp)(d-Phe)(d-Arg)(d-Asp) | KWKK |
| 168 | (Dpa)(d-Gln)(d-Nle)(Gly)(Pip)(Dpa) | KWKK |
| 169 | (Dab)(Dpa)(d-Asn)(Dpa)(d-Nle)(Abu) | KWKK |
| 170 | (d-Pro)(Gly)(d-Asn)(Hle)(Gly)(d-Asn) | KWKK |
| 171 | (Pip)(Gly)(d-His)(Nap)(Amf)(Dab) | KWKK |
| 172 | (Gly)(Gly)(ox-His)(Nap)(d-Asp)(Dpa) | KWKK |
| 173 | (d-Thr)(B)(Pip)(d-Nle)(Hle)(d-Gln) | KWKK |
| 174 | (d-Asn)(d-His)(Gly)(B)(d-Asn)(Dpa) | KWKK |
| 175 | (d-Thr)(d-Phe)(d-Asp)(Dpa)(Hle)(B) | KWKK |
| 176 | (d-Glu)(d-His)(d-Pro)(Dpa)(Hle)(B) | KWKK |
| 177 | (Abu)(B)(B)(Dpa)(Hle)(Gly) | KWKK |
| 178 | (d-Lys)(d-Pro)(d-Nle)(d-Asp)(d-Phe)(d-Nle) | KWKK |
| 179 | (Hle)(d-Pro)(d-Nle)(d-Ser)(Abu)(B) | KWKK |
| 180 | (Dpa)(d-Thr)(B)(Abu)(Dpa)(d-Asn) | KWKK |
| 181 | (Abu)(Dpa)(d-Phe)(Hle)(d-Ser)(Dpa) | KWKK |
| 182 | (d-His)(d-Pro)(d-Glu)(Dpa)(Hle)(B) | KWKK |
| 183 | (d-Nle)(Abu)(d-Thr)(d-Nle)(Gly)(d-Asn) | KWKK |
| 184 | (B)(Nap)(d-Phe)(Dpa)(Amf)(d-Glu) | KWKK |
| 185 | (Dpa)(Gly)(d-Nle)(d-His)(Dpa)(Gly) | KWKK |
| 186 | (Pip)(d-Ser)(Abu)(d-Nle)(Hle)(d-Gln) | KWKK |
| 187 | (d-Asp)(Abu)(d-Ser)(d-Nle)(Dpa)(Dpa) | KWKK |
| 188 | (d-Gln)(Abu)(d-Pro)(d-Glu)(Nap)(Hle) | KWKK |
| 189 | (Amf)(Abu)(B)(Abu)(Hle)(Dpa) | KWKK |
| 190 | (d-Nle)(Abu)(d-Nle)(Hle)(B)(Gly) | KWKK |
| 191 | (d-His)(Gly)(B)(d-Nle)(Dpa)(Gly) | KWKK |
| 192 | (Gly)(B)(d-His)(d-Nle)(Dpa)(Gly) | KWKK |
| 193 | (Pip)(d-His)(Gly)(Nap)(Amf)(Dab) | KWKK |
| 194 | (Dpa)(Hle)(Gly)(B)(Pip)(d-Phe) | KWKK |
| 195 | (d-Nle)(d-Pro)(d-Nle)(d-Glu)(Nap)(Hle) | KWKK |
| 196 | (Pip)(B)(d-Nle)(Gly)(Hle)(d-His) | KWKK |
| 197 | (Amf)(d-Ser)(Gly)(Nap)(Amf)(Dab) | KWKK |
| 198 | (Hle)(d-Pro)(B)(Gly)(Gly)(Nap) | KWKK |
| 199 | (d-Arg)(d-Lys)(d-Nle)(Hle)(d-Nle)(d-Lys) | KWKK |
| 200 | (d-Nle)(Gly)(B)(d-Arg)(d-Nle)(d-Phe) | KWKK |
| 201 | (Gly)(Gly)(Abu)(Hle)(Dpa)(Gly) | KWKK |
| 202 | (Gly)(Gly)(d-His)(d-Phe)(Hle)(d-Ser) | KWKK |
| 203 | (d-Asn)(d-Phe)(Gly)(d-Glu)(d-Pro)(Dpa) | KWKK |
| 204 | (d-Asp)(Nap)(Gly)(d-Ser)(Dpa)(Gly) | KWKK |
| 205 | (d-Thr)(ox-His)(d-Asp)(d-Gln)(d-Pro)(Dpa) | KWKK |
| 206 | (d-Asn)(Nap)(Gly)(d-Glu)(d-Pro)(Dpa) | KWKK |
| 207 | (d-Nle)(Abu)(B)(d-Ser)(d-Asn)(d-Asp) | KWKK |
| 208 | (d-Thr)(Gba)(d-Asn)(Dpa)(Abu)(Gly) | KWKK |
| 209 | (d-Thr)(Pip)(B)(d-Nle)(Hle)(d-Gln) | KWKK |
| 210 | (d-Asp)(Abu)(d-Ser)(d-Nle)(Nap)(Dpa) | KWKK |
| 211 | (B)(Gly)(d-His)(d-Nle)(Dpa)(Gly) | KWKK |
| 212 | (Nap)(Gly)(d-Asp)(d-Gln)(d-Pro)(Dpa) | KWKK |
| 213 | (d-Lys)(B)(d-Phe)(B)(Hle)(Dpa) | KWKK |
| 214 | (Gly)(d-Phe)(d-Ser)(d-Nle)(d-Nle)(d-Phe) | KWKK |
| 215 | (d-Lys)(B)(ox-His)(d-Gln)(Dpa)(d-Asn) | KWKK |
| 216 | (Gly)(Gly)(d-Asn)(d-Phe)(d-Phe)(d-Gln) | KWKK |
| 217 | (d-Asp)(Pip)(d-His)(d-Phe)(d-Arg)(d-Asp) | KWKK |
| 218 | (ox-His)(d-Gln)(d-Pro)(d-Phe)(d-Arg)(d-Asp) | KWKK |
| 219 | (Gly)(d-Ser)(Amf)(Nap)(Amf)(Dab) | KWKK |
| 220 | (Pip)(d-Asp)(d-His)(d-Phe)(d-Arg)(d-Asp) | KWKK |
| 221 | (Gly)(ox-His)(Gly)(Nap)(d-Asp)(Dpa) | KWKK |
| 222 | (B)(Dab)(d-His)(d-Phe)(Hle)(d-Ser) | KWKK |
| 223 | (Nap)(d-Glu)(d-Gln)(d-Pro)(Pip)(d-Phe) | KWKK |
| 224 | (Abu)(B)(d-Nle)(d-Ser)(d-Gln)(d-Gln) | KWKK |
| 225 | (Abu)(Dpa)(d-Asn)(d-Arg)(d-Nle)(d-Thr) | KWKK |
| 226 | (Dpa)(d-Thr)(Abu)(B)(Dpa)(d-Asn) | KWKK |
| 227 | (d-His)(Gly)(Gly)(d-Phe)(Hle)(d-Ser) | KWKK |
| 228 | (d-Pro)(d-Nle)(d-Asn)(d-Asp)(d-Pro)(Hle) | KWKK |
| 229 | (Gly)(d-His)(Gly)(Abu)(d-Lys)(Dpa) | KWKK |
| 230 | (Nap)(d-Glu)(ox-His)(B)(Hle)(d-Phe) | KWKK |
| 231 | (d-Asp)(d-Pro)(d-Asp)(d-His)(d-Asp)(Dpa) | KWKK |
| 232 | (Pip)(Pip)(d-Glu)(d-Pro)(d-Thr)(Pip) | KWKK |
| 233 | (Dpa)(d-Glu)(d-Asn)(d-Gln)(d-Pro)(Dpa) | KWKK |
| 234 | (d-Asn)(Nap)(d-Asp)(B)(d-Pro)(Dpa) | KWKK |
| 235 | (Abu)(B)(d-Asn)(Hle)(d-His)(d-Asn) | KWKK |
| 236 | (B)(d-Ser)(d-Thr)(Dab)(Dpa)(d-Asn) | KWKK |
| 237 | (d-Pro)(d-Nle)(d-Glu)(d-Pro)(d-Thr)(Pip) | KWKK |
| 238 | (d-Asn)(d-His)(B)(Dpa)(d-Phe)(d-Gln) | KWKK |
| 239 | (Dpa)(Abu)(d-Gln)(Abu)(Pip)(Dpa) | KWKK |
| 240 | (Abu)(d-Pro)(Gly)(B)(Dpa)(Nap) | KWKK |
| 241 | (d-Ser)(ox-His)(d-Glu)(d-Ser)(Dpa)(Gly) | KKKK |
| 242 | (d-Gln)(d-Gln)(d-Nle)(d-Ser)(d-Pro)(Gly) | KWKK |
| 243 | (d-Ser)(Amf)(d-Ser)(Gly)(Pip)(Dab) | KWKK |
| 244 | (d-Asn)(d-Pro)(B)(Gly)(Gly)(Dpa) | KWKK |
| 245 | (Abu)(B)(d-Nle)(d-Ser)(d-Asn)(d-Asp) | KWKK |
| 246 | (d-Asn)(Hle)(d-Pro)(B)(Nap)(d-Ser) | KWKK |
| 247 | (ox-His)(Gly)(Gly)(Nap)(d-Asp)(Dpa) | KWKK |
| 248 | (d-Nle)(Abu)(d-Ser)(B)(d-Asn)(d-Asp) | KWKK |
| 249 | (Pip)(Gly)(d-Ser)(Hle)(d-His)(d-Asn) | KWKK |
| 250 | (Dpa)(d-Lys)(B)(Gly)(Gly)(d-Pro) | KWKK |
| 251 | (B)(d-Nle)(d-Asp)(Gly)(Pip)(Pip) | KWKK |
| 252 | (d-Lys)(B)(Amf)(d-Thr)(d-Thr)(Dab) | KWKK |
| 253 | (d-Pro)(d-Glu)(Amf)(Gly)(d-Ser)(B) | KWKK |
| 254 | (Dpa)(B)(Gly)(Dpa)(d-Phe)(d-Gln) | KWKK |
| 255 | (Abu)(d-Glu)(d-His)(Dpa)(d-Phe)(d-Gln) | KWKK |
| 256 | (Abu)(B)(d-Ser)(d-Phe)(d-Phe)(d-Gln) | KWKK |
| 257 | (Gly)(Gly)(Dab)(ox-His)(Nap)(Hle) | KWKK |
| 258 | (d-Phe)(d-Asp)(Gly)(d-Gln)(d-Pro)(Dpa) | KWKK |
| 259 | (Gly)(Pip)(d-Ser)(Hle)(d-His)(d-Asn) | KWKK |
| 260 | (d-Lys)(Abu)(Amf)(d-Ser)(d-Thr)(Dab) | KWKK |
| 261 | (Abu)(Gly)(Gly)(Hle)(Dpa)(Gly) | KWKK |
| 262 | (d-Glu)(d-Ser)(B)(d-Nle)(Dpa)(Dpa) | KWKK |
| 263 | (Abu)(d-Pro)(d-Gln)(d-Glu)(Nap)(Hle) | KWKK |
| 264 | (d-Ser)(B)(Gly)(Abu)(Gly)(Hle) | KWKK |
| 265 | (d-Phe)(d-Pro)(B)(Hle)(d-Arg)(d-Asp) | KWKK |
| 266 | (B)(Dab)(ox-His)(Amf)(Gly)(Nap) | KWKK |
| 267 | (Pip)(d-Nle)(d-Glu)(d-Gln)(d-His)(d-Asn) | KWKK |
| 268 | (d-Asp)(Abu)(d-His)(Dpa)(Hle)(B) | KWKK |
| 269 | (Gly)(Gly)(d-Nle)(B)(d-His)(Dpa) | KWKK |
| 270 | (B)(d-Nle)(d-Asp)(Pip)(Gly)(Pip) | KWKK |
| 271 | (Gly)(Gly)(d-His)(Abu)(d-Lys)(Dpa) | KWKK |
| 272 | (Nap)(d-Glu)(Pip)(d-Pro)(d-Gln)(d-Phe) | KWKK |
| 273 | (d-Gln)(B)(d-Glu)(d-Lys)(d-Nle)(Nap) | KWKK |
| 274 | (d-Asn)(B)(d-Asp)(Nap)(d-Pro)(Dpa) | KWKK |
| 275 | (d-Nle)(d-Lys)(Abu)(d-Asn)(d-Glu)(Amf) | KWKK |
| 276 | (B)(ox-His)(Dab)(d-Glu)(d-Thr)(d-Ser) | KWKK |
| 277 | (d-Asn)(Pip)(d-Gln)(d-Ser)(Hle)(d-Ser) | KWKK |
| 278 | (ox-His)(d-Pro)(d-Ser)(Dpa)(Hle)(B) | KWKK |
| 279 | (d-Thr)(Gly)(d-Phe)(d-Pro)(Gly)(Dpa) | KWKK |
| 280 | (d-Asn)(Gly)(d-Glu)(Nap)(d-Pro)(Dpa) | KWKK |
| 281 | (d-Asn)(d-Asp)(Nap)(B)(d-Pro)(Dpa) | KWKK |
| 282 | (d-Nle)(B)(Gly)(d-Arg)(d-Nle)(d-Phe) | KWKK |
| 283 | (d-Thr)(d-Pro)(Dab)(d-Nle)(Hle)(d-Gln) | KWKK |
| 284 | (d-Asn)(Nap)(d-Glu)(Gly)(d-Pro)(Dpa) | KWKK |
| 285 | (d-Asn)(Gly)(Nap)(d-Glu)(d-Pro)(Dpa) | KWKK |
| 286 | (Abu)(Dpa)(d-Pro)(Gly)(Abu)(B) | KWKK |
| 287 | (d-Lys)(Dpa)(Gly)(B)(Amf)(d-Nle) | KWKK |
| 288 | (d-His)(Gly)(Amf)(d-Lys)(d-Ser)(d-Asp) | KWKK |
| 289 | (d-Gln)(Dpa)(d-Asn)(d-Nle)(d-His)(Abu) | KWKK |
| 290 | (Dpa)(d-Gln)(Pip)(d-Pro)(Hle)(d-Thr) | KWKK |
| 291 | (ox-His)(Gly)(Gly)(Gly)(Nap)(Hle) | KWKK |
| 292 | (d-Gln)(Gba)(d-Glu)(d-Ser)(d-His)(d-Phe) | KWKK |
| 293 | (Dpa)(Pip)(d-Ser)(Abu)(Pip)(Dpa) | KWKK |
| 294 | (d-Asp)(d-His)(Pip)(d-Phe)(d-Arg)(d-Asp) | KWKK |
| 295 | (Dpa)(B)(Dpa)(d-Phe)(Gly)(B) | KWKK |
| 296 | (B)(d-Gln)(d-Glu)(d-Lys)(d-Nle)(Nap) | KWKK |
| 297 | (Pip)(Gly)(Gly)(Hle)(Hle)(d-His) | KWKK |
| 298 | (Abu)(B)(Amf)(Abu)(Hle)(Dpa) | KWKK |
| 299 | (d-Asn)(d-Pro)(d-Pro)(d-Phe)(Hle)(d-Ser) | KWKK |
| 300 | (d-His)(B)(Dab)(d-Phe)(Hle)(d-Ser) | KWKK |
| 301 | (d-Glu)(d-His)(Abu)(Dpa)(d-Phe)(d-Gln) | KWKK |
| 302 | (d-Glu)(Hle)(d-Thr)(d-Gln)(Nap)(Nap) | KWKK |
| 303 | (Hle)(d-Pro)(d-Glu)(d-Pro)(d-Thr)(Pip) | KWKK |
| 304 | (Nap)(d-Asp)(Gly)(d-Ser)(Dpa)(Gly) | KKWK |
| 305 | (d-Glu)(Hle)(d-Thr)(Nap)(B)(Gly) | KWKK |
| 306 | (d-Asn)(Abu)(B)(Hle)(d-His)(d-Asn) | KWKK |
| 307 | (d-Nle)(Hle)(d-His)(d-Asp)(d-His)(Amf) | KWKK |
| 308 | (Dpa)(Amf)(d-Ser)(d-Phe)(Gly)(B) | KWKK |
| 309 | (d-Thr)(Dpa)(Abu)(B)(Dpa)(d-Asn) | KWKK |
| 310 | (Hle)(Gly)(Dpa)(d-Phe)(d-Ser)(B) | KWKK |
| 311 | (Nap)(Dab)(B)(d-Ser)(Hle)(d-Ser) | KWKK |
| 312 | (Abu)(Dpa)(d-Pro)(Gly)(B)(Abu) | KWKK |
| 313 | (Dpa)(d-Ser)(Pip)(Abu)(Pip)(Dpa) | KWKK |
| 314 | (d-Ser)(d-Glu)(d-Nle)(Gly)(Gly)(d-Gln) | KWKK |
| 315 | (d-Lys)(d-Arg)(d-Asp)(Gly)(d-Pro)(Gly) | KWKK |
| 316 | (d-Gln)(d-Pro)(Pip)(B)(d-Asn)(d-Pro) | KWKK |
| 317 | (Dpa)(Pip)(Gly)(Gly)(Hle)(Dab) | KWKK |
| 318 | (Abu)(d-Pro)(Gly)(d-Pro)(Gly)(d-His) | KWKK |
| 319 | (B)(Abu)(ox-His)(Abu)(d-Nle)(d-Phe) | KWKK |
| 320 | (Dpa)(Pip)(B)(d-Thr)(Pip)(Dpa) | KWKK |
| 321 | (d-Pro)(Abu)(Gly)(d-Pro)(Gly)(d-His) | KWKK |
| 322 | (d-Arg)(d-Gln)(d-Asp)(d-Ser)(d-Phe)(d-Gln) | KWKK |
| 323 | (Dpa)(Abu)(ox-His)(d-Nle)(Hle)(d-Thr) | KWKK |
| 324 | (d-Pro)(Hle)(d-Nle)(d-Ser)(Abu)(B) | KWKK |
| 325 | (B)(d-Nle)(Pip)(d-Asp)(Gly)(Pip) | KWKK |
| 326 | (B)(d-His)(Dab)(d-Phe)(Hle)(d-Ser) | KWKK |
| 327 | (d-Pro)(B)(d-Pro)(Nap)(Nap)(d-Asn) | KWKK |
| 328 | (Gly)(Dab)(Gly)(ox-His)(Nap)(Hle) | KWKK |
| 329 | (d-Pro)(d-Nle)(d-Asp)(B)(Gly)(Hle) | KWKK |
| 330 | (d-Lys)(d-Ser)(d-Thr)(Dab)(Dab)(Nap) | KWKK |
| 331 | (d-Phe)(Abu)(Gly)(d-Ser)(d-Phe)(Nap) | KWKK |
| 332 | (Abu)(B)(d-Pro)(d-Pro)(Abu)(Gly) | KWKK |
| 333 | (d-Asp)(Gly)(d-Phe)(d-Gln)(d-Pro)(Dpa) | KWKK |
| 334 | (Abu)(Hle)(Gly)(d-Ser)(d-Asn)(d-Asp) | KWKK |
| 335 | (d-Ser)(d-Phe)(Gly)(d-Nle)(d-Nle)(d-Phe) | KWKK |
| 336 | (Hle)(Gly)(Dpa)(d-Phe)(d-Thr)(Gly) | KWKK |
| 337 | (Abu)(d-Pro)(B)(d-Pro)(d-Ser)(d-Glu) | KWKK |
| 338 | (d-Asp)(d-Phe)(Gly)(d-Gln)(d-Pro)(Dpa) | KWKK |
| 339 | (Gly)(Gly)(Gly)(Gly)(d-Nle)(d-Asp) | KWKK |
| 340 | (d-Pro)(d-Glu)(Amf)(d-Thr)(Gly)(Gly) | KWKK |
| 341 | (Hle)(Dpa)(Gly)(B)(Pip)(d-Phe) | KWKK |
| 342 | (B)(Nap)(d-Phe)(Abu)(Hle)(Hle) | KWKK |
| 343 | (Hle)(d-Pro)(d-Glu)(Pip)(d-Pro)(d-Thr) | KWKK |
| 344 | (d-Pro)(Hle)(d-Nle)(d-Ser)(B)(Abu) | KWKK |
| 345 | (Nap)(d-Pro)(Abu)(d-Nle)(d-Nle)(Amf) | KWKK |
| 346 | (Pip)(B)(d-Thr)(d-Nle)(Hle)(d-Gln) | KWKK |
| 347 | (d-Phe)(Dpa)(Dpa)(d-Phe)(Pip)(Dpa) | KWKK |
| 348 | (d-Lys)(d-Thr)(Amf)(d-Ser)(d-His)(Amf) | KWKK |
| 349 | (Pip)(d-Pro)(d-Ser)(Pip)(Gly)(Gly) | KWKK |
| 350 | (d-Lys)(d-Lys)(B)(Gba)(d-Thr)(Nap) | KWKK |
| 351 | (d-Arg)(d-Thr)(Dpa)(Abu)(d-Asn)(d-Phe) | KWKK |
| 352 | (d-Nle)(Abu)(B)(Gly)(Dpa)(Gly) | KWKK |
| 353 | (d-Pro)(B)(d-Phe)(Abu)(Dpa)(Dpa) | KWKK |
| 354 | (d-Asp)(d-Asp)(d-Lys)(d-Nle)(Dpa)(Dpa) | KWKK |
| 355 | (d-Nle)(d-Thr)(d-Ser)(d-His)(d-Nle)(Nap) | KWKK |
| 356 | (d-Asp)(d-Phe)(d-Thr)(d-Asp)(Gly)(Gly) | KWKK |
| 357 | (Pip)(Dpa)(Gly)(Gly)(Hle)(Dab) | KWKK |
| 358 | (d-Phe)(Gly)(d-Ser)(d-Nle)(d-Nle)(d-Phe) | KWKK |
| 359 | (Hle)(d-Lys)(Dpa)(Gly)(d-Pro)(d-Thr) | KWKK |
| 360 | (Gly)(Gly)(d-Gln)(B)(Amf)(d-Asp) | KWKK |
| 361 | (B)(Gly)(Abu)(d-Glu)(Gly)(d-Phe) | KWKK |
| 362 | (B)(d-Nle)(d-Thr)(d-Thr)(Abu)(d-His) | KWKK |
| 363 | (Gly)(Dab)(d-Lys)(d-Glu)(d-Glu)(Dpa) | KWKK |
| 364 | (Gly)(Dpa)(d-Pro)(Abu)(d-Pro)(d-Asn) | KWKK |
| 365 | (d-Nle)(Abu)(B)(d-Ser)(d-Gln)(d-Gln) | KWKK |
| 366 | (Dab)(B)(ox-His)(Amf)(Gly)(Nap) | KWKK |
| 367 | (Hle)(d-Glu)(d-Thr)(Nap)(d-Gln)(Nap) | KWKK |
| 368 | (d-Ser)(d-Glu)(d-Nle)(d-Asn)(B)(Gly) | KWKK |
| 369 | (d-Lys)(d-Phe)(Gly)(Abu)(Hle)(Dpa) | KWKK |
| 370 | (d-Nle)(Gly)(d-Phe)(Dab)(Hle)(Dpa) | KWKK |
| 371 | (ox-His)(B)(Hle)(Dpa)(Hle)(d-Thr) | KWKK |
| 372 | (d-Pro)(Pip)(d-Gln)(Dpa)(Hle)(d-Thr) | KWKK |
| 373 | (Gly)(B)(Dpa)(d-Phe)(Gly)(B) | KWKK |
| 374 | (Gly)(Gly)(B)(d-Gln)(Amf)(d-Asp) | KWKK |
| 375 | (Amf)(B)(Abu)(Abu)(Hle)(Dpa) | KWKK |
| 376 | (d-Ser)(B)(d-Ser)(d-Ser)(Hle)(Hle) | KWKK |
| 377 | (B)(d-Lys)(Dab)(ox-His)(Gly)(d-Pro) | KWKK |
| 378 | (d-Lys)(Abu)(d-Ser)(Amf)(d-Thr)(Dab) | KWKK |
| 379 | (Abu)(B)(d-Ser)(Dpa)(Gly)(d-Ser) | KWKK |
| 380 | (d-Ser)(Amf)(Gly)(d-Ser)(Pip)(Dab) | KWKK |

Split-and-pool synthesis was carried out on 300 μm TentaGel resin (0.23 mmol/g) for a 95,000 member library. Splits were performed by suspending the resin in DCM and dividing it evenly (via pipetting) among 22 plastic fritted syringes on a vacuum manifold. Couplings were carried out as follows: solutions of Fmoc-protected amino acids (10 equivalents relative to the resin loading), PyAOP (0.38 M in DMF; 0.9 eq. relative to amino acid), and DI EA (1.1 eq. for his-tidine; 3 eq. for all other amino acids) were each added to individual portions of resin. Couplings were allowed to proceed for 60 min. Resin portions were recombined and washed with 0CM and DMF. Fmoc removal was carried out by treatment of the resin with 20% piperidine in DMF (1× flow wash; 2× 5 min batch treatments). Resin was washed again with DMF and DCM before the next split.

### Example 12—Activity of Peptide Libraries

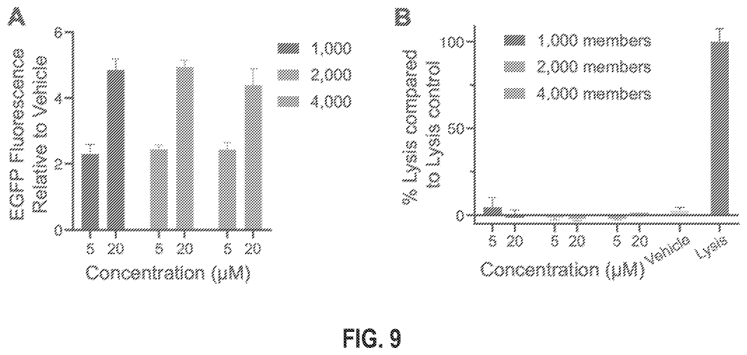

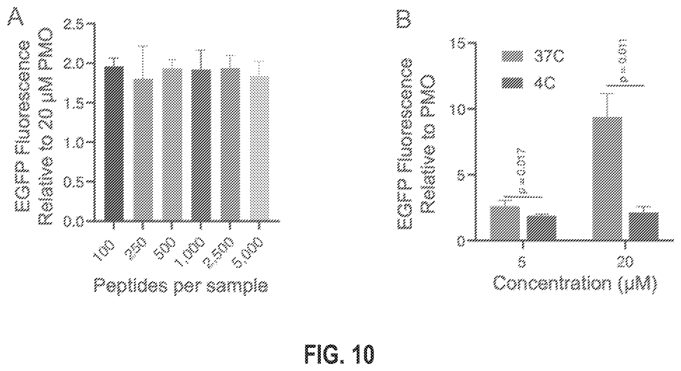

A series of activity experiments were performed to confirm that the peptides within the library had nuclear-localizing activity. The phenotypic assay used correlates with the amount of active PMO delivered to the nucleus by resulting in corrective splicing to produce an enhanced green fluorescent protein (EGFP), quantified by flow cytometry. First, PPMO-library aliquots demonstrated a concentration-dependent increase in activity (FIG. 9A). At the same time, the library at these concentrations did not exhibit any membrane disruption or toxicity as determined by a lactate dehydrogenase (LDH) release assay (FIG. 9B). Testing the same concentrations of library aliquots containing different amounts of sequences, thereby increasing diversity, showed no difference in activity (FIG. 10A).

A 1,000-member portion of the PPMO-library was incubated with cells at 4° C., conditions that arrest energy-dependent uptake. After incubation with the PMO-CPP conjugates, each well was washed extensively with PBS and heparin in order to disrupt and remove membrane-bound conjugates. The cells were warmed back up to 37° C. and the assay continued in the standard format and analyzed by flow cytometry. The significant decrease in library PMO delivery (relative to PMO alone) at 4° C. for both 5 μM and 20 μM library incubation conditions demonstrates energy-dependent uptake for the PMO-CPPs (FIG. 10B). After treatment, cells were washed with 0.1 mg/mL heparin and incubated in media for 22 h prior to flow cytometry.

TABLE 5
PPMO-Library

| SEQ | | Terminal Motif |
|---|---|---|
| ID NO. | Peptide Sequences | (d-amino acids) |
| 381 | (Hle)(Nle)(d-Glu)(d-Ser)(d-Thr)(d-Asn) | KWKK |
| 382 | (B)(B)(d-Asp)(Gly)(Dpa)(d-Asn) | KWKK |
| 383 | (Gly)(d-Ser)(Hle)(d-Phe)(d-Glu)(d-Gln) | KWKK |
| 384 | (d-Pro)(d-Thr)(d-Asp)(Gly)(d-Thr)(Gly) | KWKK |
| 385 | (d-Pro)(d-Thr)(Hle)(d-Ser)(d-Asp)(Nap) | KWKK |
| 386 | (d-Asn)(d-Ser)(d-Ser)(Pip)(Hle)(Nle) | KWKK |
| 387 | (Gly)(d-Pro)(d-Glu)(d-Glu)(Gly)(Nap) | KWKK |
| 388 | (B)(d-Asn)(Dpa)(d-Ser)(Hle)(Dab) | KWKK |
| 389 | (Gly)(d-Asp)(d-Asp)(Dpa)(d-Asn)(d-Ser) | KWKK |
| 390 | (d-Asp)(d-Thr)(Hle)(d-Pro)(d-Gln)(Hle) | KWKK |
| 391 | (d-Pro)(d-Thr)(d-Asp)(d-Asp)(d-Asn)(Hle) | KWKK |
| 392 | (d-Thr)(d-Pro)(Hle)(d-Ser)(d-Asp)(Nap) | KWKK |
| 393 | (Gly)(d-Arg)(d-Glu)(d-Thr)(Hle)(d-Asn) | KWKK |
| 394 | (B)(Hle)(d-Glu)(d-Gln)(d-Glu)(Amf) | KWKK |
| 395 | (Gly)(d-Phe)(d-Pro)(Pip)(Nle)(Gly) | KWKK |
| 396 | (B)(d-Thr)(d-Gln)(Hle)(d-Glu)(d-Asp) | KWKK |
| 397 | (d-Phe)(Hle)(d-Glu)(d-Ser)(B)(d-Thr) | KWKK |
| 398 | (d-Asn)(Gly)(Hle)(B)(Nle)(d-Asp) | KWKK |
| 399 | (B)(d-Arg)(Nle)(d-Arg)(d-Glu)(d-Gln) | KWKK |
| 400 | (Gly)(Gly)(B)(d-Phe)(d-Asn)(d-Thr) | KWKK |
| 401 | (Nle)(d-Arg)(Nle)(d-Gln)(B)(d-Asp) | KWKK |
| 402 | (d-Asp)(d-Asn)(d-Gln)(Abu)(d-Thr)(d-Phe) | KWKK |
| 403 | (Nle)(Hle)(d-Glu)(d-Ser)(d-Thr)(d-Asn) | KWKK |
| 404 | (d-Asn)(d-Ser)(Amf)(Dab)(d-Phe)(Gly) | KWKK |
| 405 | (d-Gln)(B)(d-Phe)(d-Arg)(d-Asn)(Amf) | KWKK |
| 406 | (d-Asn)(d-Lys)(d-Thr)(Gly)(Hle)(Abu) | KWKK |
| 407 | (B)(B)(B)(Hle)(d-His)(Hle) | KWKK |
| 408 | (Gly)(B)(B)(d-Glu)(Nle)(Dpa) | KWKK |
| 409 | (d-Pro)(d-Thr)(Dpa)(Abu)(B)(d-Lys) | KWKK |
| 410 | (B)(Nle)(Dab)(d-Phe)(Gly)(d-Thr) | KWKK |
| 411 | (B)(Ne)(Dab)(d-Lys)(B)(d-Phe) | KWKK |
| 412 | (d-Gln)(d-Ser)(d-Lys)(Hle)(d-Phe)(Dab) | KWKK |
| 413 | (d-Arg)(d-Phe)(d-Lys)(d-Asp)(Hle)(Gly) | KWKK |
| 414 | (Abu)(Gly)(d-Asp)(Gly)(Dpa)(d-Asn) | KWKK |
| 415 | (B)(d-Thr)(Dpa)(d-Asn)(d-Gln)(Nap) | KWKK |
| 416 | (Abu)(B)(d-Glu)(Pip)(d-Arg)(Nle) | KWKK |
| 417 | (Gly)(d-Asp)(d-Arg)(d-Ser)(Dab)(d-Phe) | KWKK |
| 418 | (B)(d-Arg)(Hle)(d-Phe)(Gly)(d-Arg) | KWKK |
| 419 | (d-Arg)(d-Asp)(Hle)(d-Ser)(Pip)(d-Gln) | KWKK |
| 420 | (Gly)(Gly)(d-Ser)(Pip)(Hle)(Nle) | KWKK |
| 421 | (d-Asn)(d-Asp)(d-Gln)(Abu)(d-Thr)(d-Phe) | KWKK |
| 422 | (B)(Nle)(B)(B)(d-Thr)(Hle) | KWKK |
| 423 | (Gly)(Dpa)(Gly)(d-Asp)(Gly)(B) | KWKK |
| 424 | (Gly)(Gly)(d-Asn)(d-Pro)(Hle)(Dab) | KWKK |
| 425 | (d-Phe)(d-Thr)(d-Lys)(d-Asn)(d-Thr)(d-Phe) | KWKK |
| 426 | (d-Asn)(d-Asp)(B)(d-Thr)(Gly)(d-Phe) | KWKK |
| 427 | (d-Asn)(Gly)(d-Gln)(d-Ser)(Nap)(d-Asp) | KWKK |
| 428 | (Gly)(Abu)(d-Asp)(Gly)(Dpa)(d-Asn) | KWKK |
| 429 | (B)(Abu)(d-Asp)(Hle)(Amf)(Nle) | KWKK |
| 430 | (d-Asn)(Gly)(d-Glu)(Nap)(d-Asn)(d-Asp) | KWKK |
| 431 | (B)(Gly)(Gly)(d-Gln)(Dpa)(d-Phe) | KWKK |
| 432 | (B)(Abu)(d-Glu)(Pip)(d-Arg)(Nle) | KWKK |
| 433 | (B)(Abu)(Nle)(d-Arg)(d-Pro)(d-Pro) | KWKK |
| 434 | (Gly)(Gly)(Nle)(d-Thr)(d-Asp)(Hle) | KWKK |
| 435 | (B)(d-Asn)(Gly)(B)(Dpa)(d-Phe) | KWKK |
| 436 | (Gly)(Gly)(Abu)(d-Glu)(Nle)(Dpa) | KWKK |
| 437 | (d-Asn)(d-Gln)(d-Glu)(d-Gln)(d-Ser)(d-Phe) | KWKK |
| 438 | (Gly)(Dpa)(Gly)(B)(d-His)(B) | KWKK |
| 439 | (Gly)(Gly)(d-Thr)(Abu)(d-Gln)(Nap) | KWKK |
| 440 | (Dab)(d-Thr)(d-Asp)(Gly)(d-Thr)(Gly) | KWKK |
| 441 | (d-Asn)(Gly)(d-Asp)(d-Asp)(d-Pro)(Nap) | KWKK |
| 442 | (d-Pro)(d-Thr)(d-Phe)(d-Glu)(Gly)(B) | KWKK |
| 443 | (d-Lys)(d-Asn)(d-Thr)(Gly)(Hle)(Abu) | KWKK |
| 444 | (d-Thr)(d-Pro)(Dpa)(Abu)(B)(d-Lys) | KWKK |
| 445 | (B)(Nle)(Amf)(B)(Gly)(d-Thr) | KWKK |
| 446 | (Nle)(Nle)(d-Arg)(d-Glu)(Gly)(B) | KWKK |
| 447 | (d-Pro)(d-Ser)(d-Pro)(d-Lys)(Pip)(d-Gln) | KWKK |
| 448 | (d-Asn)(d-Lys)(B)(d-Phe)(d-Asp)(d-Asp) | KWKK |
| 449 | (Gly)(Gly)(Dpa)(Abu)(d-Lys)(d-Ser) | KWKK |
| 450 | (B)(Abu)(Nle)(d-Pro)(d-Pro)(d-Arg) | KWKK |
| 451 | (Gly)(d-Gln)(Gba)(d-Ser)(d-Ser)(d-Asn) | KWKK |
| 452 | (d-Ser)(d-Pro)(d-Pro)(d-Glu)(d-Glu)(Dab) | KWKK |
| 453 | (Gly)(Gly)(Nle)(d-Asn)(B)(Hle) | KWKK |
| 454 | (Gly)(Gly)(d-Gln)(d-Ser)(Nap)(d-Asp) | KWKK |
| 455 | (B)(Gly)(B)(Hle)(d-Phe)(d-Ser) | KWKK |
| 456 | (d-Asn)(Nle)(d-Pro)(d-Asn)(d-Ser)(Nap) | KWKK |
| 457 | (Gly)(d-Ser)(Hle)(d-Asp)(Gly)(Gly) | KWKK |
| 458 | (Abu)(d-Pro)(Nle)(d-Pro)(d-Pro)(Gly) | KWKK |
| 459 | (Abu)(Gly)(Gly)(Gly)(d-Arg)(Dpa) | KWKK |
| 460 | (d-Gln)(Dab)(d-Asp)(Pip)(d-Gln)(Gly) | KWKK |
| 461 | (Abu)(B)(d-Lys)(B)(d-Thr)(d-Ser) | KWKK |
| 462 | (Gly)(Nle)(d-Glu)(d-His)(B)(Dab) | KWKK |
| 463 | (Abu)(d-Ser)(B)(d-Asn)(Amf)(d-Phe) | KWKK |
| 464 | (d-Asp)(Hle)(B)(Gly)(Gly)(d-Glu) | KWKK |
| 465 | (d-Asn)(Gly)(Dpa)(Gly)(B)(Gly) | KWKK |
| 466 | (d-Thr)(d-Pro)(d-Asp)(Gly)(d-Thr)(Gly) | KWKK |
| 467 | (d-Asn)(d-Asp)(Gly)(d-Asp)(d-Pro)(Nap) | KWKK |
| 468 | (d-Pro)(d-Thr)(d-Phe)(d-Glu)(B)(Gly) | KWKK |
| 469 | (d-Lys)(B)(d-Asn)(d-Phe)(d-Asp)(d-Asp) | KWKK |
| 470 | (d-Gln)(d-Glu)(d-Lys)(d-Asp)(Hle)(Gly) | KWKK |
| 471 | (Gly)(Dpa)(B)(Gly)(d-His)(B) | KWKK |
| 472 | (B)(Nle)(d-Arg)(Hle)(Gly)(B) | KWKK |
| 473 | (B)(Abu)(Gly)(Hle)(d-His)(Hle) | KWKK |
| 474 | (d-Arg)(B)(d-Ser)(d-Pro)(d-Phe)(d-Glu) | KWKK |
| 475 | (ox-His)(d-Asp)(d-Asp)(d-Glu)(Abu)(d-Ser) | KWKK |
| 476 | (Abu)(B)(d-Asp)(Pip)(d-Glu)(d-His) | KWKK |
| 477 | (d-Asp)(d-Gln)(Hle)(Gly)(Gly)(Nle) | KWKK |
| 478 | (d-Pro)(d-Gln)(Gly)(Nap)(d-Glu)(d-Asp) | KWKK |
| 479 | (d-Pro)(d-Ser)(d-Asn)(d-Asp)(d-Glu)(Pip) | KWKK |
| 480 | (Gly)(d-Asn)(d-Phe)(Gly)(d-Glu)(Nle) | KWKK |
| 481 | (Gly)(d-Asn)(Dpa)(Gly)(B)(Gly) | KWKK |
| 482 | (d-Asn)(Dab)(Dab)(d-Pro)(d-Phe)(d-Glu) | KWKK |
| 483 | (d-Asn)(B)(Gly)(d-Asn)(Nle)(d-Arg) | KWKK |
| 484 | (Gly)(B)(d-Gln)(Amf)(Nap)(d-Gln) | KWKK |
| 485 | (d-Phe)(d-Asp)(d-Ser)(B)(Hle)(Pip) | KWKK |
| 486 | (Gba)(d-Glu)(Gly)(d-Ser)(Dab)(d-Phe) | KWKK |
| 487 | (Gly)(Gly)(Gly)(Nle)(d-Pro)(d-Asp) | KWKK |
| 488 | (Gly)(Gly)(Gly)(Dpa)(d-Gln)(Gly) | KWKK |
| 489 | (Pip)(d-Thr)(d-Pro)(d-Asn)(d-Ser)(Nap) | KWKK |
| 490 | (Nle)(Abu)(d-Glu)(d-Gln)(d-Glu)(Amf) | KWKK |
| 491 | (d-Phe)(Hle)(d-Glu)(B)(d-Ser)(d-Thr) | KWKK |
| 492 | (d-Asp)(Dpa)(d-Ser)(B)(Gly)(B) | KWKK |
| 493 | (d-Asp)(d-Ser)(d-Phe)(d-Asn)(d-Lys)(d-Phe) | KWKK |
| 494 | (B)(Gly)(B)(Hle)(B)(d-Arg) | KWKK |
| 495 | (Abu)(Pip)(d-Asn)(d-Arg)(d-Ser)(Nap) | KWKK |
| 496 | (Gly)(Gly)(d-Asn)(Abu)(d-Phe)(d-Gln) | KWKK |
| 497 | (Gly)(d-Asn)(d-Asn)(Gly)(Dpa)(d-Asn) | KWKK |
| 498 | (Gly)(Pip)(d-Asp)(Nle)(B)(d-Asn) | KWKK |
| 499 | (d-Lys)(d-Pro)(d-Pro)(d-Ser)(Pip)(d-Gln) | KWKK |
| 500 | (Gly)(B)(d-Phe)(Gly)(Gly)(d-Thr) | KWKK |
| 501 | (d-Asp)(B)(Gly)(Abu)(d-Thr)(d-Phe) | KWKK |
| 502 | (Gly)(Gly)(Gly)(Nap)(d-Asp)(Dab) | KWKK |
| 503 | (Gly)(d-Asn)(d-Glu)(Nap)(d-Asn)(d-Asp) | KWKK |
| 504 | (B)(d-Arg)(Nle)(d-Pro)(Gly)(Gly) | KWKK |
| 505 | (Gly)(Gba)(d-Glu)(d-Ser)(Dab)(d-Phe) | KWKK |
| 506 | (Gly)(B)(Gly)(d-Phe)(d-Asn)(d-Thr) | KWKK |
| 507 | (d-Glu)(d-Ser)(d-Ser)(d-Thr)(Gly)(d-Phe) | KWKK |
| 508 | (B)(d-Phe)(Nle)(d-Asp)(Abu)(B) | KWKK |
| 509 | (Nle)(B)(d-Thr)(Nap)(Gly)(Gly) | KWKK |
| 510 | (B)(Dab)(Nap)(ox-His)(d-Asp)(Nap) | KWKK |
| 511 | (B)(d-Asn)(d-Pro)(Ne)(Pip)(d-Gln) | KWKK |
| 512 | (Abu)(B)(d-Arg)(d-Pro)(Pip)(d-Gln) | KWKK |
| 513 | (Dab)(d-Thr)(Gly)(d-Asp)(d-Thr)(Gly) | KWKK |
| 514 | (Nle)(d-Asp)(d-His)(d-Asp)(Nle)(d-Phe) | KWKK |
| 515 | (Gly)(d-Asn)(d-Asp)(d-Asp)(d-Pro)(Nap) | KWKK |
| 516 | (Gly)(Gly)(Nle)(Gly)(d-Phe)(d-Asn) | KWKK |
| 517 | (d-Lys)(B)(ox-Amf)(d-Asp)(B)(d-Lys) | KWKK |
| 518 | (Nle)(B)(Dab)(d-Phe)(Gly)(d-Thr) | KWKK |
| 519 | (Abu)(B)(d-Ser)(d-Asn)(Amf)(d-Phe) | KWKK |
| 520 | (Dab)(d-Ser)(d-Asn)(Pip)(Nle)(Gly) | KWKK |
| 521 | (d-Phe)(Dab)(d-Asp)(Abu)(Abu)(Dpa) | KWKK |
| 522 | (ox-Nap)(Dab)(Gly)(Abu)(d-Phe)(Gly) | KWKK |
| 523 | (Gly)(Pip)(Gba)(d-Ser)(d-Thr)(d-Gln) | KWKK |
| 524 | (d-Lys)(d-Asp)(Gly)(Gly)(Amf)(d-Phe) | KWKK |
| 525 | (B)(Abu)(d-Asn)(d-Arg)(d-Asn)(Gly) | KWKK |
| 526 | (d-Asn)(Nle)(Gly)(Gly)(B)(Hle) | KWKK |
| 527 | (d-Pro)(Gly)(d-Glu)(d-Glu)(Gly)(Nap) | KWKK |
| 528 | (Abu)(Amf)(d-His)(Gly)(d-Phe)(Gly) | KWKK |
| 529 | (Abu)(Dab)(Hle)(d-Thr)(Gly)(Abu) | KWKK |
| 530 | (Nle)(d-Arg)(Nle)(Abu)(d-Asn)(d-Asp) | KWKK |
| 531 | (d-Pro)(Gly)(B)(Gly)(Abu)(Hle) | KWKK |
| 532 | (d-Asp)(Dpa)(d-Ser)(Gly)(Gly)(Abu) | KWKK |
| 533 | (Abu)(Gba)(d-Ser)(d-Pro)(d-Phe)(d-Glu) | KWKK |
| 534 | (B)(Gly)(B)(d-Glu)(Nle)(Dpa) | KWKK |
| 535 | (d-Lys)(ox-His)(ox-His)(d-Ser)(d-Asp)(Nap) | KWKK |
| 536 | (Nle)(d-Asn)(Abu)(d-Pro)(Pip)(d-Gln) | KWKK |
| 537 | (Amf)(d-Thr)(d-Pro)(d-Pro)(B)(d-Asp) | KWKK |
| 538 | (Gly)(d-Ser)(ox-Amf)(B)(Gly)(Amf) | KWKK |
| 539 | (Gly)(Gly)(B)(d-Gln)(Dpa)(d-Phe) | KWKK |
| 540 | (d-Arg)(d-Asp)(Gly)(d-Ser)(Dab)(d-Phe) | KWKK |
| 541 | (Abu)(d-Gln)(Dab)(d-Phe)(d-Asp)(d-Asp) | KWKK |
| 542 | (d-Lys)(d-Asn)(B)(d-Phe)(d-Asp)(d-Asp) | KWKK |
| 543 | (Gly)(Gly)(d-Phe)(d-Asn)(d-Lys)(d-Phe) | KWKK |
| 544 | (Nle)(d-Glu)(B)(Gly)(Gly)(d-Glu) | KWKK |
| 545 | (d-Asn)(B)(Gly)(d-Ser)(Nap)(d-Asp) | KWKK |
| 546 | (d-Asp)(B)(Abu)(Hle)(Amf)(Nle) | KWKK |
| 547 | (B)(d-Asn)(d-Pro)(Nap)(d-Glu)(d-Asp) | KWKK |
| 548 | (Gly)(Nle)(d-Glu)(B)(d-His)(Dab) | KWKK |
| 549 | (d-Arg)(d-Pro)(d-Asp)(ox-His)(d-Asp)(Nap) | KWKK |
| 550 | (Abu)(B)(d-Thr)(Amf)(d-Phe)(d-His) | KWKK |
| 551 | (Abu)(B)(B)(d-Asn)(d-Phe)(Pip) | KWKK |
| 552 | (Gly)(Gly)(d-Pro)(B)(d-Glu)(Nle) | KWKK |
| 553 | (Gly)(Gly)(d-Ser)(Nle)(Abu)(Nap) | KWKK |
| 554 | o(d-Ser)(Hle)(B)(Gly)(Gly) | KWKK |
| 555 | (B)(d-Asn)(d-Asp)(Nap)(d-Asn)(d-Asp) | KWKK |
| 556 | (B)(d-Asp)(Amf)(Gly)(d-Ser)(d-Thr) | KWKK |
| 557 | (B)(Nle)(Dab)(B)(ox-His)(Nle) | KWKK |
| 558 | (d-Lys)(B)(B)(B)(d-Thr)(ox-His) | KWKK |
| 559 | (d-Lys)(B)(Gly)(ox-His)(d-Thr)(d-Gln) | KWKK |
| 560 | (Gly)(d-Ser)(Amf)(Gly)(d-Ser)(Amf) | KWKK |
| 561 | (Abu)(B)(Gba)(d-Ser)(d-Asp)(d-Phe) | KWKK |
| 562 | (d-Thr)(d-Phe)(d-Lys)(d-Asn)(d-Thr)(d-Phe) | KWKK |
| 563 | (Abu)(B)(Gly)(d-Ser)(Dab)(d-Thr) | KWKK |
| 564 | (B)(Gly)(Abu)(Hle)(d-His)(Hle) | KWKK |
| 565 | (Abu)(Gly)(d-Glu)(Hle)(Amf)(Nle) | KWKK |
| 566 | (d-Asp)(B)(Dab)(d-Pro)(ox-His)(d-Glu) | KWKK |
| 567 | (d-Pro)(d-Glu)(Gly)(d-Glu)(Gly)(Nap) | KWKK |
| 568 | (Nle)(Nle)(Abu)(B)(d-Glu)(d-Gln) | KWKK |
| 569 | (d-Asn)(Abu)(Hle)(B)(Abu)(B) | KWKK |
| 570 | (Gly)(Gly)(Gly)(d-Asp)(Nap)(Dab) | KWKK |
| 571 | (Gly)(d-Asn)(d-Gln)(d-Ser)(Nap)(d-Asp) | KWKK |
| 572 | (ox-Nap)(d-Pro)(d-Asp)(Gly)(Hle)(d-Pro) | KWKK |
| 573 | (B)(Gly)(B)(B)(Hle)(d-Arg) | KWKK |
| 574 | (Nle)(d-Thr)(Nle)(Gly)(Gly)(d-Thr) | KWKK |
| 575 | (d-Asn)(d-Asp)(Pip)(d-Ser)(d-Thr)(d-Phe) | KWKK |
| 576 | (d-His)(d-His)(Gly)(B)(Gly)(Gly) | KWKK |
| 577 | (Abu)(Nle)(d-Pro)(d-Pro)(d-Pro)(Gly) | KWKK |
| 578 | (Dab)(Hle)(Hle)(Abu)(B)(Gly) | KWKK |
| 579 | (Gly)(d-Glu)(Nle)(Pip)(d-Asn)(Gly) | KWKK |
| 580 | (d-Ser)(d-Pro)(d-Pro)(d-Lys)(Pip)(d-Gln) | KWKK |
| 581 | (Gly)(d-Ser)(Nle)(d-Glu)(Gly)(Gly) | KWKK |
| 582 | (B)(Ne)(B)(Abu)(Hle)(d-Gln) | KWKK |
| 583 | (Gly)(Gly)(d-Glu)(Hle)(d-Asn)(Nle) | KWKK |
| 584 | (B)(d-Asn)(Dpa)(Nle)(d-Thr)(Dab) | KWKK |
| 585 | (B)(Nle)(d-Arg)(Hle)(B)(Gly) | KWKK |
| 586 | (B)(Nle)(Abu)(B)(Hle)(d-Gln) | KWKK |
| 587 | (Gly)(Gly)(Abu)(d-Thr)(d-Gln)(Nap) | KWKK |
| 588 | (d-Asp)(d-Thr)(d-Pro)(Hle)(d-Gln)(Hle) | KWKK |
| 589 | (Nap)(Hle)(Gly)(Gly)(d-Ser)(Nap) | KWKK |
| 590 | (B)(Nle)(Dab)(d-Pro)(B)(ox-His) | KWKK |
| 591 | (B)(d-His)(Dab)(d-Phe)(d-Phe)(Gly) | KWKK |
| 592 | (Abu)(Gly)(Hle)(B)(Amf)(d-Glu) | KWKK |
| 593 | (Gly)(d-Gln)(Gba)(d-Ser)(d-Asn)(d-Ser) | KWKK |
| 594 | (d-Ser)(d-Ser)(Dpa)(B)(Gly)(Gly) | KWKK |
| 595 | (Nle)(d-Asp)(d-Asp)(Pip)(d-Gln)(Gly) | KWKK |
| 596 | (d-Pro)(B)(d-Asp)(d-Thr)(Hle)(d-Asp) | KWKK |
| 597 | (d-Asp)(d-Phe)(d-Ser)(B)(Hle)(Pip) | KWKK |
| 598 | (d-Pro)(Pip)(d-Thr)(d-Asn)(d-Ser)(Nap) | KWKK |
| 599 | (d-Gln)(ox-His)(d-Asn)(Pip)(d-Asp)(Nap) | KWKK |
| 600 | (d-Gln)(Pip)(ox-His)(d-Asn)(d-Asp)(Nap) | KWKK |
| 601 | (Abu)(Gly)(d-Asn)(Abu)(B)(Dpa) | KWKK |
| 602 | (B)(B)(Gly)(Gly)(d-Arg)(Dpa) | KWKK |
| 603 | (Gly)(d-Ser)(Gly)(d-Ser)(Amf)(Amf) | KWKK |
| 604 | (d-Asp)(d-Ser)(d-Phe)(B)(Hle)(Pip) | KWKK |
| 605 | (Gly)(Hle)(d-Thr)(Nap)(Gly)(Gly) | KWKK |
| 606 | (B)(Nap)(Dab)(d-Ser)(d-Phe)(Gly) | KWKK |
| 607 | (Dab)(d-Ser)(d-His)(Nap)(d-Asp)(Nap) | KWKK |
| 608 | (Nle)(d-Phe)(d-Asn)(B)(d-Ser)(d-Ser) | KWKK |
| 609 | (d-Asn)(B)(d-Pro)(d-Pro)(Nle)(d-His) | KWKK |
| 610 | (d-Gln)(d-Asn)(d-Glu)(d-Gln)(d-Ser)(d-Phe) | KWKK |
| 611 | (Abu)(B)(d-Lys)(Abu)(d-Ser)(d-Ser) | KWKK |
| 612 | (B)(d-Lys)(Gly)(ox-His)(d-Thr)(d-Gln) | KWKK |
| 613 | (B)(d-Asp)(d-Asn)(Pip)(Nle)(Gly) | KWKK |
| 614 | (Gly)(d-Ser)(Abu)(B)(d-Gln)(Nap) | KWKK |
| 615 | (Gly)(d-Ser)(Abu)(d-Arg)(d-Asp)(d-Phe) | KWKK |
| 616 | (Nle)(d-Lys)(Gly)(d-Glu)(ox-His)(d-Lys) | KWKK |
| 617 | (B)(d-Ser)(d-Ser)(d-Thr)(Gba)(Pip) | KWKK |
| 618 | (B)(Gly)(d-Phe)(Gly)(Gly)(d-Thr) | KWKK |
| 619 | (Abu)(Gly)(Gly)(Gly)(Gly)(d-Phe) | KWKK |
| 620 | (d-Pro)(d-Pro)(d-Ser)(d-Glu)(d-Glu)(Dab) | KWKK |
| 621 | (Gly)(Abu)(Gly)(Gly)(d-Arg)(Dpa) | KWKK |
| 622 | (d-Asp)(Abu)(B)(Hle)(Amf)(Nle) | KWKK |
| 623 | (d-Asn)(d-Pro)(d-Ser)(Nle)(d-Phe)(d-Glu) | KWKK |
| 624 | (d-Lys)(B)(B)(d-Asp)(ox-Amf)(d-Lys) | KWKK |
| 625 | (d-Gln)(B)(d-Pro)(Nle)(Pip)(d-Gln) | KWKK |
| 626 | (d-Gln)(d-Phe)(d-Asp)(d-His)(Gly)(Gly) | KWKK |
| 627 | (d-Pro)(d-Asn)(d-Ser)(Nle)(d-Phe)(d-Glu) | KWKK |
| 628 | (d-Asp)(d-Thr)(d-Gln)(Hle)(d-Pro)(Hle) | KWKK |
| 629 | (B)(d-Glu)(Gly)(d-His)(Dab)(Hle) | KWKK |
| 630 | (Nle)(d-His)(d-His)(B)(d-Glu)(Amf) | KWKK |
| 631 | (d-Thr)(Dab)(d-Asp)(Gly)(d-Thr)(Gly) | KWKK |
| 632 | (B)(Abu)(d-Phe)(Gba)(d-Ser)(d-Ser) | KWKK |
| 633 | (d-Phe)(Dab)(d-Glu)(B)(Abu)(Dpa) | KWKK |
| 634 | (Gly)(Gly)(d-Glu)(Hle)(d-His)(d-Pro) | KWKK |
| 635 | (Gly)(Dpa)(B)(Gly)(B)(d-His) | KWKK |
| 636 | (B)(Abu)(Nle)(d-Lys)(d-His)(Abu) | KWKK |
| 637 | (Gly)(d-Ser)(B)(Gly)(ox-Amf)(Amf) | KWKK |
| 638 | (Gly)(Abu)(ox-His)(d-His)(d-Ser)(d-Asn) | KWKK |
| 639 | (B)(d-Pro)(d-Asp)(Hle)(d-Thr)(d-Asp) | KWKK |
| 640 | (Gly)(Gly)(Gly)(d-Pro)(Nle)(d-Asp) | KWKK |

### Example 13—In-Cell Penetration Selection-Mass Spectrometry

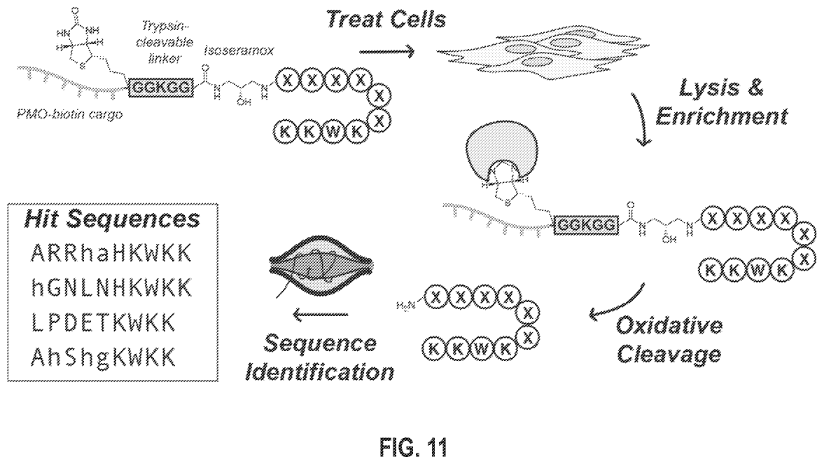

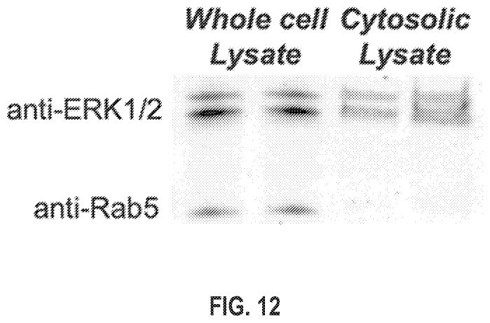

The library was subjected to the in-cell penetration selection-mass spectrometry platform (in-cell PS-MS) to discover sequences that are present in the whole cell lysate and in the cytosol (FIG. 11). Confluent HeLa cells in a 12-well plate were treated with 20 μM of peptide-library (1,000 members) or PPMO-library (˜103 members, 3.5 nmol individual peptide per bead) for 1 h at 37° C., before being washed with PBS and 0.1 mg/mL heparin to dissociate membrane-bound conjugates. Cells were then lifted and extracellular conjugates digested with Trypsin, pelleted, and washed with PBS. Cells were gently lysed using either RIPA buffer (for whole cell extraction) or digitonin buffer (for cytosolic extraction). The protein in the no treatment control lysates were analyzed via sodium dodecyl sulfate-polyacrylamide gel electrophoresis (SDS-PAGE) and Western blot to visualize the presence of ERK1/2 (cytosolic marker) and Rab5 (an endosomal marker). Rab5 is observed in the whole cell lysate but not in the cytosolic extract (FIG. 12). In addition to the experimental samples, half of the no-treatment lysates were spiked with the library as positive controls.

Biotinylated species in the lysates were affinity captured with magnetic streptavidin beads, and ultimately released by oxidative cleavage using brief incubation with sodium periodate. The isolated peptides were desalted by solid-phase extraction and analyzed via Orbitrap tandem mass spectrometry using a mixed fragmentation method optimized for cationic peptides, consisting of electron-transfer dissociation (ETD), higher-energy ETD, and higher-energy collisional dissociation (HCD). Sequences matching the library design were then identified using a Python script.

### Example 12—Microscopy

HeLa cells were plated at a density of 8,000 cells/well in a 96-well cover glass-bottomed plate the day before the experiment. For standard localization imaging, cells were treated with PMO-Sulfo-Cy5-CPP conjugates at 5 μM or 25 μM for 30 min at 37° C. and 5% CO₂. Each well was washed with media and incubated in fresh media for 1 h before Hoechst (nuclear) and Lysotracker Green (endosomal) fluorescent tracking dyes were added and imaged immediately. Cells were treated with 50 μM 7-Diethylaminocoumarin-3-carboxylic acid (DEAC)-k5 for 1 h at 37° C. and 5% CO₂ before being washed with media. Then, PMO-Sulfo-Cy5-CPP conjugates were added as before. Sytox Green was added immediately before imaging in order to exclude observation of nonviable cells. Imaging was performed at the Whitehead Institute's Keck Imaging Facility on an RPI Spinning Disk Confocal Microscope at 40× objective.

TABLE 6
Peptide-PMO Conjugate Library-Peptides
identified from experimental samples

| | | |
|---|---|---|
| | | Terminal |
| | | Motif |
| SEQ | | (d-amino |
| ID NO. | Peptide Sequences | acids) |

Whole Cell Extract

| | | |
|---|---|---|
| 641 | (Abu)(d-Pro)(d-Pro) | KWKK |
| 642 | (d-Pro)(d-Pro)(Abu) | KWKK |
| 643 | (Nle)(d-Thr)(d-Glu)(d-Arg) | KWKK |
| 644 | (Nle)(d-Pro)(d-Asp)(d-Glu)(d-Thr) | KWKK |
| 645 | (Nle)(d-Pro)(d-Asp)(d-Arg)(ox-Trp) | WKK |
| 646 | (B)(Abu)(d-Ser)(Abu)(Hle) | KWKK |
| 647 | (d-Lys)(Nap)(d-Glu)(d-Thr) | KWKK |
| 643 | (Nle)(d-Thr)(d-Glu)(d-Arg) | KKWK |
| 648 | (Nle)(d-Pro)(d-Asp)(d-Arg)W | KK |
| | (ox-Trp) | |
| 649 | (Nle)(d-Asp)(Dab)(Hle) | KWKK |
| 650 | (d-Lys)(d-Asp)(Nle)(d-Gln)(d-Gln) | KWKK |
| 651 | (Nle)(d-Thr)(d-Glu)(B)(Abu) | KWKK |
| 652 | (d-Ser)(Pip)(d-Ser)(Gly)(Hle) | KWKK |
| 653 | (Nle)(d-Thr)(d-Pro)(d-Thr)(d-Ser) | KWKK |
| 654 | (B)(Abu)(B)(d-Thr)(Hle) | KWKK |
| 655 | (d-Lys)(d-Asp)(d-Asn)(Hle) | KWKK |
| 656 | (B)(d-Glu)(Dab)(Gly)(Hle) | KWKK |

Cytosolic Extract

| | | |
|---|---|---|
| 657 | (B)(d-Arg)(d-Arg)(Abu)(Dab)(d-His) | KWKK |
| 658 | (B)(d-Arg)(Abu)(d-Arg)(Dab)(d-His) | KWKK |
| 659 | (Nle)(d-Asn)(Hle)(Nle)(d-Thr) | KWKK |
| | (d-His) | |
| 660 | (Gba)(d-Asn)(Nle)(d-Asn)(d-His) | KWKK |
| 661 | (ox-His)(d-His)(Hle) | KWKK |
| 662 | (d-Arg)(ox-Nap)(d-Asn)(d-His) | KWKK |
| 663 | (d-Lys)(B)(Abu)(d-Arg)(d-His) | KWKK |
| 664 | (Abu)(Gly)(d-Asn)(Nle)(d-Asn) | KWKK |
| | (d-His) | |
| 665 | (d-Lys)(d-Arg)(d-Arg)(d-His) | KWKK |
| 666 | (B)(d-Arg)(Nle)(Gba)(Dpa) | KWKK |
| 667 | (Nle)(d-Asn)(d-Lys)(B)(Gba)(d-His) | KWKK |
| 668 | (Nle)(Hle)(ox-Amf)(d-Pro)(d-Phe) | KWKK |
| 669 | (B)(d-Arg)(Gba)(Dpa)(Nle) | KWKK |

### Example 13—Hit Peptides

Several hit peptides were selected for experimental validation and showed differential activities depending on the fraction in which they were found. Hit PMO-delivering sequences were then identified as those peptides found only in the PPMO-library fractions that do not overlap with peptides found in the cell only control or the samples treated with the peptide-library. Two sequences found in the cytosolic extract (Pep1a, Pep1b) and two from the whole cell extract (Pep1c, Pep1d), were selected with sequences shown below.

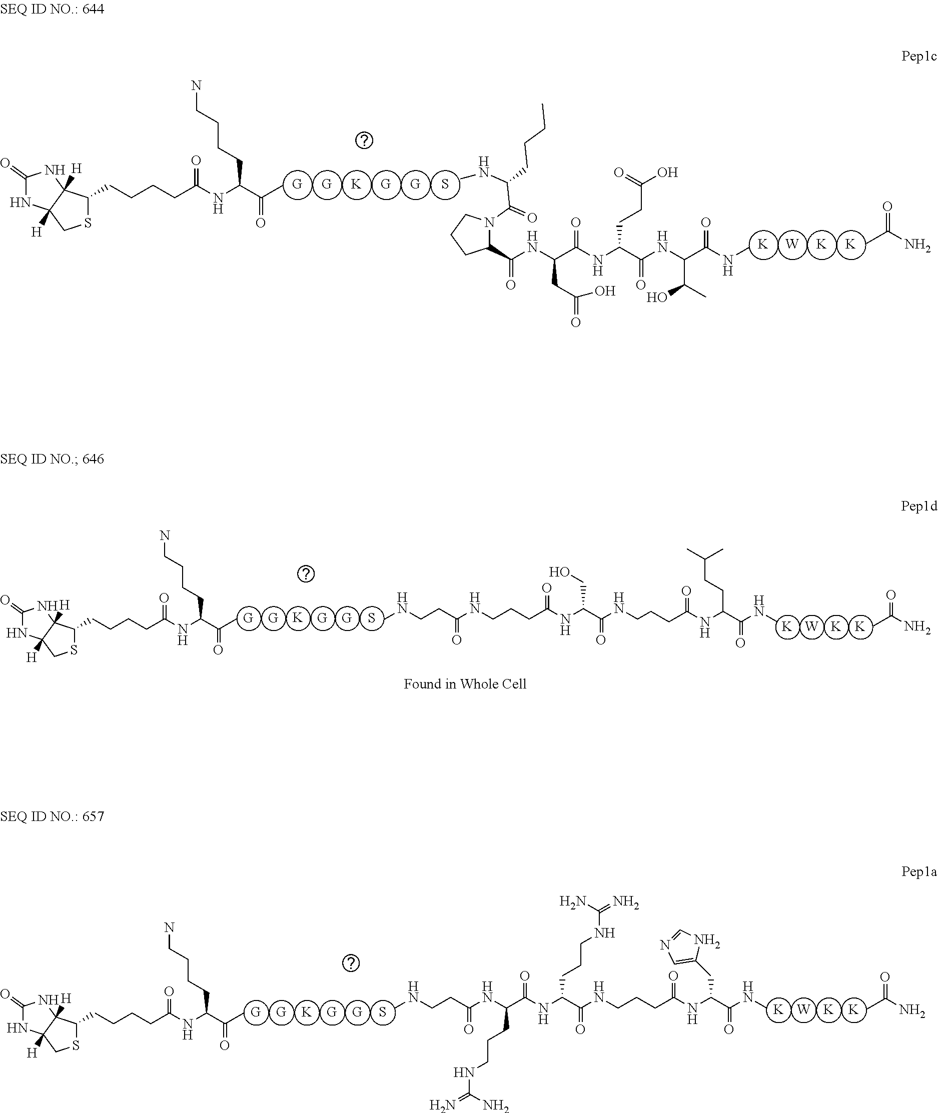

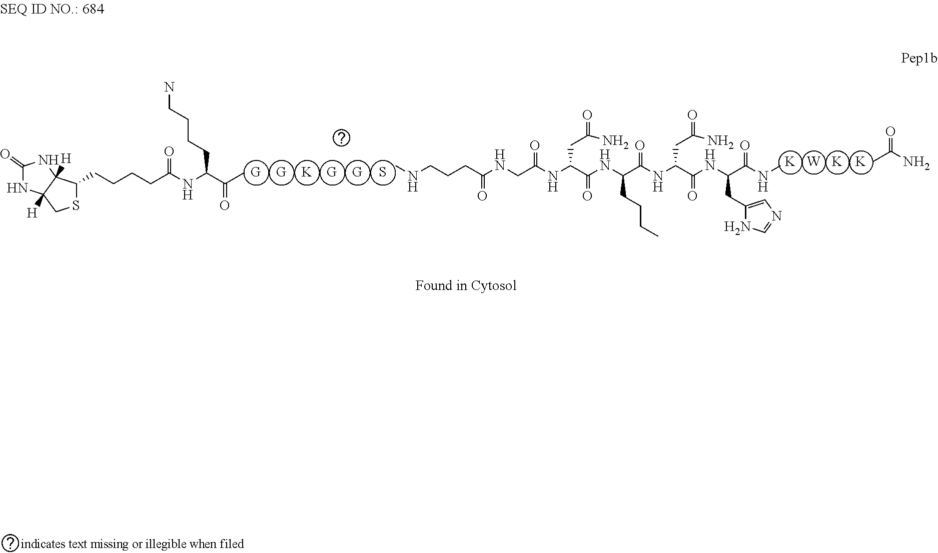

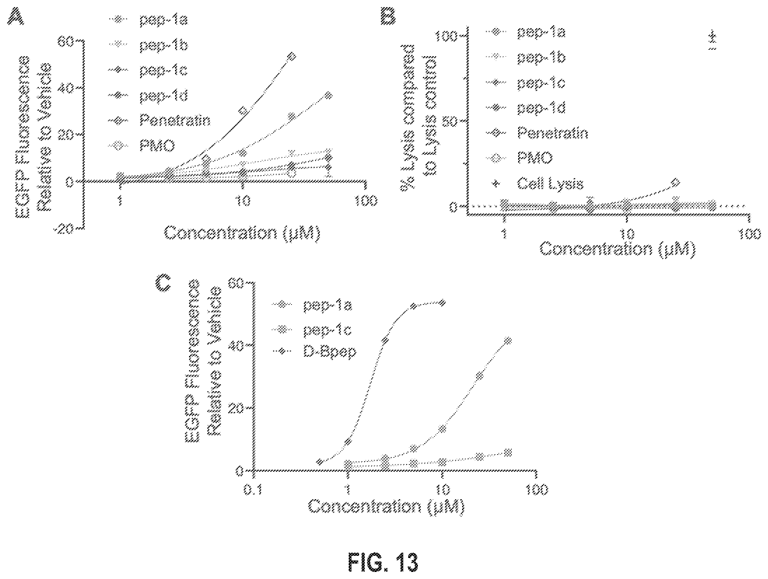

These peptides were synthesized via semi-automated solid-phase fast-flow peptide synthesis with identical sequences to the library design with the exception of a d-Ser residue to replace the isoseramox linker. These sequences were tested first in a concentration-response EGFP assay. The sequences extracted from the cytosol showed significantly increased activity compared to the sequences from the whole cell lysate, with Pep1a showing an EC50 of 43 μM compared to Pep1c with EC50 of 380 μM (FIG. 13A). It was also confirmed that these sequences did not exhibit membrane toxicity at the concentrations tested (FIG. 13B). The peptides showed a positive correlation between charge and activity, with the highest performing peptide (Pep1a) having a charge of +7, compared to Pep1c with a charge of +2.

Although the hit peptides do not show higher activity than the parent peptide penetratin, they do show significantly less toxicity and membrane disruption, demonstrating their potential utility as PMO delivery vehicles. Pep1a and Pep1c were compared to the known endosomal escape peptide Bpep, composed of eight Arg residues interspaced with non-α-backbone residues β-alanine and 6-aminohexanoic acid, which shows an EC50 closer to 3 μM (FIG. 13C).

The bioactive hit peptides are not solely responsible for the PMO delivery exhibited by the entire 1,000-member library. Within a 20 μM treatment dose of a ˜1,000 member library, each individual peptide would be present at ˜20 nM, a concentration at which no single peptide is known to deliver PMO cargo.

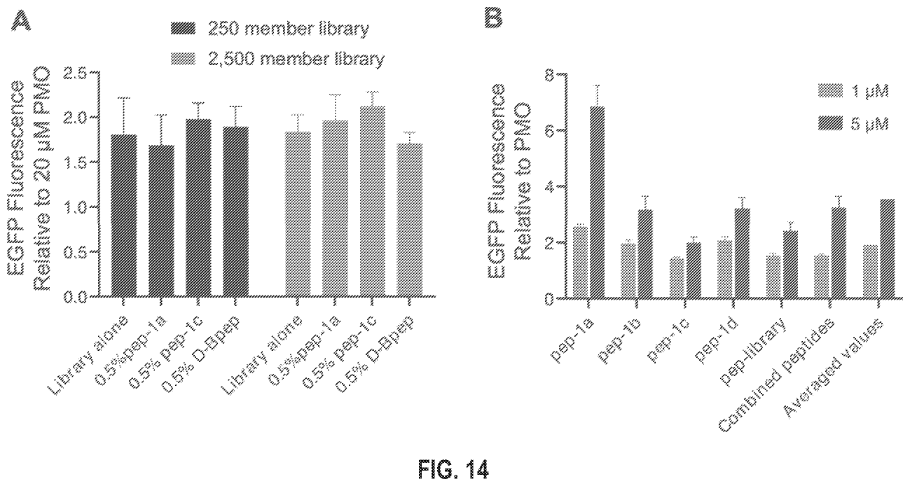

To investigate whether overall library PMO delivery efficacy could be due to a few highly active peptides, HeLa 654 cells were treated with a 250-member library at 20 μM and compared the activity to HeLa cells treated with the same library with a penetrant peptide (either Pep1a or the positive control D-Bpep) spiked in at roughly the concentration of the individual library members (FIG. 14A). There was no significant difference in PMO delivery between the 250-member library alone and the library with potent peptides spiked in, showing that the overall, combined library penetration is unlikely to be affected by the activity of a few members. To further confirm this finding, the experiment was repeated with a larger library of 2,500 members and spiked in penetrant peptides at 10-fold higher concentrations than the individual library members, which also showed no significant change in library PMO delivery.

The four hit library candidates, as well as a “library peptide” found in the quality control sequencing of the library, but not extracted from cells, were tested both individually and in combination for PMO delivery (FIG. 14B). The 5 μM “combined peptides” sample contains each individual peptide at 1 μM, yet this five-member library shows significantly more PMO delivery than any of the individual peptides at 1 μM. In fact, the PMO delivery of the peptides in combination at 5 μM total peptide more closely matches the averaged values of all five peptides individually at 5 μM, further suggesting that the activity observed from the library is due to an ensemble effect from the activity of many cationic individual peptides and not due to a few highly active sequences.

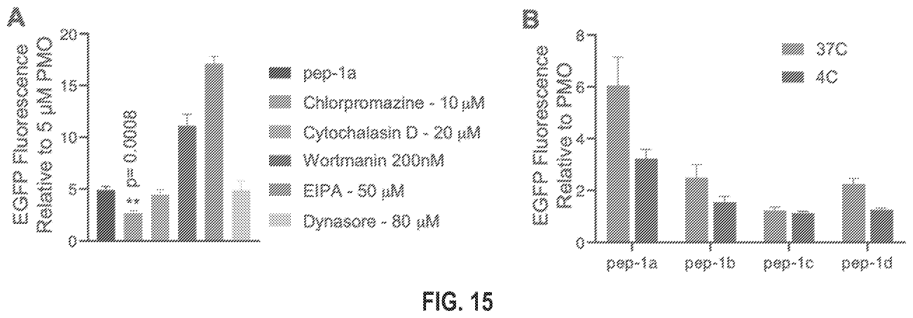

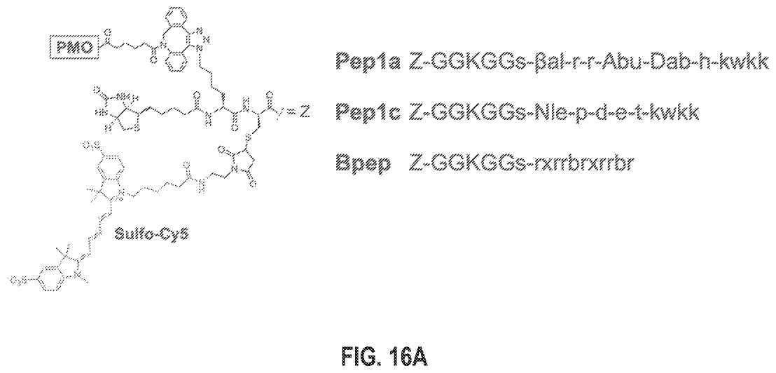

Pep1a was tested with a series of chemical endocytosis inhibitors in a pulse-chase format EGFP assay, in which HeLa 654 cells were pre-incubated with inhibitors to arrest various endocytosis pathways before PMO-CPPs were added. The cells were pre-incubated for 30 min with the indicated compound and then 5 μM PMO-Pep1a was added. After treatment with the construct for 3 h, the cells were washed with 0.1 mg/mL heparin, and the media was exchanged for fresh, untreated media for 22 h to dissociate membrane-bound constructs prior to flow cytometry. Activity of Pep1a was impacted by 10 μM chlorpromazine (FIG. 15A).

The cells were pre-incubated for 30 min at 4° C. or 37° C., followed by the addition of PMO-peptide conjugate to each well at a concentration of 5 μM. After incubation at 4° C. or 37° C. for 2 h, the cells were washed with 0.1 mg/mL heparin, and the media was exchanged for fresh, untreated media for 22 h prior to flow cytometry. The 4° C. condition significantly impacted the activities of each conjugate, indicating that, like the entire library sample (FIG. 10B), uptake of the four hit peptides is energy-dependent, and the peptides are most likely entering the cells through endocytosis (FIG. 15B).

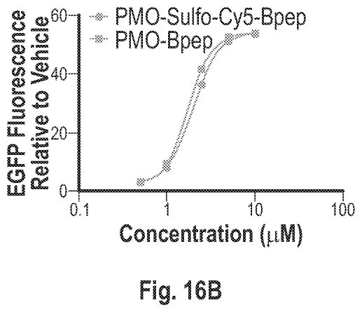

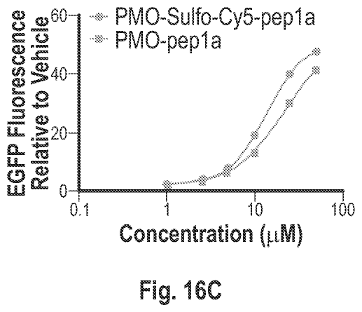

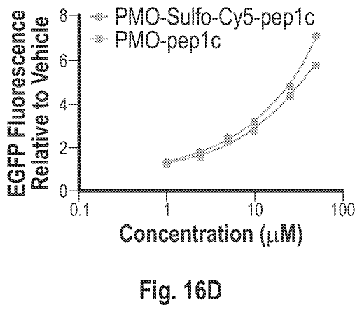

The differences in activity between the sequences found in the cytosol versus the whole cell lysate were investigated and compared to a benchmark compound, PMO-D-Bpep, using flow cytometry. For this purpose, several SulfoCy5-labeled PMO-CPPs were generated and tested to ensure the fluorophore did not impact PMO delivery activity (FIG. 16A). Comparing the results of the EGFP assay of conjugates with and without the fluorophore, no significant differences were found between the constructs' EC50 values (FIG. 16B-D).

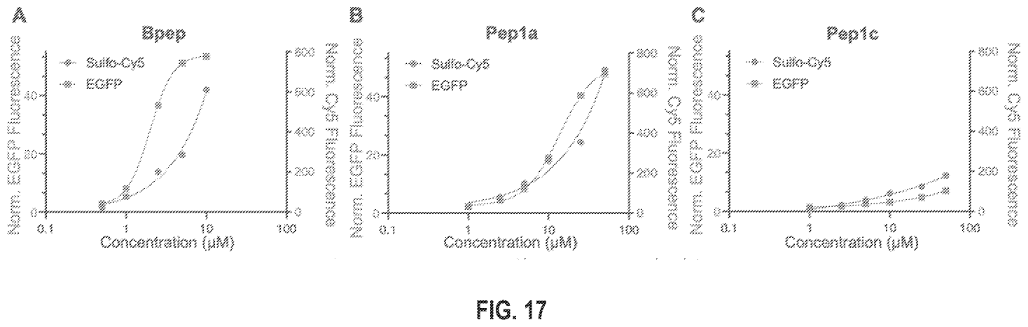

In addition, the uptake and nuclear delivery of the PMO-SulfoCy5-CPPs was analyzed by monitoring both the EGFP fluorescence from the EGFP assay and the Cy5 fluorescence from the total uptake of the fluorescent analogs. Each conjugate demonstrated similar concentration-dependent increases in both EGFP and Cy5 fluorescence (FIG. 17A-C). Pep1c showed low fluorescence signal in both the EGFP and Cy5 channels. On the other hand, Pep1a showed higher fluorescence signals in each channel, indicating greater uptake and nuclear localization compared to Pep1c. Interestingly, D-Bpep showed greater EGFP fluorescence but slightly diminished Cy5 fluorescence than Pep1a, indicating that D-Bpep may access the nucleus more efficiently once taken up in endosomes, but with a lower total uptake compared to Pep1a.

The hit peptides discovered by PS-MS likely do not permeabilize the endosomal membrane to allow release of other cargoes. HeLa cells were first preincubated with DEAC-k5, an endosomal-localizing peptide composed of D-lysine residues.⁴⁷ The DEAC-k5 is visible as blue puncta in the no-CPP treatment control, indicating the expected endosomal localization (FIG. 13). After treatment with PMO-SulfoCy5-CPP constructs, the DEAC-k5 continues to occupy endosomes, indicating that Pep1a and Pep1c do not non-specifically permeabilize the endosome to release other endosomal cargo.

Provided herein it is shown that chemical libraries may reach the diversity of other display techniques for the identification of peptide binders to proteins and that this strategy can be applied for cell-surface selection in vivo. In addition, by extracting the cytosol for in-cell selection of fully synthetic peptide libraries conjugated to a model antisense cargo and comparing these sequences to those found in a whole cell extract, sequences that accumulate in the endosomes can be excluded.

In-cell PS-MS combined with subcellular fractionation resulted in the identification of a novel, abiotic peptide capable of accessing the cytosol and delivering PMO to the nucleus. Pep1a, like the positive control peptide Bpep, was able to deliver PMO to the nucleus by escaping endosomes. Furthermore, Pep1a does not appear to permeabilize the endosome to allow the escape of other endosomal cargo, nor does it demonstrate cell membrane toxicity. All peptides discovered through this novel platform demonstrated lower toxicity than the CPP penetratin. Endowed with lower toxicity and superior chemical diversity provide by the noncanonical residues, the peptides discovered with the in-cell PS-MS platform show advantages over the library's parent peptide. The few active PMO-CPPs individually sequenced and validated are not solely responsible for the overall cell penetration of the library, however. In fact, it is more likely that the PMO delivery arises from the combined activity of a number of peptides at low concentrations, including the hits discovered with the platform herein.

What is claimed is:

1. An oligonucleotide conjugate comprising a compound of Formula I: 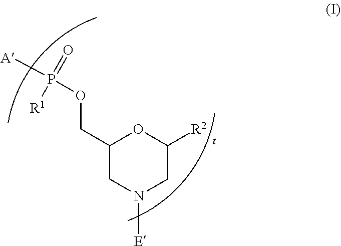
or a pharmaceutically acceptable salt thereof,
wherein:
A′ is selected from —N(H)CH₂C(O)NH₂, —N(C₁₋₆-alkyl)CH₂C(O)NH₂, 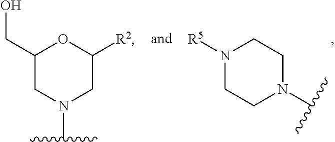
wherein
R⁵ is —C(O)(O-alkyl)ₓ-OH, wherein x is 3-10 and each alkyl group is, independently at each occurrence, C₂₋₆-alkyl,
or R⁵ is selected from H, —C(O)C₁₋₆-alkyl, trityl, monomethoxytrityl, —(C₁₋₆-alkyl)-R⁶, —(C₁₋₆-heteroalkyl)-R⁶, aryl-R⁶, heteroaryl-R⁶, —C(O)O—(C₁₋₆-alkyl)-R⁶, —C(O)O-aryl-R⁶, —C(O)O— heteroaryl-R⁶, and 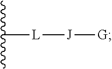
R⁶ is selected from OH, SH, and NH₂, or R⁶ is O, S, or NH, each of which is covalently linked to a solid support;
each R¹ is independently selected from OH and —N(R³)(R⁴), wherein each R³ and R⁴ are, independently at each occurrence, H or —C₁₋₆-alkyl;
each R² is independently, at each occurrence, selected from H, a nucleobase, and a nucleobase functionalized with a chemical protecting-group, wherein the nucleobase, independently at each occurrence, comprises a C₃₋₆-heterocyclic ring selected from pyridine, pyrimidine, purine, and deaza-purine;
t is 8-40;
E′ is selected from H, —C₁₋₆-alkyl, —C(O)C₁₋₆-alkyl, benzoyl, stearoyl, trityl, monomethoxytrityl, dimethoxytrityl, trimethoxytrityl, 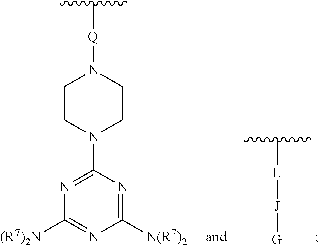
wherein
Q is —C(O)(CH₂)₆C(O)— or —C(O)(CH₂)₂S₂(CH₂)₂C(O)—;
R⁷ is —(CH₂)₂OC(O)N(R⁸)₂, wherein R⁸ is —(CH₂)₆NHC(═NH)NH₂;
L is —C(O)(CH₂)₁₋₈C(O)—C₇₋₁₈-heteroaromatic-((CH₂)₁₋₈C(O)—, —C(O)(CH₂)₁₋₈C(O)—C₇₋₁₈-heteroaromatic-(W)—; —C(O)(CH₂)₁₋₈C(O)—C₇₋₁₈-heteroaromatic-(W—W)—;
W is independently at each occurrence a linking amino acid;
J is a cell-penetrating peptide selected from d-R₈, d-BPEP, d-DPV7, d-DPV6, d-penetratin, d-Bac7, d-MPG, d-Hel11-7, d-TAT, d-TATp, d-WR₈, d-WBPEP, d-WDPV7, d-WDPV6, d-WTAT, or d-WTATp; and
G is selected from H, —C(O)C₁₋₆-alkyl, benzoyl, and stearoyl, wherein G is covalently linked to J;
provided that
A′ is 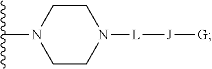
or E′ is

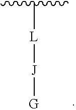

2. An oligonucleotide conjugate comprising a compound of Formula III: 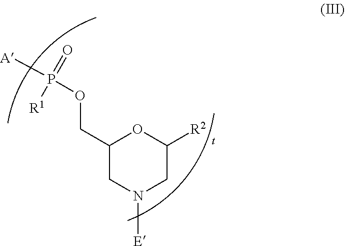
or a pharmaceutically acceptable salt thereof,
wherein:
A′ is selected from —N(H)CH₂C(O)NH₂, —N(C₁₋₆-alkyl)CH₂C(O)NH₂, 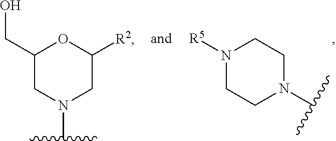
wherein
R⁵ is —C(O)(O-alkyl)ₓ-OH, wherein x is 3-10 and each alkyl group is, independently at each occurrence, C₂₋₆-alkyl,
or R⁵ is selected from H, —C(O)C₁₋₆-alkyl, trityl, monomethoxytrityl, —(C₁₋₆-alkyl)-R⁶, —(C₁₋₆-heteroalkyl)-R⁶, aryl-R⁶, heteroaryl-R⁶, —C(O)O—(C₁₋₆-alkyl)-R⁶, —C(O)O-aryl-R⁶, —C(O)O— heteroaryl-R⁶, and 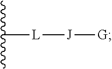
R⁶ is selected from OH, SH, and NH₂, or R⁶ is O, S, or NH, each of which is covalently linked to a solid support;
each R¹ is independently selected from OH and —N(R³)(R⁴), wherein each R³ and R⁴ are, independently at each occurrence, H or —C₁₋₆-alkyl;
each R² is independently, at each occurrence, selected from H, a nucleobase, and a nucleobase functionalized with a chemical protecting-group, wherein the nucleobase, independently at each occurrence, comprises a C₃₋₆-heterocyclic ring selected from pyridine, pyrimidine, purine, and deaza-purine;
t is 8-40;
E′ is selected from H, —C₁₋₆-alkyl, —C(O)C₁₋₆-alkyl, benzoyl, stearoyl, trityl, monomethoxytrityl, dimethoxytrityl, trimethoxytrityl, 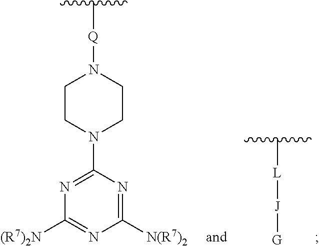
wherein
Q is —C(O)(CH₂)₆C(O)— or —C(O)(CH₂)₂S₂(CH₂)₂C(O)—;
R⁷ is —(CH₂)₂OC(O)N(R⁸)₂, wherein R⁸ is —(CH₂)₆NHC(═NH)NH₂;
L is —C(O)(CH₂)₁₋₈C(O)—C₇₋₁₈-heteroaromatic-((CH₂)₁₋₈C(O)—, —C(O)(CH₂)₁₋₈C(O)—C₇₋₁₈-heteroaromatic-(W)—; —C(O)(CH₂)₁₋₈C(O)—C₇₋₁₈-heteroaromatic-(W—W)—;
W is independently at each occurrence a linking amino acid;
J is a cell-penetrating peptide comprising 3 to 15 amino acids selected from unnatural amino acids or D-amino acids, further comprising a C-terminal sequence having a KWKK, KKWK, KWWKK, WWKK, WKK, KKKK, or KK motif; and
G is selected from H, —C(O)C₁₋₆-alkyl, benzoyl, and stearoyl, wherein G is covalently linked to J;
provided that
A′ is 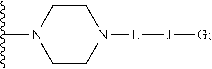
or E′ is

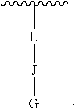

3. The oligonucleotide conjugate of any one of the above claims, wherein E′ is selected from H, —C₁₋₆-alkyl, —C(O)C₁₋₆-alkyl, benzoyl, stearoyl, trityl, monomethoxytrityl, dimethoxytrityl, trimethoxytrityl, and

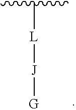

4. The oligonucleotide conjugate of any one of the above claims, wherein A′ is selected from —N(C₁₋₆-alkyl)CH₂C(O)NH₂,

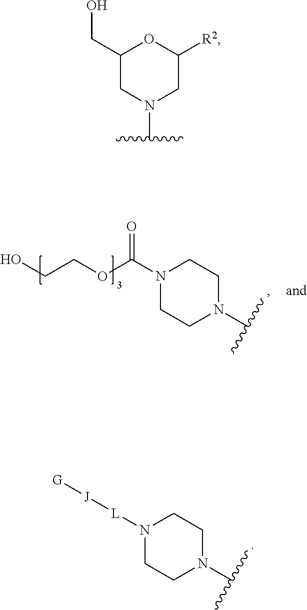

5. The oligonucleotide conjugate of any one of the above claims, wherein E′ is selected from H, —C(O)CH₃, benzoyl, stearoyl, trityl, 4-methoxytrityl, and

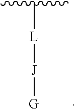

6. The oligonucleotide conjugate of any one of the above claims, wherein A′ is selected from —N(C₁₋₆-alkyl)CH₂C(O)NH₂, 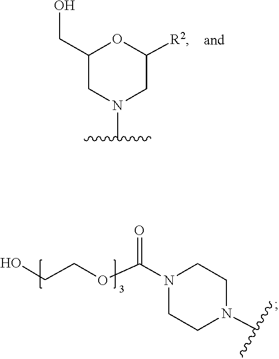
and
E′ is

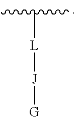

7. The oligonucleotide conjugate of any one of claims 1-5, wherein A′ is 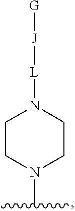
and
E′ is selected from H, —C(O)CH₃, trityl, 4-methoxytrityl, benzoyl, and stearoyl.

8. The oligonucleotide conjugate of any one of claims 1 or 2, wherein the peptide-oligonucleotide conjugate of Formula I or Formula III is a peptide-oligonucleotide conjugate selected from: 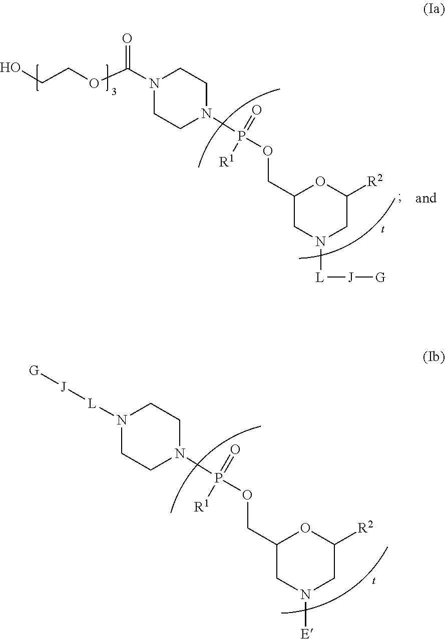
wherein E′ is selected from H, C₁₋₆-alkyl, —C(O)CH₃, benzoyl, and stearoyl.

9. The oligonucleotide conjugate of claims 1 or 8, wherein the peptide-oligonucleotide conjugate is of the Formula (Ia).

10. The oligonucleotide conjugate of any one of claims 1 or 8, wherein the peptide-oligonucleotide conjugate is of the Formula (Ib).

11. The oligonucleotide conjugate of any one of claims 1-10, wherein each R¹ is N(CH₃)₂.

12. The oligonucleotide conjugate of any one of claims 1-11, wherein each R² is a nucleobase, independently at each occurrence, selected from adenine, guanine, cytosine, 5-methyl-cytosine, thymine, uracil, and hypoxanthine.

13. The oligonucleotide conjugate of any one of claims 1-12, wherein L is —C(O)(CH₂)₁₋₈C(O)-DBCO—(CH₂)₁₋₈C(O)—.

14. The oligonucleotide conjugate of any one of claims 1-13, wherein L is

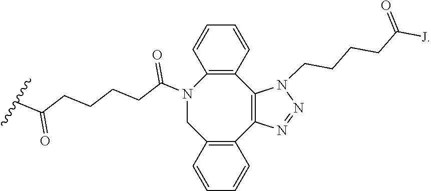

15. The oligonucleotide conjugate of any one of claims 1-12, wherein L is —C(O)(CH₂)₁₋₈C(O)-DBCO—(W)—.

16. The oligonucleotide conjugate of any one of claims 1-12, wherein L is —C(O)(CH₂)₁₋₈C(O)-DBCO—(W—W)—.

17. The oligonucleotide conjugate of any one of claims 1-12 or 15-16, wherein W is independently selected from glycine, proline, β-alanine, 6-aminohexanoic acid, or lysine.

18. The oligonucleotide conjugate of any one of claims 1-12 or 15-17, wherein W is lysine.

19. The oligonucleotide conjugate of any one of claims 1-12 or 15-17, wherein W—W is lysine-(6-amino hexanoic acid).

20. The oligonucleotide conjugate of any one of claims 1-19, wherein J is selected from d-DPV7, d-DPV6, d-penetratin, d-Bac7, d-MPG, d-Hel11-7, d-WR₈, d-WBPEP, d-WDPV7, d-d-WDPV6, d-WTAT, or d-WTATp.

21. The oligonucleotide conjugate of any one of claims 2-19, wherein the unnatural amino acids are selected from Abu (γ-aminobutyric acid), B (β-alanine), Hle (homoleucine), Nle (norleucine), Nap (naphthylalanine), Dpa (diphenylalanine), Dab (diaminobutyric acid), Pip (aminopiperidine-carboxylic acid), Amf (aminomethylphenylalanine), and Gba (2-amino-4-guanidinobutanoic acid).

22. The oligonucleotide conjugate of any one of claims 2-19 or 21, wherein the cell penetrating peptide is selected from SEQ ID NOS: 33-669.

23. The oligonucleotide conjugate of any one of claims 1-19 or 21-22, wherein the cel penetrating peptide is selected from:
SEQ ID NO.: 657
(B)(d-Arg)(d-Arg)(Abu)(Dab)(d-His);
SEQ ID NO.: 664
(Abu)(Gly)(d-Asn)(Nle)(d-Asn)(d-His);
SEQ ID NO.: 644
(Nle)(d-Pro)(d-Asp)(d-Glu)(d-Thr);
and
SEQ ID NO.: 646
(B)(Abu)(d-Ser)(Abu)(Hle).

24. The oligonucleotide conjugate of any one of claims 1-19 or 21-22, wherein the C-terminal sequence is a KWKK motif.

25. The oligonucleotide conjugate of any one of claims 1-24, wherein G is selected from H, C(O)CH₃, benzoyl, and stearoyl.

26. The oligonucleotide conjugate of any one of claims 1-25, wherein G is H or —C(O)CH₃.

27. The oligonucleotide conjugate of any one of claims 1-26, wherein G is H.

28. The oligonucleotide conjugate of any one of claims 1-26, wherein G is —C(O)CH₃.

29. A composition comprising the conjugate of any one of claims 1-28, or a pharmaceutically acceptable salt thereof, and at least one pharmaceutically acceptable carrier.

30. A method of treating a disease in a subject in need thereof, the method comprising administering a therapeutically effective amount of the conjugate of any one of claims 1-28 or the composition of claim 29 to the subject.

31. The method of claim 30, wherein the disease is a neuromuscular disease.

32. The method of claim 31, where the neuromuscular disease is Duchenne muscular dystrophy.

33. A method of identifying a peptide capable of delivering a phosphorodiamidate morpholino oligomer (PMO) into a cell, the method comprising:
(a) treating a cell with a peptide or a peptide-conjugated phosphorodiamidate morpholino oligomer (PPMO);
(b) treating the cell with a digestive enzyme;
(c) lysing the cell for whole cell extraction forming a whole cell lysate or lysing the cell for cytosolic extraction forming a cytosolic fraction; and
(d) analyzing the whole cell lysate or cytosolic fraction with mass spectrometry to identify the peptide.

34. The method of claim 33, wherein the method further comprises washing the cell.

35. The method of claim 34, wherein the cell is washed with PBS.

36. The method of any one of claims 33-35, wherein the method further comprises analyzing the whole cell lysate or cytosolic fraction via Western blot for the presence of a cytosolic marker (Erk 1/2) or a late-endosomal marker (Rab5).

37. The method of claim 36, wherein the absence of Rab5 in the Western blot of the cytosolic fraction indicates exclusion of endosomes.

38. The method of any one of claims 33-37, wherein the digestive enzyme is trypsin.

39. The method of any one of claims 33-38, wherein the method comprises lysing the whole cell with RIPA buffer.

40. The method of any one of claims 33-38, wherein the method comprises lysing the cytosol with digitonin buffer.

41. The method of any one of claims 33-40, wherein the method comprises analyzing the peptide sequence with mass spectrometry with a mixed fragmentation method optimized for cationic peptides, consisting of electron-transfer dissociation (ETD), higher-energy ETD, and higher-energy collisional dissociation (HCD).

42. The method of any one of claims 33-41, wherein the cell is a HeLa cell or a C2C12 mouse myoblast.

43. The method of any one of the claims 33-42, wherein the peptide comprises 4 to 15 amino acids selected from unnatural amino acids or D-amino acids.

44. The method of any one of the claims 33-43, wherein the peptide further comprises a C-terminal sequence having a KWKK, KKWK, KWWKK, WWKK, WKK, KKKK, or KK motif.

45. The method of any one of the claims 33-44, wherein the peptide further comprises a C-terminal sequence having a KWKK motif.

46. The method of any one of claims 33-45, wherein the unnatural amino acids are selected from Abu (γ-aminobutyric acid), B (β-alanine), Hle (homoleucine), Ne (norleucine), Nap (naphthylalanine), Dpa (diphenylalanine), Dab (diaminobutyric acid), Pip (aminopiperidine-carboxylic acid), Amf (aminomethylphenylalanine), and Gba (2-amino-4-guanidinobutanoic acid).

47. The method of any one of claims 33-46, wherein the treating of the cell comprises treating the cell with a peptide-library or a PPMO-library.


SEQUENCE LISTING (698 sequences)

SEQ ID NO: 1 - protein, 8 aa, synthetic construct
RRRRRRRR

SEQ ID NO: 2 - protein, 11 aa, synthetic construct
  MOD_RES 2: note = X is 6-aminohexanoic acid
  MOD_RES 8: note = X is 6-aminohexanoic acid
  MOD_RES 5: note = X is beta-alanine
  MOD_RES 11: note = X is beta-alanine
RXRRXRRXRXR

SEQ ID NO: 3 - protein, 14 aa, synthetic construct
KRKKKGKLGKKRDP

SEQ ID NO: 4 - protein, 17 aa, synthetic construct
GRPRESGKKRKRKRLKP

SEQ ID NO: 5 - protein, 16 aa, synthetic construct
RQIKIWFQNRRMKWKK

SEQ ID NO: 6 - protein, 24 aa, synthetic construct
RRIRPRPPRLPRPRPRPLPFPRPG

SEQ ID NO: 7 - protein, 26 aa, synthetic construct
LAFLGFLGAAGSTMGAWSQPKKKRKV

SEQ ID NO: 8 - protein, 17 aa, synthetic construct
LLKLLLKLWKKLLKLLK

SEQ ID NO: 9 - protein, 9 aa, synthetic construct
RKKRRQRRR

SEQ ID NO: 10 - protein, 13 aa, synthetic construct
GRKKRRQRRRPPQ

SEQ ID NO: 11 - protein, 9 aa, synthetic construct
WRRRRRRRR

SEQ ID NO: 12 - protein, 13 aa, synthetic construct
  MOD_RES 3: note = X is 6-aminohexanoic acid
  MOD_RES 9: note = X is 6-aminohexanoic acid
  MOD_RES 6: note = X is beta-alanine
  MOD_RES 12: note = X is beta-alanine
WRXRRXRRXRRXR

SEQ ID NO: 13 - protein, 15 aa, synthetic construct
WKRKKKGKLGKKRDP

SEQ ID NO: 14 - protein, 18 aa, synthetic construct
WGRPRESGKKRKRKRLKP

SEQ ID NO: 15 - protein, 10 aa, synthetic construct
WRKKRRQRRR

SEQ ID NO: 16 - protein, 14 aa, synthetic construct
WGRKKRRQRRRPPQ

SEQ ID NO: 17 - protein, 8 aa, synthetic construct
  SITE 1..8: note = D-Arginine
RRRRRRRR

SEQ ID NO: 18 - protein, 12 aa, synthetic construct
  MOD_RES 2: note = X is 6-aminohexanoic acid
  MOD_RES 8: note = X is 6-aminohexanoic acid
  MOD_RES 5: note = X is beta-alanine
  MOD_RES 11: note = X is beta-alanine
  SITE 1: note = D- Arginine
  SITE 3..4: note = D- Arginine
  SITE 6..7: note = D- Arginine
  SITE 9..10: note = D- Arginine
  SITE 12: note = D- Arginine
RXRRXRRXRRXR

SEQ ID NO: 19 - protein, 14 aa, synthetic construct
  SITE 1: note = D-Lysine
  SITE 3..5: note = D-Lysine
  SITE 7: note = D-Lysine
  SITE 10..11: note = D-Lysine
  SITE 2: note = D-Arginine
  SITE 12: note = D-Arginine
  SITE 8: note = D-Leucine
  SITE 13: note = D-Aspartic acid
  SITE 14: note = D-Proline
KRKKKGKLGKKRDP

SEQ ID NO: 20 - protein, 17 aa, synthetic construct
  SITE 2: note = D-Arginine
  SITE 4: note = D-Arginine
  SITE 10: note = D-Arginine
  SITE 12: note = D-Arginine
  SITE 14: note = D-Arginine
  SITE 3: note = D-Proline
  SITE 17: note = D-Proline
  SITE 5: note = D- Glutamic acid
  SITE 6: note = D-Serine
  SITE 7: note = D-Glycine
  SITE 8..9: note = D-Lysine
  SITE 11: note = D-Lysine
  SITE 13: note = D-Lysine
  SITE 16: note = D-Lysine
  SITE 15: note = D-Leucine
GRPRESGKKRKRKRLKP

SEQ ID NO: 21 - protein, 16 aa, synthetic construct
  SITE 1: note = D-Arginine
  SITE 10..11: note = D-Arginine
  SITE 2: note = D- Glutamine
  SITE 8: note = D- Glutamine
  SITE 3: note = D- Isoleucine
  SITE 5: note = D- Isoleucine
  SITE 4: note = D-Lysine
  SITE 13: note = D-Lysine
  SITE 15..16: note = D-Lysine
  SITE 6: note = D-Tryptophan
  SITE 14: note = D-Tryptophan
  SITE 7: note = D-Phenylalanine
  SITE 12: note = D-Methionine
  SITE 9: note = D-Asparagine
RQIKIWFQNRRMKWKK

SEQ ID NO: 22 - protein, 24 aa, synthetic construct
  SITE 1..2: note = D-Arginine
  SITE 4: note = D-Arginine
  SITE 6: note = D-Arginine
  SITE 9: note = D-Arginine
  SITE 12: note = D-Arginine
  SITE 14: note = D-Arginine
  SITE 16: note = D-Arginine
  SITE 22: note = D-Arginine
  SITE 3: note = D- Isoleucine
  SITE 5: note = D-Proline
  SITE 7..8: note = D-Proline
  SITE 11: note = D-Proline
  SITE 13: note = D-Proline
  SITE 15: note = D-Proline
  SITE 17: note = D-Proline
  SITE 19: note = D-Proline
  SITE 21: note = D-Proline
  SITE 23: note = D-Proline
  SITE 10: note = D-Leucine
  SITE 18: note = D-Leucine
  SITE 20: note = D-Phenylalanine
RRIRPRPPRLPRPRPRPLPFPRPG

SEQ ID NO: 23 - protein, 26 aa, synthetic construct
  SITE 1: note = D-Leucine
  SITE 4: note = D-Leucine
  SITE 7: note = D-Leucine
  SITE 2: note = D- Alanine
  SITE 9..10: note = D- Alanine
  SITE 16: note = D- Alanine
  SITE 3: note = D-Phenylalanine
  SITE 6: note = D-Phenylalanine
  SITE 8: note = D-Glycine
  SITE 12: note = D-Serine
  SITE 18: note = D-Serine
  SITE 13: note = D-Threonine
  SITE 14: note = D-Methionine
  SITE 17: note = D-Tryptophan
  SITE 19: note = D- Glutamine
  SITE 20: note = D-Proline
  SITE 21..23: note = D-Lysine
  SITE 25: note = D-Lysine
  SITE 24: note = D-Arginine
  SITE 26: note = D-Valine
LAFLGFLGAAGSTMGAWSQPKKKRKV

SEQ ID NO: 24 - protein, 17 aa, synthetic construct
  SITE 1..2: note = D-Leucine
  SITE 4..6: note = D-Leucine
  SITE 8: note = D-Leucine
  SITE 12..13: note = D-Leucine
  SITE 15..16: note = D-Leucine
  SITE 3: note = D-Lysine
  SITE 7: note = D-Lysine
  SITE 10..11: note = D-Lysine
  SITE 14: note = D-Lysine
  SITE 17: note = D-Lysine
  SITE 9: note = D-Tryptophan
LLKLLLKLWKKLLKLLK

SEQ ID NO: 25 - protein, 9 aa, synthetic construct
  SITE 1: note = D-Arginine
  SITE 4..5: note = D-Arginine
  SITE 7..9: note = D-Arginine
  SITE 2..3: note = D-Lysine
  SITE 6: note = D- Glutamine
RKKRRQRRR

SEQ ID NO: 26 - protein, 13 aa, synthetic construct
  SITE 2: note = D-Arginine
  SITE 5..6: note = D-Arginine
  SITE 8..10: note = D-Arginine
  SITE 3..4: note = D-Lysine
  SITE 7: note = D- Glutamine
  SITE 13: note = D- Glutamine
  SITE 11..12: note = D-Proline
GRKKRRQRRRPPQ

SEQ ID NO: 27 - protein, 9 aa, synthetic construct
  SITE 1: note = D-Tryptophan
  SITE 2..9: note = D-Arginine
WRRRRRRRR

SEQ ID NO: 28 - protein, 13 aa, synthetic construct
  SITE 1: note = D-Tryptophan
  SITE 2: note = D-Arginine
  SITE 4..5: note = D-Arginine
  SITE 7..8: note = D-Arginine
  SITE 10..11: note = D-Arginine
  SITE 13: note = D-Arginine
  MOD_RES 3: note = X is 6-aminohexanoic acid
  MOD_RES 9: note = X is 6-aminohexanoic acid
  MOD_RES 6: note = X is beta-alanine
  MOD_RES 12: note = X is beta-alanine
WRXRRXRRXRRXR

SEQ ID NO: 29 - protein, 15 aa, synthetic construct
  SITE 1: note = D-Tryptophan
  SITE 2: note = D-Lysine
  SITE 4..6: note = D-Lysine
  SITE 8: note = D-Lysine
  SITE 11..12: note = D-Lysine
  SITE 3: note = D-Arginine
  SITE 13: note = D-Arginine
  SITE 9: note = D-Leucine
  SITE 14: note = D-Aspartic acid
  SITE 15: note = D-Proline
WKRKKKGKLGKKRDP

SEQ ID NO: 30 - protein, 18 aa, synthetic construct
  SITE 1: note = D-Tryptophan
  SITE 3: note = D-Arginine
  SITE 5: note = D-Arginine
  SITE 11: note = D-Arginine
  SITE 13: note = D-Arginine
  SITE 15: note = D-Arginine
  SITE 4: note = D-Proline
  SITE 18: note = D-Proline
  SITE 5: note = D-Arginine
  SITE 6: note = D- Glutamic acid
  SITE 7: note = D-Serine
  SITE 8: note = D-Glycine
  SITE 9..10: note = D-Lysine
  SITE 12: note = D-Lysine
  SITE 14: note = D-Lysine
  SITE 17: note = D-Lysine
  SITE 16: note = D-Leucine
WGRPRESGKKRKRKRLKP

SEQ ID NO: 31 - protein, 10 aa, synthetic construct
  SITE 1: note = D-Tryptophan
  SITE 2: note = D-Arginine
  SITE 5..6: note = D-Arginine
  SITE 8..10: note = D-Arginine
  SITE 3..4: note = D-Lysine
  SITE 7: note = D- Glutamine
WRKKRRQRRR

SEQ ID NO: 32 - protein, 14 aa, synthetic construct
  SITE 1: note = D-Tryptophan
  SITE 3: note = D-Arginine
  SITE 6..7: note = D-Arginine
  SITE 9..11: note = D-Arginine
  SITE 4..5: note = D-Lysine
  SITE 8: note = D- Glutamine
  SITE 14: note = D- Glutamine
  SITE 12..13: note = D-Proline
WGRKKRRQRRRPPQ

SEQ ID NO: 33 - skipped

SEQ ID NO: 34 - protein, 6 aa, synthetic construct
  MOD_RES 1: note = X is Dpa
  SITE 2: note = D-Phenylalanine
  SITE 4: note = D-Phenylalanine
  SITE 3: note = D-Lysine
  SITE 5: note = D-Asparagine
  SITE 6: note = D-Threonine
XFKFNT

SEQ ID NO: 35 - skipped

SEQ ID NO: 36 - protein, 6 aa, synthetic construct
  MOD_RES 1: note = X is Dpa
  MOD_RES 5: note = X is Dpa
  SITE 2: note = D-Asparagine
  SITE 6: note = D-Asparagine
  SITE 3: note = D-Threonine
  SITE 4: note = D-Arginine
XNTRXN

SEQ ID NO: 37 - protein, 6 aa, synthetic construct
  MOD_RES 1: note = X is Nap
  MOD_RES 4: note = X is Nle
  SITE 2: note = D-Threonine
  SITE 3: note = D-Phenylalanine
  SITE 6: note = D- Glutamic acid
XTFXGE

SEQ ID NO: 38 - protein, 6 aa, synthetic construct
  MOD_RES 1: note = X is Dpa
  MOD_RES 4: note = X is Nap
  SITE 2: note = D-Phenylalanine
  SITE 3: note = D-Lysine
  SITE 5: note = D-Serine
  SITE 6: note = D-Aspartic acid
XFKXSD

SEQ ID NO: 39-41 - skipped (3 entries)

SEQ ID NO: 42 - protein, 6 aa, synthetic construct
  MOD_RES 1: note = X is Hle
  MOD_RES 3: note = X is Hle
  SITE 2: note = D-Phenylalanine
  SITE 4: note = D-Asparagine
  SITE 5: note = D-Threonine
  SITE 6: note = D- Glutamine
XFXNTQ

SEQ ID NO: 43-45 - skipped (3 entries)

SEQ ID NO: 46 - protein, 6 aa, synthetic construct
  SITE 1: note = D-Phenylalanine
  SITE 2: note = D-Serine
  MOD_RES 3: note = X is Dpa
  MOD_RES 5: note = X is Hle
  SITE 6: note = D-Arginine
FSXGXR

SEQ ID NO: 47 - skipped

SEQ ID NO: 48 - protein, 6 aa, synthetic construct
  SITE 1: note = D-Serine
  SITE 2: note = D- Glutamic acid
  MOD_RES 3: note = X is Dpa
  SITE 4: note = D-Threonine
  MOD_RES 5: note = X is Nap
SEXTXG

SEQ ID NO: 49 - skipped

SEQ ID NO: 50 - protein, 6 aa, synthetic construct
  SITE 2: note = D-Phenylalanine
  SITE 3: note = D-Proline
  MOD_RES 4: note = X is D-Nle
  SITE 5: note = D- Glutamic acid
  MOD_RES 6: note = X is Dpa
GFPXEX

SEQ ID NO: 51-52 - skipped (2 entries)

SEQ ID NO: 53 - protein, 6 aa, synthetic construct
  SITE 1: note = D-Proline
  SITE 2: note = D-Lysine
  MOD_RES 3: note = X is D-Nle
  MOD_RES 6: note = X is D-Nle
  SITE 4: note = D-Aspartic acid
  SITE 5: note = D-Phenylalanine
PKXDFX

SEQ ID NO: 54-56 - skipped (3 entries)

SEQ ID NO: 57 - protein, 6 aa, synthetic construct
  SITE 1: note = D-Threonine
  SITE 2: note = D-Asparagine
  SITE 3: note = D-Phenylalanine
  MOD_RES 4: note = X is D-Nle
  MOD_RES 6: note = X is Dpa
TNFXGX

SEQ ID NO: 58 - protein, 6 aa, synthetic construct
  SITE 1: note = D-Phenylalanine
  MOD_RES 2..3: note = X is Hle
  SITE 4: note = D-Asparagine
  SITE 5: note = D-Threonine
  SITE 6: note = D- Glutamine
FXXNTQ

SEQ ID NO: 59 - protein, 6 aa, synthetic construct
  SITE 1: note = D-Lysine
  SITE 2: note = D-Phenylalanine
  SITE 3: note = D-Serine
  MOD_RES 4: note = X is D-Nle
  SITE 5: note = D- Glutamic acid
  MOD_RES 6: note = X is Dab
KFSXEX

SEQ ID NO: 60 - protein, 6 aa, synthetic construct
  MOD_RES 1: note = X is beta-alanine
  SITE 2: note = D-Lysine
  SITE 3: note = D- Glutamine
  MOD_RES 4: note = X is Dpa
  SITE 5: note = D-Phenylalanine
  SITE 6: note = D-Asparagine
XKQXFN

SEQ ID NO: 61 - skipped

SEQ ID NO: 62 - protein, 6 aa, synthetic construct
  MOD_RES 1: note = X is Dpa
  SITE 3: note = D- Glutamic acid
  SITE 4: note = D-Asparagine
  SITE 5: note = D-Aspartic acid
  MOD_RES 6: note = X is D-Nle
XGENDX

SEQ ID NO: 63-65 - skipped (3 entries)

SEQ ID NO: 66 - protein, 6 aa, synthetic construct
  SITE 1: note = D-Phenylalanine
  SITE 3: note = D-Proline
  MOD_RES 4: note = X is D-Nle
  SITE 5: note = D- Glutamic acid
  MOD_RES 6: note = X is Dpa
FGPXEX

SEQ ID NO: 67 - skipped

SEQ ID NO: 68 - protein, 6 aa, synthetic construct
  SITE 1: note = D-Phenylalanine
  SITE 5: note = D-Phenylalanine
  SITE 2: note = D-Lysine
  SITE 6: note = D-Lysine
  MOD_RES 3: note = X is Nap
  SITE 4: note = D-Asparagine
FKXNFK

SEQ ID NO: 69 - protein, 6 aa, synthetic construct
  SITE 1: note = D-Asparagine
  SITE 2: note = D-Phenylalanine
  SITE 3: note = D-Aspartic acid
  SITE 4: note = D-Serine
  MOD_RES 5: note = X is Dpa
  MOD_RES 6: note = X is Amf
NFDSXX

SEQ ID NO: 70 - protein, 6 aa, synthetic construct
  SITE 1: note = D-Proline
  SITE 2: note = D-Serine
  SITE 3: note = D-Phenylalanine
  MOD_RES 4: note = X is Dpa
  SITE 5: note = D- Glutamic acid
  MOD_RES 6: note = X is beta-alanine
PSFXEB

SEQ ID NO: 71-77 - skipped (7 entries)

SEQ ID NO: 78 - protein, 6 aa, synthetic construct
  SITE 1: note = D-Threonine
  SITE 2: note = D-Arginine
  MOD_RES 3: note = X is Dpa
  SITE 4: note = D- Glutamine
  MOD_RES 5: note = X is beta-alanine
  SITE 6: note = D-Phenylalanine
TRXQXF

SEQ ID NO: 79-82 - skipped (4 entries)

SEQ ID NO: 83 - protein, 6 aa, synthetic construct
  MOD_RES 1: note = X is Hle
  SITE 2: note = D-Lysine
  MOD_RES 3: note = X is Dpa
  SITE 5: note = D-Threonine
  SITE 6: note = D-Proline
XKXGTP

SEQ ID NO: 84 - skipped

SEQ ID NO: 85 - protein, 6 aa, synthetic construct
  SITE 1: note = D-Asparagine
  MOD_RES 3: note = X is Dpa
  SITE 4: note = D-Threonine
  MOD_RES 5: note = X is Nap
NGXTXG

SEQ ID NO: 86 - protein, 6 aa, synthetic construct
  SITE 2: note = D-Asparagine
  MOD_RES 3: note = X is Dpa
  SITE 4: note = D-Threonine
  MOD_RES 5: note = X is Nap
GNXTXG

SEQ ID NO: 87 - protein, 6 aa, synthetic construct
  SITE 1: note = D-Serine
  SITE 2: note = D-Phenylalanine
  SITE 3: note = D- Glutamic acid
  SITE 4: note = D-Aspartic acid
SFEDGG

SEQ ID NO: 88 - protein, 6 aa, synthetic construct
  SITE 1..2: note = D-Phenylalanine
  SITE 4: note = D-Phenylalanine
  SITE 3: note = D-Threonine
  SITE 5: note = D-Threonine
  MOD_RES 6: note = X is Abu
FFTFTX

SEQ ID NO: 89-91 - skipped (3 entries)

SEQ ID NO: 92 - protein, 6 aa, synthetic construct
  SITE 1: note = D-Lysine
  MOD_RES 2: note = X is Hle
  MOD_RES 3: note = X is Dpa
  SITE 5: note = D-Threonine
  SITE 6: note = D-Proline
KXXGTP

SEQ ID NO: 93-97 - skipped (5 entries)

SEQ ID NO: 98 - protein, 6 aa, synthetic construct
  SITE 2: note = D-Lysine
  SITE 3: note = D- Glutamic acid
  MOD_RES 4: note = X is Hle
  SITE 5: note = D-Phenylalanine
  MOD_RES 6: note = X is Amf
GKEXFX

SEQ ID NO: 99-102 - skipped (4 entries)

SEQ ID NO: 103 - protein, 6 aa, synthetic construct
  SITE 1: note = D-Lysine
  SITE 3: note = D- Glutamic acid
  MOD_RES 4: note = X is Hle
  SITE 5: note = D-Phenylalanine
  MOD_RES 6: note = X is Amf
KGEXFX

SEQ ID NO: 104 - skipped

SEQ ID NO: 105 - protein, 6 aa, synthetic construct
  SITE 1: note = D-Serine
  SITE 2: note = D-Asparagine
  SITE 3: note = D-Phenylalanine
  MOD_RES 4: note = X is D-Nle
  MOD_RES 6: note = X is Dpa
SNFXGX

SEQ ID NO: 106-109 - skipped (4 entries)

SEQ ID NO: 110 - protein, 6 aa, synthetic construct
  SITE 1: note = D-Phenylalanine
  SITE 6: note = D-Phenylalanine
  MOD_RES 2: note = X is Amf
  SITE 3..4: note = D-Aspartic acid
  MOD_RES 5: note = X is Dpa
FXDDXF

SEQ ID NO: 111 - protein, 6 aa, synthetic construct
  MOD_RES 1: note = X is D-Nle
  SITE 2: note = D-Asparagine
  SITE 3: note = D-Threonine
  SITE 4: note = D- Glutamine
  MOD_RES 5: note = X is Nap
  SITE 6: note = D- Glutamic acid
XNTQXE

SEQ ID NO: 112 - protein, 6 aa, synthetic construct
  SITE 1: note = D-Phenylalanine
  SITE 2: note = D-Serine
  SITE 3: note = D- Glutamic acid
  SITE 4: note = D-Aspartic acid
FSEDGG

SEQ ID NO: 113-115 - skipped (3 entries)

SEQ ID NO: 116 - protein, 6 aa, synthetic construct
  SITE 1: note = D-Threonine
  SITE 3: note = D-Threonine
  MOD_RES 2: note = X is Pip
  SITE 4: note = D- Glutamine
  MOD_RES 5: note = X is Nap
  SITE 6: note = D- Glutamic acid
TXTQXE

SEQ ID NO: 117-120 - skipped (4 entries)

SEQ ID NO: 121 - protein, 6 aa, synthetic construct
  SITE 1: note = D- Glutamic acid
  SITE 2: note = D-Phenylalanine
  MOD_RES 5: note = X is Nap
  MOD_RES 6: note = X is Amf
EFGGXX

SEQ ID NO: 122 - protein, 6 aa, synthetic construct
  SITE 1..2: note = D-Phenylalanine
  SITE 3: note = D-Asparagine
  SITE 4: note = D-Arginine
  MOD_RES 5: note = X is Abu
  MOD_RES 6: note = X is Dpa
FFNRXX

SEQ ID NO: 123 - protein, 6 aa, synthetic construct
  SITE 1: note = D-Asparagine
  SITE 2: note = D- Glutamic acid
  SITE 3: note = D-Histidine
  SITE 4: note = D-Threonine
  MOD_RES 5: note = X is Nap
NEHTXG

SEQ ID NO: 124 - protein, 6 aa, synthetic construct
  SITE 1: note = D-Arginine
  SITE 2: note = D-Threonine
  MOD_RES 3: note = X is Dpa
  SITE 4: note = D- Glutamine
  MOD_RES 5: note = X is beta-alanine
  SITE 6: note = D-Phenylalanine
RTXQXF

SEQ ID NO: 125 - skipped

SEQ ID NO: 126 - protein, 6 aa, synthetic construct
  MOD_RES 1: note = X is Abu
  SITE 2: note = D-Phenylalanine
  SITE 3..4: note = D- Glutamic acid
  MOD_RES 5: note = X is beta-alanine
  SITE 6: note = D-Aspartic acid
XFEEXD

SEQ ID NO: 127-128 - skipped (2 entries)

SEQ ID NO: 129 - protein, 6 aa, synthetic construct
  MOD_RES 1: note = X is Abu
  SITE 2..3: note = D-Proline
  MOD_RES 4: note = X is beta-alanine
  SITE 5: note = D-Serine
  SITE 6: note = D- Glutamic acid
XPPXSE

SEQ ID NO: 130 - protein, 6 aa, synthetic construct
  SITE 1: note = D-Threonine
  SITE 2: note = D-Arginine
  MOD_RES 3: note = X is Dpa
  MOD_RES 4: note = X is Abu
  SITE 5: note = D-Asparagine
  SITE 6: note = D-Phenylalanine
TRXXNF

SEQ ID NO: 131 - protein, 6 aa, synthetic construct
  SITE 1: note = D-Proline
  SITE 3: note = D-Proline
  MOD_RES 2: note = X is Abu
  SITE 6: note = D-Histidine
PXPGGH

SEQ ID NO: 132-133 - skipped (2 entries)

SEQ ID NO: 134 - protein, 6 aa, synthetic construct
  SITE 1: note = D-Asparagine
  SITE 2: note = D-Histidine
  SITE 3: note = D- Glutamic acid
  SITE 4: note = D-Threonine
  MOD_RES 5: note = X is Nap
NHETXG

SEQ ID NO: 135 - skipped

SEQ ID NO: 136 - protein, 6 aa, synthetic construct
  MOD_RES 1: note = X is Hle
  SITE 2: note = D-Lysine
  MOD_RES 3: note = X is D-Nle
  SITE 4: note = D- Glutamic acid
  SITE 5: note = D-Serine
  SITE 6: note = D- Glutamic acid
XKXESE

SEQ ID NO: 137-139 - skipped (3 entries)

SEQ ID NO: 140 - protein, 6 aa, synthetic construct
  SITE 1: note = D- Glutamic acid
  SITE 2: note = D-Serine
  MOD_RES 3: note = ox-Histidine
  SITE 4: note = D-Serine
  MOD_RES 5: note = X is Dpa
ESHSXG

SEQ ID NO: 141-143 - skipped (3 entries)

SEQ ID NO: 144 - protein, 6 aa, synthetic construct
  MOD_RES 1: note = X is Dpa
  SITE 2: note = D- Glutamic acid
  SITE 3: note = D-Proline
  MOD_RES 4: note = X is beta-alanine
  SITE 5: note = D-Histidine
XEPXHG

SEQ ID NO: 145 - protein, 6 aa, synthetic construct
  MOD_RES 3: note = X is Hle
  SITE 4: note = D-Arginine
  MOD_RES 5: note = X is D-Nle
  SITE 6: note = D-Phenylalanine
GGXRXF

SEQ ID NO: 146 - protein, 6 aa, synthetic construct
  MOD_RES 1: note = X is Dpa
  SITE 2: note = D-Phenylalanine
  SITE 4: note = D-Phenylalanine
  SITE 3: note = D-Lysine
  SITE 5: note = D-Aspartic acid
  MOD_RES 6: note = X is Dab
XFKFDX

SEQ ID NO: 147 - protein, 6 aa, synthetic construct
  SITE 1: note = D- Glutamic acid
  SITE 2: note = D-Asparagine
  SITE 3: note = D-Histidine
  SITE 4: note = D-Threonine
  MOD_RES 5: note = X is Nap
ENHTXG

SEQ ID NO: 148 - protein, 6 aa, synthetic construct
  MOD_RES 1: note = X is D-Nle
  MOD_RES 6: note = X is Nap
  SITE 2: note = D- Glutamine
  SITE 5: note = D- Glutamine
  SITE 3: note = D-Arginine
  SITE 4: note = D- Glutamic acid
XQREQX

SEQ ID NO: 149 - protein, 6 aa, synthetic construct
  MOD_RES 1: note = X is D-Nle
  SITE 2: note = D- Glutamic acid
  SITE 3: note = D-Phenylalanine
  SITE 4: note = D-Threonine
  SITE 5: note = D-Aspartic acid
  SITE 6: note = D-Threonine
XEFTDT

SEQ ID NO: 150-152 - skipped (3 entries)

SEQ ID NO: 153 - protein, 6 aa, synthetic construct
  SITE 1: note = D-Serine
  SITE 2: note = D-Arginine
  SITE 3: note = D-Asparagine
  MOD_RES 4: note = X is Dpa
  MOD_RES 5: note = X is Abu
SRNXXG

SEQ ID NO: 154 - protein, 6 aa, synthetic construct
  MOD_RES 1: note = X is Dpa
  SITE 2: note = D-Threonine
  SITE 5: note = D-Threonine
  SITE 3: note = D-Asparagine
  SITE 4: note = D- Glutamic acid
  SITE 6: note = D-Serine
XTNETS

SEQ ID NO: 155-160 - skipped (6 entries)

SEQ ID NO: 161 - protein, 6 aa, synthetic construct
  SITE 1: note = D- Glutamic acid
  MOD_RES 2: note = ox-Histidine
  SITE 3..4: note = D-Serine
  MOD_RES 5: note = X is Dpa
EHSSXG

SEQ ID NO: 162 - protein, 6 aa, synthetic construct
  SITE 1: note = D-Arginine
  SITE 2: note = D-Asparagine
  SITE 3: note = D- Glutamic acid
  SITE 4: note = D-Serine
  SITE 5: note = D-Histidine
  SITE 6: note = D-Phenylalanine
RNESHF

SEQ ID NO: 163 - protein, 6 aa, synthetic construct
  SITE 1..2: note = D- Glutamine
  SITE 3: note = D-Serine
  SITE 4: note = D-Proline
  MOD_RES 5: note = X is D-Nle
QQSPXG

SEQ ID NO: 164 - protein, 6 aa, synthetic construct
  SITE 1: note = D-Asparagine
  SITE 3: note = D-Histidine
  SITE 4: note = D-Asparagine
  MOD_RES 5: note = X is beta-alanine
  MOD_RES 6: note = X is Dpa
NGHNXX

SEQ ID NO: 165-166 - skipped (2 entries)

SEQ ID NO: 167 - protein, 6 aa, synthetic construct
  MOD_RES 1: note = X is Pip
  SITE 2: note = D-Histidine
  SITE 3: note = D-Aspartic acid
  SITE 6: note = D-Aspartic acid
  SITE 4: note = D-Phenylalanine
  SITE 5: note = D-Arginine
XHDFRD

SEQ ID NO: 168-169 - skipped (2 entries)

SEQ ID NO: 170 - protein, 6 aa, synthetic construct
  SITE 1: note = D-Proline
  SITE 3: note = D-Asparagine
  MOD_RES 4: note = X is Hle
  SITE 6: note = D-Asparagine
PGNXGN

SEQ ID NO: 171 - skipped

SEQ ID NO: 172 - protein, 6 aa, synthetic construct
  MOD_RES 3: note = ox-Histidine
  MOD_RES 4: note = X is Nap
  SITE 5: note = D-Aspartic acid
  MOD_RES 6: note = X is Dpa
GGHXDX

SEQ ID NO: 173 - skipped

SEQ ID NO: 174 - protein, 6 aa, synthetic construct
  SITE 5: note = D-Asparagine
  SITE 1: note = D-Asparagine
  SITE 2: note = D-Histidine
  MOD_RES 4: note = X is beta-alanine
  MOD_RES 6: note = X is Dpa
NHGXNX

SEQ ID NO: 175-177 - skipped (3 entries)

SEQ ID NO: 178 - protein, 6 aa, synthetic construct
  SITE 1: note = D-Lysine
  SITE 2: note = D-Proline
  MOD_RES 3: note = X is D-Nle
  MOD_RES 6: note = X is D-Nle
  SITE 4: note = D-Aspartic acid
  SITE 5: note = D-Phenylalanine
KPXDFX

SEQ ID NO: 179-201 - skipped (23 entries)

SEQ ID NO: 202 - protein, 6 aa, synthetic construct
  SITE 3: note = D-Histidine
  SITE 4: note = D-Phenylalanine
  MOD_RES 5: note = X is Hle
  SITE 6: note = D-Serine
GGHFXS

SEQ ID NO: 203 - protein, 6 aa, synthetic construct
  SITE 1: note = D-Asparagine
  SITE 2: note = D-Phenylalanine
  SITE 4: note = D- Glutamic acid
  SITE 5: note = D-Proline
  MOD_RES 6: note = X is Dpa
NFGEPX

SEQ ID NO: 204 - protein, 6 aa, synthetic construct
  SITE 1: note = D-Aspartic acid
  MOD_RES 2: note = X is Nap
  SITE 4: note = D-Serine
  MOD_RES 5: note = X is Dpa
DXGSXG

SEQ ID NO: 205 - protein, 6 aa, synthetic construct
  SITE 1: note = D-Threonine
  MOD_RES 2: note = ox-Histidine
  SITE 3: note = D-Aspartic acid
  SITE 4: note = D- Glutamine
  SITE 5: note = D-Proline
  MOD_RES 6: note = X is Dpa
THDQPX

SEQ ID NO: 206 - protein, 6 aa, synthetic construct
  SITE 1: note = D-Asparagine
  MOD_RES 2: note = X is Nap
  SITE 4: note = D- Glutamic acid
  SITE 5: note = D-Proline
  MOD_RES 6: note = X is Dpa
NXGEPX

SEQ ID NO: 207-211 - skipped (5 entries)

SEQ ID NO: 212 - protein, 6 aa, synthetic construct
  MOD_RES 1: note = X is Nap
  SITE 3: note = D-Aspartic acid
  SITE 4: note = D- Glutamine
  SITE 5: note = D-Proline
  MOD_RES 6: note = X is Dpa
XGDQPX

SEQ ID NO: 213 - skipped

SEQ ID NO: 214 - protein, 6 aa, synthetic construct
  SITE 2: note = D-Phenylalanine
  SITE 6: note = D-Phenylalanine
  SITE 3: note = D-Serine
  MOD_RES 4..5: note = X is D-Nle
GFSXXF

SEQ ID NO: 215 - protein, 6 aa, synthetic construct
  SITE 1: note = D-Lysine
  MOD_RES 2: note = X is beta-alanine
  MOD_RES 3: note = ox-Histidine
  SITE 4: note = D- Glutamine
  MOD_RES 5: note = X is Dpa
  SITE 6: note = D-Asparagine
KXHQXN

SEQ ID NO: 216 - protein, 6 aa, synthetic construct
  SITE 3: note = D-Asparagine
  SITE 4..5: note = D-Phenylalanine
  SITE 6: note = D- Glutamine
GGNFFQ

SEQ ID NO: 217 - protein, 6 aa, synthetic construct
  SITE 1: note = D-Aspartic acid
  SITE 6: note = D-Aspartic acid
  MOD_RES 2: note = X is Pip
  SITE 3: note = D-Histidine
  SITE 4: note = D-Phenylalanine
  SITE 5: note = D-Arginine
DXHFRD

SEQ ID NO: 218 - protein, 6 aa, synthetic construct
  MOD_RES 1: note = ox-Histidine
  SITE 2: note = D- Glutamine
  SITE 3: note = D-Proline
  SITE 4: note = D-Phenylalanine
  SITE 5: note = D-Arginine
  SITE 6: note = D-Aspartic acid
HQPFRD

SEQ ID NO: 219 - skipped

SEQ ID NO: 220 - protein, 6 aa, synthetic construct
  MOD_RES 1: note = X is Pip
  SITE 2: note = D-Aspartic acid
  SITE 6: note = D-Aspartic acid
  SITE 3: note = D-Histidine
  SITE 4: note = D-Phenylalanine
  SITE 5: note = D-Arginine
XDHFRD

SEQ ID NO: 221 - protein, 6 aa, synthetic construct
  MOD_RES 2: note = ox-Histidine
  SITE 5: note = D-Aspartic acid
  MOD_RES 6: note = X is Dpa
  MOD_RES 4: note = X is Nap
GHGXDX

SEQ ID NO: 222 - skipped

SEQ ID NO: 223 - protein, 6 aa, synthetic construct
  MOD_RES 1: note = X is Nap
  SITE 2: note = D- Glutamic acid
  SITE 3: note = D- Glutamine
  SITE 4: note = D-Proline
  MOD_RES 5: note = X is Pip
  SITE 6: note = D-Phenylalanine
XEQPXF

SEQ ID NO: 224-226 - skipped (3 entries)

SEQ ID NO: 227 - protein, 6 aa, synthetic construct
  SITE 1: note = D-Histidine
  SITE 4: note = D-Phenylalanine
  MOD_RES 5: note = X is Hle
  SITE 6: note = D-Serine
HGGFXS

SEQ ID NO: 228 - protein, 6 aa, synthetic construct
  SITE 1: note = D-Proline
  SITE 5: note = D-Proline
  MOD_RES 2: note = X is D-Nle
  SITE 3: note = D-Asparagine
  SITE 4: note = D-Aspartic acid
  MOD_RES 6: note = X is Hle
PXNDPX

SEQ ID NO: 229 - protein, 6 aa, synthetic construct
  SITE 2: note = D-Histidine
  MOD_RES 4: note = X is Abu
  SITE 5: note = D-Lysine
  MOD_RES 6: note = X is Dpa
GHGXKX

SEQ ID NO: 230 - skipped

SEQ ID NO: 231 - protein, 6 aa, synthetic construct
  SITE 1: note = D-Aspartic acid
  SITE 3: note = D-Aspartic acid
  SITE 5: note = D-Aspartic acid
  SITE 2: note = D-Proline
  SITE 4: note = D-Histidine
  MOD_RES 6: note = X is Dpa
DPDHDX

SEQ ID NO: 232 - skipped

SEQ ID NO: 233 - protein, 6 aa, synthetic construct
  MOD_RES 1: note = X is Dpa
  MOD_RES 6: note = X is Dpa
  SITE 2: note = D- Glutamic acid
  SITE 3: note = D-Asparagine
  SITE 4: note = D- Glutamine
  SITE 5: note = D-Proline
XENQPX

SEQ ID NO: 234-236 - skipped (3 entries)

SEQ ID NO: 237 - protein, 6 aa, synthetic construct
  SITE 1: note = D-Proline
  SITE 4: note = D-Proline
  MOD_RES 2: note = X is D-Nle
  MOD_RES 6: note = X is Pip
  SITE 3: note = D- Glutamic acid
  SITE 5: note = D-Threonine
PXEPTX

SEQ ID NO: 238 - protein, 6 aa, synthetic construct
  SITE 1: note = D-Asparagine
  SITE 2: note = D-Histidine
  MOD_RES 3: note = X is beta-alanine
  MOD_RES 4: note = X is Dpa
  SITE 5: note = D-Phenylalanine
  SITE 6: note = D- Glutamine
NHXXFQ

SEQ ID NO: 239-240 - skipped (2 entries)

SEQ ID NO: 241 - protein, 6 aa, synthetic construct
  SITE 1: note = D-Serine
  SITE 4: note = D-Serine
  MOD_RES 2: note = ox-Histidine
  SITE 3: note = D- Glutamic acid
  MOD_RES 5: note = X is Dpa
SHESXG

SEQ ID NO: 242 - protein, 6 aa, synthetic construct
  SITE 1..2: note = D- Glutamine
  MOD_RES 3: note = X is D-Nle
  SITE 4: note = D-Serine
  SITE 5: note = D-Proline
QQXSPG

SEQ ID NO: 243 - skipped

SEQ ID NO: 244 - protein, 6 aa, synthetic construct
  SITE 1: note = D-Asparagine
  SITE 2: note = D-Proline
  MOD_RES 3: note = X is beta-alanine
  MOD_RES 6: note = X is Dpa
NPXGGX

SEQ ID NO: 245-246 - skipped (2 entries)

SEQ ID NO: 247 - protein, 6 aa, synthetic construct
  MOD_RES 1: note = ox-Histidine
  MOD_RES 4: note = X is Nap
  SITE 5: note = D-Aspartic acid
  MOD_RES 6: note = X is Dpa
HGGXDX

SEQ ID NO: 248 - skipped

SEQ ID NO: 249 - protein, 6 aa, synthetic construct
  MOD_RES 1: note = X is Pip
  MOD_RES 4: note = X is Hle
  SITE 3: note = D-Serine
  SITE 5: note = D-Histidine
  SITE 6: note = D-Asparagine
XGSXHN

SEQ ID NO: 250 - protein, 6 aa, synthetic construct
  MOD_RES 1: note = X is Dpa
  SITE 2: note = D-Lysine
  MOD_RES 3: note = X is beta-alanine
  SITE 6: note = D-Proline
XKXGGP

SEQ ID NO: 251-252 - skipped (2 entries)

SEQ ID NO: 253 - protein, 6 aa, synthetic construct
  SITE 1: note = D-Proline
  SITE 2: note = D- Glutamic acid
  MOD_RES 3: note = X is Amf
  SITE 5: note = D-Serine
  MOD_RES 6: note = X is beta-alanine
PEXGSX

SEQ ID NO: 254 - skipped

SEQ ID NO: 255 - protein, 6 aa, synthetic construct
  MOD_RES 1: note = X is Abu
  MOD_RES 4: note = X is Dpa
  SITE 2: note = D- Glutamic acid
  SITE 3: note = D-Histidine
  SITE 5: note = D-Phenylalanine
  SITE 6: note = D- Glutamine
XEHXFQ

SEQ ID NO: 256 - protein, 6 aa, synthetic construct
  MOD_RES 1: note = X is Abu
  MOD_RES 2: note = X is beta-alanine
  SITE 3: note = D-Serine
  SITE 4..5: note = D-Phenylalanine
  SITE 6: note = D- Glutamine
XXSFFQ

SEQ ID NO: 257 - skipped

SEQ ID NO: 258 - protein, 6 aa, synthetic construct
  SITE 1: note = D-Phenylalanine
  SITE 2: note = D-Aspartic acid
  SITE 4: note = D- Glutamine
  SITE 5: note = D-Proline
  MOD_RES 6: note = X is Dpa
FDGQPX

SEQ ID NO: 259 - protein, 6 aa, synthetic construct
  MOD_RES 2: note = X is Pip
  MOD_RES 4: note = X is Hle
  SITE 3: note = D-Serine
  SITE 5: note = D-Histidine
  SITE 6: note = D-Asparagine
GXSXHN

SEQ ID NO: 260-264 - skipped (5 entries)

SEQ ID NO: 265 - protein, 6 aa, synthetic construct
  SITE 1: note = D-Phenylalanine
  SITE 2: note = D-Proline
  MOD_RES 3: note = X is beta-alanine
  MOD_RES 4: note = X is Hle
  SITE 5: note = D-Arginine
  SITE 6: note = D-Aspartic acid
FPXXRD

SEQ ID NO: 266 - skipped

SEQ ID NO: 267 - protein, 6 aa, synthetic construct
  MOD_RES 1: note = X is Pip
  MOD_RES 2: note = X is D-Nle
  SITE 3: note = D- Glutamic acid
  SITE 4: note = D- Glutamine
  SITE 5: note = D-Histidine
  SITE 6: note = D-Asparagine
XXEQHN

SEQ ID NO: 268-270 - skipped (3 entries)

SEQ ID NO: 271 - protein, 6 aa, synthetic construct
  SITE 3: note = D-Histidine
  MOD_RES 4: note = X is Abu
  SITE 5: note = D-Lysine
  MOD_RES 6: note = X is Dpa
GGHXKX

SEQ ID NO: 272 - protein, 6 aa, synthetic construct
  MOD_RES 1: note = X is Nap
  MOD_RES 3: note = X is Pip
  SITE 2: note = D- Glutamic acid
  SITE 4: note = D-Proline
  SITE 5: note = D- Glutamine
  SITE 6: note = D-Phenylalanine
XEXPQF

SEQ ID NO: 273-275 - skipped (3 entries)

SEQ ID NO: 276 - protein, 6 aa, synthetic construct
  MOD_RES 1: note = X is beta-alanine
  SITE 2: note = ox-Histidine
  MOD_RES 3: note = X is Dab
  SITE 4: note = D- Glutamic acid
  SITE 5: note = D-Threonine
  SITE 6: note = D-Serine
XHXETS

SEQ ID NO: 277 - protein, 6 aa, synthetic construct
  SITE 1: note = D-Asparagine
  MOD_RES 2: note = X is Pip
  MOD_RES 5: note = X is Hle
  SITE 3: note = D- Glutamine
  SITE 4: note = D-Serine
  SITE 6: note = D-Serine
NXQSXS

SEQ ID NO: 278 - skipped

SEQ ID NO: 279 - protein, 6 aa, synthetic construct
  SITE 1: note = D-Threonine
  SITE 3: note = D-Phenylalanine
  SITE 4: note = D-Proline
  MOD_RES 6: note = X is Dpa
TGFPGX

SEQ ID NO: 280 - protein, 6 aa, synthetic construct
  SITE 1: note = D-Asparagine
  SITE 3: note = D- Glutamic acid
  MOD_RES 4: note = X is Nap
  SITE 5: note = D-Proline
  MOD_RES 6: note = X is Dpa
NGEXPX

SEQ ID NO: 281-283 - skipped (3 entries)

SEQ ID NO: 284 - protein, 6 aa, synthetic construct
  SITE 1: note = D-Asparagine
  MOD_RES 2: note = X is Nap
  SITE 3: note = D- Glutamic acid
  SITE 5: note = D-Proline
  MOD_RES 6: note = X is Dpa
NXEGPX

SEQ ID NO: 285 - protein, 6 aa, synthetic construct
  SITE 1: note = D-Asparagine
  MOD_RES 3: note = X is Nap
  SITE 4: note = D- Glutamic acid
  SITE 5: note = D-Proline
  MOD_RES 6: note = X is Dpa
NGXEPX

SEQ ID NO: 286-287 - skipped (2 entries)

SEQ ID NO: 288 - protein, 6 aa, synthetic construct
  SITE 1: note = D-Histidine
  MOD_RES 3: note = X is Amf
  SITE 4: note = D-Lysine
  SITE 5: note = D-Serine
  SITE 6: note = D-Aspartic acid
HGXLSD

SEQ ID NO: 289-290 - skipped (2 entries)

SEQ ID NO: 291 - protein, 6 aa, synthetic construct
  MOD_RES 1: note = ox-Histidine
  MOD_RES 5: note = X is Nap
  MOD_RES 6: note = X is Hle
HGGGXX

SEQ ID NO: 292 - protein, 6 aa, synthetic construct
  SITE 1: note = D- Glutamine
  MOD_RES 2: note = X is Gba
  SITE 3: note = D- Glutamic acid
  SITE 4: note = D-Serine
  SITE 5: note = D-Histidine
  SITE 6: note = D-Phenylalanine
QXESHF

SEQ ID NO: 293 - skipped

SEQ ID NO: 294 - protein, 6 aa, synthetic construct
  SITE 1: note = D-Aspartic acid
  SITE 2: note = D-Histidine
  MOD_RES 3: note = X is Pip
  SITE 4: note = D-Phenylalanine
  SITE 5: note = D-Arginine
  SITE 6: note = D-Aspartic acid
DHXFRD

SEQ ID NO: 295-298 - skipped (4 entries)

SEQ ID NO: 299 - protein, 6 aa, synthetic construct
  SITE 1: note = D-Asparagine
  SITE 2..3: note = D-Proline
  SITE 4: note = D-Phenylalanine
  MOD_RES 5: note = X is Hle
  SITE 6: note = D-Serine
NPPFXS

SEQ ID NO: 300 - skipped

SEQ ID NO: 301 - protein, 6 aa, synthetic construct
  SITE 1: note = D- Glutamic acid
  SITE 2: note = D-Histidine
  MOD_RES 3: note = X is Abu
  MOD_RES 4: note = X is Dpa
  SITE 5: note = D-Phenylalanine
  SITE 6: note = D- Glutamine
QHXXFQ

SEQ ID NO: 302 - skipped

SEQ ID NO: 303 - protein, 6 aa, synthetic construct
  MOD_RES 1: note = X is Hle
  SITE 2: note = D-Proline
  SITE 3: note = D- Glutamic acid
  SITE 4: note = D-Proline
  SITE 5: note = D-Threonine
  MOD_RES 6: note = X is Pip
XPEPTX

SEQ ID NO: 304 - protein, 6 aa, synthetic construct
  MOD_RES 1: note = X is Nap
  SITE 2: note = D-Aspartic acid
  SITE 4: note = D-Serine
  MOD_RES 5: note = X is Dpa
XDGSXG

SEQ ID NO: 305-313 - skipped (9 entries)

SEQ ID NO: 314 - protein, 6 aa, synthetic construct
  SITE 1: note = D-Serine
  SITE 2: note = D- Glutamic acid
  MOD_RES 3: note = X is D-Nle
  SITE 6: note = D- Glutamine
SEXGGQ

SEQ ID NO: 315 - protein, 6 aa, synthetic construct
  SITE 1: note = D-Lysine
  SITE 2: note = D-Arginine
  SITE 3: note = D-Aspartic acid
  SITE 5: note = D-Proline
KRDGPG

SEQ ID NO: 316 - protein, 6 aa, synthetic construct
  SITE 1: note = D- Glutamine
  SITE 2: note = D-Proline
  MOD_RES 3: note = X is Pip
  MOD_RES 4: note = X is beta-alanine
  SITE 5: note = D-Asparagine
  SITE 6: note = D-Proline
QPXXNP

SEQ ID NO: 317 - skipped

SEQ ID NO: 318 - protein, 6 aa, synthetic construct
  MOD_RES 1: note = X is Abu
  SITE 2: note = D-Proline
  SITE 4: note = D-Proline
  SITE 6: note = D-Histidine
XPGPGH

SEQ ID NO: 319-320 - skipped (2 entries)

SEQ ID NO: 321 - protein, 6 aa, synthetic construct
  SITE 1: note = D-Proline
  SITE 4: note = D-Proline
  MOD_RES 2: note = X is Abu
  SITE 6: note = D-Histidine
PXGPGH

SEQ ID NO: 322 - protein, 6 aa, synthetic construct
  SITE 1: note = D-Arginine
  SITE 2: note = D- Glutamine
  SITE 3: note = D-Aspartic acid
  SITE 4: note = D-Serine
  SITE 5: note = D-Phenylalanine
  SITE 6: note = D- Glutamine
RQDSFQ

SEQ ID NO: 323-330 - skipped (8 entries)

SEQ ID NO: 331 - protein, 6 aa, synthetic construct
  SITE 1: note = D-Phenylalanine
  SITE 5: note = D-Phenylalanine
  MOD_RES 2: note = X is Abu
  MOD_RES 6: note = X is Nap
  SITE 4: note = D-Serine
FXGSFX

SEQ ID NO: 332 - skipped

SEQ ID NO: 333 - protein, 6 aa, synthetic construct
  SITE 1: note = D-Aspartic acid
  SITE 3: note = D-Phenylalanine
  SITE 4: note = D- Glutamine
  SITE 5: note = D-Proline
  MOD_RES 6: note = X is Dpa
DGFQPX

SEQ ID NO: 334 - protein, 6 aa, synthetic construct
  MOD_RES 1: note = X is Abu
  MOD_RES 2: note = X is Hle
  SITE 4: note = D-Serine
  SITE 5: note = D-Asparagine
  SITE 6: note = D-Aspartic acid
XXGSND

SEQ ID NO: 335 - protein, 6 aa, synthetic construct
  SITE 1: note = D-Serine
  SITE 2: note = D-Phenylalanine
  MOD_RES 4..5: note = X is D-Nle
  SITE 6: note = D-Phenylalanine
SFGXXF

SEQ ID NO: 336 - protein, 6 aa, synthetic construct
  MOD_RES 1: note = X is Hle
  MOD_RES 3: note = X is Dpa
  SITE 4: note = D-Phenylalanine
  SITE 5: note = D-Threonine
XGXFTG

SEQ ID NO: 337 - protein, 6 aa, synthetic construct
  MOD_RES 1: note = X is Abu
  SITE 2: note = D-Proline
  MOD_RES 3: note = X is beta-alanine
  SITE 4: note = D-Proline
  SITE 5: note = D-Serine
  SITE 6: note = D- Glutamic acid
XPXPSE

SEQ ID NO: 338 - protein, 6 aa, synthetic construct
  SITE 1: note = D-Aspartic acid
  SITE 2: note = D-Phenylalanine
  SITE 4: note = D- Glutamine
  SITE 5: note = D-Proline
  MOD_RES 6: note = X is Dpa
DFGQPX

SEQ ID NO: 339 - protein, 6 aa, synthetic construct
  MOD_RES 5: note = X is D-Nle
  SITE 6: note = D-Aspartic acid
GGGGXD

SEQ ID NO: 340 - protein, 6 aa, synthetic construct
  SITE 1: note = D-Proline
  SITE 2: note = D- Glutamic acid
  MOD_RES 3: note = X is Amf
  SITE 4: note = D-Threonine
FEXTGG

SEQ ID NO: 341-342 - skipped (2 entries)

SEQ ID NO: 343 - protein, 6 aa, synthetic construct
  MOD_RES 1: note = X is Hle
  SITE 2: note = D-Proline
  SITE 3: note = D- Glutamic acid
  MOD_RES 4: note = X is Pip
  SITE 5: note = D-Proline
  SITE 6: note = D-Threonine
XPEXPT

SEQ ID NO: 344-347 - skipped (4 entries)

SEQ ID NO: 348 - protein, 6 aa, synthetic construct
  SITE 1: note = D-Lysine
  SITE 2: note = D-Threonine
  MOD_RES 3: note = X is Amf
  SITE 4: note = D-Serine
  SITE 5: note = D-Histidine
  MOD_RES 6: note = X is Amf
KTXSHX

SEQ ID NO: 349 - protein, 6 aa, synthetic construct
  MOD_RES 1: note = X is Pip
  SITE 2: note = D-Proline
  SITE 3: note = D-Serine
  MOD_RES 4: note = X is Pip
XPSXGG

SEQ ID NO: 350 - skipped

SEQ ID NO: 351 - protein, 6 aa, synthetic construct
  SITE 1: note = D-Arginine
  SITE 2: note = D-Threonine
  MOD_RES 3: note = X is Dpa
  MOD_RES 4: note = X is Abu
  SITE 5: note = D-Asparagine
  SITE 6: note = D-Phenylalanine
RTXXNF

SEQ ID NO: 352-355 - skipped (4 entries)

SEQ ID NO: 356 - protein, 6 aa, synthetic construct
  SITE 1: note = D-Aspartic acid
  SITE 2: note = D-Phenylalanine
  SITE 3: note = D-Threonine
  SITE 4: note = D-Aspartic acid
DFTDGG

SEQ ID NO: 357 - skipped

SEQ ID NO: 358 - protein, 6 aa, synthetic construct
  SITE 1: note = D-Phenylalanine
  SITE 3: note = D-Serine
  MOD_RES 4..5: note = X is D-Nle
  SITE 6: note = D-Phenylalanine
FGSXXF

SEQ ID NO: 359 - protein, 6 aa, synthetic construct
  SITE 2: note = D-Lysine
  SITE 5: note = D-Proline
  SITE 6: note = D-Threonine
  MOD_RES 1: note = X is Hle
  MOD_RES 3: note = X is Dpa
XKXGPT

SEQ ID NO: 360 - protein, 6 aa, synthetic construct
  SITE 3: note = D- Glutamine
  SITE 6: note = D-Aspartic acid
  MOD_RES 5: note = X is Amf
  MOD_RES 4: note = X is beta-alanine
GGQXXD

SEQ ID NO: 361 - protein, 6 aa, synthetic construct
  MOD_RES 1: note = X is beta-alanine
  MOD_RES 3: note = X is Abu
  SITE 4: note = D- Glutamic acid
  SITE 6: note = D-Phenylalanine
XGXEGF

SEQ ID NO: 362 - skipped

SEQ ID NO: 363 - protein, 6 aa, synthetic construct
  MOD_RES 2: note = X is Dab
  SITE 3: note = D-Lysine
  SITE 4..5: note = D- Glutamic acid
  MOD_RES 6: note = X is Dpa
GXKEEX

SEQ ID NO: 364 - protein, 6 aa, synthetic construct
  MOD_RES 2: note = X is Dpa
  SITE 3: note = D-Proline
  MOD_RES 4: note = X is Abu
  SITE 5: note = D-Proline
  SITE 6: note = D-Asparagine
GXPXPN

SEQ ID NO: 365-367 - skipped (3 entries)

SEQ ID NO: 368 - protein, 6 aa, synthetic construct
  SITE 1: note = D-Serine
  SITE 2: note = D- Glutamic acid
  MOD_RES 3: note = X is d-Nle
  SITE 4: note = D-Asparagine
  MOD_RES 5: note = X is beta-alanine
SEXNXG

SEQ ID NO: 369-373 - skipped (5 entries)

SEQ ID NO: 374 - protein, 6 aa, synthetic construct
  MOD_RES 3: note = X is beta-alanine
  SITE 3: note = D- Glutamine
  MOD_RES 5: note = X is Amf
  SITE 6: note = D-Aspartic acid
GGXQXD

SEQ ID NO: 375-376 - skipped (2 entries)

SEQ ID NO: 377 - protein, 6 aa, synthetic construct
  MOD_RES 1: note = X is beta-alanine
  SITE 2: note = D-Lysine
  MOD_RES 3: note = X is Dab
  MOD_RES 4: note = ox-Histidine
  SITE 6: note = D-Proline
XKXHGP

SEQ ID NO: 378-380 - skipped (3 entries)

SEQ ID NO: 381 - protein, 6 aa, synthetic construct
  MOD_RES 1: note = X is Hle
  MOD_RES 2: note = X is Nle
  SITE 3: note = D- Glutamic acid
  SITE 4: note = D-Serine
  SITE 5: note = D-Threonine
  SITE 6: note = D-Asparagine
XXESTN

SEQ ID NO: 382 - skipped

SEQ ID NO: 383 - protein, 6 aa, synthetic construct
  SITE 2: note = D-Serine
  MOD_RES 3: note = X is Hle
  SITE 4: note = D-Phenylalanine
  SITE 5: note = D- Glutamic acid
  SITE 6: note = D- Glutamine
GSXFEQ

SEQ ID NO: 384 - protein, 6 aa, synthetic construct
  SITE 1: note = D-Proline
  SITE 2: note = D-Threonine
  SITE 3: note = D-Aspartic acid
  SITE 5: note = D-Threonine
PTDGTG

SEQ ID NO: 385 - protein, 6 aa, synthetic construct
  SITE 1: note = D-Proline
  SITE 2: note = D-Threonine
  MOD_RES 3: note = X is Hle
  SITE 4: note = D-Serine
  SITE 5: note = D-Aspartic acid
  MOD_RES 6: note = X is Nap
PTXSDX

SEQ ID NO: 386 - skipped

SEQ ID NO: 387 - protein, 6 aa, synthetic construct
  SITE 2: note = D-Proline
  SITE 3..4: note = D- Glutamic acid
  MOD_RES 6: note = X is Nap
GPEEGX

SEQ ID NO: 388 - skipped

SEQ ID NO: 389 - protein, 6 aa, synthetic construct
  SITE 2..3: note = D-Aspartic acid
  MOD_RES 4: note = X is Dpa
  SITE 5: note = D-Asparagine
  SITE 6: note = D-Serine
GDDXNS

SEQ ID NO: 390 - protein, 6 aa, synthetic construct
  SITE 1: note = D-Aspartic acid
  SITE 2: note = D-Threonine
  MOD_RES 3: note = X is Hle
  MOD_RES 6: note = X is Hle
  SITE 4: note = D-Proline
  SITE 5: note = D- Glutamine
DTXPQX

SEQ ID NO: 391 - protein, 6 aa, synthetic construct
  SITE 1: note = D-Proline
  SITE 2: note = D-Threonine
  SITE 3..4: note = D-Aspartic acid
  SITE 5: note = D-Asparagine
  MOD_RES 6: note = X is Hle
PTDDNX

SEQ ID NO: 392 - protein, 6 aa, synthetic construct
  SITE 1: note = D-Threonine
  SITE 2: note = D-Proline
  MOD_RES 3: note = X is Hle
  SITE 4: note = D-Serine
  SITE 5: note = D-Aspartic acid
  MOD_RES 6: note = X is Nap
TPXSDX

SEQ ID NO: 393 - protein, 6 aa, synthetic construct
  SITE 2: note = D-Arginine
  SITE 3: note = D- Glutamic acid
  SITE 4: note = D-Threonine
  MOD_RES 5: note = X is Hle
  SITE 6: note = D-Asparagine
GRETXN

SEQ ID NO: 394 - skipped

SEQ ID NO: 395 - protein, 6 aa, synthetic construct
  SITE 2: note = D-Phenylalanine
  SITE 3: note = D-Proline
  MOD_RES 4: note = X is Pip
  MOD_RES 5: note = X is Nle
GFPXXG

SEQ ID NO: 396 - protein, 6 aa, synthetic construct
  MOD_RES 1: note = X is beta-alanine
  SITE 2: note = D-Threonine
  SITE 3: note = D- Glutamine
  MOD_RES 4: note = X is Hle
  SITE 5: note = D- Glutamic acid
  SITE 6: note = D-Aspartic acid
XTQXED

SEQ ID NO: 397 - protein, 6 aa, synthetic construct
  SITE 1: note = D-Phenylalanine
  MOD_RES 2: note = X is Hle
  SITE 3: note = D- Glutamic acid
  SITE 4: note = D-Serine
  MOD_RES 5: note = X is beta-alanine
  SITE 6: note = D-Threonine
FXESXT

SEQ ID NO: 398 - skipped

SEQ ID NO: 399 - protein, 6 aa, synthetic construct
  MOD_RES 1: note = X is beta-alanine
  SITE 2: note = D-Arginine
  MOD_RES 3: note = X is Nle
  SITE 4: note = D-Arginine
  SITE 5: note = D- Glutamic acid
  SITE 6: note = D- Glutamine
XRXREQ

SEQ ID NO: 400 - protein, 6 aa, synthetic construct
  MOD_RES 3: note = X is beta-alanine
  SITE 4: note = D-Phenylalanine
  SITE 5: note = D-Asparagine
  SITE 6: note = D-Threonine
GGXFNT

SEQ ID NO: 401 - skipped

SEQ ID NO: 402 - protein, 6 aa, synthetic construct
  SITE 1: note = D-Aspartic acid
  SITE 2: note = D-Asparagine
  SITE 3: note = D- Glutamine
  MOD_RES 4: note = X is Abu
  SITE 5: note = D-Threonine
  SITE 6: note = D-Phenylalanine
DNQXTF

SEQ ID NO: 403 - protein, 6 aa, synthetic construct
  MOD_RES 1: note = X is Nle
  MOD_RES 2: note = X is Hle
  SITE 3: note = D- Glutamic acid
  SITE 4: note = D-Serine
  SITE 5: note = D-Threonine
  SITE 6: note = D-Asparagine
XXESTN

SEQ ID NO: 404 - protein, 6 aa, synthetic construct
  SITE 1: note = D-Asparagine
  SITE 2: note = D-Serine
  MOD_RES 3: note = X is Amf
  MOD_RES 4: note = X is Dab
  SITE 4: note = D-Phenylalanine
NSXXFG

SEQ ID NO: 405 - protein, 6 aa, synthetic construct
  SITE 1: note = D- Glutamine
  MOD_RES 2: note = X is beta-alanine
  SITE 3: note = D-Phenylalanine
  SITE 4: note = D-Arginine
  SITE 5: note = D-Asparagine
  MOD_RES 6: note = X is Amf
QXFRNX

SEQ ID NO: 406 - protein, 6 aa, synthetic construct
  SITE 1: note = D-Asparagine
  SITE 2: note = D-Lysine
  SITE 3: note = D-Threonine
  MOD_RES 5: note = X is Hle
  MOD_RES 6: note = X is Abu
NKTGXX

SEQ ID NO: 407-411 - skipped (5 entries)

SEQ ID NO: 412 - protein, 6 aa, synthetic construct
  SITE 1: note = D- Glutamine
  SITE 2: note = D-Serine
  SITE 3: note = D-Lysine
  MOD_RES 4: note = X is Hle
  SITE 5: note = D-Phenylalanine
  MOD_RES 6: note = X is Dab
QSKXFX

SEQ ID NO: 413 - protein, 6 aa, synthetic construct
  SITE 1: note = D-Arginine
  SITE 2: note = D-Phenylalanine
  SITE 3: note = D-Lysine
  SITE 4: note = D-Aspartic acid
  MOD_RES 5: note = X is Hle
RFKDXG

SEQ ID NO: 414 - protein, 6 aa, synthetic construct
  MOD_RES 1: note = X is Abu
  SITE 3: note = D-Aspartic acid
  MOD_RES 5: note = X is Dpa
  SITE 6: note = D-Asparagine
XGDGXN

SEQ ID NO: 415-416 - skipped (2 entries)

SEQ ID NO: 417 - protein, 6 aa, synthetic construct
  SITE 2: note = D-Aspartic acid
  SITE 3: note = D-Arginine
  SITE 4: note = D-Serine
  MOD_RES 5: note = X is Dab
  SITE 6: note = D-Phenylalanine
GDRSXF

SEQ ID NO: 418 - protein, 6 aa, synthetic construct
  MOD_RES 1: note = X is beta-alanine
  SITE 2: note = D-Arginine
  MOD_RES 3: note = X is Hle
  SITE 4: note = D-Phenylalanine
  SITE 6: note = D-Arginine
XRXFGR

SEQ ID NO: 419 - protein, 6 aa, synthetic construct
  SITE 1: note = D-Arginine
  SITE 2: note = D-Aspartic acid
  MOD_RES 3: note = X is Hle
  SITE 4: note = D-Serine
  MOD_RES 5: note = X is Pip
  SITE 6: note = D- Glutamine
RDXSXQ

SEQ ID NO: 420 - skipped

SEQ ID NO: 421 - protein, 6 aa, synthetic construct
  SITE 1: note = D-Asparagine
  SITE 2: note = D-Aspartic acid
  SITE 3: note = D- Glutamine
  MOD_RES 4: note = X is Abu
  SITE 5: note = D-Threonine
  SITE 6: note = D-Phenylalanine
NDQXTF

SEQ ID NO: 422 - skipped

SEQ ID NO: 423 - protein, 6 aa, synthetic construct
  MOD_RES 2: note = X is Dpa
  SITE 4: note = D-Aspartic acid
  MOD_RES 6: note = X is beta-alanine
GXGDGX

SEQ ID NO: 424 - protein, 6 aa, synthetic construct
  SITE 3: note = D-Asparagine
  SITE 4: note = D-Proline
  MOD_RES 5: note = X is Hle
  MOD_RES 6: note = X is Dab
GGNPXX

SEQ ID NO: 425 - protein, 6 aa, synthetic construct
  SITE 1: note = D-Phenylalanine
  SITE 2: note = D-Threonine
  SITE 3: note = D-Lysine
  SITE 4: note = D-Asparagine
  SITE 5: note = D-Threonine
  SITE 6: note = D-Phenylalanine
FTKNTF

SEQ ID NO: 426 - protein, 6 aa, synthetic construct
  SITE 1: note = D-Asparagine
  SITE 2: note = D-Aspartic acid
  MOD_RES 3: note = X is beta-alanine
  SITE 4: note = D-Threonine
  SITE 6: note = D-Phenylalanine
NDXTGF

SEQ ID NO: 427 - protein, 6 aa, synthetic construct
  SITE 1: note = D-Asparagine
  SITE 3: note = D- Glutamine
  SITE 4: note = D-Serine
  MOD_RES 5: note = X is Nap
  SITE 6: note = D-Aspartic acid
NGQSXD

SEQ ID NO: 428 - protein, 6 aa, synthetic construct
  MOD_RES 2: note = X is Abu
  SITE 3: note = D-Aspartic acid
  MOD_RES 5: note = X is Dpa
  SITE 6: note = D-Asparagine
GXDGXN

SEQ ID NO: 429 - skipped

SEQ ID NO: 430 - protein, 6 aa, synthetic construct
  SITE 1: note = D-Asparagine
  SITE 3: note = D- Glutamic acid
  MOD_RES 4: note = X is Nap
  SITE 5: note = D-Asparagine
  SITE 6: note = D-Aspartic acid
NGEXND

SEQ ID NO: 431 - protein, 6 aa, synthetic construct
  MOD_RES 1: note = X is beta-alanine
  SITE 4: note = D- Glutamine
  MOD_RES 5: note = X is Dpa
  SITE 6: note = D-Phenylalanine
XGGQXF

SEQ ID NO: 432-433 - skipped (2 entries)

SEQ ID NO: 434 - protein, 6 aa, synthetic construct
  MOD_RES 3: note = X is Nle
  SITE 4: note = D-Threonine
  SITE 5: note = D-Aspartic acid
  MOD_RES 6: note = X is Hle
GGXTDX

SEQ ID NO: 435-436 - skipped (2 entries)

SEQ ID NO: 437 - protein, 6 aa, synthetic construct
  SITE 1: note = D-Asparagine
  SITE 2: note = D- Glutamine
  SITE 3: note = D- Glutamic acid
  SITE 4: note = D- Glutamine
  SITE 5: note = D-Serine
  SITE 6: note = D-Phenylalanine
NQEQSF

SEQ ID NO: 438 - skipped

SEQ ID NO: 439 - protein, 6 aa, synthetic construct
  SITE 3: note = D-Threonine
  MOD_RES 4: note = X is Abu
  SITE 5: note = D- Glutamine
  MOD_RES 6: note = X is Nap
GGTXQX

SEQ ID NO: 440 - protein, 6 aa, synthetic construct
  MOD_RES 1: note = X is Dab
  SITE 2: note = D-Threonine
  SITE 3: note = D-Aspartic acid
  SITE 5: note = D-Threonine
XTDGTG

SEQ ID NO: 441 - protein, 6 aa, synthetic construct
  SITE 1: note = D-Asparagine
  SITE 3..4: note = D-Aspartic acid
  SITE 5: note = D-Proline
  MOD_RES 6: note = X is Nap
NGDDPX

SEQ ID NO: 442 - protein, 6 aa, synthetic construct
  SITE 1: note = D-Proline
  SITE 2: note = D-Threonine
  SITE 3: note = D-Phenylalanine
  SITE 4: note = D- Glutamic acid
  MOD_RES 6: note = X is beta-alanine
PTFEGX

SEQ ID NO: 443 - protein, 6 aa, synthetic construct
  SITE 1: note = D-Lysine
  SITE 2: note = D-Asparagine
  SITE 3: note = D-Threonine
  MOD_RES 5: note = X is Hle
  MOD_RES 6: note = X is Abu
KNTGXX

SEQ ID NO: 444-446 - skipped (3 entries)

SEQ ID NO: 447 - protein, 6 aa, synthetic construct
  SITE 1: note = D-Proline
  SITE 2: note = D-Serine
  SITE 3: note = D-Proline
  SITE 4: note = D-Lysine
  MOD_RES 5: note = X is Pip
  SITE 6: note = D- Glutamine
PSPKXQ

SEQ ID NO: 448 - protein, 6 aa, synthetic construct
  SITE 1: note = D-Asparagine
  SITE 2: note = D-Lysine
  MOD_RES 3: note = X is beta-alanine
  SITE 4: note = D-Phenylalanine
  SITE 5..6: note = D-Aspartic acid
NKXFDD

SEQ ID NO: 449 - protein, 6 aa, synthetic construct
  MOD_RES 3: note = X is Dpa
  MOD_RES 4: note = X is Abu
  SITE 5: note = D-Lysine
  SITE 6: note = D-Serine
GGXXKS

SEQ ID NO: 450 - skipped

SEQ ID NO: 451 - protein, 6 aa, synthetic construct
  SITE 2: note = D- Glutamine
  MOD_RES 3: note = X is Gba
  SITE 4..5: note = D-Serine
  SITE 6: note = D-Asparagine
GQXSSN

SEQ ID NO: 452 - protein, 6 aa, synthetic construct
  SITE 1: note = D-Serine
  SITE 2..3: note = D-Proline
  SITE 4..5: note = D- Glutamic acid
  MOD_RES 6: note = X is Dab
SPPEEX

SEQ ID NO: 453 - skipped

SEQ ID NO: 454 - protein, 6 aa, synthetic construct
  SITE 3: note = D- Glutamine
  SITE 4: note = D-Serine
  MOD_RES 5: note = X is Nap
  SITE 6: note = D-Aspartic acid
GGQSXD

SEQ ID NO: 455 - skipped

SEQ ID NO: 456 - protein, 6 aa, synthetic construct
  SITE 1: note = D-Asparagine
  MOD_RES 2: note = X is Nle
  SITE 3: note = D-Proline
  SITE 4: note = D-Asparagine
  SITE 5: note = D-Serine
  MOD_RES 6: note = X is Nap
NXPNSX

SEQ ID NO: 457 - protein, 6 aa, synthetic construct
  SITE 2: note = D-Serine
  MOD_RES 3: note = X is Hle
  SITE 4: note = D-Aspartic acid
GSXDGG

SEQ ID NO: 458 - protein, 6 aa, synthetic construct
  MOD_RES 1: note = X is Abu
  SITE 2: note = D-Proline
  MOD_RES 3: note = X is Nle
  SITE 4..5: note = D-Proline
XPXPPG

SEQ ID NO: 459 - protein, 6 aa, synthetic construct
  MOD_RES 1: note = X is Abu
  SITE 4: note = D-Arginine
  MOD_RES 6: note = X is Dpa
  SITE 5: note = D-Arginine
XGGGRX

SEQ ID NO: 460 - protein, 6 aa, synthetic construct
  SITE 1: note = D- Glutamine
  MOD_RES 2: note = X is Dab
  SITE 3: note = D-Aspartic acid
  MOD_RES 4: note = X is Pip
  SITE 5: note = D- Glutamine
QXDXQG

SEQ ID NO: 461-463 - skipped (3 entries)

SEQ ID NO: 464 - protein, 6 aa, synthetic construct
  SITE 1: note = D-Aspartic acid
  MOD_RES 2: note = X is Hle
  MOD_RES 3: note = X is beta-alanine
  SITE 6: note = D- Glutamic acid
DXXGGE

SEQ ID NO: 465 - protein, 6 aa, synthetic construct
  SITE 1: note = D-Asparagine
  MOD_RES 3: note = X is Dpa
  MOD_RES 5: note = X is beta-alanine
NGXGXG

SEQ ID NO: 466 - protein, 6 aa, synthetic construct
  SITE 1: note = D-Threonine
  SITE 2: note = D-Proline
  SITE 3: note = D-Aspartic acid
  SITE 5: note = D-Threonine
TPDGTG

SEQ ID NO: 467 - protein, 6 aa, synthetic construct
  SITE 1: note = D-Asparagine
  SITE 2: note = D-Aspartic acid
  SITE 4: note = D-Aspartic acid
  SITE 5: note = D-Proline
  MOD_RES 6: note = X is Nap
NDGDPX

SEQ ID NO: 468 - protein, 6 aa, synthetic construct
  SITE 1: note = D-Proline
  SITE 2: note = D-Threonine
  SITE 3: note = D-Phenylalanine
  SITE 4: note = D- Glutamic acid
  MOD_RES 5: note = X is beta-alanine
PTFEXG

SEQ ID NO: 469 - protein, 6 aa, synthetic construct
  SITE 1: note = D-Lysine
  MOD_RES 2: note = X is beta-alanine
  SITE 3: note = D-Asparagine
  SITE 4: note = D-Phenylalanine
  SITE 5..6: note = D-Aspartic acid
KXNFDD

SEQ ID NO: 470 - protein, 6 aa, synthetic construct
  SITE 1: note = D- Glutamine
  SITE 2: note = D- Glutamic acid
  SITE 3: note = D-Lysine
  SITE 4: note = D-Aspartic acid
  MOD_RES 5: note = X is Hle
QEKDXG

SEQ ID NO: 471-473 - skipped (3 entries)

SEQ ID NO: 474 - protein, 6 aa, synthetic construct
  SITE 1: note = D-Arginine
  MOD_RES 2: note = X is beta-alanine
  SITE 3: note = D-Serine
  SITE 4: note = D-Proline
  SITE 5: note = D-Phenylalanine
  SITE 6: note = D- Glutamic acid
RXSPFE

SEQ ID NO: 475 - protein, 6 aa, synthetic construct
  MOD_RES 1: note = ox-Histidine
  SITE 2..3: note = D-Aspartic acid
  SITE 4: note = D- Glutamic acid
  MOD_RES 5: note = X is Abu
  SITE 6: note = D-Serine
HDDEXS

SEQ ID NO: 476 - skipped

SEQ ID NO: 477 - protein, 6 aa, synthetic construct
  SITE 1: note = D-Aspartic acid
  SITE 2: note = D- Glutamine
  MOD_RES 3: note = X is Hle
  MOD_RES 6: note = X is Nle
DQXGGX

SEQ ID NO: 478 - protein, 6 aa, synthetic construct
  SITE 1: note = D-Proline
  SITE 2: note = D- Glutamine
  MOD_RES 4: note = X is Nap
  SITE 5: note = D- Glutamic acid
  SITE 6: note = D-Aspartic acid
PQGXED

SEQ ID NO: 479 - protein, 6 aa, synthetic construct
  SITE 1: note = D-Proline
  SITE 2: note = D-Serine
  SITE 3: note = D-Asparagine
  SITE 4: note = D-Aspartic acid
  SITE 5: note = D- Glutamic acid
  MOD_RES 6: note = X is Pip
PSNDEX

SEQ ID NO: 480 - protein, 6 aa, synthetic construct
  SITE 2: note = D-Asparagine
  SITE 3: note = D-Phenylalanine
  MOD_RES 6: note = X is Nle
  SITE 5: note = D- Glutamic acid
GNFGEX

SEQ ID NO: 481 - protein, 6 aa, synthetic construct
  SITE 2: note = D-Asparagine
  MOD_RES 3: note = X is Dpa
  MOD_RES 5: note = X is beta-alanine
GNXGXG

SEQ ID NO: 482 - protein, 6 aa, synthetic construct
  SITE 1: note = D-Asparagine
  MOD_RES 2..3: note = X is Dab
  SITE 4: note = D-Proline
  SITE 5: note = D-Phenylalanine
  SITE 6: note = D- Glutamic acid
NXXPFE

SEQ ID NO: 483 - protein, 6 aa, synthetic construct
  SITE 1: note = D-Asparagine
  MOD_RES 2: note = X is beta-alanine
  SITE 4: note = D-Asparagine
  SITE 6: note = D-Arginine
  MOD_RES 5: note = X is Nle
NCGNXR

SEQ ID NO: 484-485 - skipped (2 entries)

SEQ ID NO: 486 - protein, 6 aa, synthetic construct
  MOD_RES 1: note = X is Gba
  SITE 2: note = D- Glutamic acid
  SITE 4: note = D-Serine
  MOD_RES 5: note = X is Dab
  SITE 6: note = D-Phenylalanine
XEGSXF

SEQ ID NO: 487 - protein, 6 aa, synthetic construct
  MOD_RES 4: note = X is Nle
  SITE 5: note = D-Proline
  SITE 6: note = D-Aspartic acid
GGGXPD

SEQ ID NO: 488 - protein, 6 aa, synthetic construct
  MOD_RES 4: note = X is Dpa
  SITE 5: note = D- Glutamine
GGGXQG

SEQ ID NO: 489 - protein, 6 aa, synthetic construct
  MOD_RES 1: note = X is Pip
  SITE 2: note = D-Threonine
  SITE 3: note = D-Proline
  SITE 4: note = D-Asparagine
  SITE 5: note = D-Serine
  MOD_RES 6: note = X is Nap
XTPNSX

SEQ ID NO: 490 - skipped

SEQ ID NO: 491 - protein, 6 aa, synthetic construct
  SITE 1: note = D-Phenylalanine
  MOD_RES 2: note = X is Hle
  SITE 3: note = D- Glutamic acid
  MOD_RES 4: note = X is beta-alanine
  SITE 5: note = D-Serine
  SITE 6: note = D-Threonine
FXEXST

SEQ ID NO: 492 - skipped

SEQ ID NO: 493 - protein, 6 aa, synthetic construct
  SITE 1: note = D-Aspartic acid
  SITE 2: note = D-Serine
  SITE 3: note = D-Phenylalanine
  SITE 4: note = D-Asparagine
  SITE 5: note = D-Lysine
  SITE 6: note = D-Phenylalanine
DSFNKF

SEQ ID NO: 494-495 - skipped (2 entries)

SEQ ID NO: 496 - protein, 6 aa, synthetic construct
  SITE 3: note = D-Asparagine
  MOD_RES 4: note = X is Abu
  SITE 5: note = D-Phenylalanine
  SITE 6: note = D- Glutamine
GGNXFQ

SEQ ID NO: 497 - protein, 6 aa, synthetic construct
  SITE 2..3: note = D-Asparagine
  MOD_RES 5: note = X is Dpa
  SITE 6: note = D-Asparagine
GNNGXN

SEQ ID NO: 498 - skipped

SEQ ID NO: 499 - protein, 6 aa, synthetic construct
  SITE 1: note = D-Lysine
  SITE 2..3: note = D-Proline
  SITE 4: note = D-Serine
  MOD_RES 5: note = X is Pip
  SITE 6: note = D- Glutamine
KPPSXQ

SEQ ID NO: 500 - protein, 6 aa, synthetic construct
  MOD_RES 2: note = X is beta-alanine
  SITE 3: note = D-Phenylalanine
  SITE 6: note = D-Threonine
GXFGGT

SEQ ID NO: 501 - protein, 6 aa, synthetic construct
  SITE 1: note = D-Aspartic acid
  MOD_RES 2: note = X is beta-alanine
  MOD_RES 4: note = X is Abu
  SITE 5: note = D-Threonine
  SITE 6: note = D-Phenylalanine
DXGXTF

SEQ ID NO: 502 - protein, 6 aa, synthetic construct
  MOD_RES 4: note = X is Nap
  SITE 5: note = D-Aspartic acid
  MOD_RES 6: note = X is Dab
GGGXNX

SEQ ID NO: 503 - protein, 6 aa, synthetic construct
  SITE 2: note = D-Aspartic acid
  SITE 3: note = D- Glutamic acid
  MOD_RES 4: note = X is Nap
  SITE 5: note = D-Asparagine
  SITE 6: note = D-Aspartic acid
GNQXND

SEQ ID NO: 504 - protein, 6 aa, synthetic construct
  MOD_RES 1: note = X is beta-alanine
  SITE 2: note = D-Arginine
  MOD_RES 3: note = X is Nle
  SITE 4: note = D-Proline
XRXPGG

SEQ ID NO: 505 - protein, 6 aa, synthetic construct
  MOD_RES 2: note = X is Gba
  SITE 3: note = D- Glutamic acid
  SITE 4: note = D-Serine
  MOD_RES 5: note = X is Dab
  SITE 6: note = D-Phenylalanine
GXESXF

SEQ ID NO: 506 - protein, 6 aa, synthetic construct
  MOD_RES 2: note = X is beta-alanine
  SITE 4: note = D-Phenylalanine
  SITE 5: note = D-Asparagine
  SITE 6: note = D-Threonine
GXGFNT

SEQ ID NO: 507 - protein, 6 aa, synthetic construct
  SITE 1: note = D- Glutamic acid
  SITE 2..3: note = D-Serine
  SITE 4: note = D-Threonine
  SITE 6: note = D-Phenylalanine
ESSTGF

SEQ ID NO: 508-512 - skipped (5 entries)

SEQ ID NO: 513 - protein, 6 aa, synthetic construct
  MOD_RES 1: note = X is Dab
  SITE 2: note = D-Threonine
  SITE 4: note = D-Aspartic acid
  SITE 5: note = D-Threonine
XTGDTG

SEQ ID NO: 514 - protein, 6 aa, synthetic construct
  MOD_RES 1: note = X is Nle
  MOD_RES 5: note = X is Nle
  SITE 2: note = D-Aspartic acid
  SITE 3: note = D-Histidine
  SITE 4: note = D-Aspartic acid
  SITE 6: note = D-Phenylalanine
XDHDXF

SEQ ID NO: 515 - protein, 6 aa, synthetic construct
  SITE 2: note = D-Asparagine
  SITE 3..4: note = D-Aspartic acid
  SITE 5: note = D-Proline
  MOD_RES 6: note = X is Nap
GNDDPX

SEQ ID NO: 516 - protein, 6 aa, synthetic construct
  MOD_RES 3: note = X is Nle
  SITE 5: note = D-Phenylalanine
  SITE 6: note = D-Asparagine
GGXGFN

SEQ ID NO: 517-522 - skipped (6 entries)

SEQ ID NO: 523 - protein, 6 aa, synthetic construct
  MOD_RES 2: note = X is Pip
  MOD_RES 3: note = X is Gba
  SITE 4: note = D-Serine
  SITE 5: note = D-Threonine
  SITE 6: note = D- Glutamine
GXXSTQ

SEQ ID NO: 524 - protein, 6 aa, synthetic construct
  SITE 1: note = D-Lysine
  SITE 2: note = D-Aspartic acid
  MOD_RES 5: note = X is Amf
  SITE 6: note = D-Phenylalanine
KDGGXF

SEQ ID NO: 525 - protein, 6 aa, synthetic construct
  MOD_RES 1: note = X is beta-alanine
  MOD_RES 2: note = X is Abu
  SITE 3: note = D-Asparagine
  SITE 4: note = D-Arginine
  SITE 5: note = D-Asparagine
XXNRNG

SEQ ID NO: 526 - skipped

SEQ ID NO: 527 - protein, 6 aa, synthetic construct
  SITE 1: note = D-Proline
  SITE 3..4: note = D- Glutamic acid
  MOD_RES 6: note = X is Nap
PGEEGX

SEQ ID NO: 528 - protein, 6 aa, synthetic construct
  MOD_RES 1: note = X is Abu
  MOD_RES 2: note = X is Amf
  SITE 3: note = D-Histidine
  SITE 5: note = D-Phenylalanine
XXHGFG

SEQ ID NO: 529-531 - skipped (3 entries)

SEQ ID NO: 532 - protein, 6 aa, synthetic construct
  SITE 1: note = D-Aspartic acid
  MOD_RES 2: note = X is Dpa
  SITE 3: note = D-Serine
  MOD_RES 6: note = X is Abu
DXSGGX

SEQ ID NO: 533 - protein, 6 aa, synthetic construct
  MOD_RES 1: note = X is Abu
  MOD_RES 2: note = X is Gba
  SITE 3: note = D-Serine
  SITE 4: note = D-Proline
  SITE 5: note = D-Phenylalanine
  SITE 6: note = D- Glutamic acid
XXSPFE

SEQ ID NO: 534 - skipped

SEQ ID NO: 535 - protein, 6 aa, synthetic construct
  SITE 1: note = D-Lysine
  MOD_RES 2..3: note = ox-Histidine
  SITE 4: note = D-Serine
  SITE 5: note = D-Aspartic acid
  MOD_RES 6: note = X is Nap
KHHSDX

SEQ ID NO: 536 - skipped

SEQ ID NO: 537 - protein, 6 aa, synthetic construct
  MOD_RES 1: note = X is Amf
  SITE 2: note = D-Threonine
  SITE 3..4: note = D-Proline
  MOD_RES 5: note = X is beta-alanine
  SITE 6: note = D-Aspartic acid
XTPPXD

SEQ ID NO: 538 - skipped

SEQ ID NO: 539 - protein, 6 aa, synthetic construct
  MOD_RES 3: note = X is beta-alanine
  SITE 4: note = D- Glutamine
  MOD_RES 5: note = X is Dpa
  SITE 6: note = D-Phenylalanine
GGXQXF

SEQ ID NO: 540 - protein, 6 aa, synthetic construct
  SITE 1: note = D-Arginine
  SITE 2: note = D-Aspartic acid
  SITE 4: note = D-Serine
  MOD_RES 5: note = X is Dpa
  SITE 6: note = D-Phenylalanine
RDGSXF

SEQ ID NO: 541 - protein, 6 aa, synthetic construct
  MOD_RES 1: note = X is Abu
  MOD_RES 3: note = X is Dab
  SITE 2: note = D- Glutamine
  SITE 4: note = D-Phenylalanine
  SITE 5..6: note = D-Aspartic acid
XQXFDD

SEQ ID NO: 542 - protein, 6 aa, synthetic construct
  SITE 1: note = D-Lysine
  SITE 2: note = D-Asparagine
  SITE 4: note = D-Phenylalanine
  SITE 5..6: note = D-Aspartic acid
  MOD_RES 3: note = X is beta-alanine
KNXFDD

SEQ ID NO: 543 - protein, 6 aa, synthetic construct
  SITE 3: note = D-Phenylalanine
  SITE 4: note = D-Asparagine
  SITE 5: note = D-Lysine
  SITE 6: note = D-Phenylalanine
GGFNKF

SEQ ID NO: 544 - protein, 6 aa, synthetic construct
  MOD_RES 1: note = X is Nle
  SITE 2: note = D- Glutamic acid
  MOD_RES 3: note = X is beta-alanine
  SITE 6: note = D- Glutamic acid
XEXGGE

SEQ ID NO: 545 - protein, 6 aa, synthetic construct
  SITE 1: note = D-Asparagine
  MOD_RES 2: note = X is beta-alanine
  SITE 4: note = D-Serine
  MOD_RES 5: note = X is Nap
  SITE 6: note = D-Aspartic acid
NXGSXD

SEQ ID NO: 546 - skipped

SEQ ID NO: 547 - protein, 6 aa, synthetic construct
  MOD_RES 1: note = X is beta-alanine
  SITE 2: note = D-Asparagine
  SITE 3: note = D-Proline
  MOD_RES 4: note = X is Nap
  SITE 5: note = D- Glutamic acid
  SITE 6: note = D-Aspartic acid
XNPXED

SEQ ID NO: 548 - skipped

SEQ ID NO: 549 - protein, 6 aa, synthetic construct
  SITE 1: note = D-Arginine
  SITE 2: note = D-Proline
  SITE 3: note = D-Aspartic acid
  SITE 5: note = D-Aspartic acid
  MOD_RES 4: note = ox-Histidine
  MOD_RES 6: note = X is Nap
RPDHDX

SEQ ID NO: 550-551 - skipped (2 entries)

SEQ ID NO: 552 - protein, 6 aa, synthetic construct
  SITE 3: note = D-Proline
  MOD_RES 4: note = X is beta-alanine
  SITE 5: note = D- Glutamic acid
  MOD_RES 6: note = X is Nle
GGPXEX

SEQ ID NO: 553 - skipped

SEQ ID NO: 554 - protein, 6 aa, synthetic construct
  MOD_RES 3: note = X is Hle
  SITE 2: note = D-Serine
  MOD_RES 4: note = X is beta-alanine
OSXXGG

SEQ ID NO: 555 - protein, 6 aa, synthetic construct
  MOD_RES 1: note = X is beta-alanine
  SITE 2: note = D-Asparagine
  SITE 3: note = D-Aspartic acid
  MOD_RES 4: note = X is Nap
  SITE 5: note = D-Asparagine
  SITE 6: note = D-Aspartic acid
XNDXND

SEQ ID NO: 556 - protein, 6 aa, synthetic construct
  MOD_RES 1: note = X is beta-alanine
  SITE 2: note = D-Aspartic acid
  MOD_RES 3: note = X is Amf
  SITE 5: note = D-Serine
  SITE 6: note = D-Threonine
XDXGST

SEQ ID NO: 557-558 - skipped (2 entries)

SEQ ID NO: 559 - protein, 6 aa, synthetic construct
  SITE 1: note = D-Lysine
  MOD_RES 2: note = X is beta-alanine
  MOD_RES 4: note = ox-Histidine
  SITE 5: note = D-Threonine
  SITE 6: note = D- Glutamine
KXGHTQ

SEQ ID NO: 560 - protein, 6 aa, synthetic construct
  SITE 2: note = D-Serine
  MOD_RES 3: note = X is Amf
  MOD_RES 6: note = X is Amf
  SITE 5: note = D-Serine
GSXGSX

SEQ ID NO: 561 - skipped

SEQ ID NO: 562 - protein, 6 aa, synthetic construct
  SITE 1: note = D-Threonine
  SITE 2: note = D-Phenylalanine
  SITE 3: note = D-Lysine
  SITE 4: note = D-Asparagine
  SITE 5: note = D-Threonine
  SITE 5: note = D-Phenylalanine
TFKNTF

SEQ ID NO: 563-565 - skipped (3 entries)

SEQ ID NO: 566 - protein, 6 aa, synthetic construct
  SITE 1: note = D-Aspartic acid
  MOD_RES 2: note = X is beta-alanine
  MOD_RES 3: note = X is Dab
  SITE 4: note = D-Proline
  MOD_RES 5: note = ox-Histidine
  SITE 6: note = D- Glutamic acid
DXXPHE

SEQ ID NO: 567 - protein, 6 aa, synthetic construct
  SITE 1: note = D-Proline
  SITE 2: note = D- Glutamic acid
  SITE 4: note = D- Glutamic acid
  MOD_RES 6: note = X is Nap
PGGEGX

SEQ ID NO: 568-569 - skipped (2 entries)

SEQ ID NO: 570 - protein, 6 aa, synthetic construct
  SITE 3: note = D-Aspartic acid
  MOD_RES 5: note = X is Nap
  MOD_RES 6: note = X is Dab
GGGDXX

SEQ ID NO: 571 - protein, 6 aa, synthetic construct
  SITE 2: note = D-Asparagine
  SITE 3: note = D- Glutamine
  SITE 4: note = D-Serine
  SITE 6: note = D-Aspartic acid
  MOD_RES 5: note = X is Nap
GNQSXD

SEQ ID NO: 572 - protein, 6 aa, synthetic construct
  MOD_RES 1: note = X is ox-Nap
  SITE 2: note = D-Proline
  SITE 3: note = D-Aspartic acid
  MOD_RES 5: note = X is Hle
  SITE 6: note = D-Proline
XPDGXP

SEQ ID NO: 573 - skipped

SEQ ID NO: 574 - protein, 6 aa, synthetic construct
  MOD_RES 1: note = X is Nle
  SITE 2: note = D-Threonine
  MOD_RES 3: note = X is Nle
  SITE 6: note = D-Threonine
XTXGGT

SEQ ID NO: 575 - protein, 6 aa, synthetic construct
  SITE 1: note = D-Asparagine
  SITE 2: note = D-Aspartic acid
  MOD_RES 3: note = X is Pip
  SITE 4: note = D-Serine
  SITE 5: note = D-Threonine
  SITE 6: note = D-Phenylalanine
NDXSTF

SEQ ID NO: 576 - protein, 6 aa, synthetic construct
  SITE 1..2: note = D-Histidine
  MOD_RES 4: note = X is beta-alanine
HHGXGG

SEQ ID NO: 577 - protein, 6 aa, synthetic construct
  MOD_RES 1: note = X is Abu
  MOD_RES 2: note = X is Nle
  SITE 3..5: note = D-Proline
XXPPPG

SEQ ID NO: 578 - skipped

SEQ ID NO: 579 - protein, 6 aa, synthetic construct
  SITE 2: note = D- Glutamic acid
  MOD_RES 3: note = X is Nle
  MOD_RES 4: note = X is Pip
  SITE 5: note = D-Asparagine
GEXXNG

SEQ ID NO: 580 - protein, 6 aa, synthetic construct
  SITE 1: note = D-Serine
  SITE 2..3: note = D-Proline
  SITE 4: note = D-Lysine
  MOD_RES 5: note = X is Pip
  SITE 6: note = D- Glutamine
SPPKXQ

SEQ ID NO: 581 - protein, 6 aa, synthetic construct
  SITE 2: note = D-Serine
  MOD_RES 3: note = X is Nle
  SITE 4: note = D- Glutamic acid
GSXEGG

SEQ ID NO: 582 - skipped

SEQ ID NO: 583 - protein, 6 aa, synthetic construct
  SITE 3: note = D- Glutamic acid
  MOD_RES 4: note = X is Hle
  SITE 5: note = D-Asparagine
  MOD_RES 6: note = X is Nle
GGEXNX

SEQ ID NO: 584-586 - skipped (3 entries)

SEQ ID NO: 587 - protein, 6 aa, synthetic construct
  MOD_RES 3: note = X is Abu
  MOD_RES 6: note = X is Nap
  SITE 4: note = D-Threonine
  SITE 5: note = D- Glutamine
GGXTQX

SEQ ID NO: 588 - protein, 6 aa, synthetic construct
  SITE 1: note = D-Aspartic acid
  SITE 2: note = D-Threonine
  SITE 3: note = D-Proline
  MOD_RES 4: note = X is Hle
  MOD_RES 6: note = X is Hle
  SITE 5: note = D- Glutamine
DTPXQX

SEQ ID NO: 589-590 - skipped (2 entries)

SEQ ID NO: 591 - protein, 6 aa, synthetic construct
  MOD_RES 1: note = X is beta-alanine
  SITE 2: note = D-Histidine
  MOD_RES 3: note = X is Dab
  SITE 4..5: note = D-Phenylalanine
XHXFFG

SEQ ID NO: 592 - skipped

SEQ ID NO: 593 - protein, 6 aa, synthetic construct
  SITE 2: note = D- Glutamine
  MOD_RES 3: note = X is Gba
  SITE 4: note = D-Serine
  SITE 6: note = D-Serine
  SITE 5: note = D-Asparagine
GQXSNS

SEQ ID NO: 594 - protein, 6 aa, synthetic construct
  SITE 1..2: note = D-Serine
  MOD_RES 3: note = X is Dpa
  MOD_RES 4: note = X is beta-alanine
SSXXGG

SEQ ID NO: 595 - protein, 6 aa, synthetic construct
  MOD_RES 1: note = X is Nle
  SITE 2..3: note = D-Aspartic acid
  MOD_RES 4: note = X is Pip
  SITE 5: note = D- Glutamine
XDDXQG

SEQ ID NO: 596 - protein, 6 aa, synthetic construct
  SITE 1: note = D-Proline
  MOD_RES 2: note = X is beta-alanine
  SITE 3: note = D-Aspartic acid
  SITE 4: note = D-Threonine
  MOD_RES 5: note = X is Hle
  SITE 6: note = D-Aspartic acid
PXDTXD

SEQ ID NO: 597 - skipped

SEQ ID NO: 598 - protein, 6 aa, synthetic construct
  SITE 1: note = D-Proline
  MOD_RES 2: note = X is Pip
  SITE 3: note = D-Threonine
  SITE 4: note = D-Asparagine
  SITE 5: note = D-Serine
  MOD_RES 6: note = X is Nap
PXTNSX

SEQ ID NO: 599 - protein, 6 aa, synthetic construct
  SITE 1: note = D- Glutamine
  MOD_RES 2: note = ox-Histidine
  SITE 3: note = D-Asparagine
  MOD_RES 4: note = X is Pip
  SITE 5: note = D-Aspartic acid
  MOD_RES 6: note = X is Nap
QHNXDX

SEQ ID NO: 600 - protein, 6 aa, synthetic construct
  SITE 1: note = D- Glutamine
  MOD_RES 2: note = X is Pip
  MOD_RES 3: note = ox-Histidine
  SITE 4: note = D-Asparagine
  SITE 5: note = D-Aspartic acid
  MOD_RES 6: note = X is Nap
QXHNDX

SEQ ID NO: 601-602 - skipped (2 entries)

SEQ ID NO: 603 - protein, 6 aa, synthetic construct
  SITE 2: note = D-Serine
  SITE 4: note = D-Serine
  MOD_RES 5..6: note = X is Amf
GSGSXX

SEQ ID NO: 604 - skipped

SEQ ID NO: 605 - protein, 6 aa, synthetic construct
  MOD_RES 2: note = X is Hle
  SITE 3: note = D-Threonine
  MOD_RES 4: note = X is Nap
GXTXGG

SEQ ID NO: 606-607 - skipped (2 entries)

SEQ ID NO: 608 - protein, 6 aa, synthetic construct
  MOD_RES 1: note = X is Nle
  SITE 2: note = D-Phenylalanine
  SITE 3: note = D-Asparagine
  MOD_RES 4: note = X is beta-alanine
  SITE 5..6: note = D-Serine
XFNXSS

SEQ ID NO: 609 - protein, 6 aa, synthetic construct
  SITE 1: note = D-Asparagine
  MOD_RES 2: note = X is beta-alanine
  SITE 3..4: note = D-Proline
  MOD_RES 5: note = X is Nle
  SITE 6: note = D-Histidine
NXPPXH

SEQ ID NO: 610 - protein, 6 aa, synthetic construct
  SITE 1: note = D- Glutamine
  SITE 2: note = D-Asparagine
  SITE 3: note = D- Glutamic acid
  SITE 4: note = D- Glutamine
  SITE 5: note = D-Serine
  SITE 6: note = D-Phenylalanine
QNEQSF

SEQ ID NO: 611 - skipped

SEQ ID NO: 612 - protein, 6 aa, synthetic construct
  MOD_RES 1: note = X is beta-alanine
  SITE 2: note = D-Lysine
  MOD_RES 4: note = ox-Histidine
  SITE 5: note = D-Threonine
  SITE 6: note = D- Glutamine
XKGHTQ

SEQ ID NO: 613-614 - skipped (2 entries)

SEQ ID NO: 615 - protein, 6 aa, synthetic construct
  SITE 2: note = D-Serine
  MOD_RES 3: note = X is Abu
  SITE 4: note = D-Arginine
  SITE 5: note = D-Aspartic acid
  SITE 6: note = D-Phenylalanine
GSXRDF

SEQ ID NO: 616 - protein, 6 aa, synthetic construct
  MOD_RES 1: note = X is Nle
  SITE 2: note = D-Lysine
  SITE 4: note = D- Glutamic acid
  MOD_RES 5: note = ox-Histidine
  SITE 6: note = D-Lysine
XKGEHK

SEQ ID NO: 617 - skipped

SEQ ID NO: 618 - protein, 6 aa, synthetic construct
  MOD_RES 1: note = X is beta-alanine
  SITE 3: note = D-Phenylalanine
  SITE 6: note = D-Threonine
XGFGGT

SEQ ID NO: 619 - protein, 6 aa, synthetic construct
  MOD_RES 1: note = X is Abu
  SITE 6: note = D-Phenylalanine
XGGGGF

SEQ ID NO: 620 - protein, 6 aa, synthetic construct
  SITE 1..2: note = D-Proline
  SITE 3: note = D-Serine
  SITE 4..5: note = D- Glutamic acid
  MOD_RES 6: note = X is Dab
PPSEEX

SEQ ID NO: 621 - protein, 6 aa, synthetic construct
  MOD_RES 2: note = X is Abu
  SITE 5: note = D-Arginine
  MOD_RES 6: note = X is Dpa
GXGGRX

SEQ ID NO: 622 - skipped

SEQ ID NO: 623 - protein, 6 aa, synthetic construct
  SITE 1: note = D-Asparagine
  SITE 2: note = D-Proline
  SITE 3: note = D-Serine
  MOD_RES 4: note = X is Nle
  SITE 5: note = D-Phenylalanine
  SITE 6: note = D- Glutamic acid
NPSXFE

SEQ ID NO: 624-625 - skipped (2 entries)

SEQ ID NO: 626 - protein, 6 aa, synthetic construct
  SITE 1: note = D- Glutamine
  MOD_RES 2: note = X is beta-alanine
  SITE 3: note = D-Proline
  MOD_RES 4: note = X is Nle
  MOD_RES 5: note = X is Pip
  SITE 6: note = D- Glutamine
QFDHGG

SEQ ID NO: 627 - protein, 6 aa, synthetic construct
  SITE 1: note = D-Proline
  SITE 2: note = D-Asparagine
  SITE 3: note = D-Serine
  MOD_RES 4: note = X is Nle
  SITE 5: note = D-Phenylalanine
  SITE 6: note = D- Glutamic acid
PNSXFE

SEQ ID NO: 628 - protein, 6 aa, synthetic construct
  SITE 1: note = D-Aspartic acid
  SITE 2: note = D-Threonine
  SITE 3: note = D- Glutamine
  MOD_RES 4: note = X is Hle
  MOD_RES 6: note = X is Hle
  SITE 5: note = D-Proline
DTQXPX

SEQ ID NO: 629-630 - skipped (2 entries)

SEQ ID NO: 631 - protein, 6 aa, synthetic construct
  SITE 1: note = D-Threonine
  MOD_RES 2: note = X is Dab
  SITE 3: note = D-Aspartic acid
  SITE 5: note = D-Threonine
TXDGTG

SEQ ID NO: 632-633 - skipped (2 entries)

SEQ ID NO: 634 - protein, 6 aa, synthetic construct
  SITE 3: note = D- Glutamic acid
  MOD_RES 4: note = X is Hle
  SITE 5: note = D-Histidine
  SITE 6: note = D-Proline
GGEXHP

SEQ ID NO: 635-637 - skipped (3 entries)

SEQ ID NO: 638 - protein, 6 aa, synthetic construct
  MOD_RES 2: note = X is Abu
  MOD_RES 3: note = ox-Histidine
  SITE 4: note = D-Histidine
  SITE 5: note = D-Serine
  SITE 6: note = D-Asparagine
GXHHSN

SEQ ID NO: 639 - protein, 6 aa, synthetic construct
  MOD_RES 1: note = X is beta-alanine
  SITE 2: note = D-Proline
  SITE 3: note = D-Aspartic acid
  MOD_RES 4: note = X is Hle
  SITE 5: note = D-Threonine
  SITE 6: note = D-Aspartic acid
XPDXTD

SEQ ID NO: 640 - protein, 6 aa, synthetic construct
  SITE 4: note = D-Proline
  SITE 6: note = D-Aspartic acid
  MOD_RES 5: note = X is Nle
GGGPXD

SEQ ID NO: 641-643 - skipped (3 entries)

SEQ ID NO: 644 - protein, 5 aa, synthetic construct
  MOD_RES 1: note = X is Nle
  SITE 2: note = D-Proline
  SITE 3: note = D-Aspartic acid
  SITE 4: note = D- Glutamic acid
  SITE 5: note = D-Threonine
XPDET

SEQ ID NO: 645 - protein, 5 aa, synthetic construct
  MOD_RES 1: note = X is Nle
  SITE 2: note = D-Proline
  SITE 3: note = D-Aspartic acid
  SITE 4: note = D-Arginine
  MOD_RES 5: note = ox-Tryptophan
XPDRW

SEQ ID NO: 646-647 - skipped (2 entries)

SEQ ID NO: 648 - protein, 6 aa, synthetic construct
  MOD_RES 1: note = X is Nle
  SITE 2: note = D-Proline
  SITE 3: note = D-Aspartic acid
  SITE 4: note = D-Arginine
  MOD_RES 6: note = ox-Tryptophan
XPDRWW

SEQ ID NO: 649 - skipped

SEQ ID NO: 650 - protein, 5 aa, synthetic construct
  SITE 1: note = D-Lysine
  SITE 2: note = D-Aspartic acid
  MOD_RES 3: note = X is Nle
  SITE 4..5: note = D- Glutamine
KDXQQ

SEQ ID NO: 651-652 - skipped (2 entries)

SEQ ID NO: 653 - protein, 5 aa, synthetic construct
  MOD_RES 1: note = X is Nle
  SITE 2: note = D-Threonine
  SITE 3: note = D-Proline
  SITE 4: note = D-Threonine
  SITE 5: note = D-Serine
XTPTS

SEQ ID NO: 654-663 - skipped (10 entries)

SEQ ID NO: 664 - protein, 6 aa, synthetic construct
  MOD_RES 1: note = X is Abu
  SITE 3: note = D-Asparagine
  MOD_RES 4: note = X is Nle
  SITE 5: note = D-Asparagine
  SITE 6: note = D-Histidine
XGNXNH

SEQ ID NO: 665 - protein, 4 aa, synthetic construct
  SITE 1: note = D-Lysine
  SITE 2..3: note = D-Arginine
  SITE 4: note = D-Histidine
KRRH

SEQ ID NO: 666-669 - skipped (4 entries)

SEQ ID NO: 670 - protein, 4 aa, synthetic construct
KWKK

SEQ ID NO: 671 - protein, 4 aa, synthetic construct
KKWK

SEQ ID NO: 672 - protein, 5 aa, synthetic construct
KWWKK

SEQ ID NO: 673 - protein, 4 aa, synthetic construct
WWKK

SEQ ID NO: 674 - protein, 4 aa, synthetic construct
KKKK

SEQ ID NO: 675 - protein, 4 aa, synthetic construct
  SITE 1: note = D-Lysine
  SITE 3..4: note = D-Lysine
  SITE 2: note = D-Tryptophan
KWKK

SEQ ID NO: 676 - protein, 4 aa, synthetic construct
  SITE 1..2: note = D-Lysine
  SITE 4: note = D-Lysine
  SITE 3: note = D-Tryptophan
KKWK

SEQ ID NO: 677 - protein, 5 aa, synthetic construct
  SITE 1: note = D-Lysine
  SITE 4..5: note = D-Lysine
  SITE 2..3: note = D-Tryptophan
KWWKK

SEQ ID NO: 678 - protein, 4 aa, synthetic construct
  SITE 1..2: note = D-Tryptophan
  SITE 3..4: note = D-Lysine
WWKK

SEQ ID NO: 679 - protein, 4 aa, synthetic construct
  SITE 1..4: note = D-Lysine
KKKK

SEQ ID NO: 680 - protein, 16 aa, synthetic construct
RQIKIWFQNRRMKWKK

SEQ ID NO: 681 - protein, 5 aa, synthetic construct
GGKGG

SEQ ID NO: 682 - DNA, 18 bp, synthetic construct
  source 1..18: mol_type = other DNA
gctattaccttaacccag

SEQ ID NO: 683 - protein, 9 aa, synthetic construct
  SITE 6..9: note = D-Arginine
GGKGGRRRR

SEQ ID NO: 684 - protein, 11 aa, synthetic construct
  SITE 6..11: note = D-Arginine
GGKGGRRRRRR

SEQ ID NO: 685 - protein, 13 aa, synthetic construct
  SITE 6..13: note = D-Arginine
GGKGGRRRRRRRR

SEQ ID NO: 686 - protein, 15 aa, synthetic construct
  SITE 6..15: note = D-Arginine
GGKGGRRRRRRRRRR

SEQ ID NO: 687 - protein, 6 aa, synthetic construct
  SITE 6: note = D-Arginine
GGKGGR

SEQ ID NO: 688 - protein, 15 aa, synthetic construct
  SITE 6: note = D-Serine
  MOD_RES 7: note = X is Nle
  SITE 8: note = D-Proline
  SITE 9: note = D-Aspartic acid
  SITE 10: note = D- Glutamic acid
  SITE 11: note = D-Threonine
GGKGGSXPDETKWKK

SEQ ID NO: 689 - protein, 15 aa, synthetic construct
  SITE 6: note = D-Serine
  MOD_RES 7: note = X is beta-alanine
  MOD_RES 8: note = X is Abu
  SITE 9: note = D-Serine
  MOD_RES 10: note = X is Abu
  MOD_RES 11: note = X is Hle
GGKGGSXXSXXKWKK

SEQ ID NO: 690 - protein, 16 aa, synthetic construct
  SITE 6: note = D-Serine
  MOD_RES 7: note = X is beta-alanine
  SITE 8..9: note = D-Arginine
  MOD_RES 10: note = X is Abu
  MOD_RES 11: note = X is Dab
  SITE 12: note = D-Histidine
GGKGGSXRRXXHKWKK

SEQ ID NO: 691 - protein, 16 aa, synthetic construct
  SITE 6: note = D-Serine
  MOD_RES 7: note = X is Abu
  SITE 9: note = D-Asparagine
  MOD_RES 10: note = X is Nle
  SITE 11: note = D-Asparagine
  SITE 12: note = D-Histidine
GGKGGSXGNXNHKWKK

SEQ ID NO: 692 - protein, 8 aa, synthetic construct
  SITE 6..8: note = D-Arginine
GGKGGRRR

SEQ ID NO: 693 - protein, 10 aa, synthetic construct
  SITE 6..10: note = D-Arginine
GGKGGRRRRR

SEQ ID NO: 694 - protein, 10 aa, synthetic construct
ARRHAHKWKK

SEQ ID NO: 695 - protein, 10 aa, synthetic construct
HGNLNHKWKK

SEQ ID NO: 696 - protein, 9 aa, synthetic construct
LPDETKWKK

SEQ ID NO: 697 - protein, 9 aa, synthetic construct
AHSHGKWKK

SEQ ID NO: 698 - protein, 18 aa, synthetic construct
  MOD_RES 8: note = X is 6-aminohexanoic acid
  MOD_RES 11: note = X is beta-alanine
  MOD_RES 14: note = X is 6-aminohexanoic acid
  MOD_RES 17: note = X is beta-alanine
GGKGGSRXRRXRRXRRXR